SEQUENCE LISTING (332 sequences)

SEQ ID NO: 1 - DNA, 905 bp, Arabidopsis thaliana
  CBF1 gene
aaaaagaatctacctgaaaagaaaaaaaagagagagagatataaatagcttaccaagacagatatactatcttttattaatccaaaaagactgagaactctagtaactacgtactacttaaaccttatccagtttcttgaaacagagtactctgatcaatgaactcattttcagctttttctgaaatgtttggctccgattacgagcctcaaggcggagattattgtccgacgttggccacgagttgtccgaagaaaccggcgggccgtaagaagtttcgtgagactcgtcacccaatttacagaggagttcgtcaaagaaactccggtaagtgggtttctgaagtgagagagccaaacaagaaaaccaggatttggctcgggactttccaaaccgctgagatggcagctcgtgctcacgacgtcgctgcattagccctccgtggccgatcagcatgtctcaacttcgctgactcggcttggcggctacgaatcccggagtcaacatgcgccaaggatatccaaaaagcggctgctgaagcggcgttggcttttcaagatgagacgtgtgatacgacgaccacggatcatggcctggacatggaggagacgatggtggaagctatttatacaccggaacagagcgaaggtgcgttttatatggatgaggagacaatgtttgggatgccgactttgttggataatatggctgaaggcatgcttttaccgccgccgtctgttcaatggaatcataattatgacggcgaaggagatggtgacgtgtcgctttggagttactaatattcgatagtcgtttccatttttgtactatagtttgaaaatattctagttccttttttagaatggttccttcattttattttattttattgttgtagaaacgag

SEQ ID NO: 2 - protein, 213 aa, Arabidopsis thaliana
  CBF1 polypeptide
MNSFSAFSEMFGSDYEPQGGDYCPTLATSCPKKPAGRKKFRETRHPIYRGVRQRNSGKWVSEVREPNKKTRIWLGTFQTAEMAARAHDVAALALRGRSACLNFADSAWRLRIPESTCAKDIQKAAAEAALAFQDETCDTTTTDHGLDMEETMVEAIYTPEQSEGAFYMDEETMFGMPTLLDNMAEGMLLPPPSVQWNHNYDGEGDGDVSLWSY

SEQ ID NO: 3 - DNA, 27 bp, Artificial Sequence
  MT50 Description of Artificial Sequence C-repeat/DRE
gatcatttcatggccgacctgcttttt

SEQ ID NO: 4 - DNA, 28 bp, Artificial Sequence
  MT52 Description of Artificial Sequence C-repeat/DRE
cacaatttcaagaattcactgctttttt

SEQ ID NO: 5 - DNA, 27 bp, Artificial Sequence
  MT80 Description of Artificial Sequence C-repeat/DRE
gatcatttcatggtatgtctgcttttt

SEQ ID NO: 6 - DNA, 27 bp, Artificial Sequence
  MT125 Description of Artificial Sequence C-repeat/DRE
gatcatttcatggaatcactgcttttt

SEQ ID NO: 7 - DNA, 27 bp, Artificial sequence
  MT68 Description of Artificial Sequence C-repeat/DRE
gatcacttgatggccgacctctttttt

SEQ ID NO: 8 - DNA, 27 bp, Artificial Sequence
  MT66 Description of Artificial Sequence C-repeat/DRE
gatcaatatactaccgacatgagttct

SEQ ID NO: 9 - DNA, 25 bp, Artificial Sequence
  MT86 Description of Artificial Sequence C-repeat/DRE
actaccgacatgagttccaaaaagc

SEQ ID NO: 10 - protein, 60 aa, Arabidopsis thaliana
  AP2 domain of 24 kDa polypeptide
IYRGVRQRNSGKWVSEVREPNKKTRIWLGTFQTAEMAARAHDVAALALRGRSACLNFADS

SEQ ID NO: 11 - protein, 61 aa, Nicotiana tabacum
  AP2 domain of tobacco DNA binding protein EREBP2
HYRGVRQRPWGKFAAEIRDPAKNGARVWLGTYETAEEAALAYDKAAYRMRGSKALLNFPHR

SEQ ID NO: 12 - DNA, 651 bp, Arabidopsis thaliana
  CBF2 gene
atgaactcattttctgccttttctgaaatgtttggctccgattacgagtctccggtttcctcaggcggtgattacagtccgaagcttgccacgagctgccccaagaaaccagcgggaaggaagaagtttcgtgagactcgtcacccaatttacagaggagttcgtcaaagaaactccggtaagtgggtgtgtgagttgagagagccaaacaagaaaacgaggatttggctcgggactttccaaaccgctgagatggcagctcgtgctcacgacgtcgccgccatagctctccgtggcagatctgcctgtctcaatttcgctgactcggcttggcggctacgaatcccggaatcaacctgtgccaaggaaatccaaaaggcggcggctgaagccgcgttgaattttcaagatgagatgtgtcatatgacgacggatgctcatggtcttgacatggaggagaccttggtggaggctatttatacgccggaacagagccaagatgcgttttatatggatgaagaggcgatgttggggatgtctagtttgttggataacatggccgaagggatgcttttaccgtcgccgtcggttcaatggaactataattttgatgtcgagggagatgatgacgtgtccttatggagctattaa

SEQ ID NO: 13 - protein, 216 aa, Arabidopsis thaliana
  CBF2 polypeptide
MNSFSAFSEMFGSDYESPVSSGGDYSPKLATSCPKKPAGRKKFRETRHPIYRGVRQRNSGKWVCELREPNKKTRIWLGTFQTAEMAARAHDVAAIALRGRSACLNFADSAWRLRIPESTCAKEIQKAAAEAALNFQDEMCHMTTDAHGLDMEETLVEAIYTPEQSQDAFYMDEEAMLGMSSLLDNMAEGMLLPSPSVQWNYNFDVEGDDDVSLWSY

SEQ ID NO: 14 - DNA, 651 bp, Arabidopsis thaliana
  CBF3 gene
atgaactcattttctgctttttctgaaatgtttggctccgattacgagtcttcggtttcctcaggcggtgattatattccgacgcttgcgagcagctgccccaagaaaccggcgggtcgtaagaagtttcgtgagactcgtcacccaatatacagaggagttcgtcggagaaactccggtaagtgggtttgtgaggttagagaaccaaacaagaaaacaaggatttggctcggaacatttcaaaccgctgagatggcagctcgagctcacgacgttgccgctttagcccttcgtggccgatcagcctgtctcaatttcgctgactcggcttggagactccgaatcccggaatcaacttgcgctaaggacatccaaaaggcggcggctgaagctgcgttggcgtttcaggatgagatgtgtgatgcgacgacggatcatggcttcgacatggaggagacgttggtggaggctatttacacggcggaacagagcgaaaatgcgttttatatgcacgatgaggcgatgtttgagatgccgagtttgttggctaatatggcagaagggatgcttttgccgcttccgtccgtacagtggaatcataatcatgaagtcgacggcgatgatgacgacgtatcgttatggagttattaa

SEQ ID NO: 15 - protein, 216 aa, Arabidopsis thaliana
  CBF3 polypeptide
MNSFSAFSEMFGSDYESSVSSGGDYIPTLASSCPKKPAGRKKFRETRHPIYRGVRRRNSGKWVCEVREPNKKTRIWLGTFQTAEMAARAHDVAALALRGRSACLNFADSAWRLRIPESTCAKDIQKAAAEAALAFQDEMCDATTDHGFDMEETLVEAIYTAEQSENAFYMHDEAMFEMPSLLANMAEGMLLPLPSVQWNHNHEVDGDDDDVSLWSY

SEQ ID NO: 16 - DNA, 18 bp, Nicotiana tabacum
  MT117 primer
ttggcggctacgaatccc

SEQ ID NO: 17 - protein, 210 aa, Brassica napus
  CAN1 polypeptide
HPIYRGVRLRKSGKWVCEVREPNKKSRIWLGTFKTAEMAARAHDVAALALRGRGACLNYADSAWRLRIPETTCHKDIQKAAAEAALAFEAEKSDVTMQNGQNMEETTAVASQAEVNDTTTEHGMNMEEATAVASQAEVNDTTTDHGVDMEETMVEAVFTGEQSEGFNMAKESTVEAAVVTEEPSKGSYMDEEWMLEMPTLLADMAEGMLL

SEQ ID NO: 18 - DNA, 632 bp, Brassica napus
  CAN1 gene
cacccgatataccggggagttcgtctgagaaagtcaggtaagtgggtgtgtgaagtgagggaaccaaacaagaaatctagaatttggcttggaactttcaaaacagctgagatggcagctcgtgctcacgacgtcgctgccctagccctccgtggaagaggcgcctgcctcaattatgcggactcggcttggcggctccgcatcccggagacaacctgccacaaggatatccagaaggctgctgctgaagccgcattggcttttgaggctgagaaaagtgatgtgacgatgcaaaatggccagaacatggaggagacgacggcggtggcttctcaggctgaagtgaatgacacgacgacagaacatggcatgaacatggaggaggcaacggcagtggcttctcaggctgaggtgaatgacacgacgacggatcatggcgtagacatggaggagacaatggtggaggctgtttttactggggaacaaagtgaagggtttaacatggcgaaggagtcgacggtggaggctgctgttgttacggaggaaccgagcaaaggatcttacatggacgaggagtggatgctcgagatgccgaccttgttggctgatatggcagaagggatgctcctgcc

SEQ ID NO: 19 - DNA, 36 bp, Artificial Sequence
  Dreb2a-reverse PCR primer
gcccaagcttcaagtttagtgagcactatgtgctcg

SEQ ID NO: 20 - DNA, 34 bp, Artificial Sequence
  Dreb2a-forward PCR primer
ggaagatctccttcccagaaacaacacaatctac

SEQ ID NO: 21 - DNA, 35 bp, Artificial Sequence
  P5CS-reverse PCR primer
gcccaagcttgtttcattttctccatgaaggagat

SEQ ID NO: 22 - DNA, 39 bp, Artificial Sequence
  P5CS-forward PCR primer
ggaagatcttatcgtcgtcgtcgtctaccaaaaccacac

SEQ ID NO: 23 - DNA, 32 bp, Artificial Sequence
  Rd22-reverse PCR primer
gctctaagcttcacaaggggttcgtttggtgc

SEQ ID NO: 24 - DNA, 40 bp, Artificial Sequence
  Rd22-forward PCR primer
ggggtaccttttgggagttggaatagaaatgggtttgatg

SEQ ID NO: 25 - DNA, 36 bp, Artificial Sequence
  Rd29a-reverse PCR primer
gcccaagcttaattttactcaaaatgttttggttgc

SEQ ID NO: 26 - DNA, 44 bp, Artificial Sequence
  Rd29a-forward PCR primer
ccggtacctttccaaagatttttttctttccaatagaagtaatc

SEQ ID NO: 27 - DNA, 30 bp, Artificial Sequence
  Rd29b-reverse PCR primer
gcggaagcttcattttctgctacagaagtg

SEQ ID NO: 28 - DNA, 40 bp, Artificial Sequence
  Rd29b-forward PCR primer
ccggtacctttccaaagctgtgttttctctttttcaagtg

SEQ ID NO: 29 - DNA, 42 bp, Artificial Sequence
  Rab18-reverse PCR primer
gcccaagcttcaaattctgaatattcacatatcaaaaaagtg

SEQ ID NO: 30 - DNA, 40 bp, Artificial Sequence
  Rab18-forward PCR primer
ggaagatctgttcttcttgtcttaagcaaacactttgagc

SEQ ID NO: 31 - DNA, 41 bp, Artificial Sequence
  Cor47-reverse PCR primer
gcccaagctttcgtctgttatcatacaaggcacaaaacgac

SEQ ID NO: 32 - DNA, 42 bp, Artificial Sequence
  Cor47-forward PCR primer
ggaagatctagttaatcttgatttgattaaaagtttatatag

SEQ ID NO: 33 - DNA, 25 bp, Artificial Sequence
  E9.1 primer PCR primer
caaactcagtaggattctggtgtgt

SEQ ID NO: 34 - DNA, 38 bp, Artificial Sequence
  cbf1-reverse 1 PCR primer
ggaagatcttgaaacagagtactctgatcaatgaactc

SEQ ID NO: 35 - DNA, 42 bp, Artificial Sequence
  cbf1-forward 1 PCR primer
cgcggatccctcgtttctacaacaataaaataaaataaaatg

SEQ ID NO: 36 - DNA, 37 bp, Artificial Sequence
  cbf1-reverse 2 PCR primer
ggggtacctgaaacagagtactctgatcaatgaactc

SEQ ID NO: 37 - DNA, 41 bp, Artificial Sequence
  cbf1-forward 2 PCR primer
gctctagactcgtttctacaacaataaaataaaataaaatg

SEQ ID NO: 38 - DNA, 577 bp, Brassica juncea
  bjCBF1 gene
tttcaccctatctaccggggagttcgcctgagaaagtcaggtaagtgggtgtgtgaagtgagggagccaaacaagaaatctaggatttggcttggaactttcaaaaccgcagagatcgctgctcgtgctcacgacgttgccgccttagccctccgtggaagagcggcctgtctcaacttcgccgactcggcttggcggctccgtatcccggagacaacttgcgccaaggatatccagaaggctgctgctgaagctgcgttggcttttggggccgaaaagagtgataccacgacgaatgatcaaggcatgaacatggaggagatgacggtggtggcttctcaggctgaggtgagcgacacgacgacatatcatggcctggacatggaggagactatggtggaggctgtttttgctgaggaacagagagaagggttttacttggcggaggagacgacggtggagggtgttgttacggaggaacagagcaaagggttttatatgtacgaggagtggacgttcgggatgcagtcctttttggccgatatggctgaaggcatgctcttttcaaagggcgaat

SEQ ID NO: 39 - protein, 130 aa, Brassica juncea
  bjCBF1 polypeptide
LPGVRLRKSGKWVCEVREPNKKSRIWLGTFKTAEIAARAHDVAALALRGRAACLNFADSAWRLRIPETTCAKDIQKAAAEAALAFGAEKSDTTTNDQGMNMEEMTAVASQAEVSDTTTYHGLDMEETMVD

SEQ ID NO: 40 - DNA, 431 bp, Brassica juncea
  bjCBF2 gene
catccgatctacaggggagttcgtctgagaaaatcaggtaagtgggtgtgtgaagtgagggaaccaaacaagagatctaggatctggctcggtactttcctaaccgccgagatcgcagctcgcgctcacgacgtcgccgccatagccctccgtggcaaatccgcatgtctcaatttcgctgactcggcttggcggctccgtatctcggagacaacatgccctaaggagattcagaaggctgctgctgaagccgcggtggcttttcaggctgagctaaatgatacgacggccgatcatggccttgacgtggaggagacgatcgtggaggctattttcacggaggaaagcagcgaagggttttatatggacgaggagttcatgttcgggatgccgaccttgtgggctagtatggcagaagggatgcttcttcc

SEQ ID NO: 41 - protein, 143 aa, Brassica juncea
  bjCBF2 polypeptide
HPIYRGVRLRKSGKWVCEVREPNKRSRIWLGTFLTAEIAARAHDVAAIALRGKSACLNFADSAWRLRISETTCPKEIQKAAAEAAVAFQAELNDTTADHGLDVEETIVEAIFTEESSEGFYMDEEFMFGMPTLWASMAEGMLL

SEQ ID NO: 42 - DNA, 431 bp, Brassica juncea
  bjCBF3 gene
catccaatttaccgtggagttcgtctgagaaaatcaggtaagtgggtgtgtgaagtgagggagccaaacaagaaatctaggatctggcccggtactttcctaaccgccgagatcgcagctcgcgctcacgacgtcgccgccatagccctccgtggcaaatccgcatgtctcaatttcgctgactcggcttggcggctccgtatcccggagacaacatgccctaaggagattcagaaggctgctgctgaagccgcggtggcttttcaggctgagctaaatgatacgacggccgatcatggccttgacgtggaggagacgatcgtggaggctattttcacggaggaaagcagcgaagggttttatatggacgaggagttcatgttcgggatgccgaccttgtgggctagtatggcggagggcatgctccttcc

SEQ ID NO: 43 - protein, 143 aa, Brassica juncea
  bjCBF3- polypeptide
HPIYRGVRLRKSGKWVCEVREPNKKSRIWPGTFLTAEIAARAHDVAAIALRGKSACLNFADSAWRLRIPETTCPKEIQKAAAEAAVAFQAELNDTTADHGLDVEETIVEAIFTEESSEGFYMAEEFMFGMPTLWASVAEGMLL

SEQ ID NO: 44 - DNA, 425 bp, Brassica juncea
  bjCBF4 gene
catccaatctaccggggtgttcgacagagaaactcagggaaatgggtttgtgaagttagggagcctaataagaaatctaggatctggttagggacttttccgaccgtcgaaatggccgctcgtgctcacgacgtcgccgctttagcccttcgtggccgctccgcttgtcttaatttcgccgactcggcgtggtgtctacggattcccgagtctacttgtcctaaagagattcagaaagctgcggccgaagctgcaatggcgtttcagaacgagacggctacgactgagacgactatggttgagggagtcataccggcggaggagacggtggggcagacgcgtgtggagacagcagaggagaacggtgtgttttatatggacgatccaaggtttcttgagaatatggcagagggcatgttcctacc

SEQ ID NO: 45 - protein, 142 aa, Brassica juncea
  bjCBF4- polypeptide
HPIYRGVRQRNSGKWVCEVREPNKKSRIWLGTFPTVEMAARAHDVAALALRGRSACLNFADSAWCLRIPESTCPKEIQKAAAEAAMAFQNEETATTETTMVEGVIPAEETVGQTRVETAEENGVEYMDDPRFLENMAEGMLF

SEQ ID NO: 46 - DNA, 632 bp, Brassica napus; identical to SEQ ID NO: 18
  bnCBF1 gene

SEQ ID NO: 47 - protein, 210 aa, Brassica napus; identical to SEQ ID NO: 17
  bnCBF1- polypeptide

SEQ ID NO: 48 - DNA, 876 bp, Brassica napus
  bnCBF2 gene
accgctcgagcaacaatgaacacattccctgcttccactgaaatggttggctccgagaacgagtctccggttactacggtagtaggaggtgattattatcccatgttggcggcaagctgtccgaagaagccagcgggtaggaagaagtttcaggagacacgtcaccccatttaccgaggagttcgtctgagaaagtcaggtaagtgggtgtgtgaagtgagggaaccaaacaagaaatctagaatttggcccggaactttcaaaacagctgagatggcagctcgtgctcacgacgtcgctgccctagccctccgtggaagaggcgcctgcctcaattatgcggactcggcttggcggctccgcatcccggaaacaacctgccacaaggatatccagaaggctgctgctgaagccgcattggcttttgaggctgagaaaagtgatgtgacgatgcaaaatggcctgaacatggaggagacgacggcggtggcttctcaggctgaagtgaatgacacgacgacagaacatggcatgaacatggaggaggcaacagcggtggcttctcaggctgaggtgaatgacacgacgacagatcatggcgtagacatggaggagacgatggtggaggctgtttttacggaggaacaaagtgaagggttcaacatggcggaggagtcgacggtggaggctgctgttgttacggatgaactgagcaaaggattttacatggacgaggagtggacgtacgagatgccgaccttgttggctgatatggcggcagggatgcttttgccgccaccatctgtacaatggggacataatgatgacttggaaggagatgcggacatgaacctctggagttattaaggatccgcg

SEQ ID NO: 49 - protein, 283 aa, Brassica napus
  bnCBF2- polypeptide
MNTFPASTEMVGSENESPVTTVVGGDYYPMLAASCPKKPAGRKKFQETRHPIYRGVRLRKSGKWVCEVREPNKKSRIWPGTFKTAEMAARAHDVAALALRGRGACLNYADSAWRLRIPETTCHKDIQKAAAEAALAFEAEKSDVTMQNGLNMEETTAVASQAEVNDTTTEHGMNMEEATAVASQAEVNDTTTDHGVDMEETMVEAVFTEEQSEGFNMAEESTVEAAVVTDELSKGFYMDEEWTYEMPTLLADMAAGMLLPPPSVQWGHNDDLEGDADMNLWSY

SEQ ID NO: 50 - DNA, 884 bp, Brassica napus
  bnCBF3 gene
actacactcagccttatccagtttttttcaaaagatttttcaacaatgaacacattccctgcgtccactgaaatggttggctccgagaacgagtctccggttactacggtagcaggaggtgattattatcccatgttggcggcaagctgtccgaagaagccagcaggtaggaagaagtttcaggagacacgtcaccccatttaccgaggagttcgtctgagaaagtcaggtaagtgggtgtgtgaagtgagggaaccaaacaagaaatctagaatttggcccggaactttcaaaacagctgagatggcagctcgtgctcacgacgtcgctgccctagccctccgtggaagaggcgcctgcctcaattatgcggactcggcttggcggctccgcatcccggagacaacctgccacaaggatatccagaaggctgctgctgaagccgcattggcttttgaggctgagaaaagtgatgtgacgatgcaaaatggcctgaacatggaggagacgacggcggtggcttctcaggctgaagtgaatgacacgacgacagaacatggcatgaacatggaggaggcaacggcagtggcttctcaggctgaggtgaatgacacgacgacggatcatggcgtagacatggaggagacaatggtggaggctgtttttactggggaacaaagtgaagggtttaacatggcgaaggagtcgacggtggaggctgctgttgttacggaggaaccgagcaaaggatcttacatggacgaggagtggatgctcgagatgccgaccttgttggctgatatggcggaagggatgcttttgccgccgccgtccgtacaatggggacagaatgatgacttcgaaggagatgctgacatgaacct

SEQ ID NO: 51 - protein, 279 aa, Brassica napus
  bnCBF3- polypeptide
MNTFPASTEMVGSENESPVTTVAGGDYYPMLAASCPKKPAGRKKFQETRHPIYRGVRLRKSGKWVCEVREPNKKSRIWPGTFKTAEMAARAHDVAALALRGRGACLNYADSAWRLRIPETTCHKDIQKAAAEAALAFEAEKSDVTMQNGLNMEETTAVASQAEVNDTTTEHGMNMEEATAVASQAEVNDTTTDHGVDMEETMVEAVFTGEQSEGFNMAKESTVEAAVVTEEPSKGSYMDEEWMLEMPTLLADMAEGMLLPPPSVQWGQNDDFEGDADMN

SEQ ID NO: 52 - DNA, 874 bp, Brassica napus
  bnCBF4 gene
gtaattcgattaccgctcgagtacttactatactacactcagccttatccagtttttcaaaagaagttttcaactatgaactcagtctctactttttctgaacttcttggctctgagaacgagtctccggtaggtggtgattactgtcccatgttggcggcgagctgtccgaagaagccggcgggtaggaagaagtttcgggagacacgtcaccccatttaccgaggagttcgccttagaaaatcaggtaagtgggtgtgtgaagtgagggaaccaaacaaaaaatctaggatttggctcggaactttcaaaacagctgagatcgcagctcgtgctcacgacgtcgccgccttagctctccgtggaagaggcgcctgcctcaacttcgccgactcggcttggcggctccgtatcccggagacaacctgcgccaaggatatccagaaggctgctgctgaagccgcattggcttttgaggccgagaagagtgataccacgacgaatgatcatggcatgaacatggcttctcaggccgaggttaatgacacaacggatcatggcctggacatggaggagacgatggtggaggctgtttttactgaggagcagagagacgggttttacatggcggaggagacgacggtggagggtgttgttccggaggaacagatgagcaaagggttttacatggacgaggagtggatgttcgggatgccgaccttgttggctgatatggcggcagggatgctcttaccgccgccgtccgtacaatggggacataatgatgacttcgaaggagatgttgacatgaacctctggaattattagtactcatatttttttaaattattttttgaacgaataatattttatt

SEQ ID NO: 53 - protein, 250 aa, Brassica napus
  bnCBF4- polypeptide
MNSVSTFSELLGSENESPVGGDYCPMLAASCPKKPAGRKKFRETRHPIYRGVRLRKSGKWVCEVREPNKKSRIWLGTFKTAEIAARAHDVAALALRGRGACLNFADSAWRLRIPETTCAKDIQKAAAEAALAFEAEKSDTTTNDHGMNMASQAEVNDTTDHGLDMEETMVEAVFTEEQRDGFYMAEETTVEGVVPEEQMSKGFYMDEEWMFGMPTLLADMAAGMLLPPPSVQWGHNDDFEGDVDMNLWNY

SEQ ID NO: 54 - DNA, 898 bp, Brassica napus
  bnCBF5 gene
aataaatatcttatcaaaccagtcagaacagagatcttgttacttactatactacactcagccttatccagttttcaaaaaaagtattcaacgatgaactcagtctctactttttctgaactgctccgctccgagaacgagtctccggttaatacggaaggtggtgattacattttggcggcgagctgtcccaagaaacctgctggtaggaagaagtttcaggagacacgccaccccatttacagaggagttcgtctgaggaagtcaggtaagtgggtgtgtgaagtgagggaaccaaacaagaaatctagaatttggctcggaactttcaaaacagctgagatcgcagctcgtgctcacgacgttgccgccttagctctccgtggaagaggcgcctgcctcaacttcgccgactcggcttggcggctccgtatcccggagacgacctgcgccaaggatatccagaaggctgctgctgaagccgcattggcttttgaggccgagaagagtgataccacgacgaatgatcatggcatgaacatggcttctcaggttgaggttaatgacacgacggatcatgacctggacatggaggagacgatagtggaggctgtttttagggaggaacagagagaagggttttacatggcggaggagacgacggttgtgggtgttgttccggaggaacagatgagcaaagggttttacatggacgaggagtggatgttcgggatgccgaccttgttggctgatatggcggcagggatgctcttaccgctgccgtccgtacaatggggacataatgatgacttcgaaggagatgctgacatgaacctctggaattattagtactcatatttttttaaattattttttgaacgaataatattttattgaa

SEQ ID NO: 55 - protein, 251 aa, Brassica napus
  bnCBF5- polypeptide
MNSVSTFSELLRSENESPVNTEGGDYILAASCPKKPAGRKKFQETRHPIYRGVRLRKSGKWVCEVREPNKKSRIWLGTFKTAEIAARAHDVAALALRGRGACLNFADSAWRLRIPETTCAKDIQKAAAEAALAFEAEKSDTTTNDHGMNMASQVEVNDTTDHDLDMEETIVEAVFREEQREGFYMAEETTVVGVVPEEQMSKGFYMDEEWMFGMPTLLADMAAGMLLPLPSVQWGHNDDFEGDADMNLWNY

SEQ ID NO: 56 - DNA, 1132 bp, Brassica napus
  bnCBF6 gene
gattaccgctcgagtacttactatactacactcagccttatccagtttttctcaaaagatttttcaacaatgaacacattccctgcttccactgaaatggttggctccgagaacgagtctccggttactacggtagtaggaggtgattattatcccatgttggcggcaagctgtccgaagaagccagcgggtaggaagaagtttcaggagacacgtcaccccatttaccgaggagttcgtctgagaaagtcaggtaagtgggtgtgtgaagtgagggaaccaaacaagaaatctagaatttggcttggaactttcaaaacagctgagatggcagctcgtgctcacgacgtggctgccctagccctccgtggaagaggcgcctgcctcaattatgcggactcggcttcgcggctccgcatcccggagacaacctgccacaaggatatccagaaggctgctgctgaagccgcattggcttttgaggctgagaaaagtgatgtgacgatggaggagacgatggcggtggcttctcaggctgaagtgaatgacacgacgacagatcatggcatgaacatggaggaggcaacagcggtggcttctcaggctgaggtgaatgacacgacgacagatcatggcgtagacatggaggagacgatggtggaggctgtttttacggaggaacaaagtgaagggttcaacatggcggaggagtcgacggtggaggctgctgttgttacggatgaactgagcaaaggattttacatggacgaggagtggacgtacgagatgccgaccttgttggctgatatggcggcagggatgcttttgccgccaccatctgtacaatggggacataatgatgacttggaaggagatgctgacatgaacctctggaattattaatactcgtgttttaaaaattatacattgtgcaataatattttatcgaatttctaattctgcctttaacttttaatggggatctttattagtgtaggaaacgagtgtaaatgttccgccgtggtgttgtcaaaatgctgattattttttgtgtgcagcataatcacgtttggtttcctttacactccaaatttagttgaaatacaaatagaatagaaaagtgaaaaaatgt

SEQ ID NO: 57 - protein, 277 aa, Brassica napus
  bnCBF6- polypeptide
MNTFPASTEMVGSENESPVTTVVGGDYYPMLAASCPKKPAGRKKFQETRHPIYRGVRLRKSGKWVCEVREPNKKSRIWLGTFKTAEMAARAHDVAALALRGRGACLNYADSASRLRIPETTCHKDIQKAAAEAALAFEAEKSDVTMEETMAVASQAEVNDTTTDHGMNMEEATAVASQAEVNDTTTDHGVDMEETMVEAVFTEEQSEGFNMAEESTVEAAVVTDELSKGFYMDEEWTYEMPTLLADMAAGMLLPPPSVQWGHNDDLEGDADMNLWNY

SEQ ID NO: 58 - DNA, 768 bp, Brassica napus
  bnCBF7 gene
agtgatgtttttcaaaagaagttttcaactatgaactcagtctctactttttctgaacttcttggctctgagaacgagtctccggtaggtggtgattactgtcccatgttggcggcgagctgtccgaagaagccggcgggtaggaagaagtttcgggagacacgtcaccccatttaccgaggagttcgccttagaaaatcaggtaagtgggtgtgtgaagtgagggagccaaacaagaaatctaggatttggctcggtactttcctaacagccgagatcgcagcccgtgctcacgacgtcgccgccatagccctccgcggcaaatcagcttgtctcaattttgccgactccgcttggcggctccgtatcccggagacaacatgccccaaggagattcagaaggcggctgctgaagccgcggtggcttttaaggctgagataaataatacgacggcggatcatggcattgacgtggaggagacgatcgttgaggctattttcacggaggaaaacaacgatggtttttatatggacgaggaggagtccatgttcgggatgccggccttgttggctagtatggctgaaggaatgcttttgccgcctccgtccgtacaattcggacatacctatgactttgacggagatgctgacgtgtccctttggagttattagtacaaagattttttatttccatttttggtataatacttctttttgattttcggattctacctttttatgggtatcattttttttttaggaaacggg

SEQ ID NO: 59 - protein, 213 aa, Brassica napus
  bnCBF7 polypeptide
MNSVSTFSELLGSENESPVGGDYCPMLAASCPKKPAGRKKFRETRHPIYRGVRLRKSGKWVCEVREPNKKSRIWLGTFLTAEIAARAHDVAAIALRGKSACLNFADSAWRLRIPETTCPKEIQKAAAEAAVAFKAEINNTTADHGIDVEETIVEAIFTEENNDGFYMDEEESMFGMPALLASMAEGMLLPPPSVQFGHTYDFDGDADVSLWSY

SEQ ID NO: 60 - DNA, 953 bp, Brassica napus
  bnCBF8 gene
accgctcgagcaacaatgaacacattccctgcttccactgaaatggttggctccgagaacgagtctccggttactacggtagcaggaggtgattattatcccatgttggcggcaagctgtccgaagaagccagcgggtaggaagaagtttcaggagacacgtcaccccatttaccgaggagttcgtctgagaaagtcaggtaagtgggtgtgtgaagtgagggaaccaaacaagaaatctagaatttggcttggaactttcaaaacagctgagatggcagctcgtgctcacgacgtggctgccctagccctccgtggaagaggcgcctgcctcaattatgcggactcggcttcgcggctccgcatcccggagacaacctgccacaaggatatccagaaggctgctgctgaagccgcattggcttttgaggctgagaaaagtgatgtgacgatggaggagacgatggcggtggcttctcaggctgaagtgaatgacacgacgacagatcatggcatgaacatggaggaggcaacggcagtggcttctcaggctgaggtgaatgacacgacgacggatcatggcgtagacatggaggagacaatggtggaggctgtttttactggggaacaaagtgaagggtttaacatggcgaaggagtcgacggtggaggctgctgttgttacggaggaaccgagcaaaggatcttacatggacgaggagtggatgctcgagatgccgaccttgttggctgatatggcggaagggatgcttttgccgccgccgtccgtacaatggggacagaatgatgacttcgaaggagatgcggacatgaacctctggagttattaatactcgtatttttaaaattatttattgtgcaataattttttatcgaatttcgaattctgcctttaatttttaatggggatctttatttgccaaaaaaaaaaaaa

SEQ ID NO: 61 - protein, 277 aa, Brassica napus
  bnCBF8 polypeptide
MNTFPASTEMVGSENESPVTTVAGGDYYPMLAASCPKKPAGRKKFQETRHPIYRGVRLRKSGKWVCEVREPNKKSRIWLGTFKTAEMAARAHDVAALALRGRGACLNYADSASRLRIPETTCHKDIQKAAAEAALAFEAEKSDVTMEETMAVASQAEVNDTTTDHGMNMEEATAVASQAEVNDTTTDHGVDMEETMVEAVFTGEQSEGFNMAKESTVEAAVVTEEPSKGSYMDEEWMLEMPTLLADMAEGMLLPPPSVQWGQNDDFEGDADMNLWSY

SEQ ID NO: 62 - DNA, 889 bp, Brassica napus
  bnCBF9 gene
ctagtgattaccgctcgagcaacaatgaacacattccctgcttccactgaaatggttggctccgagaacgagtctccggttactacggtagcaggaggtgattattatcccatgttggcggcaagctgtccgaagaagccagcgggtaggaagaagtttcaggagacacgtcaccccatttaccgaggagttcgtctgagaaagtcaggtaagtgggtgtgtgaagtgagggaaccaaacaagaaatctagaatttggcccggaactttcaaaacagctgagatggcagctcgtgctcacgacgtcgctgccctagccctccgtggaagaggcgcccgcctcaattatgcggactcagcttggcggctccgcatcccggagacaacctgccacaaggatatccagaaggctgctgctgaagccgcattggcttttgaggctgagaaaagtgatgtgacgatgcaaaatggcctgaacatggaggagacgacggcggtggcttctcaggctgaagtgaatgacacgacgacagaacatggcatgaacatggaggaggcaacggcagtggcttctcaggctgaggtgaatgacacgacgacggatcatggcgtagacatggaggagacaatggtggaggctgtttttactggggaacaaagtgaagggtttaacatggcgaaggagtcgacggtggaggctgctgttgttacggaggaaccgagcaaaggatcttacatggacgaggagtggatgctcgagatgccgaccttgttggctgatatggcggaagggatgcttttgccgccgccgtccgtacaatggggacagaatgatgacttcgaaggagatgcgcacatgaacctctggagttattaaggatccgcgaatc

SEQ ID NO: 63 - protein, 283 aa, Brassica napus
  bnCBF9 polypeptide
MNTFPASTEMVGSENESPVTTVAGGDYYPMLAASCPKKPAGRKKFQETRHPIYRGVRLRKSGKWVCEVREPNKKSRIWPGTFKTAEMAARAHDVAALALRGRGARLNYADSAWRLRIPETTCHKDIQKAAAEAALAFEAEKSDVTMQNGLNMEETTAVASQAEVNDTTTEHGMNMEEATAVASQAEVNDTTTDHGVDMEETMVEAVFTGEQSEGFNMAKESTVEAAVVTEEPSKGSYMDEEWMLEMPTLLADMAEGMLLPPPSVQWGQNDDFEGDAHMNLWSY

SEQ ID NO: 64 - DNA, 563 bp, Brassica oleracea
  boCBF1 gene
caccctatctaccggggagttcgcctgagaaagtcaggtaagtgggtgtgtgaagtgagggagccaaacaagaaatctaggatttggcttggaactttcaaaaccgcagagatcgctgctcgtgctcacgacgttgccgccttagccctccgtggaagagcggcctgtctcaacttcgccgactcggcttggcggctccgtatcccggagacaacttgcgccaaggatatccagaaggctgctgctgaagctgcgttggcttttggggccgaaaagagtgataccacgacgaatgatcaaggcatgaacatggaggagatgacggtggtggcttctcaggctgaggtgagcgacacgacgacatatcatggcctggacatggaggagactatggtggaggctgtttttgctgaggaacagagagaagggttttacttggcggaggagacgacggtggagggtgttgttacggaggaacagagcaaagggttttatatggacgaggagtggacgttcgggatgcagtcctttttggccgatatggctgaaggcatgctctttcc

SEQ ID NO: 65 - protein, 188 aa, Brassica oleracea
  boCBF1 polypeptide
HPIYRGVRLRKSGKWVCEVREPNKKSRIWLGTFKTAEIAARAHDVAALALRGRAACLNFADSAWRLRIPETTCAKDIQKAAAEAALAFGAEKSDTTTNDQGMNMEEMTVVASQAEVSDTTTYHGLDMEETMVEAVFAEEQREGFYLAEETTVEGVVTEEQSKGFYMDEEWTFGMQSFLADMAEGMLFP

SEQ ID NO: 66 - DNA, 533 bp, Brassica oleracea
  boCBF2 gene
gaaacatagatctttgtacttactatacttcaccttatccagttttatttttttatttataaagagttttcaacaatgacctcattttctaccttttctgaactgttgggctccgagcatgagtctccggttacattaggcgaagagtattgtccgaagctggccgcaagctgtccgaagaaaccagccggccggaagaagtttcgagagacgcgtcacccagtttacagaggagttcgtctgagaaactcaggtaagtgggtgtgtgaagtgagggagccaaacaagaaatctaggatttggctcggtactttcctaacagccgagatcgcagcccgtgctcacgacgtcgccgccatagccctccgcggcaaatcagcttgtctcaattttgccgactccgcttggcggctccgtatcccggagacaacatgccccaaggagattcagaaggcggctgctgaagccgcggtggcttttaaggctgagataaataatacgacggcggatcacggcctcgacatggaagagac

SEQ ID NO: 67 - protein, 152 aa, Brassica oleracea
  boCBF2 polypeptide
MTSFSTFSELLGSEHESPVTLGEEYCPKLAASCPKKPAGRKKFRETRHPVYRGVRLRNSGKWVCEVREPNKKSRIWLGTFLTAEIAARAHDVAAIALRGKSACLNFADSAWRLRIPETTCPKEIQKAAAEAAVAFKAEINNTTADHGLDMEE

SEQ ID NO: 68 - DNA, 887 bp, Brassica oleracea
  boCBF3 gene
actcagccttatccagtttttctcaaaagatttttcaacaatgaacacattccctgcttccactgaaatggttggctccgagaacgagtctccggttactacggtagtaggaggtgattattatcccatgttggcggcaagctgtccgaagaagccagcgggtaggaagaagtttcaggagacacgtcaccccatttaccgaggagttcgtctgagaaagtcaggtaagtgggtgtgtgaagtgagggaaccaaacaagaaatctagaatttggcttggaactttcaaaacagctgagatggcagctcgtgctcacgacgtggctgccctagccctccgtggaagaggcgcctgcctcaattatgcggactcggcttggcggctccgcatcccggagacaacctgccacaaggatatccagaaggctgctgctgaagccgcattggcttttgaggctgagaaaagtgatgtgacgatggaggagacgatggcggtggcttctcaggctgaagtgaatgacacgacgacagatcatggcatgaacatggaggaggcaacagcggtggcttctcaggctgaggtgaatgacacgacgacagatcatggcgtagacatggaggagacgatggtggaggctgtttttacggaggaacaaagtgaagggttcaacatggcggaggagtcgacggtggaggctgctgttgttacggatgaactgagcaaaggattttacatggacgaggagtggacgtacgagatgccgaccttgttggctgatatggcggcagggatgcttttgccgccaccatctgtacaatggggacataatgatgacttggaaggagatgcggacatgaacctctggagttattaatactcgtattttt

SEQ ID NO: 69 - protein, 277 aa, Brassica oleracea
  boCBF3 polypeptide
MNTFPASTEMVGSENESPVTTVVGGDYYPMLAASCPKKPAGRKKFQETRHPIYRGVRLRKSGKWVCEVREPNKKSRIWLGTFKTAEMAARAHDVAALALRGRGACLNYADSAWRLRIPETTCHKDIQKAAAEAALAFEAEKSDVTMEETMAVASQAEVNDTTTDHGMNMEEATAVASQAEVNDTTTDHGVDMEETMVEAVFTEEQSEGFNMAEESTVEAAVVTDELSKGFYMDEEWTYEMPTLLADMAAGMLLPPPSVQWGHNDDLEGDADMNLWSY

SEQ ID NO: 70 - DNA, 950 bp, Brassica oleracea
  boCBF4 gene
ctgaaaagaagataaaagagagagaaataaatatcttatcaaaccagacagaacagagatcttgttacttactatactacactcagccttatccagtttttcaaaagaagttttcaactatgaactcagtctctactttttctgaacttcttggctctgagaacgagtctccggtaggtggtgattactgtcccatgttggcggcgagctgtccgaagaagccggcgggtaggaagaagtttcgggagacacgtcaccccatttaccgaggagttcgccttagaaaatcaggtaagtgggtgtgtgaagtgagggaaccaaacaaaaaatctaggatttggctcggaactttcaaaacagctgagatcgcagctcgtgctcacgacgtcgccgccttagctctccgtggaagaggcgcctgcctcaacttcgccgactcggcttggcggctccgtatcccggagacaacctgcgccaaggatatccagaaggctgctgctgaagccgcattggcttttgaggccgagaagagtgataccacgacgaatgatcatggcatgaacatggcttctcaggctgaggttaatgacacgacggatcatggcctggacatggaggagacgatggtggaggctgtttttactgaggagcagagagacgggttttacatggcggaggagacgacggtggagggtgttgttccggaggaacagatgagcaaagggttttacatggacgaggagtggatgttcgggatgccgaccttgttggctgatatggcggcagggatgctcttaccgccgccgtccgtacaatggggacataatgatgacttcgaaggagatgctgacatgaacctctggaattattagtactcgtatttttttaaattattttttgaacgaataatattttattgaattcggattctacctgtttttttaatggat

SEQ ID NO: 71 - protein, 250 aa, Brassica oleracea
  boCBF4 polypeptide
MNSVSTFSELLGSENESPVGGDYCPMLAASCPKKPAGRKKFRETRHPIYRGVRLRKSGKWVCEVREPNKKSRIWLGTFKTAEIAARAHDVAALALRGRGACLNFADSAWRLRIPETTCAKDIQKAAAEAALAFEAEKSDTTTNDHGMNMASQAEVNDTTDHGLDMEETMVEAVFTEEQRDGFYMAEETTVEGVVPEEQMSKGFYMDEEWMFGMPTLLADMAAGMLLPPPSVQWGHNDDFEGDADMNLWNY

SEQ ID NO: 72 - DNA, 877 bp, Brassica oleracea
  boCBF5 gene
accgctcgagcaacaatgaacacattccctgcttccactgaaatggttagctccgagaacgagtctccggttactacggtagtaggaggtgattattatcccatgttggcggcaagctgtccgaagaagccagcgggtaggaagaagtttcaggagacacgtcaccccatttaccgaggagttcgtctgagaaagtcaggtaagtgggtgtgtgaagtgagggaactaaacaagaaatctagaatttggcttggaactttcaaaacagctgagatggcagctcgtgctcacgacgtggctgccctagccctccgtggaagaggcgcctgcctcaattatgcggactcggcttggcggctccgcatcccggagacaacctgccacaaggatatccagaaggctgctgctgaagccgcattggcttttgaggctgagaagagtgatgcgacgatgcaaaatggcctgaacatggaggagacgacggcggcggcttctcagactgaagtgagtgacacgacgacagatcatggcatgaacatggaggagacaacggcggtggcttctcaggctgaggtgaatgacacgacgacagatcatggcgtagacatggaggagacgatggtggaggctgtttttactgaggaacaaagtgaagggttcaacatggcgaaggagtcgacggcggaggctgctgttgttacggaggaactgagcaaaggagtttacatggacgaggagtggacgtacgagatgccgaccttgttggctgatatggcggcagggatgcttttgccgccaccatctgtacaatggggacataatgatgacttggaaggagatgcggacatgaacctactggagttattaaggatccgcg

SEQ ID NO: 73 - protein, 287 aa, Brassica oleracea
  boCBF5 polypeptide
MNTFPASTEMVSSENESPVTTVVGGDYYPMLAASCPKKPAGRKKFQETRHPIYRGVRLRKSGKWVCEVRELNKKSRIWLGTFKTAEMAARAHDVAALALRGRGACLNYADSAWRLRIPETTCHKDIQKAAAEAALAFEAEKSDATMQNGLNMEETTAAASQTEVSDTTTDHGMNMEETTAVASQAEVNDTTTDHGVDMEETMVEAVFTEEQSEGFNMAKESTAEAAVVTEELSKGVYMDEEWTYEMPTLLADMAAGMLLPPPSVQWGHNDDLEGDADMNLLELLRIR

SEQ ID NO: 74 - DNA, 374 bp, Brassica rapa
  brCBF1 gene
catcccatttacaggggggttcgtttaagaaagtcaggtaagtgggtgtgtgaagtgagggaaccaaacaagaaatctaggatttggctcggaactttcaaaaccgctgagatcgctgctcgtgctcacgacgttgctgccttagccctccgcgggagaggcgcctgcctcaacttcgccgactcggcttggcggctccgtatcccggagacaacctgcgccaaggacatccagaaggcggctgctgaagctgcattggcttttgaggccgagaagagtgatcatggcatgaacatcaagaatactacggcggtggtttctcaggttgaggtgaatgacacgacgacggaccacggcttggacatggaggagac

SEQ ID NO: 75 - protein, 124 aa, Brassica rapa
  brCBF1 polypeptide
HPIYRGVRLRKSGKWVCEVREPNKKSRIWLGTFKTAEIAARAHDVAALALRGRGACLNFADSAWRLRIPETTCAKDIQKAAAEAALAFEAEKSDHGMNIKNTTAVVSQVEVNDTTTDHGLDMEE

SEQ ID NO: 76 - DNA, 884 bp, Brassica rapa
  brCBF2 gene
tacactcagccttatccagtttttttcaaaagacttttcaacaatgaacacattccctgcgtccactgaaatggttggctccgagaacgagtctccggttactacggtagcaggaggtgattattatcccatgttggcggcaagctgtccgaagaagccagcgggtaggaagaagtttcaggagacacgtcaccccatttaccgaggagttcgtctgagaaagtcaggtaagtgggtgtgtgaagtgagggaaccaaacaagaaatctagaatttggcttggaactttcaaaacagctgagatggcagctcgtgctcacgacgtcgctgccctagccctccgtggaagaggcgcctgcctcaattatgcggactcggcttggcggctccgcatcccggagacaacctgccacaaggatatccagaaggctgctgctgaagccgcattggcttttgaggctgagaaaagtgatgtgacgatgcaaaatggcctgaacatggaggagatgacggcggtggcttctcaggctgaagtgaatgacacgacgacagaacatggcatgaacatggaggaggcaacggcagtggcttctcaggctgaggtgaatgacacgacgacggatcatggcgtagacatggaggagacaatggtggaggctgtttttactgaggaacaaagtgaagggtttaacatggcgaaggagtcgacggtggaggctgctgttgttacggaggaaccgagcaaaggatcttacatggacgaggagtggatgctcgagatgccgaccttgttggctgatatggcggaagggatgcttttgccgccgccgtccgtacaatggggacagaatgatgacttcgaaggagatgctgacatgaacctct

SEQ ID NO: 77 - protein, 280 aa, Brassica rapa
  brCBF2 polypeptide
MNTFPASTEMVGSENESPVTTVAGGDYYPMLAASCPKKPAGRKKFQETRHPIYRGVRLRKSGKWVCEVREPNKKSRIWLGTFKTAEMAARAHDVAALALRGRGACLNYADSAWRLRIPETTCHKDIQKAAAEAALAFEAEKSDVTMQNGLNMEEMTAVASQAEVNDTTTEHGMNMEEATAVASQAEVNDTTTDHGVDMEETMVEAVFTEEQSEGFNMAKESTVEAAVVTEEPSKGSYMDEEWMLEMPTLLADMAEGMLLPPPSVQWGQNDDFEGDADMNL

SEQ ID NO: 78 - DNA, 806 bp, Brassica rapa
  brCBF3 gene
acactcagccttatccagttttcaaaaaaagtattcaacgatgaactcagtctctactttttctgaactgctctgctccgagaacgagtctccggttaatacggaaggtggtgattacattttggcggcgagctgtcccaagaaacctgctggtaggaagaagtttcaggagacacgccaccccatttacagaggagttcgtctgaggaagtcaggtaagtgggtgtgtgaagtgagggaaccaaacaagaaatctagaatttggctcggaactttcaaaacagctgagatcgcagctcgtgctcacgacgttgccgccttagctctccgtggaagaggcgcctgcctcaacttcgccgactcggcttggcggctccgtatcccggagacgacctgcgccaaggatatccagaaggctgctgctgaagccgcattggcttttgaggccgagaagagtgataccacgacgaatgatcgtggcatgaacatggaggagacgtcggcggtggcttctccggctgagttgaatgatacgacggcggatcatggcctggacatggaggagacgatggtggaggctgtttttagggaggaacagagagaagggttttacatggcggaggagacgacggtggagggtgttgttccggagtaacagatgagcaaagggttttacatggacgaggagtggacgttcgagatgccgaggttgttggctgatatggcggaagggatgcttttgccgcccccgtccgtacaatggggacataacgatgacttcgaaggagatgctgacatgaacctct

SEQ ID NO: 79 - protein, 204 aa, Brassica rapa
  brCBF3 polypeptide
MNSVSTFSELLCSENESPVNTEGGDYILAASCPKKPAGRKKFQETRHPIYRGVRLRKSGKWVCEVREPNKKSRIWLGTFKTAEIAARAHDVAALALRGRGACLNFADSAWRLRIPETTCAKDIQKAAAEAALAFEAEKSDTTTNDRGMNMEETSAVASPAELNDTTADHGLDMEETMVEAVFREEQREGFYMAEETTVEGVVPE

SEQ ID NO: 80 - DNA, 755 bp, Brassica rapa
  brCBF4 gene
accgctcgagtacttactatactacactcagccttatccagtttttcttccaacgatggactcaatctctacttttcctgaactgcttggctcagagaacgagtctccggttactacggtagtaggaggtgattattgtcccaggttggcggcaagctgtccgaagaagccagcgggtaggaagaagtttcaggagacacgtcaccccatttaccgtggagttcgtttaagaaagtccggtaagtgggtgtgtgaagtgagggaaccaaacaagaaatctaggatttggctcggaactttcaaaaccgctgagatcgctgctcgtgctcacgacgttgctgccttagccctccgcggaagaggcgcctgcctcaacttcgccgactcggcttgacggctccgtatcccggagacaacctgcgccaaggatatccagaaggctgctgctgaagctgcattggcttttgaggccgagaagagtgatcatggcatgaacatgaagaatactacggcggtggcttctcaggttgaggtgaatgatacgacgacggaccatggcgtggacatggaggagacgagggtggagggtgttgttacggaggaacagaacaattggttttacatggacgaggagtggatgtttgggatgccgacgttgttggttgatatggcggaagggatgcttataccgcggcagtccgtacaatcgggacactacgatgacttcgaaggagatgctgacatgaacctctgga

SEQ ID NO: 81 - protein, 112 aa, Brassica rapa
  brCBF4 polypeptide
MDSISTFPELLGSENESPVTTVVGGDYCPRLAASCPKKPAGRKKFQETRHPIYRGVRLRKSGKWVCEVREPNKKSRIWLGTFKTAEIAARAHDVAALALRGRGACLNFADSA

SEQ ID NO: 82 - DNA, 832 bp, Brassica rapa
  brCBF5 gene
accgctcgagtacttactatactacactcagccttatccagtttttcttccaacgatggactcaatctctacttttcctgaactgcttggctcagagaacgagtctccggttactacggtagtaggaggtgattattgtcccaggttggcggcaagctgtccgaagaagccagcgggtaggaagaagtttcaggagacacgtcaccccatttaccgtggagttcgtttaagaaagtccggtaagtgggtgtgtgaagtgagggaaccaaacaagaaatctaggatttggctcggaactttcaaaaccgctgagatcgctgctcgtgctcacgacgttgctgccttagccctccgcggaagaggcgcctgcctcaacttcgccgactcggcttggcggctccgtatcccggagacaacctgcgccaaggatatccagaaggctgctgctgaagctgctttggcttttgaggccgagaagagtgatcatggcatgaacatgaagaatactacggcggtggcttctcaggttgaggtgaatgatacgacgacggaccatggcgtggacatggaggagacgttggtggaggctgtttttacggaggaacagagagaagggttttacatgacggaggagacgagggtggagggtgttgttacggaggaacagaacaattggttttacatggacgaggagtggatgtttgggatgccgacgttgttggttgatatggcggaagggatgcttataccgcggcagtccgtacaatcgggacactacgatgacttcgaaggagatgctgacatgaacctctggaattattagggatccgcg

SEQ ID NO: 83 - protein, 255 aa, Brassica rapa
  brCBF5 polypeptide
MDSISTFPELLGSENESPVTTVVGGDYCPRLAASCPKKPAGRKKFQETRHPIYRGVRLRKSGKWVCEVREPNKKSRIWLGTFKTAEIAARAHDVAALALRGRGACLNFADSAWRLRIPETTCAKDIQKAAAEAALAFEAEKSDHGMNMKNTTAVASQVEVNDTTTDHGVDMEETLVEAVFTEEQREGFYMTEETRVEGVVTEEQNNWFYMDEEWMFGMPTLLVDMAEGMLIPRQSVQSGHYDDFEGDADMNLWNY

SEQ ID NO: 84 - DNA, 830 bp, Brassica rapa
  brCBF6 gene
tactacactcagccttatccagttttcaaaaaaagtattcaactatgaactcagtctctactttttctgaactgctctgctccgagaacaagtctccggttaatacggaaggtggtgattacattttggcggcgagctgtcccaagaaacctgctggtaggaagaagtttcaggagacacgccaccccatttacagaggagttcgcctaagaaagtcaggtaagtgggtgtgtgaagtgagggaaccaaacaagaaatctagaatttggctcggaactttcaaaacagctgagatagcagctcgtgctcacgacgtcgccgccttagctctccgtggaagaggcgcctgcctcaacttcgccgactcggcttggcggctccgtatcccagagacaacctgcgccaaggatatccagaaggctgctgctgaagccgcattggcttttgaggccgagaagagtgataccacgacgaatgatcgtggcatgaacatggaggagacgtccgcggtggcttctccggctgagttgaatgatacgacggcggatcatggcctggacatggaggagacgatggtggaggctgtttttagggacgaacagagagaagggttttacatggcggaggagacgacggtggagggtgttgttccggaggaacagatgagcaaagggttttacatggacgaggagtggacgttcgagatgccgaggttgttggctgatatggcggaagggatgcttctgcctcccccgtccgtacaatggggacataacgatgacttcgaaggagatgctgacatgaacctctggaattattagggatccgcg

SEQ ID NO: 85 - protein, 258 aa, Brassica rapa
  brCBF6- polypeptide
MNSVSTFSELLCSENKSPVNTEGGDYILAASCPKKPAGRKKFQETRHPIYRGVRLRKSGKWVCEVREPNKKSRIWLGTFKTAEIAARAHDVAALALRGRGACLNFADSAWRLRIPETTCAKDIQKAAAEAALAFEAEKSDTTTNDRGMNMEETSAVASPAELNDTTADHGLDMEETMVEAVFRDEQREGFYMAEETTVEGVVPEEQMSKGFYMDEEWTFEMPRLLADMAEGMLLPPPSVQWGHNDDFEGDADMNLWNY

SEQ ID NO: 86 - DNA, 854 bp, Brassica rapa
  brCBF7 gene
ctatactacacacagccttatccagccgctcgagtacttactatactacactcagccttttccagtttttcaaaagaagttttcaacgatgaactcagtctctactctttctgaagttcttggctcccagaacgagtctcccgtaggtggtgattactgtcccatgttggcggcgagctgtccgaagaagccggcgggtaggaagaagtttcgggagacacgtcaccccatttacagaggagttcgtcttagaaagtcaggtaagtgggtgtgtgaagtgagggaaccaaacaagaaatctaggatttggctcggaactttcaaaacagctgagatcgcagctcgtgctcacgacgttgccgccttagctctccgtggaagaggcgcctgcctcaacttcgccgactcggcttggcggctccgtatcccggagacaacctgcgccaaggatatccagaaggctgctgctgaagccgcattggcttttgaggcggagaagagtgataccacgacgacgaatgatcatggcatgaacatggcttctcaggttgaggttaatgacacgacggatcatgacctggacatggaggagacgatggtggaggctgtttttagggaggaacagagagaagggttttacatggcggaggagacgacggtggagggtattgttccggaggaacagatgagcaaagggttttacatggacgaggagtggatgttcgggatgccgaccttgttggctgatatggcggcagggatgctcttaccgccgccgtccgtacaatggggacataatgatgacttcgaaggagatgctgacatgaacctctggaattattaagggatccgcg

SEQ ID NO: 87 - protein, 251 aa, Brassica rapa
  brCBF7 polypeptide
MNSVSTLSEVLGSQNESPVGGDYCPMLAASCPKKPAGRKKFRETRHPIYRGVRLRKSGKWVCEVREPNKKSRIWLGTFKTAEIAARAHDVAALALRGRGACLNFADSAWRLRIPETTCAKDIQKAAAEAALAFEAEKSDTTTTNDHGMNMASQVEVNDTTDHDLDMEETMVEAVFREEQREGFYMAEETTVEGIVPEEQMSKGFYMDEEWMFGMPTLLADMAAGMLLPPPSVQWGHNDDFEGDADMNLWNY

SEQ ID NO: 88 - DNA, 738 bp, Glycine max
  gmCBF1 gene
catccgatttatagtggcgtgaggaggaggaacacggataagtgggtaagtgaggtgagggagcccaacaaaaagaccaggatttggctggggacttttcccacgccggagatggcggcacgggcccacgacgtggcggcaatggccctgaggggccggtatgcctgtctcaacttcgctgactcgacgtggcggttaccaattcccgccactgctaacgcaaaggatatacagaaagcagcagcagaggctgccgaggctttcagaccaagtcagaccttagaaaatacgaatacaaagcaagagtgtgtaaaagtggtgacgacaacaacgatcacagaacaaaaacgaggaatgttttatacggaggaagaagagcaagtgttagatatgcctgagttgcttaggaatatggtgcttatgtccccaacacattgcatagggtatgagtatgaagatgctgacttggatgctcaagatgctgaggtgtccctatggagtttctcaatttaataacgtgcttttggtttggttttttatgttagttttggagtgtgactgtctgtactggttttttattagtagtacggatactagctataggtggcagattgaaagggaccaaaaggaattttcttttgaaaccctttttgtcaaagtaatcaatcgcgtatcatcaagtgaatcccttgatcaagtttatgtatgaattaaataaaagaagaatctagttttggt

SEQ ID NO: 89 - protein, 170 aa, Glycine max
  gmCBF1 polypeptide
HPIYSGVRRRNTDKWVSEVREPNKKTRIWLGTFPTPEMAARAHDVAAMALRGRYACLNFADSTWRLPIPATANAKDIQKAAAEAAEAFRPSQTLENTNTKQECVKVVTTTTITEQKRGMFYTEEEEQVLDMPELLRNMVLMSPTHCIGYEYEDADLDAQDAEVSLWSFSI

SEQ ID NO: 90 - DNA, 793 bp, Raphanus sativus
  rsCBF1 gene
actacactcagccttatccagtttttcttccaacgatggactcaatctctactttttctgaactgcttggctccgagaacgagtctccggttactacggtagtaggaggtgattattttcccaggttggcggcaagctgtccgaagaagccagcgggtaggaagaagtttcaggagacacgtcaccccatttaccgcggagttcgtttaagaaagtcaggtaagtgggtgtgtgaagtgagggaaccaaacaagaaatctaggatttggctcggaactttcaaaaccgctgagatcgctgctcgtgctcacgacgttgctgccttagccctccgcggaagaggcgcctgcctcaacttcgccgactcggcttggcggctccgtatcccggagacaacctgcgccaaggatatccagaaggctgctgctgaagctgcattggcttttgaggccgagaagagtgatcatggcatgaacatgaagaatactacggcggtggcttctcaggttgaggtgaatgacacgacgacggaccatggcgtggacatggaggagacgttggtggaggctgtttttacggaggaacagagagaagggttttacatgacggaggagacgagggtggagggtgttgttacggaggaacagaacaattggttttacatggacgaggagtggatgtttgggatgccgacgttgttggttgatatggcggaagggatgcttttaccgcggccgtccgtacaatcgggacactacgatgacttcgaaggagatgctgacatgaacctctg

SEQ ID NO: 91 - protein, 252 aa, Raphanus sativus
  rsCBF1 polypeptide
MDSISTFSELLGSENESPVTTVVGGDYFPRLAASCPKKPAGRKKFQETRHPIYRGVRLRKSGKWVCEVREPNKKSRIWLGTFKTAEIAARAHDVAALALRGRGACLNFADSAWRLRIPETTCAKDIQKAAAEAALAFEAEKSDHGMNMKNTTAVASQVEVNDTTTDHGVDMEETLVEAVFTEEQREGFYMTEETRVEGVVTEEQNNWFYMDEEWMFGMPTLLVDMAEGMLLPRPSVQSGHYDDFEGDADMNL

SEQ ID NO: 92 - DNA, 682 bp, Raphanus sativus
  rsCBF2 gene
acacctaaaccttatccaggtttaactttttttttcataaagagttttcaacaatgaccacattttctaccttttccgaaatgttgggctccgagtacgagtctccggttacattaggcggagagtattgtccgaagctggccgcgagctgtccgaagaaaccagctggtcgtaagaagtttcgagagacgcgccacccaatatacagaggagttcgtctgagaaactcaggtaagtgggtgtgtgaagtgagggagccaaacaagaaatctaggatttggctcggtactttcctaaccgccgagatcgcagcgcgtgcccacgacgtcgccgccatagccctccgcggcaaatccgcatgtctcaatttcgctgactcggcttggcggctccgtatcccggagacaacatgccccaaggatatacagaaggcggctgctgaagccgcggtggcttttcaggctgagataaatgatacgacgacggatcatggcctggacttggaggagacgatcgtggaggctatttttacggaggtaaacaacgatgagttttatatggacgaggagtccatgttcgggatgccgtctttgttggctagtatggcggaagggatgcttttgccgctgccgtccgtacaatctgaacataactgtgacttcgacggagatgctgacatgaa

SEQ ID NO: 93 - protein, 209 aa, Raphanus sativus
  rsCBF2- polypeptide
MTTFSTFSEMLGSEYESPVTLGGEYCPKLAASCPKKPAGRKKFRETRHPIYRGVRLRNSGKWVCEVREPNKKSRIWLGTFLTAEIAARAHDVAAIALRGKSACLNFADSAWRLRIPETTCPKDIQKAAAEAAVAFQAEINDTTTDHGLDLEETIVEAIFTEVNNDEFYMDEESMFGMPSLLASMAEGMLLPLPSVQSEHNCDFDGDADM

SEQ ID NO: 94 - DNA, 349 bp, Zea mays
  zmCBF1 gene
cggagtccgcggacggcggcggcggcggcgacgacgagtacgcgacggtgctgtcggcgccacccaagcggccggcggggcggaccaagttccgggagacgcggcaccccgtgtaccgcggcgtgcggcggcgcgggcccgcggggcgctgggtgtgcgaggtccgcgagcccaacaagaagtcgcgcatctggctcggcaccttcgccacccccgaggccgccgcgcgcgcgcacgacgtggccgcgctggccctgcggggccgcgccgcgtgcctcaacttcgccgactcggcgcgcctgctccaagtcgaccccgccacgctcgccacccccgacgacatccgccg

SEQ ID NO: 95 - protein, 115 aa, Zea mays
  zmCBF1- polypeptide
ESADGGGGGDDEYATVLSAPPKRPAGRTKFRETRHPVYRGVRRRGPAGRWVCEVREPNKKSRIWLGTFATPEAAARAHDVAALALRGRAACLNFADSARLLQVDPATLATPDDIR

SEQ ID NO: 96 - DNA, 675 bp, Arabidopsis thaliana
  G912 gene
ttactcgtcaaaactccagagtgacacgtcacccactccgtcaaagtcgttatgattccagccaacttccggcggcggcaaaagcatcccctccgccatattctcaaaaaagttgggcatccccaaaagcgcctcatcatccatataaaacacaccaccattctgctcctccgccctcctctccccctccctcaccccctcccctgccgcctcctctgcctccgccgcagttttagatccctccgtcgtagtctcattctgaaacgccattgcagcttcagacgcagctttctgaatctccttaggacaagtagtctcaggaatacgaagccgccaagcagaatcagcgaaattgagacaagcagagcgaccacgaagagctaaagcagcaacatcatgagcacgagcagccatttcaaccgtcggaaaagtacctaaccaaatcctagatttcttattaggctctctaacttcacaaacccatttaccagaattcctctgacgaactcctctgtaaatcggatgacgtgtctcacgaaacttcttcctcccagctcgtttctttggacaacttgaagctaactttggtgaacactcactactgtctgaaaccggagatctatgatcggagattgagagaaacgagtctgggaatgtagagtaaaatggattcat

SEQ ID NO: 97 - protein, 224 aa, Arabidopsis thaliana
  G912 polypeptide
MNPFYSTFPDSFLSISDHRSPVSDSSECSPKLASSCPKKRAGRKKFRETRHPIYRGVRQRNSGKWVCEVREPNKKSRIWLGTFPTVEMAARAHDVAALALRGRSACLNFADSAWRLRIPETTCPKEIQKAASEAAMAFQNETTTEGSKTAAEAEEAAGEGVREGERRAEEQNGGVFYMDDEALLGMPNFFENMAEGMLLPPPEVGWNHNDFDGVGDVSLWSFDE

SEQ ID NO: 98 - DNA, 630 bp, Arabidopsis thaliana
  G2513 gene
atgaataatgatgatattattctggcggagatgaggcctaagaagcgtgcgggaaggagagtgtttaaggagacacgtcacccagtttacagaggcataaggcggaggaacggtgacaaatgggtctgcgaagtcagagaaccgacgcaccaacgccgcatttggctcgggacttatcccacagcagatatggcagcgcgtgcacacgacgtggcggttttagctctgcgtgggagatccgcatgtttgaatttcgccgactccgcttggcggcttccggtgccggaatccaatgatccggatgtgataagaagagttgcggcggaagctgcggagatgtttaggccggtggatttagaaagtggaattacggttttgccttgtgcgggagatgatgtggatttgggttttggttcgggttccggctctggttcgggatcggaggagaggaattcttcttcgtatggatttggagactacgaagaagtctcaacgacgatgatgagactcgcggaggggccactaatgtcgccgccgcgatcgtatatggaagacatgactcctactaatgtttacacggaagaagagatgtgttatgaagatatgtcattgtggagttacagatattaa

SEQ ID NO: 99 - protein, 209 aa, Arabidopsis thaliana
  G2513 polypeptide
MNNDDIILAEMRPKKRAGRRVFKETRHPVYRGIRRRNGDKWVCEVREPTHQRRIWLGTYPTADMAARAHDVAVLALRGRSACLNFADSAWRLPVPESNDPDVIRRVAAEAAEMFRPVDLESGITVLPCAGDDVDLGFGSGSGSGSGSEERNSSSYGFGDYEEVSTTMMRLAEGPLMSPPRSYMEDMTPTNVYTEEEMCYEDMSLWSYRY

SEQ ID NO: 100 - DNA, 546 bp, Arabidopsis thaliana
  G2107 gene
ttagtaactccaaagtgacaaatcttcgtaacacatttcttcgtccacgtacacactcgtattcatatcaatgtacgatcttggcggcgacatcaacggctcctccgcgagcctcatcatcattccagcgactccttcatccgacgtgtcaaactcactggctgagggtaaaaccgtaattcctgtactaaactccggcggcctgaacatctccgctgcttcggccgccgtgcgcctgatcgtgtccggatcagtggatgccggcaccggcaacctccaagcagaatcggagaaattcaaacacgcggatctcccgcgcagagcaagaaccgccacgtcgtgagcacgtgcggccatatctgccgtcggataagttccgagccagactcgacgctgatgaatcggttcacggacttcgcatacccatttgtcgccgtccctacgccgcacgcctctgtagattgggtgacgtgtctccttgaaaatcctccgtccagcacgcttctttggcttcatctccgccacggtgatatcgtcgttttccat

SEQ ID NO: 101 - protein, 181 aa, Arabidopsis thaliana
  G2107 polypeptide
MENDDITVAEMKPKKRAGRRIFKETRHPIYRGVRRRDGDKWVCEVREPIHQRRVWLGTYPTADMAARAHDVAVLALRGRSACLNFSDSAWRLPVPASTDPDTIRRTAAEAAEMFRPPEFSTGITVLPSASEFDTSDEGVAGMMMRLAEEPLMSPPRSYIDMNTSVYVDEEMCYEDLSLWSY

SEQ ID NO: 102 - DNA, 888 bp, Arabidopsis thaliana
  G21
tcattcagatagaaaaaacggctcttcaagccgaaacccagcatcggctccacaaagctgccacgtggacgagtagtagcaaaacgcatcgtttcgtatcatcatctcattctcatcggtaaacaaatccggcaaatcaaacagcttctcttcctcactgtctttgtccgtacacgccgaagtcgaagcacacgaagcttccgaatactcttgactctgagtcgtcgtcgtcgtgcttgtgtccgaagaaaacaactgagccaccacggctcgactcggctcggcttcaactatttcagccacttcagaattactcacatcgttgaccgaatcttgccagttaacggccgctaaagaggcggcggcttgaatgtctttaggagaatttgtgactggacgaggaagctcgccggctaacttgggaaaattgaggtaagccgttgtacctttaatggctaaagccgctacgtcatgagctcgagctgccatctcagccgttggataagtcccgagccagattcttgatttctttctcggctctctaatctccgacacccattttccccaactcctcatcctcactcctctatacgtcggatgtttatctccaccgttggtctttcgccgtttcccacctccaccgttttgatcatcatcgtcttcaacggagactaacgatgacaatgatcttcgagatgcttttcttttagagttgtcttcctcaatgaagttagaatttgttgatgacgaagaagaggaagatgtagtaggtgaagaggacaatgaggcggaagcggtgatggaggaggaggaagatacggcggggatggcggaggagataaaggtaacttgagaaacactactctctatgttgatttgtcttgccat

SEQ ID NO: 103 - protein, 295 aa, Arabidopsis thaliana
  G21 polypeptide
MARQINIESSVSQVTFISSAIPAVSSSSSITASASLSSSPTTSSSSSSSTNSNFIEEDNSKRKASRRSLSSLVSVEDDDDQNGGGGKRRKTNGGDKHPTYRGVRMRSWGKWVSEIREPRKKSRIWLGTYPTAEMAARAHDVAALAIKGTTAYLNFPKLAGELPRPVTNSPKDIQAAASLAAVNWQDSVNDVSNSEVAEIVEAEPSRAVVAQLFSSDTSTTTTTQSQEYSEASCASTSACTDKDSEEEKLFDLPDLFTDENEMMIRNDAFCYYSSTWQLCGADAGFRLEEPFFLSE

SEQ ID NO: 104 - DNA, 20 bp, Artificial Sequence
  PCR primer O368
cayccnathtaymgnggngt

SEQ ID NO: 105 - DNA, 26 bp, Artificial Sequence
  PCR primer O376
gcngcytcngcngcngcyttytgdat

SEQ ID NO: 106 - DNA, 24 bp, Artificial Sequence
  PCR primer O2953
aaraarttymgngaracnmgncay

SEQ ID NO: 107 - DNA, 25 bp, Artificial Sequence
  PCR primer O5436
ggaggaacacggataagtgggtaag

SEQ ID NO: 108 - DNA, 23 bp, Artificial Sequence
  APCR primer O5437
aggatttggctggggacttttcc

SEQ ID NO: 109 - DNA, 35 bp, Artificial Sequence
  PCR primer O18016
acgcgtcgacccatcatcaccgagatcgactcgac

SEQ ID NO: 110 - DNA, 37 bp, Artificial Sequence
  PCR primer O18017
ataagaatgcggccgctcattgttcgctcactgggag

SEQ ID NO: 111 - DNA, 20 bp, Artificial Sequence
  PCR primer O18035
gctgacagaacgggtgccga

SEQ ID NO: 112 - DNA, 20 bp, Artificial Sequence
  PCR primer O18036
tgaccgtttctggataggca

SEQ ID NO: 113 - DNA, 24 bp, Artificial Sequence
  PCR primer O18065
ggccggcggggcgaaccaagttcc

SEQ ID NO: 114 - DNA, 24 bp, Artificial Sequence
  PCR primer O18066
aggcagagtcggcgaagttgaggc

SEQ ID NO: 115 - DNA, 670 bp, Secale cereale
  Rye CBF20 gene
cgtcgacccacgcgtccggatccatcgatcaaacctctcaacacagctgctgattcttccagcactccacacttacaagcagcctcgatctccgctagctctagacctagatgccgtctggtcaggaggggcaacggcacaggacggtgaggtcggagccgccgggcggtgggtctgcgaggtgcgcgtgctcgggatgaggggctccaggctctggcttggcaccttcgtcaccgcggagatggcggcgcgcgcccacgacgccgccgtgctcgcgctctccggccgcaaggcctgcctcaacttcgccgactccgcctggcggatgctgcccgtgctcgcggctggctccttcggcttcggcagcgcgcgggagatcaagaccgccgtcgccgtcgccgtcctcgcgttccagcggcagcagatcattcttccagtagcccgcccggcggaggagccggccgacgtcccgagcggcgcgctgttctccatgtcatcaggcgacttgctggagctcgacgaggagcagtggtttggcggcatggttgccgggtcctactacgagagcttggcgcaggggatgctcgtcgagccgccggacgccggagcgtggcgagaggacagcgagcacagcggcgtggcggagacgcagacgccgttgtggagctaa

SEQ ID NO: 116 - protein, 222 aa, Secale cereale
  Rye CBF20 polypeptide
VDPRVRIHRSNLSTQLLILPALHTYKQPRSPLALDLDAVWSGGATAQDGEVGAAGRWVCEVRVLGMRGSRLWLGTFVTAEMAARAHDAAVLALSGRKACLNFADSAWRMLPVLAAGSFGFGSAREIKTAVAVAVLAFQRQQIILPVARPAEEPADVPSGALFSMSSGDLLELDEEQWFGGMVAGSYYESLAQGMLVEPPDAGAWREDSEHSGVAETQTPLWS

SEQ ID NO: 117 - DNA, 813 bp, Secale cereale
  Rye CBF28 gene
atggacgtcgccgacatcgcctccccgtctggccagcaggagcaggggcaccggacggtgtcgtcggagccgccgaagcgccccgcggggcggaccaagttccacgagacgcgccacccgctgtaccgcggcgtgcggcgccgtggccgcgtcgggcagtgggtgtgcgaggtgcgcgtgcccgggatcaagggctccaggctctggctcggcaccttcaacacggccgagatggcggcgcgcgcccacgacgcagccgtgctcgcgctctcctgccgcgccgcctgcctcaacttcgccgactccgcctggcggatgctgcccgtgctcgcggccgggtcgttcgggttcggcagcccgcgggagatcaaggcagccgtcgccgtcgccgtcatcgcgttccagcggaagcagattattccggtcgccgtcgccgtcgtggcgctccagcagcagcaggttccagtcgccgtcgccgtcgttgcgctaaagcagaagcaggttccggtcgctgtggccgtcgtggcgctccagcagctgcatgttccggtagccgtcgccgtcgtggcgctccagcagcagcagattattcttccagtcgcgtgcctggcgcccgagttttacatgtcttccggcgacctgctggagctcgacgaggagcactggtttggcggcatggacgccgggtcgtactacgcgagcttggcgcaggggatgctcgtggctccgccggacgaaagagcgaggccggagaacggcgagcaagagcggcgtccagacgccgctatggagctgtttgttcgactaatttag

SEQ ID NO: 118 - protein, 270 aa, Secale cereale
  Rye CBF28 polypeptide
MDVADIASPSGQQEQGHRTVSSEPPKRPAGRTKFHETRHPLYRGVRRRGRVGQWVCEVRVPGIKGSRLWLGTFNTAEMAARAHDAAVLALSCRAACLNFADSAWRMLPVLAAGSFGFGSPREIKAAVAVAVIAFQRKQIIPVAVAVVALQQQQVPVAVAVVALKQKQVPVAVAVVALQQLHVPVAVAVVALQQQQIILPVACLAPEFYMSSGDLLELDEEHWFGGMDAGSYYASLAQGMLVAPPDERARPENGEQERRPDAAMELFVRLI

SEQ ID NO: 119 - DNA, 807 bp, Secale cereale
  Rye CBF46 gene
atggacgtcgccgacatcgcctcccggtctggccagcagcagcaggggcaccggaccgtgtcgtcggagccgccgaagcgccccgcggggaggaccaagttccacgagacgcgccacccgctgtaccgcggcgtgcggcgccgtggccgcgtcgggcagtgggtgtgcgaggtgcgcgttcccgggatcaagggctccaggctctggctcggcaccttcaacacggccgagatggcggcgcgcgcgcacgacgccgccgtgctcgcgctctccggccgcaaagcctgcctcaacttcgccgactccgcctggcggatgctgcccgtgctcgcggccggctccttcggctttgatagcgcgcgggaggtcaaggccgccgtcgccgtcgccgtcgtcgcgttccagcggaaacagattattccagtcgccgtcgctgtcgttgctctccagaagcagcaggttccggtcgccgtggccatcgtggcgctccagcagaggcaggttccggtcgccgtcgccgtcgtggcgctccagaagctgcaggttccggtcgccgtcgccgtcgtagcgctccagaagaagcagattattcttccagccgcgtgcctggcgccggagttttacatgtcttccggcgacctgttggagctcgacgaggagcagtggtttggcggcatggacgccgggtcgtactacgccagcttggcgcaggggatgctcgtggcgccgccggacgacagagcgaggccggagaacggcgagcagagcggcgtccagactccgctatggagctgcttgttcgactaa

SEQ ID NO: 120 - protein, 268 aa, Secale cereale
  Rye CBF46 polypeptide
MDVADIASRSGQQQQGHRTVSSEPPKRPAGRTKFHETRHPLYRGVRRRGRVGQWVCEVRVPGIKGSRLWLGTFNTAEMAARAHDAAVLALSGRKACLNFADSAWRMLPVLAAGSFGFDSAREVKAAVAVAVVAFQRKQIIPVAVAVVALQKQQVPVAVAIVALQQRQVPVAVAVVALQKLQVPVAVAVVALQKKQIILPAACLAPEFYMSSGDLLELDEEQWFGGMDAGSYYASLAQGMLVAPPDDRARPENGEQSGVQTPLWSCLFD

SEQ ID NO: 121 - DNA, 639 bp, Secale cereale
  Rye CBF7 gene
atggacgccgccgacgccggctccccccgttttgggcacaggacggtgtgctcggagccgcccaagaggccggcagggcggaccaagtttaaggagacccgccacccgctgtaccgcggcgtgcggcggcggggtcggctcgggcagtgggtgtgcgaggtgcgcgtgcgcggcgcgcaagggtacaggctctggctcggcacattcaccaccgccgagatggcggcgcgcgcgcacgactccgccgtgctcgcgctcctcgaccgcgccgcttgcctcaacttcgccgactccgcctggcggatgctgcccgtcctcgcggcaggctcgtcccgcttcagcagcgcgcgggaaatcaaggacgccgtcgccgtcgccgtcgtggagttccagcggcagcgccccttcgtgtccacgtcggagacggccgacggcgagaaggacgtccaaggctcgccgaggccgagcgagctgtccacgtccagcgacttgttggacgagcactggtttagcggcatggacgccggctcttactacgcgagcttggcgcaggggatgctcatggagccgccggccgccagagcgtggagcgaggatggcggcgaatacagcggcgtccacacgccgctttggaactag

SEQ ID NO: 122 - protein, 212 aa, Secale cereale
  Rye CBF7 polypeptide
MDAADAGSPRFGHRTVCSEPPKRPAGRTKFKETRHPLYRGVRRRGRLGQWVCEVRVRGAQGYRLWLGTFTTAEMAARAHDSAVLALLDRAACLNFADSAWRMLPVLAAGSSRFSSAREIKDAVAVAVVEFQRQRPFVSTSETADGEKDVQGSPRPSELSTSSDLLDEHWFSGMDAGSYYASLAQGMLMEPPAARAWSEDGGEYSGVHTPLWN

SEQ ID NO: 123 - DNA, 807 bp, Secale cereale
  Rye CBF71
atggacgtcgccgacatcgcctcccggtctggccagcagcagcaggggcaccggaccgtgtcgtcggagccgccgaagcgccccgcggggaggaccaagttccacgagacgcgccacccgctgtaccgcggcgtgcggcgccgtggccgcgtcgggcagtgggtgtgcgaggtgcgcgtgcccgggatcaagggctccaggctctggctcggcaccttcaacacggccgagatggcggcgcgcgcgcacgacgctgccgtgctcgcgctctccggccgcgccgcctgcctcaacttcgccgactccgcctggcggatgctgcccgtgctcgcggccggctccttcggctttgatagcgcgcgggaggtcaaggccgccgtcgccgtcgccgtcgtcgcgttccagcggaaacagattattccagtcgccgtcgctgtcgttgctctccagaagcagcaggttccggtcgccgtggccgtcgtggcgctccagcagaggcaggttccggtcaccgtcgccgtcgtggcgctccagaagctgcaggttccggtcgccgtcgccgtcgtggcgctccagaagaagcagattattcttccagccgcgtgtctggcgccggagttttacatgtcttccggcgacctgttggagctcgacgaggagcagtggtttggcggcatggacgccgggtcgtactacgccagcttggcgcaggggatgctcgtggcgccgccggacgacagagcgaggccggagaacggcgagcagagcggcgtccagactccgctatggagctgcttgttcgactaa

SEQ ID NO: 124 - protein, 268 aa, Secale cereale
  Rye CBF71 polypeptide
MDVADIASRSGQQQQGHRTVSSEPPKRPAGRTKFHETRHPLYRGVRRRGRVGQWVCEVRVPGIKGSRLWLGTFNTAEMAARAHDAAVLALSGRAACLNFADSAWRMLPVLAAGSFGFDSAREVKAAVAVAVVAFQRKQIIPVAVAVVALQKQQVPVAVAVVALQQRQVPVTVAVVALQKLQVPVAVAVVALQKKQIILPAACLAPEFYMSSGDLLELDEEQWFGGMDAGSYYASLAQGMLVAPPDDRARPENGEQSGVQTPLWSCLFD

SEQ ID NO: 125 - DNA, 240 bp, Triticum aestivum
  Wheat CBF gene
agtgattggccggcggggcgaaccaagttccgtgagacacgacacccgctgtaccgcggcgtgcggcgccgtggccgggtcgggcagtgggtgtgcgaggtgcgcgtgccaggagtgaagggctccaggctctggctcggcaccttcaccaccgccgagatggcggcgcgcgcgcacgacgccgcggtgctcgcgctctccggccgcgccgcctgcctcaacttcgccgactctgcctaa

SEQ ID NO: 126 - protein, 76 aa, Triticum aestivum
  Wheat CBF polypeptide
PAGRTKFRETRHPLYRGVRRRGRVGQWVCEVRVPGVKGSRLWLGTFTTAEMAARAHDAAVLALSGRAACLNFADSA

SEQ ID NO: 127 - DNA, 705 bp, Glycine max
  Soy CBF gene
atgtttaccttgaatcattcttctgatttgtaccatgtttcccctgagctctcatcttccttggacacatcctcgccggcttcggagggctctcgtggcgtggcattttccgacgaggaggtgcggctggcggtgaggcacccgaagaagcgggcaggtcggaagaagttccgggagacgcgccacccggtgtaccggggggtgaggaggaggaactcggataagtgggtgtgtgaggtgagggagcccaacaagaagaccaggatttggctggggactttccccacgccggagatggcggctcgggcgcacgacgtggcggcaatggccctgaggggccggtatgcctgtctaaactttgctgactcggcctggcggttacctgttcccgccacggccgaggcaaaggatatacagaaggcagcagcagaagctgcccaggctttcagaccagatcaaaccttaaaaaatgctaatacaaggcaggagtgtgtggaggcggtggcggtggcggtggcggagacaacaacggcgacggcacaaggggtgttttatatggaggaagaagagcaggtgttggatatgcctgagttgcttaggaatatggtgctcatgtccccaacacattgcttagggtatgagtatgaagatgctgacttggatgcccaagatgctgaggtgtcactatggaatttctcaatttaa

SEQ ID NO: 128 - protein, 234 aa, Glycine max
  Soy CBF polypeptide
MFTLNHSSDLYHVSPELSSSLDTSSPASEGSRGVAFSDEEVRLAVRHPKKRAGRKKFRETRHPVYRGVRRRNSDKWVCEVREPNKKTRIWLGTFPTPEMAARAHDVAAMALRGRYACLNFADSAWRLPVPATAEAKDIQKAAAEAAQAFRPDQTLKNANTRQECVEAVAVAVAETTTATAQGVFYMEEEEQVLDMPELLRNMVLMSPTHCLGYEYEDADLDAQDAEVSLWNFSI

SEQ ID NO: 129 - DNA, 2396 bp, Arabidopsis thaliana
  Artificial fusion between rab18 promoter and CBF1 gene
aagcttcaaattctgaatattcacatatcaaaaaagtgatgcgagggagagaagggtatttcatcttacttgcagcgatagaactccttgtggtagaagtgacatataactttttcactccttgtgacttctcccaagaagtcgccttcgctaacttctcggtattcaccatgtccttgtcttttgaatgcttctctcttttccacttctctctgtccagacacatcttgggattagatcagtccatactaatgaagacaaaagtgttaaccaaatatagataatgagagaatgtggggggtgaagttgaaccctgagtgctgcaatcctatctgcgtgcaacttttctagctctggatcctacagaacaagagcaagtcagtctcccatagagacagaagagaaacataacatttcattgtacagattataaaagaacaattcaaagagagcccccattgaacttacatccatcaattcgtcaagatcaacttcctcgttgacaggtcttgatccttgtgccttttcatttgcaagaacttcctgcacaacaaaaatacggaaatgtttttgatccaagaacatcaaactctaattgcaattgtaaccaacaaaaaaacacttaaaattcgctatacactatcaaatttcctgacacgtagacctcatgtcagcacaaatagaatctagcctatctctgttaattgggttaccaaacaacatgagattgattatgtggaaaaccaagcacattatttaccctttgaaaatacgcgaaatcaagatccaagaagaatttatggagaacagcttcgaagtacgaaataaacaatgaagattaaattacctttttataatctctagcagctgccgccaatacattcccgaatgccagattcgagagggtcgacttcaccgtatccggatccatctctttaccaaccaactaatccaactcagaaaattttaaaatctcaatcaaaaatccctctaagatagccagagaagagattgtaaacaaggatttgaaatctggtgcagagaggagaaactccccgacaatgaacaccaacgatctaaacgcggcgtttggtaaaagttgagtaaattttgttagggcttagttttagtccatgggctaattagtaagtgatttacggcccacacatgagcccaaatgtttcagacccagccaagtttcttcaaattcacccaatcaacgacgatgtacgtgtgtatgaaaatcattaacacgacgcatcgctttcgaggaggagcattacgtgtcctgttagctacgataatgttagtaccgccacaaagaaaaggatagatattttgctttccagcaccctgtcatgggattgatatgaacacgtacttggtatcgacatgaaagctcaaaaataaattcaatccgattcctttagtgatatcagaagttcattttaaatacgaacacgtatggcgaaacaccacgccgacattttctgctgctgccacgcgtcactttccaaatattgattcattaaactaatagttgatccatatccgaaaccggactataaaactatcttcaatgcgttaacgaatcttcatcgatcaaactcatcaaagtctaatatcacaaagaaagagtttttttaactagcttagctcaaagtgtttgcttaagacaagaagaacgaattcaatgaactcattttcagctttttctgaaatgtttggctccgattacgagcctcaaggcggagattattgtccgacgttggccacgagttgtccgaagaaaccggcgggccgtaagaagtttcgtgagactcgtcacccaatttacagaggagttcgtcaaagaaactccggtaagtgggtttctgaagtgagagagccaaacaagaaaaccaggatttggctcgggactttccaaaccgctgagatggcagctcgtgctcacgacgtcgctgcattagccctccgtggccgatcagcatgtctcaacttcgctgactcggcttggcggctacgaatcccggagtcaacatgcgccaaggatatccaaaaagcggctgctgaagcggcgttggcttttcaagatgagacgtgtgatacgacgaccacggatcatggcctggacatggaggagacgatggtggaagctatttatacaccggaacagagcgaaggtgcgttttatatggatgaggagacaatgtttgggatgccgactttgttggataatatggctgaaggcatgcttttaccgccgccgtctgttcaatggaatcataattatgacggcgaaggagatggtgacgtgtcgctttggagttactaatattcgatagtcgtttccatttttgtactatagtttgaaaatattctagttccgcggccgc

SEQ ID NO: 130 - DNA, 2655 bp, Arabidopsis thaliana
  Artificial fusion between DREB2a promoter and CBF1 gene
aagcttgtaatcgataacctaaatcattgtaatgaatgcgttcctccctatcgatcctaggccttagacaatgctgaatgattcatagccacgcgaataacctattcatactaatatggaaagaaagaagccaaacttacagagctcttctcacggtcgtcgaagaaaagagagtctacagtcagcaacaaaattagtgttgccatcatggcatcatcttgcagctttttccgcacaaataatttatcatccaaatgttgttcactaaaccaaaaacaaacaagcattacagcgaagaaaccaactcgtagatacagatttccaaattcatgccttatttagaccaataaaaactgaaatttctcttcagcgaaaaaaaaaacaaacaaactgaaatttcatatagatccagaagatagaaacttgtaggctcaatcgactagactagaagatgctcacccgatcgtgcttgagatagcgagatagtagcaacaccgacggtagaaataaaatggacgacacccatccaatgggctaatttagattaacgggctttaagggtttgataatggatgttaattaactgaggcacatgggattgtatcacgtaggcaatgggtttgataatggatgttaagtaactaaggcccatgaggttgaagtacgtaggcaattgcgtgagcttacgttagcgatgccgttagagacacgtagtggatgaagtggctttggttagcaaaggacacatgaggcacatgcaaaggctataaatgactgctgctttgctacaacttgcgattcccaaattttataaggtaatggaccctatcacctctgctcgaagctaagccacccaagtttgagcttcaccatttgacacgtctctagctaatacttaatgcttaaactttaaactaatacttcatgtttaagacatttctggctgacacatttatgaattcgctctatgtcgtacgtacaccggaaacctttaattttacaatattaaccgtgatctttttaaaaatatctatataaacgataagtatctcatcaaaacaaaattgaataatgtgcacgtttgtaaaaactagaaaaacaggcaataaacatcatcatccaattacacatctagtaagtgtgatgcagtggcaaactggcaataaaccaagaaaaatcgagaaagagcagatgagacagtgttgtgttgtcagggttaataaaaaaaaaatgaagatattttaaaatttcataatatttaaataatgaagtagttttatgatcttatccataaatcaattttaaaaaggtttaaactttacttttccgtatcaacagcgtgtttcgagaagattcgggaggacactcgtcgaacggaaaagtcgtctaagcctttatgttcgaatcaaaaactgacacgtaaccttgctctcaaacagaaaaataaaataatgttagaaaaatctagagaaggctataaatactccgtagatactttgtcttccttagatattttgatttctgctaaagctgtctgataaaaagaagaggaaaactcgaaaaagctacacacaagaagaagaagaaaagatacgagcaagaagactaaacacgaaagcgatttatcaactcgaaggaagagactttgattttcaaatttcgtcccctataggtacgttgatttctgatttcttacaaattttcaattaatttctctgattaggtctcgtcaattggtgattctagggtttatcttcgtctttaggttttcgattatttccttttaggtatagctgtgaaattgggaaattttaggtgttcctaatttgataacaaataagtaaatcgatctgattttgaaccaagtttaagttctcttgccttatgtttcagcttgtgttctgatattgaagtgttgttgtattgtagattgtgttgtttctgggaaggccatggactcattttcagctttttctgaaatgtttggctccgattacgagcctcaaggcggagattattgtccgacgttggccacgagttgtccgaagaaaccggcgggccgtaagaagtttcgtgagactcgtcacccaatttacagaggagttcgtcaaagaaactccggtaagtgggtttctgaagtgagagagccaaacaagaaaaccaggatttggctcgggactttccaaaccgctgagatggcagctcgtgctcacgacgtcgctgcattagccctccgtggccgatcagcatgtctcaacttcgctgactcggcttggcggctacgaatcccggagtcaacatgcgccaaggatatccaaaaagcggctgctgaagcggcgttggcttttcaagatgagacgtgtgatacgacgaccacggatcatggcctggacatggaggagacgatggtggaagctatttatacaccggaacagagcgaaggtgcgttttatatggatgaggagacaatgtttgggatgccgactttgttggataatatggctgaaggcatgcttttaccgccgccgtctgttcaatggaatcataattatgacggcgaaggagatggtgacgtgtcgctttggagttactaatattcgatagtcgtttccatttttgtactatagtttgaaaatattctagttccgcggccgc

SEQ ID NO: 131 - DNA, 21 bp, Artificial sequence
  O6792 Synthetic oligonucleotide primer
gcttgtccattatcctagaat

SEQ ID NO: 132 - DNA, 28 bp, Artificial sequence
  O6793 Synthetic oligonucleotide primer
cggaattcgttcttcttgtcttaagcaa

SEQ ID NO: 133 - DNA, 35 bp, Artificial sequence
  O6805 Synthetic oligonucleotide primer
cggaattcaatgaactcattttcagctttttctga

SEQ ID NO: 134 - DNA, 43 bp, Artificial sequence
  O6806 Synthetic oligonucleotide primer
ataagaatgcggccgcggaactagaatattttcaaactatagt

SEQ ID NO: 135 - DNA, 32 bp, Artificial sequence
  O6798  Synthetic oligonucleotide primer
gcccaagcttgtaatcgataacctaaatcatt

SEQ ID NO: 136 - DNA, 31 bp, Artificial sequence
  O6799 Synthetic oligonucleotide primer
aactgccatggccttcccagaaacaacacaa

SEQ ID NO: 137 - protein, 62 aa, Arabidopsis thaliana
HPIYRGVRQRNSGKWVCELREPNKKTRIWLGTFQTAEMAARAHDVAAIALRGRSACLNFADS

SEQ ID NO: 138 - protein, 62 aa, Arabidopsis thaliana
HPIYRGVRRRNSGKWVCEVREPNKKTRIWLGTFQTAEMAARAHDVAALALRGRSACLNFADS

SEQ ID NO: 139 - protein, 62 aa, Arabidopsis thaliana
HPIYRGVRQRNSGKWVSEVREPNKKTRIWLGTFQTAEMAARAHDVAALALRGRSACLNFADS

SEQ ID NO: 140 - protein, 62 aa, Brassica juncea
HPIYRGVRQRNSGKWVCEVREPNKKSRIWLGTFPTVEMAARAHDVAALALRGRSACLNFADS

SEQ ID NO: 141 - protein, 62 aa, Brassica oleracea
HPVYRGVRLRNSGKWVCEVREPNKKSRIWLGTFLTAEIAARAHDVAAIALRGKSACLNFADS

SEQ ID NO: 142 - protein, 62 aa, Raphanus sativus
HPIYRGVRLRNSGKWVCEVREPNKKSRIWLGTFLTAEIAARAHDVAAIALRGKSACLNFADS

SEQ ID NO: 143 - protein, 62 aa, Brassica juncea
HPIYRGVRLRKSGKWVCEVREPNKRSRIWLGTFLTAEIAARAHDVAAIALRGKSACLNFADS

SEQ ID NO: 144 - protein, 62 aa, Brassica napus
HPIYRGVRLRKSGKWVCEVREPNKKSRIWLGTFLTAEIAARAHDVAAIALRGKSACLNFADS

SEQ ID NO: 145 - protein, 62 aa, Brassica juncea
HPIYRGVRLRKSGKWVCEVREPNKKSRIWPGTFLTAEIAARAHDVAAIALRGKSACLNFADS

SEQ ID NO: 146 - protein, 62 aa, Brassica napus
HPIYRGVRLRKSGKWVCEVREPNKKSRIWPGTFKTAEMAARAHDVAALALRGRGACLNYADS

SEQ ID NO: 147 - protein, 62 aa, Brassica napus; identical to SEQ ID NO: 146

SEQ ID NO: 148 - protein, 62 aa, Brassica napus
HPIYRGVRLRKSGKWVCEVREPNKKSRIWLGTFKTAEMAARAHDVAALALRGRGACLNYADS

SEQ ID NO: 149 - protein, 62 aa, Brassica napus; identical to SEQ ID NO: 148

SEQ ID NO: 150 - protein, 62 aa, Brassica napus; identical to SEQ ID NO: 148

SEQ ID NO: 151 - protein, 62 aa, Brassica oleracea; identical to SEQ ID NO: 148

SEQ ID NO: 152 - protein, 62 aa, Brassica rapa; identical to SEQ ID NO: 148

SEQ ID NO: 153 - protein, 62 aa, Brassica napus
HPIYRGVRLRKSGKWVCEVREPNKKSRIWLGTFKTAEIAARAHDVAALALRGRGACLNFADS

SEQ ID NO: 154 - protein, 62 aa, Brassica napus; identical to SEQ ID NO: 153

SEQ ID NO: 155 - protein, 62 aa, Brassica oleracea; identical to SEQ ID NO: 153

SEQ ID NO: 156 - protein, 62 aa, Brassica rapa; identical to SEQ ID NO: 153

SEQ ID NO: 157 - protein, 62 aa, Brassica rapa; identical to SEQ ID NO: 153

SEQ ID NO: 158 - protein, 62 aa, Brassica rapa; identical to SEQ ID NO: 153

SEQ ID NO: 159 - protein, 62 aa, Brassica rapa; identical to SEQ ID NO: 153

SEQ ID NO: 160 - protein, 62 aa, Brassica rapa; identical to SEQ ID NO: 153

SEQ ID NO: 161 - protein, 62 aa, Brassica rapa; identical to SEQ ID NO: 153

SEQ ID NO: 162 - protein, 62 aa, Raphanus sativus; identical to SEQ ID NO: 153

SEQ ID NO: 163 - protein, 62 aa, Brassica oleracea
HPIYRGVRLRKSGKWVCEVREPNKKSRIWLGTFKTAEIAARAHDVAALALRGRAACLNFADS

SEQ ID NO: 164 - protein, 62 aa, Brassica oleracea
HPIYRGVRLRKSGKWVCEVRELNKKSRIWLGTFKTAEMAARAHDVAALALRGRGACLNYADS

SEQ ID NO: 165 - protein, 62 aa, Brassica napus
HPIYRGVRLRKSGKWVCEVREPNKKSRIWPGTFKTAEMAARAHDVAALALRGRGARLNYADS

SEQ ID NO: 166 - protein, 63 aa, Zea mays
HPVYRGVRRRGPAGRWVCEVREPNKKSRIWLGTFATPEAAARAHDVAALALRGRAACLNFADS

SEQ ID NO: 167 - protein, 62 aa, Glycine max
HPIYSGVRRRNTDKWVSEVREPNKKTRIWLGTFPTPEMAARAHDVAAMALRGRYACLNFADS

SEQ ID NO: 168 - protein, 63 aa, Nicotiana tabacum
GRHYRGVRQRPWGKFAAEIRDPAKNGARVWLGTYETAEEAALAYDKAAYRMRGSKALLNFPHR

SEQ ID NO: 169 - protein, 62 aa, Arabidopsis thaliana
RCSFRGVRQRIWGKWVAEIREPNRGSRLWLGTFPTAQEAASAYDEAAKAMYGPLARLNFPRS

SEQ ID NO: 170 - protein, 62 aa, Arabidopsis thaliana
HCSFRGVRQRIWGKWVAEIREPKIGTRLWLGTFPTAEKAASAYDEAATAMYGSLARLNFPQS

SEQ ID NO: 171 - protein, 62 aa, Arabidopsis thaliana
HPVYRGVRKRNWGKWVSEIREPRKKSRIWLGTFPSPEMAARAHDVAALSIKGASAILNFPDL

SEQ ID NO: 172 - protein, 46 aa, Brassica rapa
MNSVSTFSELLCSENESPVNTEGGDYILAASCPKKPAGRKKFQETR

SEQ ID NO: 173 - protein, 46 aa, Brassica rapa
MNSVSTFSELLCSENKSPVNTEGGDYILAASCPKKPAGRKKFQETR

SEQ ID NO: 174 - protein, 46 aa, Brassica napus
MNSVSTFSELLRSENESPVNTEGGDYILAASCPKKPAGRKKFQETR

SEQ ID NO: 175 - protein, 47 aa, Arabidopsis thaliana
MNSFSAFSEMFGSDYESPVSSGGDYSPKLATSCPKKPAGRKKFRETR

SEQ ID NO: 176 - protein, 47 aa, Arabidopsis thaliana
MNSFSAFSEMFGSDYESSVSSGGDYIPTLASSCPKKPAGRKKFRETR

SEQ ID NO: 177 - protein, 44 aa, Arabidopsis thaliana
MNSFSAFSEMFGSDYEPQGGDYCPTLATSCPKKPAGRKKFRETR

SEQ ID NO: 178 - protein, 49 aa, Brassica napus
MNTFPASTEMVGSENESPVTTVVGGDYYPMLAASCPKKPAGRKKFQETR

SEQ ID NO: 179 - protein, 49 aa, Brassica napus; identical to SEQ ID NO: 178

SEQ ID NO: 180 - protein, 49 aa, Brassica oleracea; identical to SEQ ID NO: 178

SEQ ID NO: 181 - protein, 49 aa, Brassica napus
MNTFPASTEMVGSENESPVTTVAGGDYYPMLAASCPKKPAGRKKFQETR

SEQ ID NO: 182 - protein, 49 aa, Brassica napus; identical to SEQ ID NO: 181

SEQ ID NO: 183 - protein, 49 aa, Brassica napus; identical to SEQ ID NO: 181

SEQ ID NO: 184 - protein, 49 aa, Brassica rapa; identical to SEQ ID NO: 181

SEQ ID NO: 185 - protein, 49 aa, Brassica oleracea
MNTFPASTEMVSSENESPVTTVVGGDYYPMLAASCPKKPAGRKKFQETR

SEQ ID NO: 186 - protein, 47 aa, Brassica oleracea
MTSFSTFSELLGSEHESPVTLGEEYCPKLAASCPKKPAGRKKFRETR

SEQ ID NO: 187 - protein, 47 aa, Raphanus sativus
MTTFSTFSEMLGSEYESPVTLGGEYCPKLAASCPKKPAGRKKFRETR

SEQ ID NO: 188 - protein, 45 aa, Brassica napus
MNSVSTFSELLGSENESPVGGDYCPMLAASCPKKPAGRKKFRETR

SEQ ID NO: 189 - protein, 45 aa, Brassica napus; identical to SEQ ID NO: 188

SEQ ID NO: 190 - protein, 45 aa, Brassica oleracea; identical to SEQ ID NO: 188

SEQ ID NO: 191 - protein, 45 aa, Brassica rapa
MNSVSTLSEVLGSQNESPVGGDYCPMLAASCPKKPAGRKKFRETR

SEQ ID NO: 192 - protein, 49 aa, Brassica rapa
MDSISTFPELLGSENESPVTTVVGGDYCPRLAASCPKKPAGRKKFQETR

SEQ ID NO: 193 - protein, 49 aa, Brassica rapa; identical to SEQ ID NO: 192

SEQ ID NO: 194 - protein, 49 aa, Raphanus sativus
MDSISTFSELLGSENESPVTTVVGGDYFPRLAASCPKKPAGRKKFQETR

SEQ ID NO: 195 - protein, 50 aa, Brassica napus
AWRLRIPETTCHKDIQKAAAEAALAFEAEKSDVTMQNGLNMEETTAVASQ

SEQ ID NO: 196 - protein, 50 aa, Brassica napus; identical to SEQ ID NO: 195

SEQ ID NO: 197 - protein, 50 aa, Brassica rapa
AWRLRIPETTCHKDIQKAAAEAALAFEAEKSDVTMQNGLNMEEMTAVASQ

SEQ ID NO: 198 - protein, 50 aa, Brassica napus
AWRLRIPETTCHKDIQKAAAEAALAFEAEKSDVTMQNGQNMEETTAVASQ

SEQ ID NO: 199 - protein, 44 aa, Brassica napus
ASRLRIPETTCHKDIQKAAAEAALAFEAEKSDVTMEETMAVASQ

SEQ ID NO: 200 - protein, 44 aa, Brassica napus; identical to SEQ ID NO: 199

SEQ ID NO: 201 - protein, 44 aa, Brassica oleracea
AWRLRIPETTCHKDIQKAAAEAALAFEAEKSDVTMEETMAVASQ

SEQ ID NO: 202 - protein, 50 aa, Brassica napus; identical to SEQ ID NO: 195

SEQ ID NO: 203 - protein, 50 aa, Brassica oleracea
AWRLRIPETTCHKDIQKAAAEAALAFEAEKSDATMQNGLNMEETTAAASQ

SEQ ID NO: 204 - protein, 31 aa, Brassica rapa
AWRLRIPETTCAKDIQKAAAEAALAFEAEKS

SEQ ID NO: 205 - protein, 31 aa, Raphanus sativus; identical to SEQ ID NO: 204

SEQ ID NO: 206 - protein, 33 aa, Brassica napus
AWRLRIPETTCAKDIQKAAAEAALAFEAEKSDT

SEQ ID NO: 207 - protein, 33 aa, Brassica oleracea; identical to SEQ ID NO: 206

SEQ ID NO: 208 - protein, 33 aa, Brassica napus; identical to SEQ ID NO: 206

SEQ ID NO: 209 - protein, 34 aa, Brassica rapa
AWRLRIPETTCAKDIQKAAAEAALAFEAEKSDTT

SEQ ID NO: 210 - protein, 33 aa, Brassica rapa; identical to SEQ ID NO: 206

SEQ ID NO: 211 - protein, 33 aa, Brassica oleracea
AWRLRIPETTCAKDIQKAAAEAALAFGAEKSDT

SEQ ID NO: 212 - protein, 26 aa, Brassica juncea
AWRLRISETTCPKEIQKAAAEAAVAF

SEQ ID NO: 213 - protein, 26 aa, Brassica juncea
AWRLRIPETTCPKEIQKAAAEAAVAF

SEQ ID NO: 214 - protein, 26 aa, Brassica napus
AWRLRIPETTCPKEIQKAAAEAAVAF

SEQ ID NO: 215 - protein, 26 aa, Raphanus sativus
AWRLRIPETTCPKDIQKAAAEAAVAF

SEQ ID NO: 216 - protein, 26 aa, Arabidopsis thaliana
AWRLRIPESTCAKDIQKAAAEAALAF

SEQ ID NO: 217 - protein, 26 aa, Arabidopsis thaliana
AWRLRIPESTCAKEIQKAAAEAALNF

SEQ ID NO: 218 - protein, 26 aa, Arabidopsis thaliana
AWRLRIPESTCAKDIQKAAAEAALAF

SEQ ID NO: 219 - protein, 49 aa, Brassica napus
AEVNDTTTEHGMNMEEATAVASQAEVNDTTTDHGVDMEETMVEAVFTGE

SEQ ID NO: 220 - protein, 49 aa, Brassica napus; identical to SEQ ID NO: 219

SEQ ID NO: 221 - protein, 49 aa, Brassica rapa
AEVNDTTTEHGMNMEEATAVASQAEVNDTTTDHGVDMEETMVEAVFTEE

SEQ ID NO: 222 - protein, 49 aa, Brassica napus; identical to SEQ ID NO: 219

SEQ ID NO: 223 - protein, 49 aa, Brassica napus
AEVNDTTTDHGMNMEEATAVASQAEVNDTTTDHGVDMEETMVEAVFTGE

SEQ ID NO: 224 - protein, 49 aa, Brassica napus
AEVNDTTTDHGMNMEEATAVASQAEVNDTTTDHGVDMEETMVEAVFTEE

SEQ ID NO: 225 - protein, 49 aa, Brassica oleracea; identical to SEQ ID NO: 224

SEQ ID NO: 226 - protein, 49 aa, Brassica napus; identical to SEQ ID NO: 221

SEQ ID NO: 227 - protein, 49 aa, Brassica oleracea
  VARIANT (1)...(49): Xaa = Any Amino Acid
TEVSDTTTDHGMNMEETTAVASQAEVNDXXTDHGVDMEETMVEAVFTEE

SEQ ID NO: 228 - protein, 41 aa, Brassica rapa
DHGMNMKNTTAVASQVEVNDTTTDHGVDMEETLVEAVFTEE

SEQ ID NO: 229 - protein, 41 aa, Raphanus sativus; identical to SEQ ID NO: 228

SEQ ID NO: 230 - protein, 37 aa, Brassica napus
TTNDHGMNMASQAEVNDTTDHGLDMEETMVEAVFTEE

SEQ ID NO: 231 - protein, 37 aa, Brassica oleracea; identical to SEQ ID NO: 230

SEQ ID NO: 232 - protein, 37 aa, Brassica napus
TTNDHGMNMASQVEVNDTTDHDLDMEETIVEAVFREE

SEQ ID NO: 233 - protein, 37 aa, Brassica rapa
TTNDHGMNMASQVEVNDTTDHDLDMEETMVEAVFREE

SEQ ID NO: 234 - protein, 44 aa, Brassica rapa
TTNDRGMNMEETSAVASPAELNDTTADHGLDMEETMVEAVFRDE

SEQ ID NO: 235 - protein, 44 aa, Brassica oleracea
TTNDQGMNMEEMTVVASQAEVSDTTTYHGLDMEETMVEAVFAEE

SEQ ID NO: 236 - protein, 26 aa, Brassica juncea
QAELNDTTADHGLDVEETIVEAIFTE

SEQ ID NO: 237 - protein, 26 aa, Brassica juncea
QAELNDTTADHGLDVEETIVEAIFTE

SEQ ID NO: 238 - protein, 26 aa, Brassica napus
KAEINNTTADHGIDVEETIVEAIFTE

SEQ ID NO: 239 - protein, 26 aa, Raphanus sativus
QAEINDTTTDHGLDIEETIVEAIFTE

SEQ ID NO: 240 - protein, 28 aa, Arabidopsis thaliana
QDETCDTTTTDHGLDMEETMVEAIYTPE

SEQ ID NO: 241 - protein, 28 aa, Arabidopsis thaliana
QDEMCHMTTDAHGLDMEETLVEAIYTPE

SEQ ID NO: 242 - protein, 27 aa, Arabidopsis thaliana
QDEMCDATTDHGFDMEETLVEAIYTAE

SEQ ID NO: 243 - protein, 49 aa, Brassica napus
QSEGFNMAKESTVEAAVVTEEPSKGSYMDEEWMLEMPTLLADMAEGMLL

SEQ ID NO: 244 - protein, 49 aa, Brassica napus; identical to SEQ ID NO: 243

SEQ ID NO: 245 - protein, 49 aa, Brassica rapa; identical to SEQ ID NO: 243

SEQ ID NO: 246 - protein, 49 aa, Brassica napus; identical to SEQ ID NO: 243

SEQ ID NO: 247 - protein, 49 aa, Brassica napus; identical to SEQ ID NO: 243

SEQ ID NO: 248 - protein, 49 aa, Brassica napus
QSEGFNMAEESTVEAAVVTDELSKGFYMDEEWTYEMPTLLADMAAGMLL

SEQ ID NO: 249 - protein, 49 aa, Brassica oleracea; identical to SEQ ID NO: 248

SEQ ID NO: 250 - protein, 49 aa, Brassica napus; identical to SEQ ID NO: 248

SEQ ID NO: 251 - protein, 49 aa, Brassica oleracea
QSEGFNMAKESTAEAAVVTEELSKGVYMDEEWTYEMPTLLADMAAGMLL

SEQ ID NO: 252 - protein, 48 aa, Brassica rapa
QREGFYMTEETRVEGVVTEEQNNWFYMDEEWMFGMPTLLVDMAEGMLI

SEQ ID NO: 253 - protein, 48 aa, Raphanus sativus
QREGFYMTEETRVEGVVTEEQNNWFYMDEEWMFGMPTLLVDMAEGMLL

SEQ ID NO: 254 - protein, 49 aa, Brassica napus
QRDGFYMAEETTVEGVVPEEQMSKGFYMDEEWMFGMPTLLADMAAGMLL

SEQ ID NO: 255 - protein, 49 aa, Brassica oleracea; identical to SEQ ID NO: 254

SEQ ID NO: 256 - protein, 49 aa, Brassica napus
QREGFYMAEETTVVGVVPEEQMSKGFYMDEEWMFGMPTLLADMAAGMLL

SEQ ID NO: 257 - protein, 49 aa, Brassica rapa
QREGFYMAEETTVEGIVPEEQMSKGFYMDEEWMFGMPTLLADMAAGMLL

SEQ ID NO: 258 - protein, 49 aa, Brassica rapa
QREGFYMAEETTVEGVVPEEQMSKGFYMDEEWTFEMPRLLADMAEGMLL

SEQ ID NO: 259 - protein, 48 aa, Brassica oleracea
QREGFYLAEETTVEGVVTEEQSKGFYMDEEWTFGMQSFLADMAEGMLF

SEQ ID NO: 260 - protein, 29 aa, Brassica juncea
ESSEGFYMDEEFMFGMPTLWASMAEGMLL

SEQ ID NO: 261 - protein, 29 aa, Brassica juncea
ESSEGFYMAEEFMFGMPTLWASVAEGMLL

SEQ ID NO: 262 - protein, 30 aa, Brassica napus
ENNDGFYMDEEESMFGMPALLASMAEGMLL

SEQ ID NO: 263 - protein, 29 aa, Raphanus sativus
VNNDEFYMDEESMFGMPSLLASMAEGMLL

SEQ ID NO: 264 - protein, 29 aa, Arabidopsis thaliana
QSEGAFYMDEETMFGMPTLLDNMAEGMLL

SEQ ID NO: 265 - protein, 29 aa, Arabidopsis thaliana
QSQDAFYMDEEAMLGMSSLLDNMAEGMLL

SEQ ID NO: 266 - protein, 29 aa, Arabidopsis thaliana
QSENAFYMHDEAMFEMPSLLANMAEGMLL

SEQ ID NO: 267 - protein, 50 aa, Brassica napus; identical to SEQ ID NO: 195

SEQ ID NO: 268 - protein, 50 aa, Brassica napus; identical to SEQ ID NO: 195

SEQ ID NO: 269 - protein, 50 aa, Brassica rapa; identical to SEQ ID NO: 197

SEQ ID NO: 270 - protein, 50 aa, Brassica napus; identical to SEQ ID NO: 198

SEQ ID NO: 271 - protein, 44 aa, Brassica napus; identical to SEQ ID NO: 199

SEQ ID NO: 272 - protein, 44 aa, Brassica napus; identical to SEQ ID NO: 199

SEQ ID NO: 273 - protein, 44 aa, Brassica oleracea; identical to SEQ ID NO: 201

SEQ ID NO: 274 - protein, 50 aa, Brassica napus; identical to SEQ ID NO: 195

SEQ ID NO: 275 - protein, 50 aa, Brassica oleracea; identical to SEQ ID NO: 203

SEQ ID NO: 276 - protein, 50 aa, Brassica napus
AEVNDTTTEHGMNMEEATAVASQAEVNDTTTDHGVDMEETMVEAVFTGEQ

SEQ ID NO: 277 - protein, 50 aa, Brassica napus; identical to SEQ ID NO: 276

SEQ ID NO: 278 - protein, 50 aa, Brassica rapa
AEVNDTTTEHGMNMEEATAVASQAEVNDTTTDHGVDMEETMVEAVFTEEQ

SEQ ID NO: 279 - protein, 50 aa, Brassica napus; identical to SEQ ID NO: 276

SEQ ID NO: 280 - protein, 50 aa, Brassica napus
AEVNDTTTDHGMNMEEATAVASQAEVNDTTTDHGVDMEETMVEAVFTGEQ

SEQ ID NO: 281 - protein, 50 aa, Brassica napus
AEVNDTTTDHGMNMEEATAVASQAEVNDTTTDHGVDMEETMVEAVFTEEQ

SEQ ID NO: 282 - protein, 50 aa, Brassica oleracea; identical to SEQ ID NO: 281

SEQ ID NO: 283 - protein, 50 aa, Brassica napus; identical to SEQ ID NO: 278

SEQ ID NO: 284 - protein, 50 aa, Brassica oleracea
TEVSDTTTDHGMNMEETTAVASQAEVNDTTTDHGVDMEETMVEAVFTEEQ

SEQ ID NO: 285 - protein, 50 aa, Brassica napus
SEGFNMAKESTVEAAVVTEEPSKGSYMDEEWMLEMPTLLADMAEGMLLPP

SEQ ID NO: 286 - protein, 50 aa, Brassica rapa; identical to SEQ ID NO: 285

SEQ ID NO: 287 - protein, 50 aa, Brassica napus; identical to SEQ ID NO: 285

SEQ ID NO: 288 - protein, 48 aa, Brassica napus
SEGFNMAKESTVEAAVVTEEPSKGSYMDEEWMLEMPTLLADMAEGMLL

SEQ ID NO: 289 - protein, 50 aa, Brassica napus; identical to SEQ ID NO: 285

SEQ ID NO: 290 - protein, 50 aa, Brassica napus
SEGFNMAEESTVEAAVVTDELSKGFYMDEEWTYEMPTLLADMAAGMLLPP

SEQ ID NO: 291 - protein, 50 aa, Brassica oleracea; identical to SEQ ID NO: 290

SEQ ID NO: 292 - protein, 50 aa, Brassica napus; identical to SEQ ID NO: 290

SEQ ID NO: 293 - protein, 50 aa, Brassica oleracea
SEGFNMAKESTAEAAVVTEELSKGVYMDEEWTYEMPTLLADMAAGMLLPP

SEQ ID NO: 294 - protein, 205 aa, Medicago truncatula
  G3362 polypeptide
MFTMNQFSESHDPCSSSSERFLAETMPKKRAGRKKFRETRHPVYRGVRKRDSGKWVCEVREPNKKTRIWLGTFPTPEMAARAHDVAAIALRGRSACLNFADSAWKLPVPATSEARDIQKAAAEAAEAFRPESVFENSEERKDSEPSSTVAVSETVMEQREEEEDTVPEYLRNMVLMSPAHYWGSDCGVADVEFDETEVSLWSYSF

SEQ ID NO: 295 - protein, 215 aa, Medicago truncatula
  G3364 polypeptide
MFTTNNSSYSHSISSEASSSYYNSLPESEIRLAASNPKKRAGRKIFKETRHPVYRGVRKRNLDKWVCEMREPNTKTRIWLGTFPTAEMAARAHDVAAMALRGRYACLNFADSVWRLPIPATSSIKDIQKAATKAAEAFRPDNTIMITNIETVVAVVATKELNMFCVEEEEEMLNMPEFWRNMALMSPTHSFEYHDQYEDFHFQDFQDDEVSLWNF

SEQ ID NO: 296 - protein, 220 aa, Medicago truncatula
  G3365 polypeptide
MKSSLDESSYVENNSSSSEILLASEQPKKRAGRRKFKETRHPVYRGVRRRNNNNNKWVCEVRVPNDKSTRIWLGTYPTPEMAARAHDVAALALRGKSACLNFADSAWRLALPATNNAKEIRKMAAEAALAFAVVADSKEQTMISNCDVNSVGVMEVDNKPLQGLCVEVPEEEMLHDWFRSMADEPLRSPITPFIRHGRDQWNNVDIDQVDAEVSLWNFTI

SEQ ID NO: 297 - protein, 227 aa, Medicago truncatula
  G3366 polypeptide
MYPTTNSVSSSSSDMSLPNSEGSHWMSICNEEMRLAATTPKKRAGRKKFKETRHPVYRGVRKRNLDKWVCEMREPNKKTKIWLGTFPTAEMAARAHDVAAMALRGRYACLNFADSAWRLPKPATTQAKDIQKAATEAAEAFRPDKTLLTNHNDNDNDNDKENDMAVVATATEEQSMICMEEKEEGVMNMQEMWSNMALMSPTHSLGYYEYQYINEDFQDEEVSLWSF

SEQ ID NO: 298 - protein, 248 aa, Medicago truncatula
  G3367 polypeptide
MISTNNSSYSHSISSKDFSPFDASSPDSEVRLASSNPKKRAGRKIFKETRHPVYRGVRKRNLNKWVCEMREPNTKNRIWLGTFPTAEMAARAHDVAAIALRGRYACLNFADSVWRLPIPATSAIKDIQKAATKAAEAFRPDNTLMTSDIDTVVAVVATQELNMFRVEVEEEEVLNMPELWRNMALMSPTHSFGYHDQYEDIHIQDFQDDEDFKKRSVTTIWAVTSIGVHSLHFTVISRIVMRTLLLCV

SEQ ID NO: 299 - protein, 229 aa, Medicago truncatula
  G3368 polypeptide
MDFFMSSFSDYSDTSSSETASSNRTSSSEVILAPARPKKRAGRRVFKETRHPVYRGVRRRKNNKWVCEMRVPNNIVNKNNKSRIWLGTYPTPEMAARAHDVAALTLKGKSACLNFADSAWRLRLPESNDATEIRRAAMEAAQLFAVEDKQCCVTVEDGVFMDMEDSKNMLEAQVPVVSSEFEDMHHLLLSIANEPLRSAPPSPTNYGSYNWGDMEIFDTQLVSLWNFSI

SEQ ID NO: 300 - protein, 260 aa, Medicago truncatula
  G3369 polypeptide
MDMFTNNNSYSHPFSPTCSESSFPNSEGSQGMSISNEEVRLAATTPKKRAGRKKFKETRHPVYRGVRKRNLDKWVCEMREPNKKTKIWLGTFPTAEMAARAHDVAAMALRGRYACLNFADSAWRLPIPATTQAKDIQKAAAQAAEAFRPDKTSITNDIDTAISTSATAEQSRTFMEEEEEGVMNMPELLRNMALMSPTHSSGYNEYENIHVQDFQDLQDFQDEEVLIKHKVLLIPSISIYERRIEVWYVKISVNFISYLN

SEQ ID NO: 301 - protein, 218 aa, Oryza sativa
  G3370 polypeptide
MEVEEAAYRTVWSEPPKRPAGRTKFRETRHPVYRGVRRRGGRPGAAGRWVCEVRVPGARGSRLWLGTFATAEAAARAHDAAALALRGRAACLNFADSAWRMPPVPASAALAGARGVRDAVAVAVEAFQRQSAAPSSPAETFADDGDEEEDNKDVLPVAAAEVFDAGAFELDDGFRFGGMDAGSYYASLAQGLLVEPPAAGAWWEDGELAGSDMPLWSY

SEQ ID NO: 302 - protein, 253 aa, Oryza sativa
  G3371 polypeptide
MEKNTAASGQLMTSSAEATPSSPKRPAGRTKFQETRHLVFRGVRWRGCAGRWVCKVRVPGSRGDRFWIGTSDTAEETARTHDAAMLALCGASASLNFADSAWLLHVPRAPVVSGLRPPAARCATRCLQGHRRVPAPGRGSNATATATSGDAASTAPPSAPVLSAKQCEFIFLSSLDCWMLMSKLISSSRAKGSLCLRKNPISFCMVTNSYTALLLEYIILQMNSMIVLIHELSKYQVFLLLTMITHHLFQWRR

SEQ ID NO: 303 - protein, 214 aa, Oryza sativa
  G3372 polypeptide
MEYYEQEEYATVTSAPPKRPAGRTKFRETRHPVYRGVRRRGPAGRWVCEVREPNKKSRIWLGTFATAEAAARAHDVAALALRGRGACLNFADSARLLRVDPATLATPDDIRRAAIELAESCPHDAAAAAASSSAAAVEASAAAAPAMMMQYQDDMAATPSSYDYAYYGNMDFDQPSYYYDGMGGGGEYQSWQMDGDDDGGAGGYGGGDVTLWSY

SEQ ID NO: 304 - protein, 219 aa, Oryza sativa
  G3373 polypeptide
MDTEDTSSASSSSVSPPSSPGGGHHHRLPPKRRAGRKKFRETRHPVYRGVRARAGGSRWVCEVREPQAQARIWLGTYPTPEMAARAHDVAAIALRGERGAELNFPDSPSTLPRARTASPEDIRLAAAQAAELYRRPPPPLALPEDPQEGTSGGGATATSGRPAAVFVDEDAIFDMPGLIDDMARGMMLTPPAIGRSLDDWAAIDDDDDHYHMDYKLWMD

SEQ ID NO: 305 - protein, 251 aa, Oryza sativa
  G3374 polypeptide
MCTSKLEEITGEWPPPALQAASTTSSSEPCRRLSPPSSKRPAGRTKFHETRHPVFRGVRRRGRAGRWVCEVRVPGRRGCRLWLGTFDAADAAARAHDAAMLALRGRAAACLNFADSAWLLAVPPPATLRCAADVQRAVARALEDFEQRESSSSVFPLAIDVVAEDAMSATSEPSAASDDDAVTSSSSTTDADEEASPFELDVVSDMGWSLYYASLAEGLLMEPPASGASSDDDDDAIVDSGDIADVSLWSY

SEQ ID NO: 306 - protein, 219 aa, Oryza sativa
  G3375 polypeptide
MEWAYYGSGYSSSGTPSPVGGDGDEDSYMTVSSAPPKRRAGRTKFKETRHPVYKGVRSRNPGRWVCEVREPHGKQRIWLGTFETAEMAARAHDVAAMALRGRAACLNFADSPRRLRVPPLGAGHEEIRRAAVEAAELFRPAPGQHNAAAEAAAAVAAQATAASAELFADFPCYPMDGLEFEMQGYLDMAQGMLIEPPPLAGQSTWAEEDYDCEVNLWSY

SEQ ID NO: 307 - protein, 224 aa, Oryza sativa
  G3376 polypeptide
MDVSAALSSDYSSGTPSPVAADADDGSSAYMTVSSAPPKRRAGRTKFKETRHPVFKGVRRRNPGRWVCEVREPHGKQRIWLGTFETAEMAARAHDVAALALRGRAACLNFADSPRRLRVPPIGASHDDIRRAAAEAAEAFRPPPDESNAATEVAAAASGATNSNAEQFASHPYYEVMDDGLDLGMQGYLDMAQGMLIDPPPMACDPAVGGGEDDNDGEVQLWSY

SEQ ID NO: 308 - protein, 236 aa, Oryza sativa
  G3377 polypeptide
MEKNTTAMGQLMSSSATTAATATGPASPKRPAGRTKFQETRHPVFRGVRRRGRAGRWVCEVRVPGSRGDRLWVGTFDTAEEAARAHDAAMLALCGASASLNFADSAWLLHVPRAPVASGHDQLPDVQRAASEAVAEFQRRGSTAATATATSGDAASTAPPSSSPVLSPNDDNASSASTPAVAAALDHGDMFGGMRTDLYFASLAQGLLIEPPPPPTTAEGFCDDEGCGGAEMELWS

SEQ ID NO: 309 - protein, 286 aa, Oryza sativa
  G3378 polypeptide
MHTYIYTPRAAELEHSHSASATRSHSLGQAPPSLDRSRAAMDMAGHEVNSSSSSSGAESSSSSSGRQQYKKRPAGRTKFRETRHPVYRGVRRRGGAGRWVCEVRVPGKRGARLWLGTYVTAEAAARAHDAAMIALRGGAGGGGAACLNFQDSAWLLAVPPAAPSDLAGVRRAATEAVAGFLQRNKTTNGASVAEAIDEATSGVSKPPPLANNADSSETPGPSSIDGTADTAAGAALDMFELDFFGEMDYDTYYASLAEGLLMEPPPAATALWDNGDEGADIALWSY

SEQ ID NO: 310 - protein, 238 aa, Oryza sativa
  G3379 polypeptide
MCGIKQEMSGESSGSPCSSASAERQHQTVWTAPPKRPAGRTKFRETRHPVFRGVRRRGNAGRWVCEVRVPGRRGCRLWLGTFDTAEGAARAHDAAMLAINAGGGGGGGACCLNFADSAWLLAVPRSYRTLADVRHAVAEAVEDFFRRRLADDALSATSSSSTTPSTPRTDDDEESAATDGDESSSPASDLAFELDVLSDMGWDLYYASLAQGMLMEPPSAALGDDGDAILADVPLWSY

SEQ ID NO: 311 - protein, 246 aa, Zea mays
  G3438 polypeptide
MDMGRHQLQLQHAASSSSTSASSSSEQDKPLCCSGPKKRPAGRTKFRETRHPVFRGVRRRGAAGRWVCEVRVPGRRGARLWLGTYLGAEAAARAHDAAMLALGRGAACLNFPDSAWLLAVPPPPALSGGLDGARRAALEAVAEFQRRRFGAAAADEATSGTSPPSSSSSATKPAPAIERVPVEASETVALDGAVFEPDWFGDMDLDLYYASLAEGLLVEPPPPPPPAAWDHGDCCDSGADVALWSY

SEQ ID NO: 312 - protein, 256 aa, Zea mays
  G3439 polypeptide
MDMGRLQLQLQHAASSSSTSASSSSSSEQNKLAWSPSSPQPPKKRPAGRTKFRETRHPVFRGVRRRGAAGRWVCEVRVPGRRGARLWLGTYLGAEAAARAHDAAMLALGRGAACLNFPDSAWLLAVPPPPALSGGLDGARRAALEAVAEFQRRRFGAVAADEATSGTSPPSSSSSPSGTYVSQAPAPAIERVPVEASETAALDGAVFEPDWFRDMDLDLYYASLAEGLLVEPPPPPAAWDHGDCSHSGADVALWSY

SEQ ID NO: 313 - protein, 231 aa, Zea mays
  G3440 polypeptide
MCPTKKGMTGEPSSPCSSASASTLPEHHQTVWTSPPKRPAGRTKFRETRHPVFRGVRRRGSAGRWVCEVRVPGRRGCRLWLGTFDTAEAAARAHDAAMLALAGAGACCLNFADSAWLLAVPASCASLAEVRHAVADAVDDFLRHQLVPEDDALAATPSSPSSEDGNTSDGGESSSDSSPPTGASPFEFDVFNDMSWDLHYASLAQGLLVEPPSAVTAFMDEGFADVPLWSY

SEQ ID NO: 314 - protein, 231 aa, Zea mays
  G3441 polypeptide
MEYAAVGYGYGYGYDERQEPAESADGGGGGDDEYATVLSAPPKRPAGRTKFRETRHPVYRGVRRRGPAGRWVCEVREPNKKSRIWLGTFATPEAAARAHDVAALALRGRAACLNFADSARLLQVDPATLATPDDIRRAAIQLADAASQQDETAAVAADVVAPSQADDVAAAAAAAAAAAMYGGGMEFDHSYCYDDGMVSGSSDCWQSGGGGWHSSVDGDDDGAGDMTLWSY

SEQ ID NO: 315 - protein, 267 aa, Zea mays
  G3442 polypeptide
MDTAGLVQHATSSSSTSTSASSSSSEQQSRKAAWPPSTASSPQQPPKKRPAGRTKFRETRHPVFRGVRRRGAAGRWVCEVRVPGRRGARLWLGTYLAAEAAARAHDAAILALQGRGAGRLNFPDSARLLAVPPPSALPGLDDARRAALEAVAEFQRRSGSGSGAADEATSGASPPSSSPSLPDVSAAGSPAAALEHVPVKADEAVALDLDGDVFGPDWFGDMGLELDAYYASLAEGLLVEPPPPPAAWDHGDCCDSGAADVALWSYY

SEQ ID NO: 316 - protein, 215 aa, Medicago sativa
  G3497 polypeptide
MLTTNNSSNSQSISSTASSSYDMSTPNLEVRLAASNPKKRAGRKIFKETRHPVYRGVRKRNLDKWVCEMREPNTKTRIWLGTFPTAEMAAQAHDVAAMALRGRYACVNFADSVWRLPIPATSKIKDIQKAAAEAAEAFRPDKTLMTNDIDTVVAVVATKELNMFCVEVEDDVLNMPELWRNMALMSRTHSFGYDDQYEDIHVQDFQDDEVSLWNF

SEQ ID NO: 317 - protein, 214 aa, Medicago sativa
  G3498 polypeptide
MFTTNNSSYSHSISSKASSSYYNSLPDSEIRLAASNPKKRAGRKIFKETRHPVYRGVRKRNLDKWVCEMREPNMKTRIWLGTFPTADMAARAHDVAAKALRGRYACLNFAYSVWRLPIPATSSIKDIQKAATKAAEAFRPDHTIMITDIETVVAVVATKDLNIFCGEEEHEMLDMSELWRNMALMSPTHSFSNDHYEDIQAQDFQDDEVSLWNY

SEQ ID NO: 318 - protein, 215 aa, Medicago sativa
  G3499 polypeptide
MKSSLDESSYVENNSSSSETSCYEEILLASERPKKPAGRRKFKETRHPVYRGVRRRNNNKWVCEVRVPNDKSTRIWLGTYPTPEMAARAHDVAALALRGKSACLNFANSAWRLALPETNNAKEIRKMAAEAALAFAVEDSKEQIMISNCDVCSSDNVMEVDNKPLQGLCVEVPEQEMLHDWFRSMADEPLRSPMTPFIRYGIGRDHSNNVDVDPC

SEQ ID NO: 319 - protein, 7 aa, Artificial sequence
  oligopeptide
PKXXAGR

SEQ ID NO: 320 - protein, 6 aa, Artificial sequence
  oligopeptide
AGRXKF

SEQ ID NO: 321 - protein, 5 aa, oligopeptide
  oligopeptide
ETRHP

SEQ ID NO: 322 - protein, 7 aa, Artificial sequence
  oligopeptide
PKKXAGR

SEQ ID NO: 323 - protein, 7 aa, Artificial sequence
  oligopeptide
PKKRAGR

SEQ ID NO: 324 - protein, 6 aa, Artificial sequence
  oligopeptide
AGRKXF

SEQ ID NO: 325 - protein, 6 aa, Artificial sequence
  oligopeptide
AGRKKF

SEQ ID NO: 326 - protein, 7 aa, Artificial sequence
  oligopeptide
PKKPAGR

SEQ ID NO: 327 - protein, 79 aa, Arabidopsis thaliana
  CBF1 AP2 domain and flanking signature sequences
PKKPAGRKKFRETRHPIYRGVRQRNSGKWVSEVREPNKKTRIWLGTFQTAEMAARAHDVAALALRGRSACLNFADSAWR

SEQ ID NO: 328 - protein, 79 aa, Arabidopsis thaliana
  CBF2 AP2 domain and flanking signature sequences
PKKPAGRKKFRETRHPIYRGVRQRNSGKWVCELREPNKKTRIWLGTFQTAEMAARAHDVAAIALRGRSACLNFADSAWR

SEQ ID NO: 329 - protein, 79 aa, Arabidopsis thaliana
  CBF3 AP2 domain and flanking signature sequences
PKKPAGRKKFRETRHPIYRGVRRRNSGKWVCEVREPNKKTRIWLGTFQTAEMAARAHDVAALALRGRSACLNFADSAWR

SEQ ID NO: 330 - protein, 79 aa, Arabidopsis thaliana
  G912 AP2 domain and flanking signature sequences
PKKRAGRKKFRETRHPIYRGVRQRNSGKWVCEVREPNKKSRIWLGTFPTVEMAARAHDVAALALRGRSACLNFADSAWR

SEQ ID NO: 331 - protein, 210 aa, Lycopersicon esculentum
  Lycopersicon esculentum CBF1 polypeptide
MNIFETYYSDSLILTESSSSSSSSSFSEEEVILASNNPKKPAGRKKFRETRHPIYRGIRKRNSGKWVCEVREPNKKTRIWLGTFPTAEMAARAHDVAALALRGRSACLNFSDSAWRLPIPASSNSKDIQKAAAQAVEIFRSEEVSGESPETSENVQESSDFVDEEAIFFMPGLLANMAEGLMLPPPQCAEMGDHCVETDAYMITLWNYSI

SEQ ID NO: 332 - protein, 220 aa, Lycopersicon esculentum
  Lycopersicon esculentum CBF2 polypeptide
MDIFESYYSNSFVESLLSSSLSISDTNNLNHYSPNEEVIILASNNPKKPAGRKKFRETRHPVYRGIRKRNSGKWVCEVREPNKKTRIWLGTFPTAEMAARAHDVAAIALRGRSACLNFADSYWRLPIPASSNSKDIQKAAAEAAEIFRSEEVSGESPETSENVQESSDFVDEEALFSMPGLLANMAEGLMLPPPQCLEIGDHYVELADVHAYMPLWNYSI

(19) United States
(12) Patent Application Publication
(10) Pub. No.: US 2003/0233680 A1
(43) Pub. Date: Dec. 18, 2003

(54) Methods for modifying plant biomass and cell protectant levels

(76) Inventors: Michael Thomashow, East Lansing, MI (US); Eric J. Stockinger, Shreve, OH (US); Kirsten Jaglo, San Francisco, CA (US); Sarah Jane Gilmour, Mason, MI (US); Daniel Zarka, Lansing, MI (US); Cai-Zhong Jiang, Fremont, CA (US); James Zhang, Palo Alto, CA (US); Volker Haake, Menlo Park, CA (US); Jose Luis Riechmann, Pasadena, CA (US); Bradley K. Sherman, Berkeley, CA (US); Michael Fromm, Lincoln, NE (US); Karen Century, Albany, CA (US)

Correspondence Address: MATTHEW KASER, Mendel Biotechnology, Inc., 21375 Cabot Blvd., Hayward, CA, 94545, US

(21) Appl. No.: 10/421,138

(22) Filed: Apr. 23, 2003

Related U.S. Application Data
(63) Continuation-in-part of application No. 09/580,377, filed on May 26, 2000, now abandoned.
(63) Continuation-in-part of application No. 09/773,990, filed on Feb. 1, 2001, now abandoned.
(63) Continuation-in-part of application No. 09/996,140, filed on Nov. 26, 2001.
(63) Continuation-in-part of application No. 09/601,802, filed on Sep. 15, 2000.
(63) Continuation-in-part of application No. PCT/US99/01895, filed on Jan. 28, 1999.
(63) Continuation-in-part of application No. 09/198,119, filed on Nov. 23, 1998, now Pat. No. 6,417,428.
(63) Continuation-in-part of application No. 09/018,233, filed on Feb. 3, 1998, now abandoned.
(63) Continuation-in-part of application No. 09/017,816, filed on Feb. 3, 1998, now abandoned.
(63) Continuation-in-part of application No. 09/018,235, filed on Feb. 3, 1998, now abandoned.
(63) Continuation-in-part of application No. 09/017,575, filed on Feb. 3, 1998, now abandoned.
(63) Continuation-in-part of application No. 09/018,227, filed on Feb. 3, 1998, now abandoned.
(63) Continuation-in-part of application No. 09/018,234, filed on Feb. 3, 1998, now abandoned.
(63) Continuation-in-part of application No. 08/706,270, filed on Sep. 4, 1996, now Pat. No. 5,892,009.
(63) Continuation-in-part of application No. 09/713,994, filed on Nov. 16, 2000.
(60) Provisional application No. 60/197,899, filed on Apr. 17, 2000.
(60) Provisional application No. 60/227,439, filed on Aug. 22, 2000.
(60) Provisional application No. 60/166,228, filed on Nov. 17, 1999.

(51) Int. Cl. A01H 1/00; C12N 15/82

(52) U.S. Cl. 800/289

(57) ABSTRACT

Methods for increasing the biomass of a plant and levels of a cell protectant in a plant, such as sucrose or proline, or plant cell are provided. The methods comprise altering expression levels of a polynucleotide comprising a sequence encoding a C-repeat/DRE binding factor (CBF)-related polypeptide in a plant and identifying plants so altered.. These methods may be used to enhance yield, or cold, freezing, drought, and high salt tolerance of a plant or plant cell.

### RELATED APPLICATION INFORMATION

This application is a continuation-in-part of the following U.S. applications: U.S. application Ser. No. 09/773,990, filed: Feb. 1, 2001, which in turn claimed priority from U.S. application Ser. No. 09/580,377, filed: May 26, 2000, which in turn claimed priority from U.S. provisional Application Serial No. 60/165,860, filed: Nov. 16, 1999, now abandoned; U.S. application Ser. No. 09/996,140, filed Nov. 26, 2001; U.S. application Ser. No. 09/601,802, filed: Sep. 15, 2000, which in turn claimed priority from PCT application No.: PCT/US99/01895, filed: Jan. 28, 1999, now abandoned, which in turn claimed priority from U.S. application Ser. No. 09/198,119, filed: Nov. 23, 1998, which issued as U.S. Pat. No. 6,417,428, which, in turn, claimed priority in part to U.S. application Ser. No. 09/018,233, filed: Feb. 3, 1998, now abandoned, U.S. application Ser. No. 09/017,816, filed: Feb. 3, 1998, now abandoned, U.S. application Ser. No. 09/018,235, filed: Feb. 3, 1998, now abandoned, U.S. application Ser. No. 09/017,575, filed: Feb. 3, 1998, now abandoned, U.S. application Ser. No. 09/018,227, filed: Feb. 3, 1998, now abandoned, and U.S. application Ser. No. 09/018,234, filed: Feb. 3, 1998, now abandoned, all six of which claimed priority in part to U.S. application Ser. No. 08/706,270, filed: Sep. 4, 1996, which issued as U.S. Pat. No. 5,892,009; U.S. application Ser. No. 09/627,348, filed Jul. 28, 2000, which in turn claimed priority from No. 60/148,200 filed Aug. 10, 1999, now abandoned, and U.S. provisional Application Serial No. 60/165,860 filed Nov. 16, 1999, now abandoned; U.S. application Ser. No. 09/713,994, filed: Nov. 16, 2000, which in turn claimed priority from No. 60/197,899 filed Apr. 17, 2000, now abandoned, U.S. provisional Application Serial No. 60/227,439 filed Aug. 22, 2000, now abandoned, and from U.S. provisional Application Serial No. 60/166,228 filed Nov. 17, 1999, now abandoned; each of which are incorporated herein by reference in their entirety.

This invention was supported by a subcontract under a USDA/CSREES Cooperative Agreement and an NSF/SBIR grant. The US Government has certain rights in this invention.

### FIELD OF THE INVENTION

The present invention relates to a method for modifying the biomass of a plant. The method is particularly useful for modifying the leaf or root biomass of a plant. The invention is also useful for modifying the levels of a cell protectant, such as a cryoprotectant, an osmoprotectant, or the like, in a cell or a plant to improve its response to environmental stresses, including but not limited to cold or freezing stress, drought stress, or salt stress.

### BACKGROUND OF THE INVENTION

Increasing the biomass of a plant has several commercial applications. For example, increasing plant leaf biomass may increase the yield of leafy vegetables for human or animal consumption. Additionally, increasing leaf biomass can be used to increase production of plant-derived pharmaceutical or industrial products. By increasing plant biomass, increased production levels of the products may be obtained from the plants. Tobacco leaves, in particular, have been employed as plant factories to generate such products. Furthermore, it may be desirable to increase crop yields of plants by increasing total plant photosynthesis. An increase in total plant photosynthesis is typically achieved by increasing leaf area of the plant. Additional photosynthetic capacity may be used to increase the yield derived from particular plant tissue, including the leaves, roots, fruits or seed. In addition, the ability to modify the biomass of the leaves may be useful for permitting the growth of a plant under decreased light intensity or under high light intensity. Modification of the biomass of another tissue, such as roots, may be useful to improve a plant's ability to grow under harsh environmental conditions, including drought or nutrient deprivation, because the roots may grow deeper into the ground.

Due to the commercial consequences of environmental stress damage to crops, there is an interest in understanding how to improve a plant's tolerance to environmental stresses. By improving a plant's performance or survival in response to different environmental stresses, the weather-related losses in productivity and risks to farming can be greatly reduced. Modifying a plant's tolerance to environmental stresses also allows a plant to be grown in regions where a plant or plant variety is typically unable to grow.

Many biochemical changes occur in a plant when a plant becomes tolerant to an environmental stress. For example, for cold or freezing stress tolerance, it is well documented that lipid composition changes occur during cold acclimation in a wide range of plants and there is compelling data to indicate that this contributes to greater freezing tolerance (Steponkus et al. (1993) Advances in Low-Temperature Biology, Vol. 2, P. L. Steponkus, editor, (London: JAI Press), pp. 211-312).

Similarly, the levels of proline and sucrose increase in Arabidopsis (McKown et al. (1996) J. Exp. Bot. 47: 1919-1925; Wanner and Junttila (1999) Plant Physiol. 120: 391-400) and other plants (Guy et al. (1992) Plant Physiol. 100: 502-508; Koster and Lynch (1992) Plant Physiol. 98: 108-113) during cold acclimation and likely have roles in freezing tolerance. There is evidence that proline can protect both membranes and proteins against freeze-induced damage in vitro (Rudolph and Crowe (1985) Cryobiol. 22: 367-377; Carpenter and Crowe (1988) Cryobiol. 25: 244-255) and direct evidence that increased levels of proline enhances whole plant freezing tolerance (Nanjo et al. (1999) FEBS Lett. 461: 205-210).

Sucrose and other simple sugars have also been shown to be effective cryoprotectants in vitro (Strauss et al. (1986) Proc. Natl. Acad. Sci. 83: 2422-2426; Carpenter and Crowe (1988) supra) and there is correlative evidence indicating a role in freezing tolerance in cold-acclimated plants (Guy et al. (1992) supra; Koster and Lynch (1992) supra; Wanner and Junttila (1999) supra).

Similarly, tolerance to drought or water stress is associated with the accumulation of a variety of osmolytes, including sugar alcohols such as mannitol, amino acids such as proline, and glycine betaine (Greenway et al. (1980) Ann. Rev. Plant Physiol. 31: 149-190; Yancey et al. (1982) Science 217: 1214-1222).

Cold acclimation in plants is associated with the expression of cold-regulated (COR) genes that encode many polypeptides of unknown function. One of the more intriguing attributes of the COR genes is that many encode polypeptides that appear to have biochemical similarities with putative cryoprotective proteins (Volger and Heber (1975) Biochim. Biophys. Acta 412: 335-349). Volger and Heber have reported that the leaves of cold-acclimated cabbage and spinach, but not nonacclimated plants, contain proteins that are effective in protecting isolated thylakoid membranes against in vitro freeze-thaw damage. Subsequently, Hincha et al. (1990) reported that the cryoprotective proteins act by reducing membrane permeability during freezing and increasing membrane expandability during thawing (Hincha et al. (1990) Planta 180: 416-419). The biochemical properties of highly enriched fractions of the cryoprotective proteins suggest that the cryoprotective proteins have a number of properties in common with the polypeptides encoded by many of the novel COR genes (Artus et al. (1996) Proc. Natl. Acad. Sci. 93: 13404-13409).

The cold- and drought-regulated COR15a gene of A. thaliana encodes a 15-kDa polypeptide, COR15a, that is targeted to the stromal compartment of chloroplasts (Lin and Thomashow (1992) Plant Physiol. 99: 519-525). During import, COR15a is processed to a mature 9.4-kDa polypeptide, COR15am, that is hydrophilic, remains soluble upon boiling, has a simple amino acid composition (it is rich in both alanine and lysine and devoid of proline, methionine, tryptophan, cysteine, glutamine, arginine, and histidine), is composed largely of a 13-amino acid motif that is repeated four times, and is predicted to form an amphipathic α-helix. The relatively simple amino acid composition of COR15am together with its predicted secondary structure suggests that the polypeptide might have a nonenzymatic function and was shown to have the properties of a cryoprotective protein. (See Artus et al. (1996) supra; Steponkus et al. (1998) Proc. Natl. Acad. Sci. 95: 14570-14575.)

Thus, the present invention provides a method for modifying the plant biomass by modifying the size or number of leaves or roots of a plant, and for increasing the levels of cell protectants in a cell to allow the cell to tolerate greater environmental stresses.

### SUMMARY OF THE INVENTION

In one aspect, the present invention provides a method for modifying plant biomass and the level of a cell protectant in a cell or a plant. The method comprises transforming a plant cell or plant with a recombinant polynucleotide comprising a sequence encoding a C-repeat/DRE binding factor (CBF)-related polypeptide and expressing the CBF-related polypeptide in the plant cell. Expression of the CBF-related polypeptide modifies biomass and the level of the cell protectant in the cell or plant. The method may optionally comprise cold-acclimating the cell to increase the levels of cell protectants in the transformed cell or plant even further.

In one embodiment, the recombinant polynucleotide encodes a CBF-related polypeptide comprising the AP2 domain comprising amino acids 45, 46, 48, 50-52, 54, 59, 60, 62, 64, 65, 67, 68, 71-73, 75-77, 79, 81, 83-91, 93-96, 99, 101, 102, and 104-106 of CBF1 (SEQ ID NO: 2). In a second embodiment, the recombinant polynucleotide encodes a CBF-related polypeptide comprising a CBF-related polypeptide and comprises one or more of the following peptides: PKXXAGR (SEQ ID NO: 319; amino acids 31-37 of SEQ ID NO. 2; ‘X’ may represent any amino acid) or AGRXKF (SEQ ID NO: 320; amino acids 35-40 of SEQ ID NO. 2; ‘X’ may represent any amino acid) or ETRHP (SEQ ID NO: 321; amino acids 42-46 of SEQ ID NO. 2). In a third embodiment, the recombinant polynucleotide encodes a CBF-related polypeptide comprising a CBF-related polypeptide and comprises one or more of the following peptides: PKKXAGR (SEQ ID NO: 322; amino acids 31-37 of SEQ ID NO. 2; ‘X’ may represent any amino acid) or AGRKXF (SEQ ID NO: 324; amino acids 35-40 of SEQ ID NO. 2; ‘X’ may represent any amino acid) or ETRHP (SEQ ID NO: 321; amino acids 42-46 of SEQ ID NO. 2). In a fourth embodiment, the recombinant polynucleotide encodes a CBF-related polypeptide comprising a CBF-related polypeptide and comprises one or more of the following peptides: PKKRAGR (SEQ ID NO: 323; amino acids 31-37 of SEQ ID NO. 2) or AGRKKF (SEQ ID NO: 325; amino acids 35-40 of SEQ ID NO. 2) or ETRHP (SEQ ID NO: 321; amino acids 42-46 of SEQ ID NO. 2). In a fifth embodiment, the recombinant polynucleotide encodes a CBF-related polypeptide comprising a CBF-related polypeptide and comprises one or more of the following peptides: PKKPAGR (SEQ ID NO: 326; amino acids 31-37 of SEQ ID NO. 2) or AGRKKF (SEQ ID NO: 325; amino acids 35-40 of SEQ ID NO. 2) or ETRHP (SEQ ID NO: 321; amino acids 42-46 of SEQ ID NO. 2). In another embodiment, the recombinant polynucleotide encodes a CBF-related polypeptide comprising SEQ ID NO:15. In yet another embodiment, the recombinant polynucleotide comprises SEQ ID NO:14.

Additionally, the recombinant polynucleotide may comprise a regulatory region operably linked to the sequence encoding the CBF-related polypeptide. The regulatory region may comprise a constitutive promoter, an inducible promoter, a tissue specific promoter or a developmental stage specific promoter. The leaf or root biomass of a plant may be modified. Cell protectants whose levels may be modified include proline, sugars, such as sucrose, or lipids, such as fatty acids, or cryoprotective proteins. As a result of the increased levels of any of these cell protectants, or of a combination of any these cell protectants, the environmental stress tolerance of a cell is increased. The environmental stresses may be cold or freezing tolerance, drought tolerance or high salinity tolerance.

In a second aspect, the present invention is another method for modifying the biomass of a plant. This method comprises altering the levels of a polynucleotide comprising a sequence encoding a C-repeat/DRE binding factor (CBF)-related polypeptide in a plant and identifying a plant so modified. The altered polynucleotide expression modifies the biomass of the plant.

In another aspect, the present invention is a method for improving the tolerance of a cell or plant to an environmental stress. The method comprises transforming the cell or plant with a recombinant polynucleotide comprising a sequence encoding a C-repeat/DRE binding factor (CBF)-related polypeptide and expressing the CBF-related polypeptide in the transformed cell or plant. Expression of the CBF-related polypeptide typically increases cell protectant levels at least 1.5 fold in the transformed cell or plant compared with cell protectant levels in an untransformed cell or plant. The enhanced cell protectant levels improve the environmental stress tolerance of the cell or plant.

In one embodiment, the recombinant polynucleotide encodes a CBF-related polypeptide comprising the AP2 domain comprising amino acids 45, 46, 48, 50-52, 54, 59, 60, 62, 64, 65, 67, 68, 71-73, 75-77, 79, 81, 83-91, 93-96, 99, 101, 102 and 104-106 of CBF1 (SEQ ID NO: 2). In a second embodiment, the CBF-related polypeptide may comprise one or more of the following peptides: PKXXAGR (SEQ ID NO: 319; amino acids 31-37 of SEQ ID NO. 2; ‘X’ may represent any amino acid) or AGRXKF (SEQ ID NO: 320; amino acids 35-40 of SEQ ID NO. 2; ‘X’ may represent any amino acid) or ETRHP (SEQ ID NO: 321; amino acids 42-46 of SEQ ID NO. 2). In a third embodiment, the recombinant polynucleotide encodes a CBF-related polypeptide comprising a CBF-related polypeptide and comprises one or more of the following peptides: PKKXAGR (SEQ ID NO: 322; amino acids 31-37 of SEQ ID NO. 2; ‘X’ may represent any amino acid) or AGRKXF (SEQ ID NO: 324; amino acids 35-40 of SEQ ID NO. 2; ‘X’ may represent any amino acid) or ETRHP (SEQ ID NO: 321; amino acids 42-46 of SEQ ID NO. 2). In a fourth embodiment, the recombinant polynucleotide encodes a CBF-related polypeptide comprising a CBF-related polypeptide and comprises one or more of the following peptides: PKKKAGR (SEQ ID NO: 323; amino acids 31-37 of SEQ ID NO. 2) or AGRKKF (SEQ ID NO: 325; amino acids 35-40 of SEQ ID NO. 2) or ETRHP (SEQ ID NO: 321; amino acids 42-46 of SEQ ID NO. 2). In another embodiment, the recombinant polynucleotide encodes a CBF-related polypeptide comprising SEQ ID NO:15. In yet another embodiment, the recombinant polynucleotide comprises SEQ ID NO:14.

Additionally, the recombinant polynucleotide may comprise a regulatory region operably linked to the sequence encoding the CBF-related polypeptide. The regulatory region may comprise a constitutive promoter, an inducible promoter, a tissue specific promoter or a developmental stage specific promoter. Cell protectants whose levels may be modified include proline, sugars, such as sucrose, or lipids, such as fatty acids, or crypoprotective proteins. As a result of the increased levels of any of these cell protectants or the combination of any of these cell protectants, the environmental stress tolerance of a cell or plant is improved.

In a further aspect, the present invention is a method for producing a cell protectant. The method comprises transforming a cell or plant with a recombinant polynucleotide comprising a sequence encoding a C-repeat/DRE binding factor (CBF)-related polypeptide, expressing said recombinant polypeptide in the transformed cell so as to increase the levels of the cell protectant in the cell or plant, and then isolating the cell protectant from the transformed cell or plant. In one embodiment, the recombinant polynucleotide encodes a CBF-related polypeptide comprising SEQ ID NO:15. In another embodiment, the recombinant polynucleotide comprises SEQ ID NO:14.

### BRIEF DESCRIPTION OF THE SEQUENCE LISTING AND DRAWINGS

The file of this patent contains at least one drawing executed in clor. Copies of this patent with clor drawing(s) will be provided by the Patent and Trademark Office upon request and payment of the necessary fee.

The Sequence Listing provides exemplary polynucleotide and polypeptide sequences of the invention. The traits associated with the use of the sequences are included in the Examples.

FIGS. 1A and 1B show how the yeast reporter strains were constructed.

FIG. 1A is a schematic diagram showing the screening strategy.

FIG. 1B is a chart showing activity of the positive cDNA clones in yeast reporter strains.

FIGS. 2A, 2B, 2C and 2D provide an analysis of the pACT-11 cDNA clone.

FIG. 2A is a schematic drawing of the pACT-11 cDNA insert indicating the location and 5′ to 3′ orientation of the 24 kDa polypeptide and 25s rRNA sequences.

FIG. 2B is a DNA and amino acid sequence of the 24 kDa polypeptide (SEQ ID NO: 1 and SEQ ID NO: 2).

FIG. 2C is a schematic drawing indicating the relative positions of the potential nuclear localization signal (NLS), the AP2 domain and the acidic region of the 24 kDa polypeptide.

FIG. 2D is a chart showing comparison of the AP2 domain of the 24 kDa polypeptide (SEQ ID NO:10) with that of the tobacco DNA binding protein EREBP2 (SEQ ID NO:11).

FIG. 3 is a chart showing activation of reporter genes by the 24 kDa polypeptide.

FIG. 4 is a photograph of an electrophoresis gel showing expression of the recombinant 24 kDa polypeptide in E. coli.

FIG. 5 is a photograph of a gel for shift assays indicating that CBF1 binds to the C-repeat/DRE.

FIG. 6 is a photograph of a Southern blot analysis indicating CBF1 is a unique or low copy number gene.

FIGS. 7A, 7B and 7C relate to CBF1 transcripts in control and cold-treated Arabidopsis plants.

FIG. 7A is a photograph of a Northern blot analysis of RNA isolated from Arabidopsis plants that were grown at 22° C. (0 h) or grown at 22° C. and transferred to 2.5° C. for the indicated times (2, 4, 8, or 24 h).

FIG. 7B is a graph showing relative transcript levels of CBF1 in control and cold-treated plants.

FIG. 7C is a graph showing relative transcript levels of COR15a in control and cold-treated plants.

FIG. 8 is a Northern blot showing CBF1 and COR transcript levels in RLD and transgenic Arabidopsis plants.

FIG. 9 is an immunoblot showing COR15am protein levels in RLD and transgenic Arabidopsis plants.

FIGS. 10A and 11B are graphs showing freezing tolerance of leaves from RLD and transgenic Arabidopsis plants.

FIG. 11 is a photograph showing freezing survival of RLD and A6 Arabidopsis plants.

FIG. 12 shows the DNA sequence for CBF2 (SEQ ID NO:12) encoding the polypeptide sequence CBF2 (SEQ ID NO:13).

FIG. 13 shows the DNA sequence for CBF3 (SEQ ID NO:14) encoding the polypeptide sequence CBF3 (SEQ ID NO:15).

FIG. 14 shows the amino acid alignment of proteins CBF1 (SEQ ID NO:2), CBF2 (SEQ ID NO:13), and CBF3 (SEQ ID NO:15).

FIG. 15 is a graph showing induction of reporter genes in yeast that carry the C-repeat/DRE regulatory element CBF1, CBF2, or CBF3.

FIG. 16 shows the amino acid sequence of a portion of a canola CBF homolog (SEQ ID NO:17) and its alignment to the amino acid sequence of CBF1 (SEQ ID NO:2).

FIGS. 17A, 17B, 17C, 17D, 17E, 17F and 17G show restriction maps of plasmids pMB12008, pMB12009, pMB12010, pMB12011, pMB12012, pMB12013, and pMB12014, respectively.

FIG. 18A shows the DNA sequences for the CBF homologs from Brassica juncea, Brassica napus, Brassica oleracea, Brassica rapa, Glycine max, Raphanus sativus, and Zea mays.

FIG. 18B shows the amino acid sequences (one-letter abbreviations) encoded by the DNA sequences (shown in FIG. 18A) for CBF homologs from Brassica juncea, Brassica napus, Brassica oleracea, Brassica rapa, Glycine max, Raphanus sativus, and Zea mays.

FIG. 19A shows an amino acid alignment of the AP2 domains of several CBF proteins (SEQ ID NOs: 137 through 167) with the consensus sequence between the proteins highlighted as well as a comparison of the AP2 domains with that of the tobacco DNA binding protein EREBp2 (SEQ ID NO: 168).

FIG. 19B shows an amino acid alignment of the AP2 domains of several CBF proteins (SEQ ID NOs: 137 through 167) including dreb2a (SEQ ID NO: 169) and dreb2b (SEQ ID NO: 170) with the consensus sequence between the proteins highlighted.

FIG. 19C shows an amino acid alignment of the AP2 domains of several CBF proteins (SEQ ID NOs: 137 through 167) including dreb2a (SEQ ID NO: 169), dreb2b (SEQ ID NO: 170), and tiny (SEQ ID NO: 171) with the consensus sequence between the proteins highlighted.

FIG. 19D shows a difference between the consensus sequence shown in FIG. 19A (SEQ ID NOs: 137 through 167) and tiny (SEQ ID NO: 171).

FIG. 19E shows a difference between the consensus sequence shown in FIG. 19B (SEQ ID NOs: 137 through 167) and tiny (SEQ ID NO: 171).

FIG. 20 shows an amino acid alignment of the amino terminus of several CBF proteins (SEQ ID NOs: 172 through 194) with their consensus sequence highlighted.

FIG. 21A (SEQ ID NOs: 195 through 266) and FIG. 21B (SEQ ID NOs: 267 through 293) show an amino acid alignment of the carboxy terminus of several CBF proteins, with their consensus sequences highlighted.

FIG. 22 shows the effect of CBF3 expression on proline levels. Free proline levels were determined in leaf tissue from control Ws-2 and B6 plants and CBF3-expressing A40, A30 and A28 plants grown at 20° C. (warm) or plants grown at 20° C. and cold-treated at 5° C. for 7 days (7 d cold).

FIG. 23 shows the effect of CBF3 expression on transcript levels of genes involved in proline and sugar metabolism. Northern analysis of total RNA (20 μg for CBF3; 5 μg for other genes) isolated from control Arabidopsis Ws-2 and B6 plants and from CBF3-expressing A40, A30 and A28 plants. Plants were grown at 20° C., then cold-treated at 5° C. for the times indicated. The blots were hybridized with probes for CBF3, COR78, P5CS2, Suc synthase (SuSy), Suc-phosphate synthase (SPS), and elF4a, a constitutively expressed gene used as a loading control (Metz et al. Gene 120: 313 (1992)).

FIG. 24 shows the effect of CBF3 Expression on levels of total soluble sugars. Total soluble sugars were determined for leaf tissue from control Ws-2 and B6 plants and CBF3-expressing A40, A30 and A28 plants grown at 20° C. (warm) or plants grown at 20° C. and cold-treated at 5° C. for 7 days (7 d cold).

FIG. 25 shows the effect of CBF3 expression on fatty acid composition. The fatty acid profiles of total lipids extracted from leaf tissue of control Ws-2 plants and CBF3-expressing A28 plants were determined for plants grown at 20° C. (unfilled bars) or at 20° C. followed by 7 days at 5° C. (filled bars).

FIG. 26 shows the effect of CBF3 expression on freezing tolerance. (A) Seedlings of Ws-2 and A30 were grown at 20° C. on solid medium and frozen at −2° C. for 24 hours followed by 24 hours at −6+ C.; (B) Control Ws-2 and CBF3-expressing transgenic A40, A30 and A28 plants were grown at 20° C. and the freezing tolerance of leaves was measured using the electrolyte leakage test; (C and D) Same as (B) except that plants were grown at 20° C. followed by 7 days cold acclimation at 5° C.

FIGS. 27A-C show a comparison of leaf number to bolting time (A), biomass to bolting time (B) and days to bolt (C) for Arabidopsis thaliana plants overexpressing a CBF-related polypeptide (13-4, 2-1, 16-3, 16-2, 17-10) compared with a wild-type plant (wt-control).

FIG. 28 shows a comparison of the leaves of two plants one overexpressing a polynucleotide encoding a CBF-related polypeptide (A) and a second plant not overexpressing the polynucleotide (B).

FIG. 29 shows a comparison of the roots of two plants one overexpressing a polynucleotide encoding a CBF-related polypeptide (A) and a second plant not overexpressing the polynucleotide (B).

FIG. 30 shows a comparison of the % electrolyte leakage for a plant overexpressing CBF3 (SEQ ID NO: 15) (A40a2, A28a3, A28a3) or a control plant (WS-2), none of which have been cold acclimated.

FIG. 31 shows a comparison of the % electrolyte leakage for a plant overexpressing CBF3 (SEQ ID NO: 15) (A28a3, A30a1) or a control plant (WS-2) after cold acclimation.

FIG. 32 shows a comparison of the % electrolyte leakage for a plant overexpressing CBF2 (SEQ ID NO: 13) (E24, E8 or E2) or a control plant (WS-2, B6), none of which have been cold acclimated.

FIG. 33 shows a comparison of the % electrolyte leakage for a plant overexpressing CBF2 (SEQ ID NO: 13) (E24, E8 or E2) or a control plant (WS-2, B6), after cold acclimation.

FIGS. 34A through 34D shows an alignment of CBF sequences from Arabidopsis (SEQ ID NOs: 2, 15, 13, and 97) and from Medicago trunculata (SEQ ID NOs: 294 through 300). Amino acid residues with identity between aligned sequences are boxed. The consensus sequence is shown below each set of alignments.

FIGS. 35A through 35D shows an alignment of CBF sequences from Arabidopsis (SEQ ID NOs: 2, 15, 13, and 97) and from Oryza sativa (SEQ ID NOs: 301 through 310). Amino acid residues with identity between aligned sequences are boxed. The consensus sequence is shown below each set of alignments.

FIGS. 36A through 36C shows an alignment of CBF sequences from Arabidopsis (SEQ ID NOs: 2, 15, 13, and 97) and from Zea mays (SEQ ID NOs: 311 through 315). Amino acid residues with identity between aligned sequences are boxed. The consensus sequence is shown below each set of alignments.

FIG. 37 shows that CBFs from Medicago trunculata, Oryza sativa, and Arabidopsis (CBF3) activate transcription from the Arabidopsis COR gene promoter from the RD29a gene, as measured by fold-increase over control (pMEN65 plasmid control=1 unit). The CBF sequences are: Arabidopsis CBF3 (SEQ ID NO:15; CBF3), Medicago trunculata CBF (SEQ ID NO:294; mt G3362), Medicago trunculata CBF (SEQ ID NO:295; mt G3364), Medicago trunculata CBF (SEQ ID NO:296; mt G3365), Medicago trunculata CBF (SEQ ID NO:297; mt G3366), Medicago trunculata CBF (SEQ ID NO:298; mt G3367), Medicago trunculata CBF (SEQ ID NO:299; mt G3368), Medicago trunculata CBF (SEQ ID NO:300; mt G3369), Oryza sativa CBF (SEQ ID NO:301; os G3370), Oryza sativa CBF (SEQ ID NO:302; os G3371), Oryza sativa CBF (SEQ ID NO:303; os G3372), Oryza sativa CBF (SEQ ID NO:304; os G3373), Oryza sativa CBF (SEQ ID NO:308; os G3377), Oryza sativa CBF (SEQ ID NO:309; os G3378), and Oryza sativa CBF (SEQ ID NO:310; os G3379).

### DETAILED DESCRIPTION OF THE INVENTION

Definitions

“Environmental stress tolerance” refers to a decrease in the extent of a cell's injury or growth inhibition or an increase in survival rate after exposure to cold or freezing temperatures, drought conditions, high salinity environments or the like.

A “cell protectant” refers to a compound that improves the environmental stress tolerance of a cell. The cell protectant may be a cryoprotectant or an osmoprotectant. The cell protectant may be proline or any metabolically related compound, sugars or any metabolically related compound, and a variety of lipids, including fatty acids, which protect a cell's integrity during an environmental stress.

A “polynucleotide” is a nucleotide sequence comprising a gene coding sequence or a fragment thereof (comprising at least 18 consecutive nucleotides, preferably at least 30 consecutive nucleotides, and more preferably at least 50 consecutive nucleotides). Additionally, the polynucleotide may comprise a promoter, an intron, an enhancer region, a polyadenylation site, a translation initiation site, 5′ or 3′ untranslated regions, a reporter gene, a selectable marker or the like. The polynucleotide may comprise single stranded or double stranded DNA or RNA. The polynucleotide may comprise modified bases or a modified backbone. The polynucleotide may be genomic, a transcript (such as an mRNA) or a processed nucleotide sequence (such as a cDNA). The polynucleotide may comprise a sequence in either sense or antisense orientations.

A “recombinant polynucleotide” is a polynucleotide that is not in its native state, for example, the polynucleotide is comprised of a nucleotide sequence not found in nature or the polynucleotide is separated from nucleotide sequences with which it typically is in proximity or is next to nucleotide sequences with which it typically is not in proximity or is expressed at different levels.

A “consensus sequence”, with regard to nucleotide sequences, refers to a nucleotide sequence that serves to represent a family of similar, experimentally-derived sequences. Each position in the consensus sequence is assigned a base that corresponds to the most frequently occurring nucleotide in the experimentally-derived sequences, when sequences are compared in an alignment. A “consensus sequence”, with regard to polypeptide sequences, refers to a polypeptide sequence that serves to represent a family of similar, experimentally-derived sequences. Each position in the consensus sequence is assigned an amino acid residue that corresponds to the most frequently occurring amino acid residue in the experimentally-derived sequences, when sequences are compared in an alignment.

A “transformed plant” refers to a plant that contains genetic material not normally found in a wild type plant and which has been introduced into a plant by human manipulation. A transformed plant is a plant that may contain an expression vector or cassette. The expression cassette comprises a gene coding sequence and allows for the expression of the gene coding sequence. The expression cassette may be introduced into a plant by transformation or by breeding after transformation of a parent cell. In particular, the transformed plant may refer to a whole plant as well as to a plant part, such as flower, seed, fruit, leaf, or root, plant tissue, plant cells or any other plant material, and progeny thereof.

A “transformed cell” refers to a cell that contains genetic material not normally found in a wild type cell and which has been introduced into the cell by human manipulation. A transformed cell is a cell that may contain an expression vector or cassette. The expression cassette comprises a gene coding sequence and allows for the expression of the gene coding sequence. The expression cassette may be introduced into a cell by transformation or by breeding after transformation of a parent cell. A transformed cell may refer to a cell from any organism, including mammalian cells, plant cells, bacterial cells, and the like. In particular, the transformed cell is a plant cell and may refer to a whole plant as well as to a plant part, such as seed, fruit, leaf, or root, plant tissue, plant cells or any other plant material, and progeny thereof.

The term “modified expression” in reference to polynucleotide or polypeptide expression refers to an expression pattern in a transformed cell or plant that is different from the expression pattern in the wild type cell or plant; for example, by expression in a cell type or plant tissue other than a cell type or plant tissue in which the polynucleotide or polypeptide is naturally expressed, or by expression at a time other than at the time the polynucleotide or polypeptide is expressed in the wild type cell or plant, or by a response to different inducible agents, such as hormones or environmental signals, or at different expression levels (either higher or lower) compared to those observed a wild type cell or plant. The term may also refer to lowering the levels of expression to below the detection level or completely abolishing expression. The resulting expression pattern may be transient or stable, constitutive or inducible.

A “CBF-related polypeptide” or “CBF” is a protein transcription factor that binds to a promoter comprising a cold- and dehydration-responsive DNA regulatory element known as the CRT (C-repeat)/DRE (dehydration responsive element) (Baker et al. (1994) i Plant. Mol. Biol. 24: 701-713; Yamaguchi-Shinozaki and Shinozaki (1994) Plant Cell 6: 251-264). The terms “CBF-related polypeptide” and “CBF” are interchangeable. These proteins comprise an AP2/EREBP DNA binding motif (Riechmann and Meyerowitz (1998) Biol. Chem. 379: 633-646) and are transcription factors (Stockinger et al. (1997) Proc. Natl. Acad. Sci. 94: 1035-1040). The AP2 domain, which may also be referred to as the ERF domain (Hao et al. (1998) J. Biol. Chem 273: 26857-26861) may comprise amino acids 45, 46, 48, 50-52, 54, 59, 60, 62, 64, 65, 67, 68, 71-73, 75-77, 79, 81, 83-91, 93-96, 99, 101, 102 and 104-106 of CBF1 (G40; SEQ ID NO: 2). Additionally, the CBF-related polypeptide may comprise one or more of the following peptides: PKXXAGR (SEQ ID NO: 319; amino acids 31-37 of SEQ ID NO. 2; ‘X’ may represent any amino acid) or AGRXKF (SEQ ID NO: 320; amino acids 35-40 of SEQ ID NO. 2; ‘X’ may represent any amino acid) or ETRHP (SEQ ID NO: 321; amino acids 42-46 of SEQ ID NO: 2).

A “regulatory region” is a region that can regulate the transcription of a gene coding sequence. The regulatory region may be a promoter or an enhancer. The regulatory region sequence is “operably linked” when it is placed into a functional relationship with the gene coding sequence. For example, a promoter or enhancer is operably linked to a gene coding sequence if the presence of the promoter or enhancer increases the level of expression of the gene coding sequence. Promoters may increase transcription of a gene at all times (constitutive promoter), increase transcription only in the presence of specific agents or events (inducible promoter), increase transcription in specific tissue(s) (tissue specific promoter) or during specific stages of cell or tissue or organism development (developmental stage specific promoter).

“Plant biomass modification” refers to a detectable difference in the size of one or more plant tissues, such as the leaves, roots, stem, fruit or flowers of a transformed plant expressing a polynucleotide or polypeptide of the present invention compared with a plant not doing so. The modification may entail at least a 5% increase or decrease in an observed trait (difference), at least a 10% difference, at least a 20% difference, at least a 30%, at least a 50%, at least a 70%, at least a 100% or a greater difference. It is known that there may be a natural variation in plant biomass. Therefore, the modification observed entails a change in the normal distribution of the trait in transformed plants compared with the distribution observed in wild type plants.

“Cell protectant level modification” refers to a detectable difference in cell protectant levels in a transformed cell expressing a polynucleotide or polypeptide of the present invention compared with a cell not doing so, such as a wild type cell. The trait modification may entail at least a 5% increase or decrease in an observed trait (difference), at least a 10% difference, at least a 20% difference, at least a 30%, at least a 50%, at least a 70%, at least a 100% or a greater difference. It is known that there may be a natural variation in the modified cell protectant levels. Therefore, the cell protectant level modification observed entails a change in the normal distribution of the levels in transformed cells compared with the distribution observed in wild type cells.

“Cold-acclimating a cell” and “cold acclimation” refers to a process whereby a transformed cell is exposed to temperatures below about 12° C. for different periods of time to elicit higher levels of a cell protectant compared with cell protectant levels in a cell that has not been cold acclimated.

Traits that May Be Modified

Trait modifications of particular interest include those to seed (such as embryo or endosperm), fruit, root, flower, leaf, stem, shoot, seedling or the like, including: enhanced tolerance to environmental conditions including freezing, chilling, heat, drought, water saturation, radiation and ozone; improved tolerance to microbial, fungal or viral diseases; improved tolerance to pest infestations, including nematodes, mollicutes, parasitic higher plants or the like; decreased herbicide sensitivity; improved tolerance of heavy metals or enhanced ability to take up heavy metals; improved growth under poor photoconditions (for example, low light and/or short day length), or changes in expression levels of genes of interest. Other phenotype that can be modified relate to the production of plant metabolites, such as variations in the production of taxol, tocopherol, tocotrienol, sterols, phytosterols, vitamins, wax monomers, anti-oxidants, amino acids, lignins, cellulose, tannins, prenyllipids (such as chlorophylls and carotenoids), glucosinolates, and terpenoids, enhanced or compositionally altered protein or oil production (especially in seeds), or modified sugar (insoluble or soluble) and/or starch composition. Physical plant characteristics or traits that can be modified include cell development (such as the number of trichomes), fruit and seed size and number, yields of plant parts such as stems, leaves, inflorescences, and roots, the stability of the seeds during storage, characteristics of the seed pod (for example, susceptibility to shattering), root hair length and quantity, internode distances, or the quality of seed coat. Plant growth characteristics that can be modified include growth rate, germination rate of seeds, vigor of plants and seedlings, leaf and flower senescence, male sterility, apomixis, flowering time, flower abscission, rate of nitrogen uptake, osmotic sensitivity to soluble sugar concentrations, biomass or transpiration characteristics, as well as plant architecture characteristics such as apical dominance, branching patterns, number of organs, organ identity, organ shape or size.

Transcription Factors Modify Expression of Endogenous Genes

Expression of genes that encode transcription factors that modify expression of endogenous genes, polynucleotides, and proteins are well known in the art. In addition, transgenic plants comprising isolated polynucleotides encoding transcription factors may also modify expression of endogenous genes, polynucleotides, and proteins. Examples include Peng et al. (1997) Genes and Development 11: 3194-3205) and Peng et al. (1999) Nature 400: 256-261). In addition, many others have demonstrated that an Arabidopsis transcription factor expressed in an exogenous plant species elicits the same or very similar phenotypic response. See, for example, Fu et al. (2001) Plant Cell 13: 1791-1802); Nandi et al. (2000) Curr. Biol. 10: 215-218); Coupland (1995) Nature 377: 482-483); and Weigel et al. (1995) Nature 377: 482-500).

In another example, Mandel et al. (1992) Cell 71: 133-143) and Suzuki et al. (2001) i Plant J. 28: 409-418) teach that a transcription factor expressed in another plant species elicits the same or very similar phenotypic response of the endogenous sequence, as often predicted in earlier studies of Arabidopsis transcription factors in Arabidopsis. Other examples can be found in the teachings of Müller et al. (2001, Plant J. 28: 169-179); Kim et al. (2001) Plant J. 25: 247-259); Kyozuka and Shimamoto (2002) Plant Cell Physiol. 43: 130-135); Boss and Thomas (2002) Nature, 416: 847-850); He et al. (2000) Transgenic Res. 9: 223-227); and Robson et al. (2001) Plant J. 28: 619-631).

In yet another example, Gilmour et al. (1998) Plant J. 16: 433-442) and Jaglo-Ottosen et al. (1998) Science 280: 104-106) teach an Arabidopsis AP2 transcription factor, CBF1, which, when overexpressed in transgenic plants, increases plant freezing tolerance. An alignment of the CBF proteins from Arabidopsis, B. napus, l wheat, rye, and tomato revealed the presence of conserved amino acid sequences, PKK/RPAGRxKFxETRHP and DSAWR, that flank the AP2/EREBP DNA binding domains of the proteins and distinguish them from other members of the AP2/EREBP protein family.

Polypeptides and Polynucleotides of the Invention

The present invention provides, among other things, transcription factors (TFs), and transcription factor homolog polypeptides, and isolated or recombinant polynucleotides encoding the polypeptides, or novel variant polypeptides or polynucleotides encoding novel variants of transcription factors derived from the specific sequences provided here. These polypeptides and polynucleotides may be employed to modify a plant's characteristic.

Exemplary polynucleotides encoding the polypeptides of the invention were identified by screening an Arabidopsis cDNA expression library for clones encoding C-repeat/DRE binding domains (Stockinger et al. (1997) Proc. Natl. Acad. Sci. 94: 1035-1040). The cDNA library harbored in Escherichia coli BNN132 was amplified, and plasmid DNA was isolated and transformed into yeast GGY1 reporter strains. CBF genes were expressed in E. coli and yeast, and gel shift assays, in which orientation and concatenation number of inserts were determined by dideoxy DNA sequence analysis, were conducted to confirm that the CBF gene products bound to the C-repeat/DRE (Stockinger (1997) supra). Sequences initially identified were then further characterized to identify sequences comprising specified sequence strings corresponding to sequence motifs present in families of known transcription factors. In addition, further exemplary polynucleotides encoding the polypeptides of the invention were identified in the plant GenBank database using publicly available sequence analysis programs and parameters. Sequences initially identified were then further characterized to identify sequences comprising specified sequence strings corresponding to sequence motifs present in families of known transcription factors. Polynucleotide sequences meeting such criteria were confirmed as transcription factors.

Additional polynucleotides of the invention were identified by screening Arabidopsis thaliana and/or other plant cDNA libraries with probes corresponding to known transcription factors under low stringency hybridization conditions. Additional sequences, including full length coding sequences were subsequently recovered by the rapid amplification of cDNA ends (RACE) procedure, using a commercially available kit according to the manufacturer's instructions. Where necessary, multiple rounds of RACE are performed to isolate 5′ and 3′ ends. The full-length cDNA was then recovered by a routine end-to-end polymerase chain reaction (PCR) using primers specific to the isolated 5′ and 3′ ends. Exemplary sequences are provided in the Sequence Listing.

The polynucleotides of the invention can be or were ectopically expressed in overexpressor or knockout plants and the changes in the characteristic(s) or trait(s)characteristics or traits of the plants observed. Therefore, the polynucleotides and polypeptides can be employed to improve the characteristics or traits of plants.

The polynucleotides of the invention can be or were ectopically expressed in overexpressor plant cells and the changes in the expression levels of a number of genes, polynucleotides, and/or proteins of the plant cells observed. Therefore, the polynucleotides and polypeptides can be employed to change expression levels of a genes, polynucleotides, and/or proteins of plants.

Producing Polypeptides

The polynucleotides of the invention include sequences that encode transcription factors and transcription factor homolog polypeptides and sequences complementary thereto, as well as unique fragments of coding sequence, or sequence complementary thereto. Such polynucleotides can be, for example, DNA or RNA, for example, mRNA, cRNA, synthetic RNA, genomic DNA, cDNA synthetic DNA, oligonucleotides, etc. The polynucleotides are either double-stranded or single-stranded, and include either, or both sense (i.e., coding) sequences and antisense (i.e., non-coding, complementary) sequences. The polynucleotides include the coding sequence of a transcription factor, or transcription factor homolog polypeptide, in isolation, in combination with additional coding sequences (for example, a purification tag, a localization signal, as a fusion-protein, as a pre-protein, or the like), in combination with non-coding sequences (for example, introns or inteins, regulatory elements such as promoters, enhancers, terminators, and the like), and/or in a vector or host environment in which the polynucleotide encoding a transcription factor or transcription factor homolog polypeptide is an endogenous or exogenous gene.

A variety of methods exist for producing the polynucleotides of the invention. Procedures for identifying and isolating DNA clones are well known to those of skill in the art, and are described in, for example, Berger and Kimmel, Guide to Molecular Cloning Techniques Methods in Enzymology volume 152; Academic Press, Inc., San Diego, Calif. (“Berger”); Sambrook et al. Molecular Cloning—A Laboratory Manual (2nd edition), Vol. 1-3, Cold Spring Harbor Laboratory, Cold Spring Harbor, N.Y., (1989) (“Sambrook”) and Current Protocols in Molecular Biology, F. M. Ausubel et al., editors, Current Protocols, a joint venture between Greene Publishing Associates, Inc. and John Wiley & Sons, Inc., (supplemented through 2000) (“Ausubel”).

Alternatively, polynucleotides of the invention, can be produced by a variety of in vitro amplification methods adapted to the present invention by appropriate selection of specific or degenerate primers. Examples of protocols sufficient to direct persons of skill through in vitro amplification methods, including the polymerase chain reaction (PCR) the ligase chain reaction (LCR), Qbeta-replicase amplification and other RNA polymerase mediated techniques (for example, NASBA), for example, for the production of the homologous nucleic acids of the invention are found in Berger (supra), Sambrook (supra) and Ausubel (supra), as well as Mullis et al. (1987) PCR Protocols A Guide to Methods and Applications, Innis et al., editors, (1990) Academic Press Inc. San Diego, Calif. (“Innis”). Improved methods for cloning in vitro amplified nucleic acids are described in Wallace et al. U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, for example, Ausubel, Sambrook and Berger, all supra.

Alternatively, polynucleotides and oligonucleotides of the invention can be assembled from fragments produced by solid-phase synthesis methods. Typically, fragments of up to approximately 100 bases are individually synthesized and then enzymatically or chemically ligated to produce a desired sequence, for example, a polynucleotide encoding all or part of a transcription factor. For example, chemical synthesis using the phosphoramidite method is described, for example, by Beaucage et al. (1981) Tetrahedron Letters 22: 1859-1869; and Matthes et al. (1984) EMBO J. 3: 801-805. According to such methods, oligonucleotides are synthesized, purified, annealed to their complementary strand, ligated and then optionally cloned into suitable vectors. And if so desired, the polynucleotides and polypeptides of the invention can be custom ordered from any of a number of commercial suppliers.

Homologous Sequences

Sequences homologous, i.e., that share significant sequence identity or similarity, to those provided in the Sequence Listing, derived from Arabidopsis thaliana or from other plants of choice are also an aspect of the invention. Homologous sequences can be derived from any plant including monocots and dicots and in particular agriculturally important plant species, including but not limited to, crops such as soybean, wheat, corn, potato, cotton, rice, rape, oilseed rape (including rapeseed and canola), sunflower, alfalfa, sugarcane and turf; or fruits and vegetables, such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, watermelon, rosaceous fruits (such as apple, peach, pear, cherry and plum) and vegetable brassicas (such as broccoli, cabbage, cauliflower, Brussels sprouts, and kohlrabi). Other crops, fruits and vegetables whose phenotype can be changed include barley, rye, millet, sorghum, currant, avocado, citrus fruits such as oranges, lemons, grapefruit and tangerines, artichoke, cherries, nuts such as the walnut and peanut, endive, leek, roots, such as arrowroot, beet, cassava, turnip, radish, yam, and sweet potato, and beans. The homologous sequences may also be derived from woody species, such pine, poplar and eucalyptus, or mint or other labiates.

Orthologs and Paralogs

Several different methods are known by those of skill in the art for identifying and defining these functionally homologous sequences. Three general methods for defining paralogs and orthologs are described; a paralog or ortholog or homolog may be identified by one or more of the methods described below.

Orthologs and paralogs are evolutionarily related genes that have similar sequence and similar functions. Orthologs are structurally related genes in different species that are derived by a speciation event. Paralogs are structurally related genes within a single species and that are derived by a duplication event.

Within a single plant species, gene duplication may cause two copies of a particular gene, giving rise to two or more genes with similar sequence and similar function known as paralogs. A paralog is therefore a similar gene with a similar function within the same species. Paralogs typically cluster together or in the same clade (a group of similar genes) when a gene family phylogeny is analyzed using programs such as CLUSTAL (Thompson et al. (1994) Nucleic Acids Res. 22: 4673-4680; Higgins et al. (1996) Methods Enzymol. 266 383-402). Groups of similar genes can also be identified with pair-wise BLAST analysis (Feng et al. (1987) J. Mol. Evol. 25: 351-360). For example, a clade of very similar MADS domain transcription factors from Arabidopsis all share a common function in flowering time (Ratcliffe et al. (2001) Plant Physiol. 126: 122-132), and a group of very similar AP2 domain transcription factors from Arabidopsis are involved in tolerance of plants to freezing (Gilmour et al. (1998) supra). Analysis of groups of similar genes with similar function that fall within one clade can yield sub-sequences that are particular to the lade. These sub-sequences, known as consensus sequences, can not only be used to define the sequences within each clade, but define the functions of these genes; genes within a clade may contain paralogous sequences, or orthologous sequences that share the same function. (See also, for example, Mount, D. W. (2001) Bioinformatics: Sequence and Genome Analysis Cold Spring Harbor Laboratory Press, Cold Spring Harbor, N.Y., page 543.)

Speciation, the production of new species from a parental species, can also give rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species. Once a phylogenic tree for a gene family of one species has been constructed using a program such as CLUSTAL (Thompson et al. (1994) Nucleic Acids Res. 22: 4673-4680; Higgins et al. (1996) Methods Enzymol. 266: 383-402), potential orthologous sequences can placed into the phylogenetic tree and its relationship to genes from the species of interest can be determined. Once the ortholog pair has been identified, the function of the test ortholog can be determined by determining the function of the reference ortholog.

Transcription factors that are homologous to the listed sequences will typically share at least about 30% amino acid sequence identity, or at least about 30% amino acid sequence identity outside of a known consensus sequence or consensus DNA-binding site. More closely related transcription factors can share at least about 50%, about 60%, about 65%, about 70%, about 75% or about 80% or about 90% or about 95% or about 98% or more sequence identity with the listed sequences, or with the listed sequences but excluding or outside a known consensus sequence or consensus DNA-binding site, or with the listed sequences excluding one or all conserved domain. Factors that are most closely related to the listed sequences share, for example, at least about 85%, about 90% or about 95% or more % sequence identity to the listed sequences, or to the listed sequences but excluding or outside a known consensus sequence or consensus DNA-binding site or outside one or all conserved domain. At the nucleotide level, the sequences will typically share at least about 40% nucleotide sequence identity, preferably at least about 50%, about 60%, about 70% or about 80% sequence identity, and more preferably about 85%, about 90%, about 95% or about 97% or more sequence identity to one or more of the listed sequences, or to a listed sequence but excluding or outside a known consensus sequence or consensus DNA-binding site, or outside one or all conserved domain. The degeneracy of the genetic code enables major variations in the nucleotide sequence of a polynucleotide while maintaining the amino acid sequence of the encoded protein. Conserved domains within a transcription factor family may exhibit a higher degree of sequence homology, such as at least 65% sequence identity including conservative substitutions, and preferably at least 80% sequence identity, and more preferably at least 85%, or at least about 86%, or at least about 87%, or at least about 88%, or at least about 90%, or at least about 95%, or at least about 98% sequence identity. Transcription factors that are homologous to the listed sequences should share at least 30%, or at least about 60%, or at least about 75%, or at least about 80%, or at least about 90%, or at least about 95% amino acid sequence identity over the entire length of the polypeptide or the homolog. In addition, transcription factors that are homologous to the listed sequences should share at least 30%, or at least about 60%, or at least about 75%, or at least about 80%, or at least about 90%, or at least about 95% amino acid sequence similarity over the entire length of the polypeptide or the homolog.

Percent identity can be determined electronically, for example, by using the MEGALIGN program (DNASTAR, Inc. Madison, Wis.). The MEGALIGN program can create alignments between two or more sequences according to different methods, for example, the clustal method (see, for example, Higgins et al. (1988) Gene 73: 237-244). The clustal algorithm groups sequences into clusters by examining the distances between all pairs. The clusters are aligned pairwise and then in groups. Other alignment algorithms or programs may be used, including FASTA, BLAST, or ENTREZ, FASTA and BLAST. These are available as a part of the GCG sequence analysis package (University of Wisconsin, Madison, Wis.), and can be used with or without default settings. ENTREZ is available through the National Center for Biotechnology Information. In one embodiment, the percent identity of two sequences can be determined by the GCG program with a gap weight of 1, for example, each amino acid gap is weighted as if it were a single amino acid or nucleotide mismatch between the two sequences (see U.S. Pat. No. 6,262,333).

Other techniques for alignment are described in Methods in Enzymology, vol. 266: Computer Methods for Macromolecular Sequence Analysis (1996), editor, Doolittle, Academic Press, Inc., San Diego, Calif. Preferably, an alignment program that permits gaps in the sequence is utilized to align the sequences. The Smith-Waterman is one type of algorithm that permits gaps in sequence alignments. See Methods Mol. Biol. 70: 173-187 (1997). Also, the CLUSTALW alignment algorithm (for example, in the MACVECTOR 6.0 or MACVECTOR 6.5 applications, Accelrys, San Diego Calif.) may be utilized to align sequences. Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The percentage similarity between two polypeptide sequences, for example, sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between polynucleotide sequences can also be counted or calculated by other methods known in the art, for example, the Jotun Hein method (see, for example, Hein (1990) Methods Enzymol. 183: 626-645). Identity between sequences can also be determined by other methods known in the art, for example, by varying hybridization conditions (see U.S. patent application No. 20010010913).

Thus, the invention provides methods for identifying a sequence similar or paralogous or orthologous or homologous to one or more polynucleotides as noted herein, or one or more target polypeptides encoded by the polynucleotides, or otherwise noted herein and may include linking or associating a given plant phenotype or gene function with a sequence. In the methods, a sequence database is provided (locally or across an inter or intra net) and a query is made against the sequence database using the relevant sequences herein and associated plant phenotypes or gene functions.

In addition, one or more polynucleotide sequences or one or more polypeptides encoded by the polynucleotide sequences may be used to search against a BLOCKS (Bairoch et al. (1997) Nucleic Acids Res. 25: 217-221), PFAM, and other databases which contain previously identified and annotated motifs, sequences and gene functions. Methods that search for primary sequence patterns with secondary structure gap penalties (Smith et al. (1992) Protein Eng. 5: 35-51) as well as algorithms such as Basic Local Alignment Search Tool (BLAST; Altschul, S. F. (1993) J. Mol. Evol. 36:290-300; Altschul et al. (1990) supra), BLOCKS (Henikoff and Henikoff (1991) Nucleic Acids Res. 19: 6565-6572), Hidden Markov Models (HMM; Eddy (1996) Curr. Opin. Str. Biol. 6: 361-365; Sonnhammer et al. (1997) Proteins 28: 405-420), and the like, can be used to manipulate and analyze polynucleotide and polypeptide sequences encoded by polynucleotides. These databases, algorithms and other methods are well known in the art and are described in Ausubel et al. (1997; Short Protocols in Molecular Biology, John Wiley & Sons, New York N.Y., unit 7.7) and in Meyers (1995; Molecular Biology and Biotechnology, Wiley VCH, New York N.Y., pages 856-853).

Furthermore, methods using manual alignment of sequences similar or homologous to one or more polynucleotide sequences or one or more polypeptides encoded by the polynucleotide sequences may be used to identify regions of similarity and conserved domains. Such manual methods are well-known of those of skill in the art and can include, for example, comparisons of tertiary structure between a polypeptide sequence encoded by a polynucleotide which comprises a known function with a polypeptide sequence encoded by a polynucleotide sequence which has a function not yet determined. Such examples of tertiary structure may comprise predicted alpha helices, beta-sheets, amphipathic helices, leucine zipper motifs, zinc finger motifs, proline-rich regions, cysteine repeat motifs, and the like.

CBF Genes and Related Sequences

Many plants, including Arabidopsis, show increased resistance to freezing after they have been exposed to low, non-freezing temperatures. This cold-acclimation response is associated with the induction of COR (cold-regulated) genes mediated by the C-repeat/drought-responsive element (CRT/DRE) DNA regulatory element) (Baker et al. (1994) Plant. Mol. Biol. 24: 701-713; Yamaguchi-Shinozaki and Shinozaki (1994) Plant Cell 6: 251-264). Increased expression of Arabidopsis CBF genes (transcriptional activators that bind to the CRT/DRE sequence), induce COR gene expression and increase the freezing tolerance of non-cold acclimated Arabidopsis plants. CBF genes are thus regulators of the cold acclimation response, and act by controlling the level of COR gene expression, which in turn promotes tolerance to freezing. CBF genes have also been shown to influence cell protectant levels such as proline in plants, which can lead to increased plant biomass (Kishor et al. (1995) Plant Physiol. 108: 1387-1394).

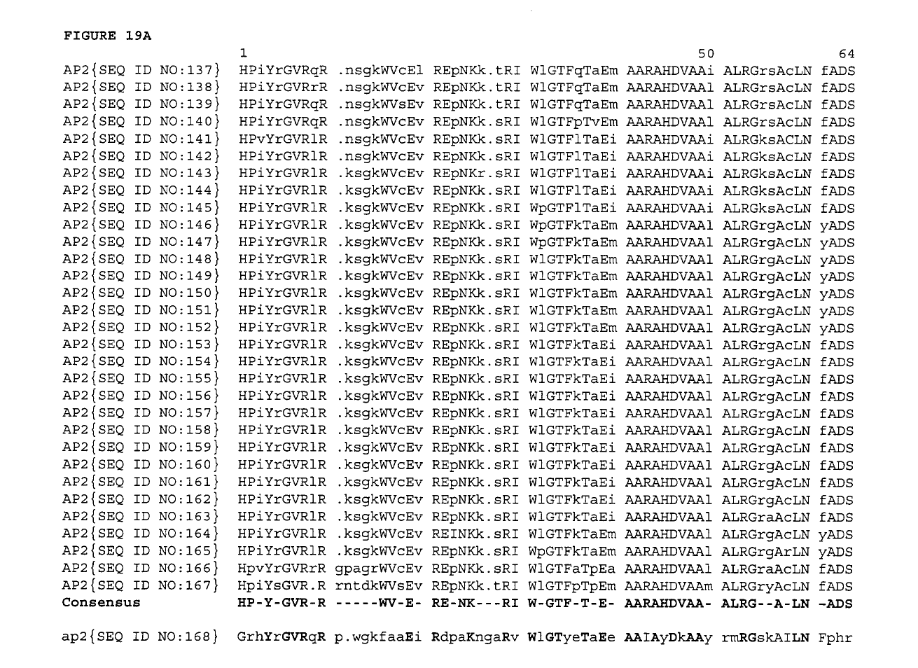

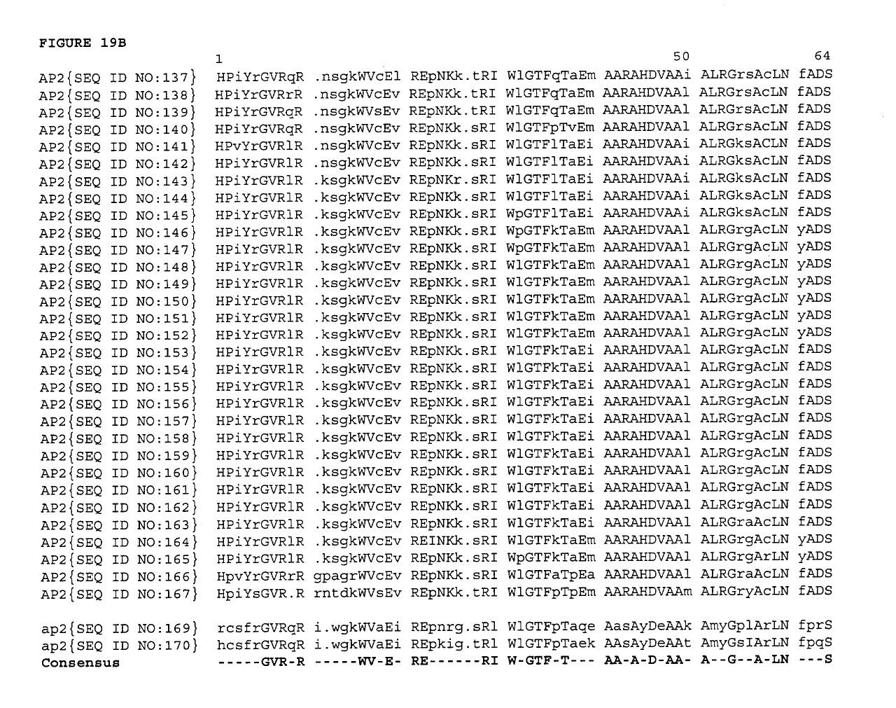

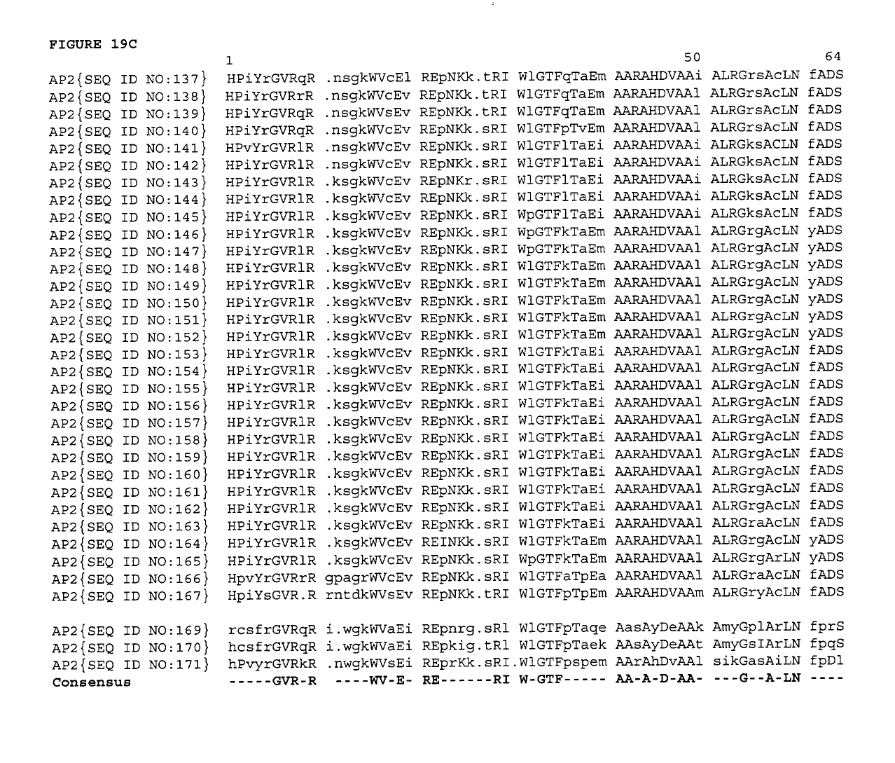

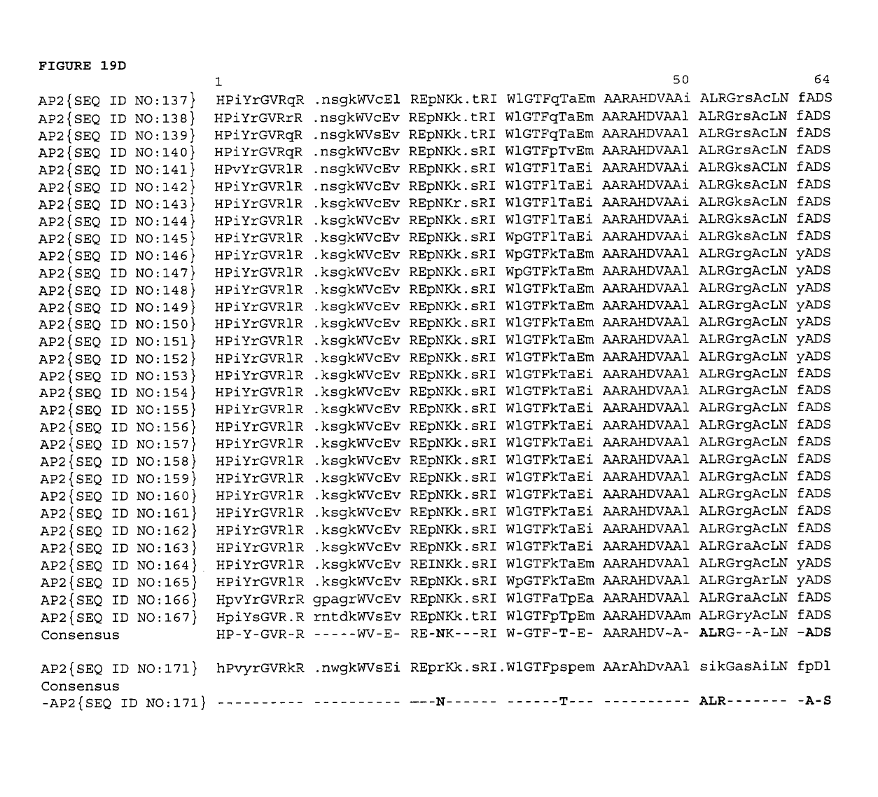

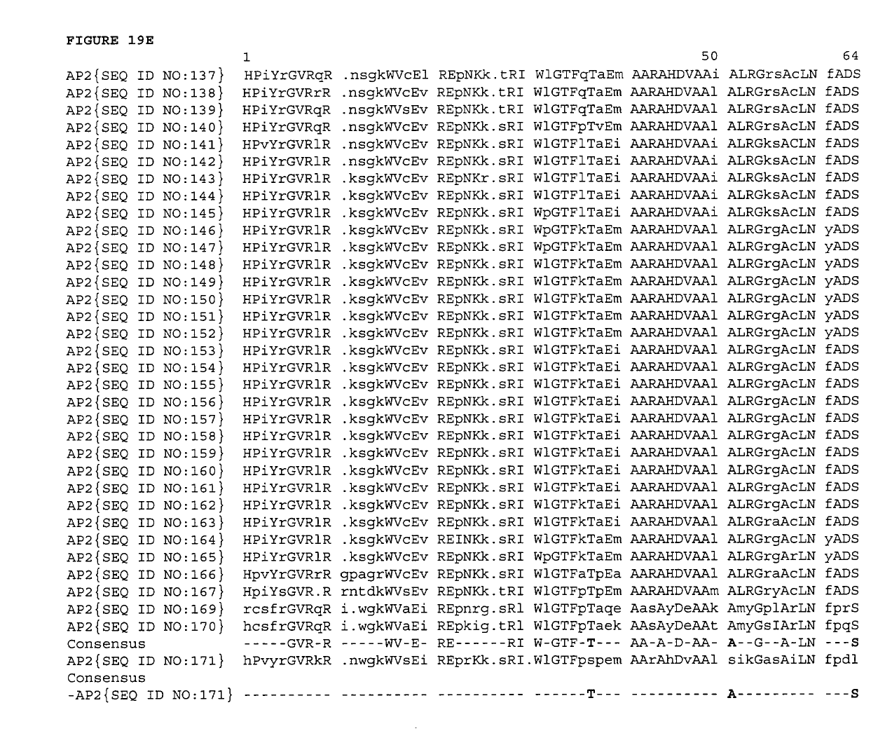

It is believed that a significant class of environmental stress tolerance and biomass regulatory genes encode for binding proteins with an AP2 domain capable of binding to a DNA regulatory sequence. Each of the presently disclosed CBF gene sequences encodes a binding protein that includes an AP2 domain, the latter being a DNA-binding motif similar to those present in Arabidopsis proteins APETALA2, AINTEGUMENTA and TINY, the tobacco ethylene response element binding proteins, and numerous other plant proteins. The AP2 domains of CBF binding proteins in general, including the CBF binding proteins described herein, share significant homology, and comprise a consensus sequence sufficiently homologous to any one of the consensus sequences shown in FIG. 19A, 19B, or 19C that the binding protein is capable of binding to a CCG regulatory sequence, preferably a CCGAC regulatory sequence. Specifically, CBF proteins comprise an AP2/EREBP DNA binding motif (Riechmann and Meyerowitz (1998) Biol. Chem. 379: 633-646) and are transcription factors (Stockinger et al. (1997) supra). The AP2 domain of CBF proteins may comprise amino acids 45, 46, 48, 50-52, 54, 59, 60, 62, 64, 65, 67, 68, 71-73, 75-77, 79, 81, 83-91, 93-96, 99, 101, 102 and 104-106 of CBF1 (G40; SEQ ID NO: 2), or the consensus sequence:

H P Xₙ Y Xₙ G V R Xₙ R Xₙ W V Xₙ E Xₙ R E Xₙ N K Xₙ R I W Xₙ G T F Xₙ T Xₙ E Xₙ A A R A H D V A A Xₙ A L R G Xₙ A Xₙ L N Xₙ A D S

where X is any amino acid residue and n is any number of amino acid residues.

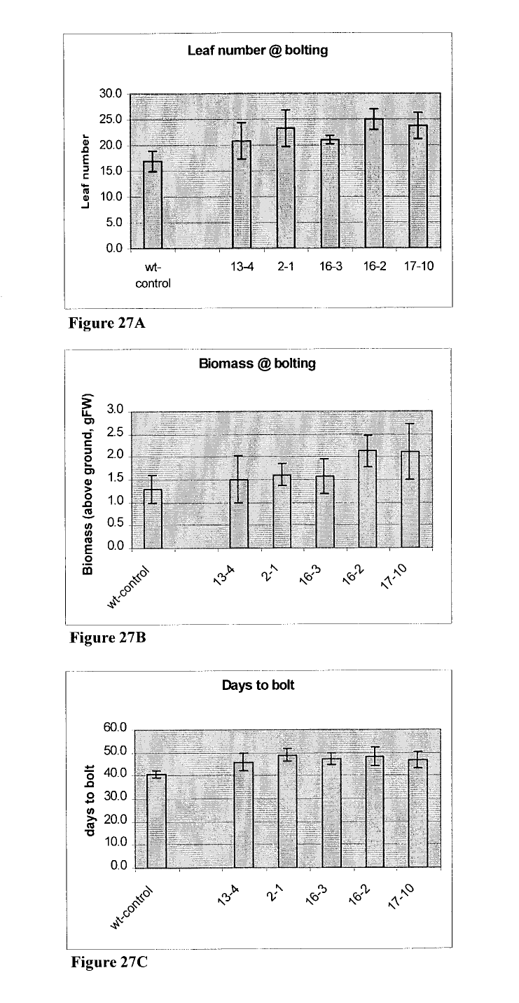

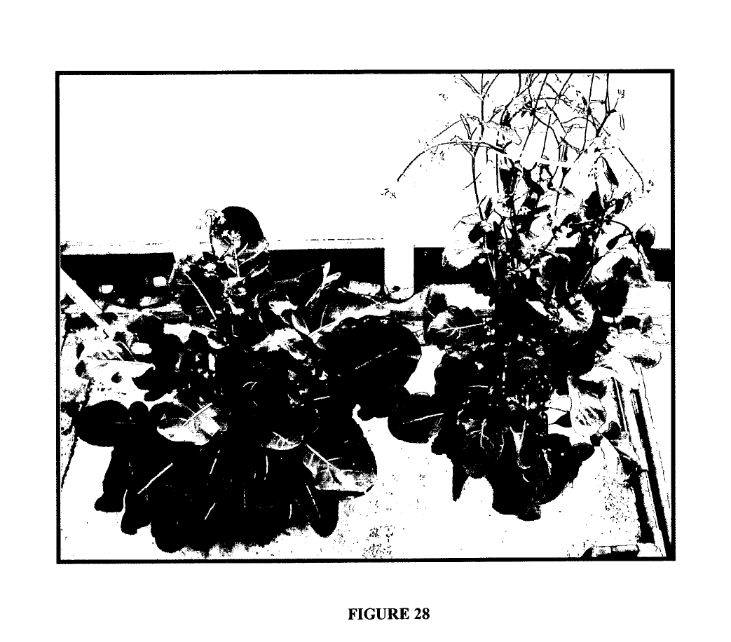

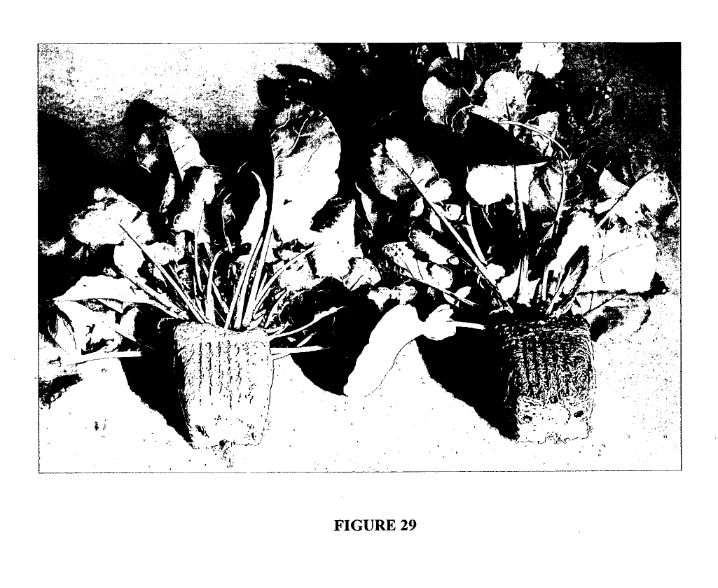

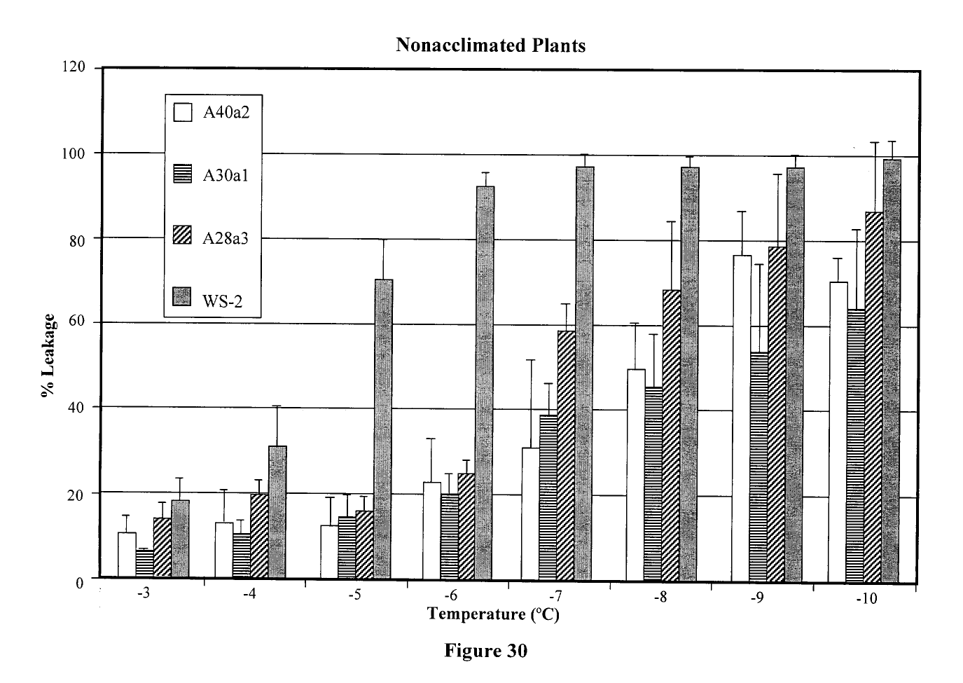

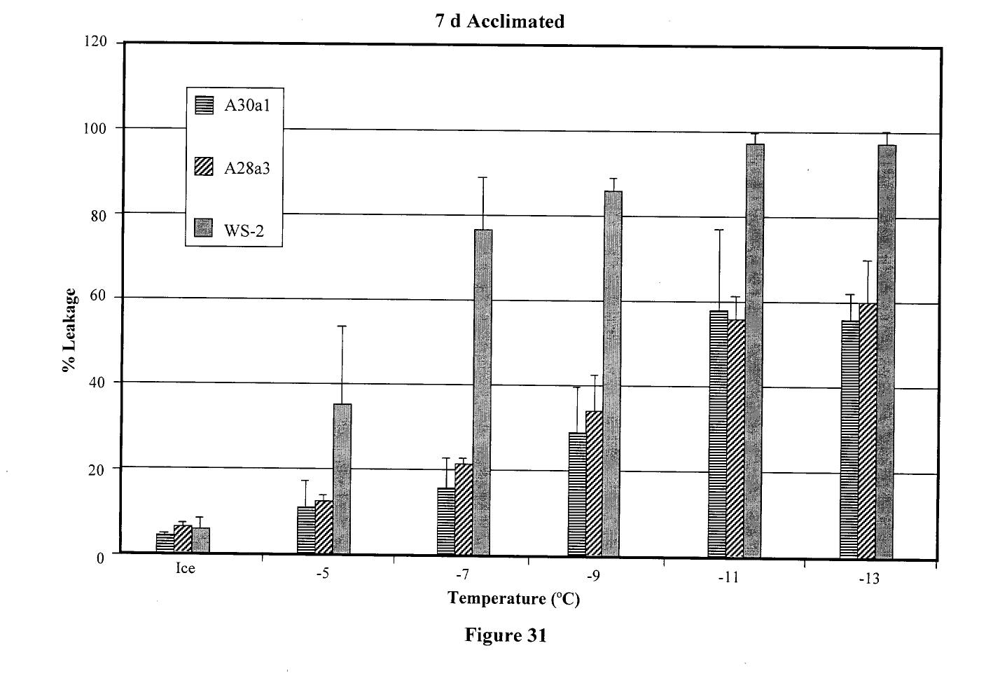

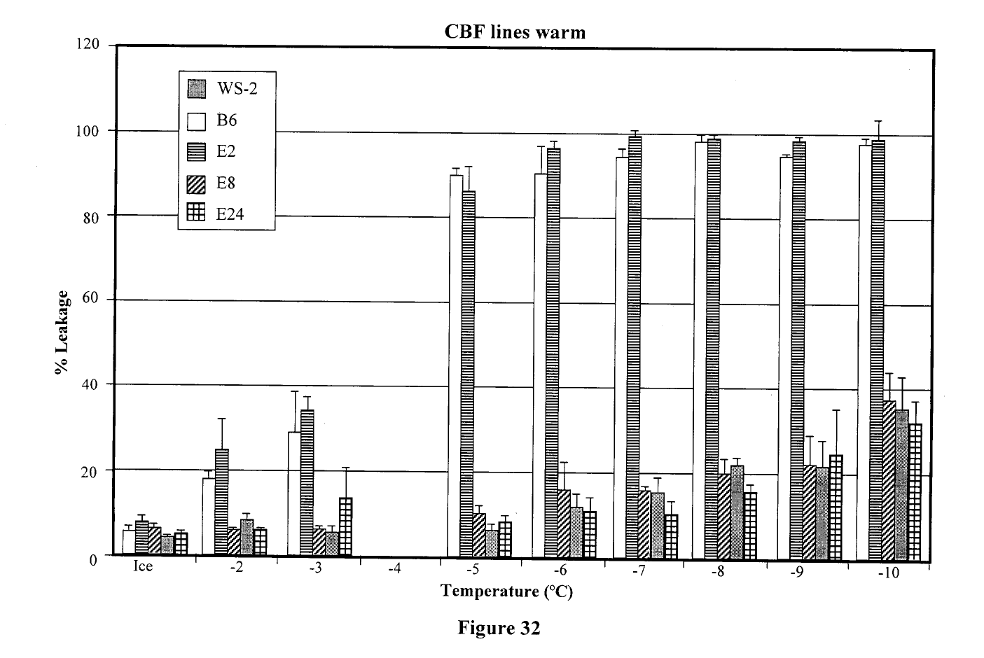

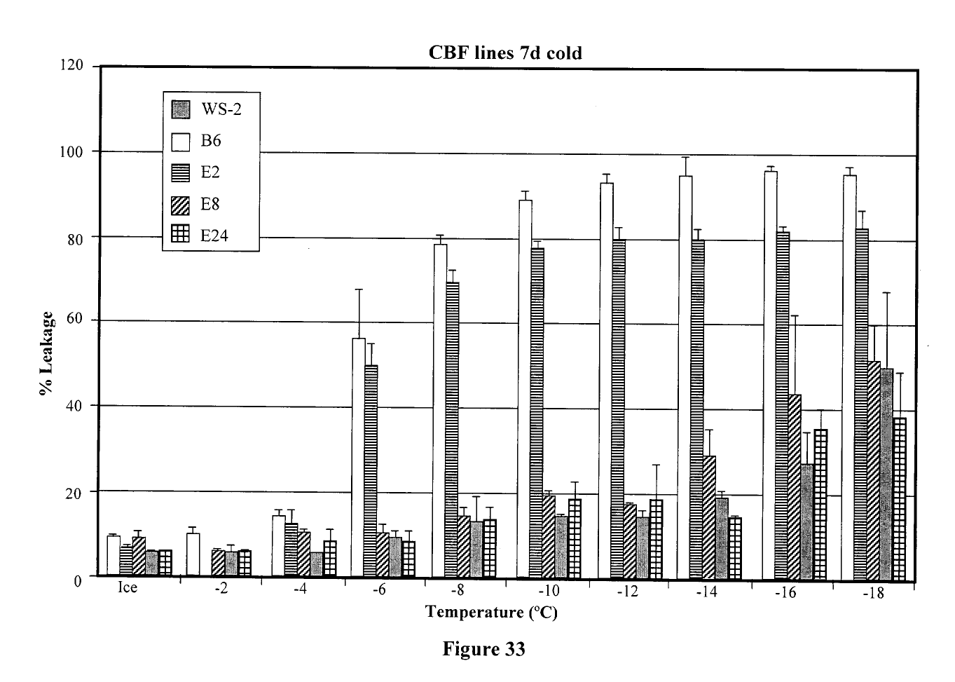

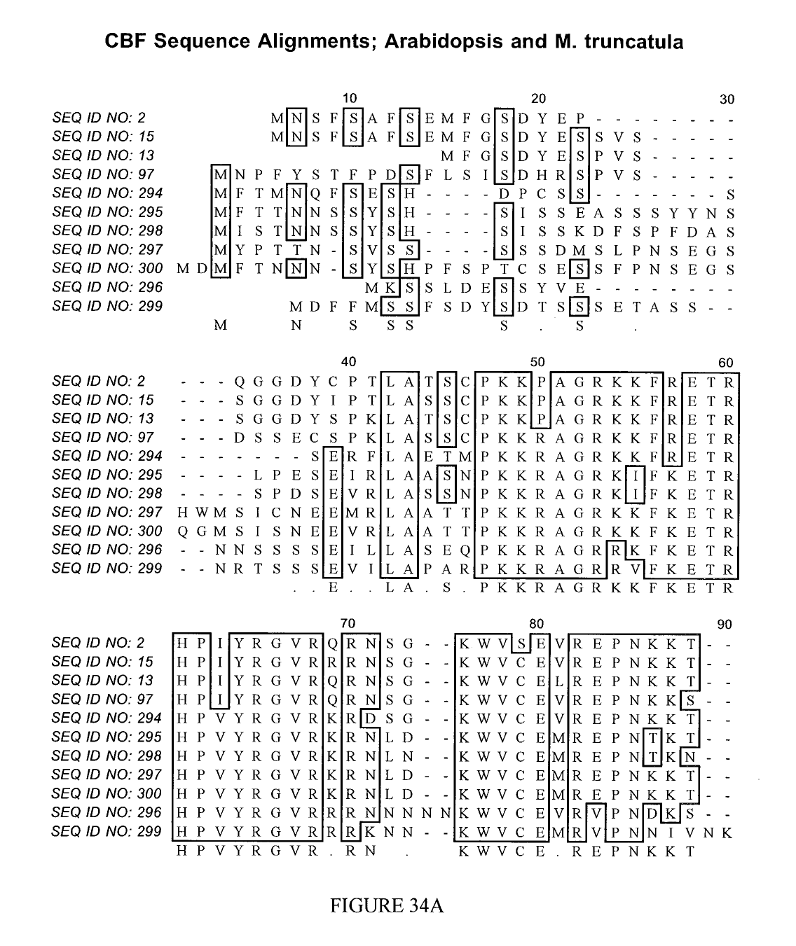

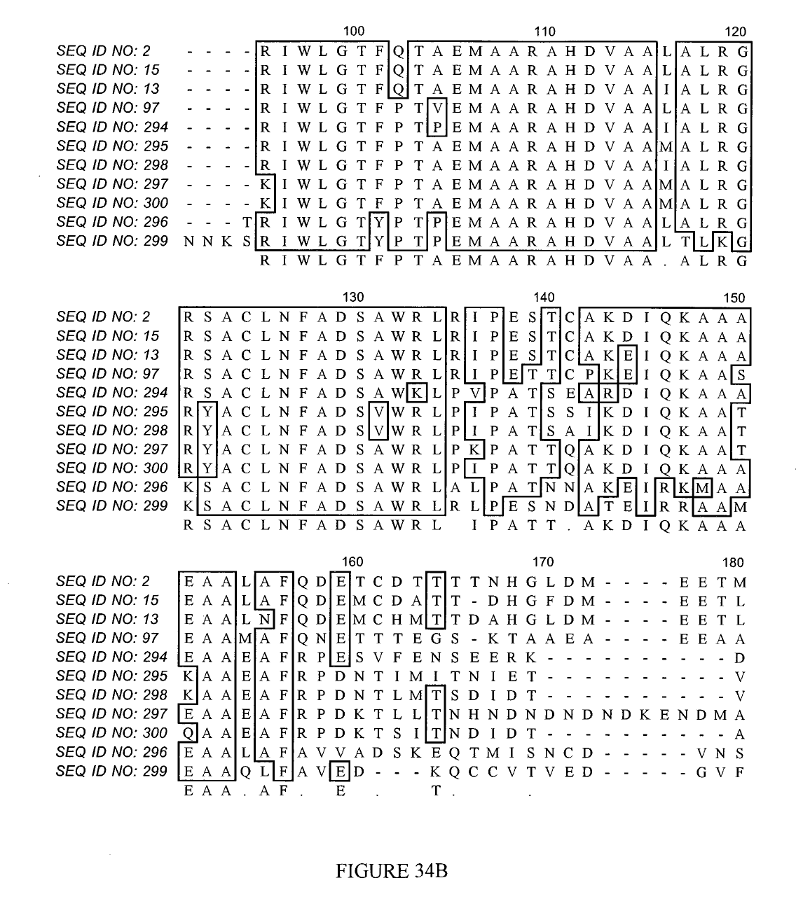

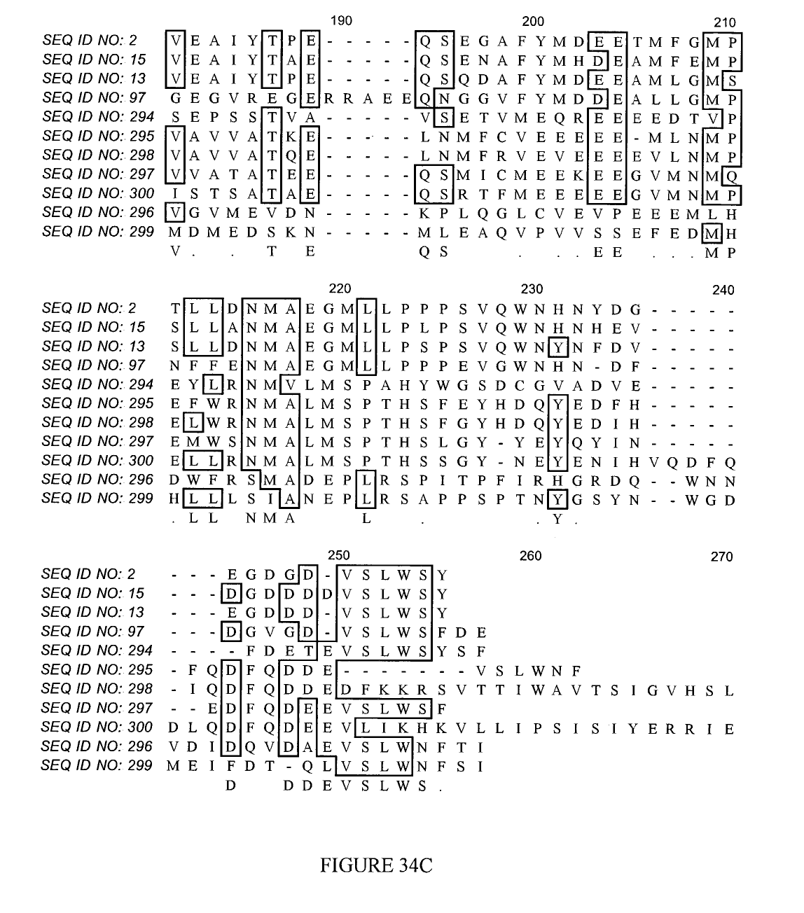

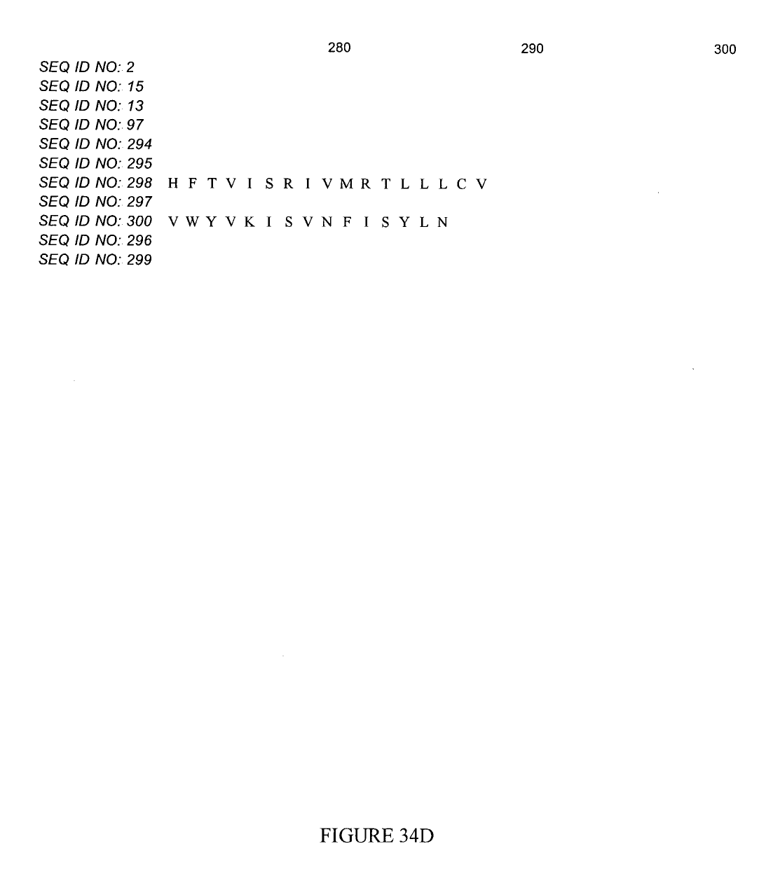

CBF1 (G40; SEQ ID NO: 2), CBF2 (G41; SEQ ID NO: 13) and CBF3 (G42; SEQ ID NO: 15) have similar sequences, particularly in the AP2 domain (defined by the consensus sequence defined above and bounded by: HP Xₙ Y Xₙ GVR Xₙ ADS). CBF2 has 95% sequence identity with CBF1 in the AP2 domain, and CBF3 shares 96% sequence identity with CBF1 in the AP2 domain. These three and related genes can be used to prepare transgenic plants and plants with altered traits. CBF1, for example, was studied using transgenic plants in which the gene encoding the protein was expressed under the control of the 35S promoter (for a more complete discussion of studies involving CBF1, CBF2 and CBF3 experiments, see examples below). CBF1 was shown to improve tolerance to freezing and salt stress in Arabidopsis and Canola. CBF1 could thus be used to manipulate those tolerances, and to generate plants that might germinate and survive under such adverse conditions. For example, evaporation from the soil surface causes upward water movement and salt accumulation in the upper soil layer where the seeds are placed. Thus, germination normally takes place at a salt concentration much higher than the mean salt concentration in the whole soil profile. Increased salt tolerance during the germination stage of a crop plant would impact survivability and yield. If the activity of CBF1 is regulated at a post-translational level (for example, by being phosphorylated), it might be possible to engineer constitutively active versions of the protein that protect the plant under adverse environmental conditions As seen in FIGS. 27A through 27C, CBF1-overexpressing plants grew more slowly, than the control plants. However, as the transgenic plants flowered later they grew for a longer period of time prior to bolting; at this developmental stage the transgenic plants had more leaves and higher biomass than the control plants at the same developmental stage (which the control plant reached a few days earlier).

Similarly, overexpression of CBF3 in transformed plants increased the time to flowering; the plants grew for a longer period of time prior to bolting and increased the number of rosette leaves per plant (see Example 22, below). Thus, CBF polypeptide overexpression could be used to manipulate time to flowering, leaf number, and plant biomass. If the activity of CBF polypeptides is regulated at a post-translational level (for example, by being phosphorylated), it might be possible to engineer constitutively active versions of the protein that increase leaf number and/or biomass.

G912 (CBF4; SEQ ID NO: 97). G912 was recognized by Applicants as the AP2/EREBP gene most closely related to Arabidopsis CBF1, CBF2, and CBF3 (SEQ ID NO: 2, 13, and 15, respectively) (Haake et al. (2002) Plant Physiol. 130: 639-648; Stockinger et al. (1997) supra); Gilmour et al. (1998) supra), G912 shares 93%, 91 and 93% sequence identity with CBF1, CBF2 and CBF3, respectively, in the AP2 domain. G912 sequence similarity with CBF1, 2 and 3 extends beyond the conserved AP2 domain. This AP2/EREBP transcription factor is also closely related to the members of the CBF-like subgroup of AP2/EREBP proteins from other plants, such as AF084185 Brassica napus dehydration responsive element binding protein. G912 was identified in the sequence of P1 clone MSG15 (GenBank accession number AB015478; gene MSG15.6; no published information is available about the functions of G912).

G912 expression appears to be induced by cold, drought, and osmotic stress. The function of G912 was studied using transgenic plants in which this gene was expressed under the control of the 35S promoter. As with plants overexpressing CBF1, CBF2 and CBF3, plants overexpressing G912 were dark green, and flowered later than non-transformed control plants. Plants overexpressing G912 were more freezing and drought tolerant than the wild-type controls.

All these results mirror the extensive body of work presented herein that has shown that related genes CBF1, CBF2, and CBF3 are involved in the control of the low-temperature response in Arabidopsis, and that those genes can be used to improve freezing, drought, and salt tolerance in plants (Stockinger et al. (1997) supra; Gilmour et al. (1998) supra; Jaglo-Ottosen et al. (1998) Science 280: 104-106; Liu et al. (1998) Plant Cell 10: 1391-1406, Kasuga et al. (1999) Nature Biotechnol. 17: 287-291). In addition, G912 overexpressing plants also exhibit a sugar sensing phenotype: i.e., reduced seedling vigor and cotyledon expansion upon germination on high glucose media.

Polypeptide Transcription Factors from other Plant Species (Odd Numbered SEQ ID NO: 39-95 and Even numbered 116-128).

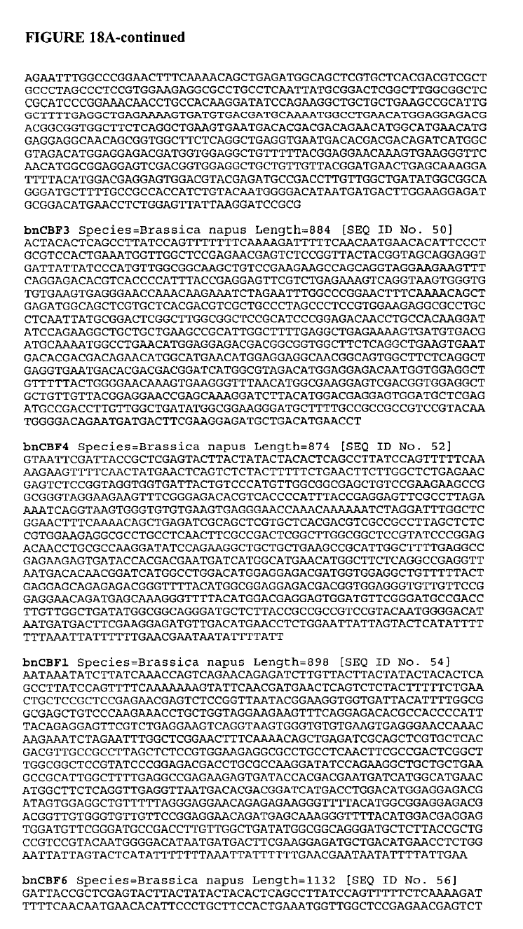

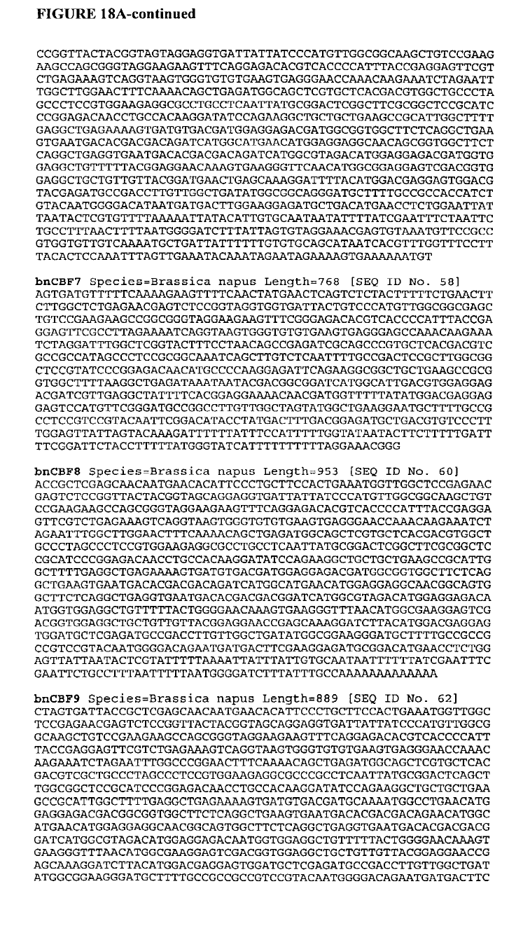

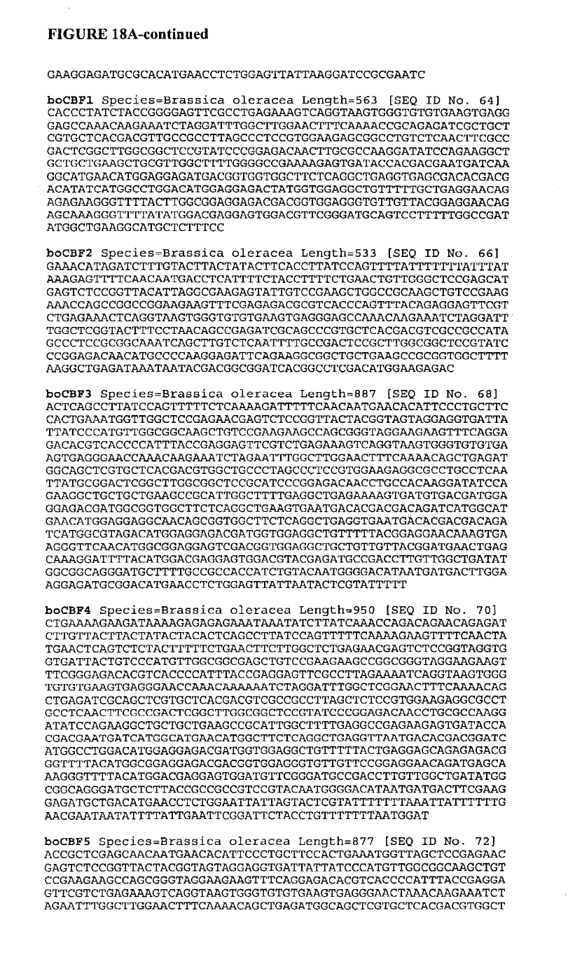

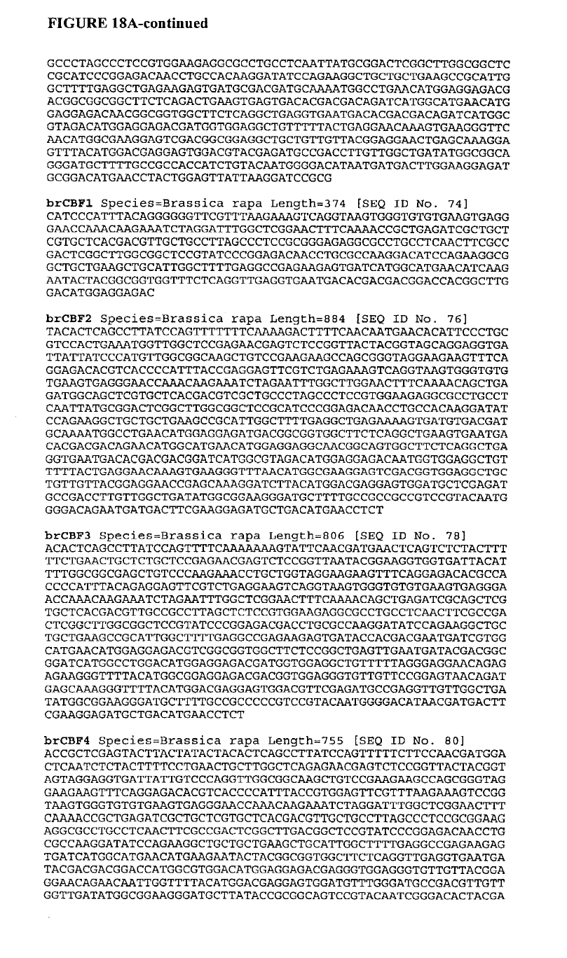

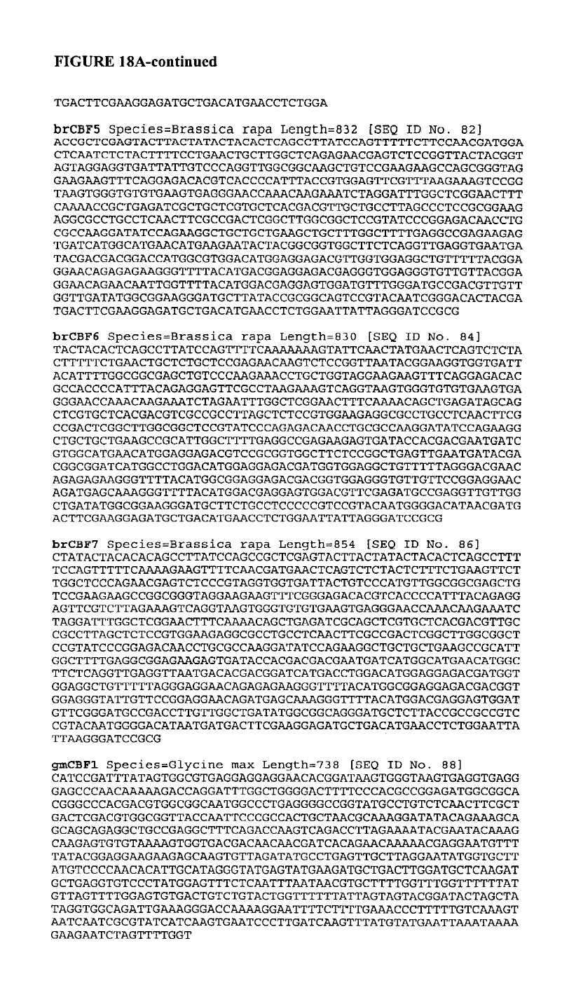

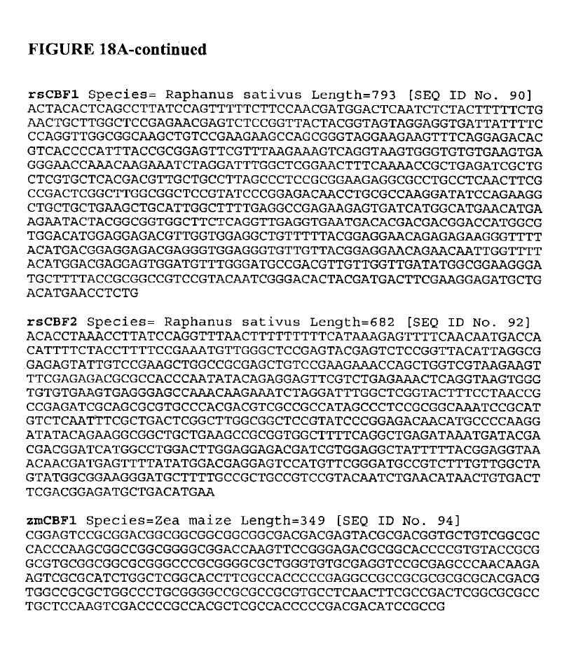

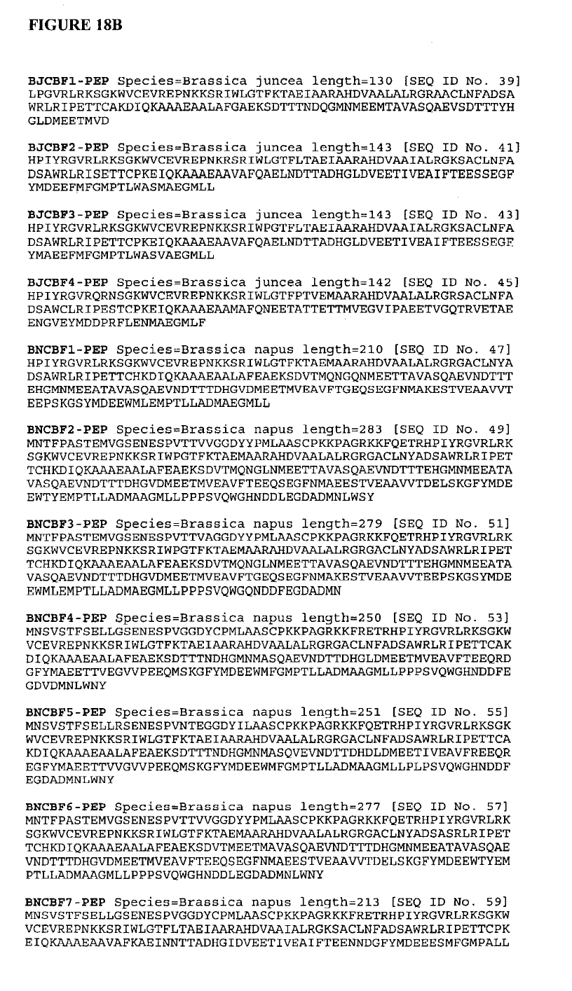

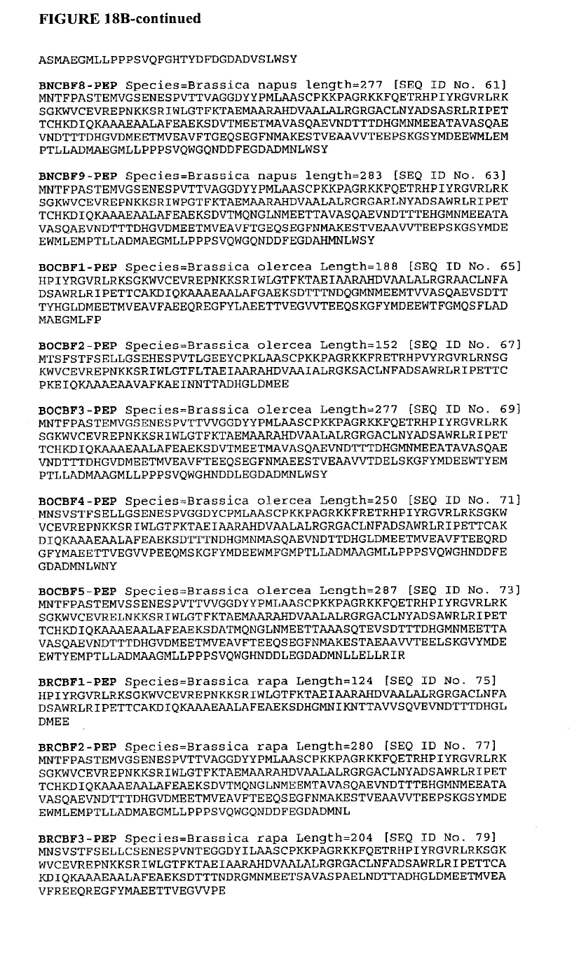

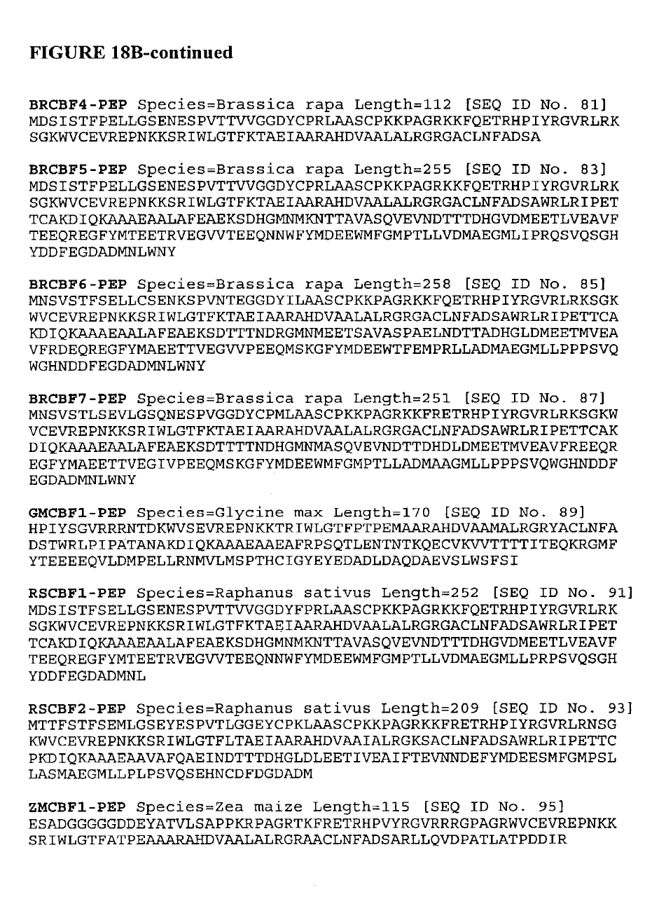

As described in more detail in Example 15, below, a PCR strategy was used to isolate CBF homologs from a number of species of plants both related and diverse from Arabidopsis. These species included Brassica juncea, Brassica napus, Brassica oleracea, Brassica rapa, Glycine max, Raphanus sativus, Secale cereale, Triticum aestivum, and Zea mays. The nucleotide (for example, bjCBF1) and peptide sequences (for example, BJCBF 1-PEP) of these isolated CBF homologs are shown in FIGS. 18A and 18B, respectively. Table 11 (which may be found in Example 15) lists the sequence names and SEQ ID NO: of these isolated CBF homologs. The percentage sequence identity of each of the AP2 domains of the sequences from other species with the Arabadopsis CBF1 AP2 domain (subsequence of G40, SEQ ID NO: 2) is also shown and is 80% identity for the Zea mays AP2 domain and from 85-93% for the other six species.

SEQ ID NOs: 39-45 are from B. juncea. The AP2 regions present in each of these sequences, and which may be found as subsequences in the corresponding sequences in the Sequence Listing, are:

SEQ ID NO: 39 (percent sequence identity with CBF1: 87% (50/57)): PGVRLRKSGKWVCEVREPNKKSRIWLGTFKTAEIAARAHDVAALALRGRAACLNFADS

SEQ ID NO: 143 (percent sequence identity with CBF1: 85% (53/62)): HPIYRGVRLRKSGKWVCEVREPNKRSRIWLGTFLTAEIAARAHDVAAIALRGKSACLNFADS

SEQ ID NO: 145 (percent sequence identity with CBF1: 85% (53/62)): HPIYRGVRLRKSGKWVCEVREPNKKSRIWPGTFLTAEIAARAHDVAAIALRGKSACLNFADS

SEQ ID NO: 140 (percent sequence identity with CBF1: 93% (58/62)): HPIYRGVRQRNSGKWVCEVREPNKKSRIWLGTFPTVEMAARAHDVAALALRGRSACLNFADS

SEQ ID NOs: 47-63 are from B. napus. The AP2 regions present in each of these sequences are:

SEQ ID NO: 148 (percent sequence identity with CBF1: 88% (55/62)): HPIYRGVRLRKSGKWVCEVREPNKKSRIWLGTFKTAEMAARAHDVAALALRGRGACLNYADS

SEQ ID NO: 146 (percent sequence identity with CBF1: 87% (54/62)): HPIYRGVRLRKSGKWVCEVREPNKKSRIWPGTFKTAEMAARAHDVAALALRGRGACLNYADS

SEQ ID NO: 147 (percent sequence identity with CBF1: 87% (54/62)): HPIYRGVRLRKSGKWVCEVREPNKKSRIWPGTFKTAEMAARAHDVAALALRGRGACLNYADS

SEQ ID NO: 153 (percent sequence identity with CBF1: 88% (55/62)): HPIYRGVRLRKSGKWVCEVREPNKKSRIWLGTFKTAEIAARAHDVAALALRGRGACLNFADS

SEQ ID NO: 154 (percent sequence identity with CBF1: 88% (55/62)): HPIYRGVRLRKSGKWVCEVREPNKKSRIWLGTFKTAEIAARAHDVAALALRGRGACLNFADS

SEQ ID NO: 149 (percent sequence identity with CBF1: 88% (55/62)): HPIYRGVRLRKSGKWVCEVREPNKKSRIWLGTFKTAEMAARAHDVAALALRGRGACLNYADS

SEQ ID NO: 144 (percent sequence identity with CBF1: 87% (54/62)): HPIYRGVRLRKSGKWVCEVREPNKKSRIWLGTFLTAEIAARAHDVAAIALRGKSACLNFADS

SEQ ID NO: 150 (percent sequence identity with CBF1: 88% (55/62)): HPIYRGVRLRKSGKWVCEVREPNKKSRIWLGTFKTAEMAARAHDVAALALRGRGACLNYADS

SEQ ID NO: 165 (percent sequence identity with CBF1: 85% (53/62)): HPIYRGVRLRKSGKWVCEVREPNKKSRIWPGTFKTAEMAARAHDVAALALRGRGARLNYADS

SEQ ID NOs: 65-73 are from B. oleracea. The AP2 regions present in each of these sequences are:

SEQ ID NO: 163 (percent sequence identity with CBF1: 88% (55/62)): HPIYRGVRLRKSGKWVCEVREPNKKSRIWLGTFKTAEIAARAHDVAALALRGRAACLNFADS

SEQ ID NO: 141 (percent sequence identity with CBF1: 87% (54/62)): HPVYRGVRLRNSGKWVCEVREPNKKSRIWLGTFLTAEIAARAHDVAAIALRGKSACLNFADS

SEQ ID NO: 151 (percent sequence identity with CBF1: 88% (55/62)): HPIYRGVRLRKSGKWVCEVREPNKKSRIWLGTFKTAEMAARAHDVAALALRGRGACLNYADS

SEQ ID NO: 155 (percent sequence identity with CBF1: 88% (55/62)): HPIYRGVRLRKSGKWVCEVREPNKKSRIWLGTFKTAEIAARAHDVAALALRGRGACLNFADS

SEQ ID NO: 164 (percent sequence identity with CBF1: 87% (54/62)): HPIYRGVRLRKSGKWVCEVRELNKKSRIWLGTFKTAEMAARAHDVAALALRGRGACLNYADS

SEQ ID NOs: 75-87 are from B. rapa. The AP2 regions present in each of these sequences are:

SEQ ID NO: 156 (percent sequence identity with CBF1: 88% (55/62)): HPIYRGVRLRKSGKWVCEVREPNKKSRIWLGTFKTAEIAARAHDVAALALRGRGACLNFADS

SEQ ID NO: 152 (percent sequence identity with CBF1: 88% (55/62)): HPIYRGVRLRKSGKWVCEVREPNKKSRIWLGTFKTAEMAARAHDVAALALRGRGACLNYADS

SEQ ID NO: 157 (percent sequence identity with CBF1: 88% (55/62)): HPIYRGVRLRKSGKWVCEVREPNKKSRIWLGTFKTAEIAARAHDVAALALRGRGACLNFADS

SEQ ID NO: 158 (percent sequence identity with CBF1: 88% (55/62)): HPIYRGVRLRKSGKWVCEVREPNKKSRIWLGTFKTAEIAARAHDVAALALRGRGACLNFADS

SEQ ID NO: 159 (percent sequence identity with CBF1: 88% (55/62)): HPIYRGVRLRKSGKWVCEVREPNKKSRIWLGTFKTAEIAARAHDVAALALRGRGACLNFADS

SEQ ID NO: 160 (percent sequence identity with CBF1: 88% (55/62)): HPIYRGVRLRKSGKWVCEVREPNKKSRIWLGTFKTAEIAARAHDVAALALRGRGACLNFADS

SEQ ID NO: 161 (percent sequence identity with CBF1: 88% (55/62)): HPIYRGVRLRKSGKWVCEVREPNKKSRIWLGTFKTAEIAARAHDVAALALRGRGACLNFADS

SEQ ID NO: 89 is from Glycine max. The AP2 region present in this sequence is:

SEQ ID NO: 167 (percent sequence identity with CBF1: 87%): HPIYSGVRRRNTDKWVSEVREPNKKTRIWLGTFPTPEMAARAHDVAAMALRGRYACLNFADS

SEQ ID NO: 91-93 are from Raphanus sativus. The AP2 regions present in each of these sequences are:

SEQ ID NO: 162 (percent sequence identity with CBF1: HPIYRGVRLRKSGKWVCEVREPNKKSRIWLGTFKTAEIAARAHDVAALALRGRGACLNFADS

SEQ ID NO: 142 (percent sequence identity with CBF1: 88% (55/62)):

HPIYRGVRLRNSGKWVCEVREPNKKSRIWLGTFLTAEIAARAHDVAAIALRGKSACLNFADS

SEQ ID NO: 95 is from Zea mays. The AP2 region present in this sequence is:

SEQ ID NO: 166 (percent sequence identity with CBF1: 80% (51/63)): HPVYRGVRRRGPAGRWVCEVREPNKKSRIWLGTFATPEAAARAHDVAALALRGRAACLNFADS

SEQ ID NO: 116-124 are from Secale cereale. The AP2 regions present in each of these sequences are:

SEQ ID NO: 116 (percent sequence identity with CBF1: 71% (37/52)) ATAQDGEVGAAGRWVCEVRVLGMRGSRLWLGTFVTAEMAARAHDAAVLALSGRKACLNFADS

SEQ ID NO: 118 (percent sequence identity with CBF1: 72% (46/64)) HPLYRGVRRRGRVGQWVCEVRVPGIKGSRLWLGTFNTAEMAARAHDAAVLALSCRAACLNFADS

SEQ ID NO: 120 (percent sequence identity with CBF1: 73% (47/64)) HPLYRGVRRRGRVGQWVCEVRVPGIKGSRLWLGTFNTAEMAARAHDAAVLALSGRKACLNFADS

SEQ ID NO: 122 (percent sequence identity with CBF1: 69%(44/64)) HPLYRGVRRRGRLGQWVCEVRVRGAQGYRLWLGTFTTAEMAARAHDSAVLALLDRAACLNFADS

SEQ ID NO: 124 (percent sequence identity with CBF1: (73% (47/64) HPLYRGVRRRGRVGQWVCEVRVPGIKGSRLWLGTFNTAEMAARAHDAAVLALSGRAACLNFADS

SEQ ID NO: 126 is from Triticum aestivum. The AP2 region present in this sequence is:

SEQ ID NO: 126 (percent sequence identity with CBF1: (73% (47/64) HPLYRGVRRRGRVGQWVCEVRVPGVKGSRLWLGTFTTAEMAARAHDAAVLALSGRAACLNFADS

SEQ ID NO: 128 is found in Glycine max. The AP2 region present in this sequence is:

SEQ ID NO: 128 (percent sequence identity with CBF1: (89% (57/64) HPVYRGVRRRNSDKWVCEVREPNKKTRIWLGTFPTPEMAARAHDVAAMALRGRYACLNFADS

Other Arabidopsis Homologs

G2513 (SEQ ID NO: 99) G2513 is also closely related to CBF1, CBF2, and CBF3 (SEQ ID NO: 2, 13, and 15, respectively). G2513 shares 73% sequence identity with CBF1, 73% sequence identity with CBF2, and 52%% sequence identity with CBF3. In the AP2 domain G2513 shares 77%, 75%, and 80% identity with CBF1, CBF2 and CBF3, respectively.

G2513 corresponds to gene T12C24.14 (AAF88096). G2513 shows sequence similarity, outside of the conserved AP2 domain, with a protein from Nicotiana tabacum (gi12003384 AF211531_{—}1 Avr9/Cf-9 rapidly elicited protein 111B [Nicotiana tabacum]). No published information is available about the functions of G2513. G2513-overexpressing plants were initially small with narrow dark green leaves, grew slowly and initiated floral buds several weeks later than in wild-type controls.

G2513 forms part of a monophyletic group within the Arabidopsis AP2/EREBP family that also includes G40 (SEQ ID NO: 2), G41 (SEQ ID NO: 13), G41 (SEQ ID NO: 15), and G912 (SEQ ID NO: 97), (CBF1, CBF2, CBF3 and CBF4, respectively). However, the clade is divided into two subgroups, one comprised by the four CBF genes (SEQ ID NOs: 2, 13, 15, and 97) and the other by G2107 (SEQ ID NO:101) and G2513 (SEQ ID NO:99).

G2513 is ubiquitously expressed, at significantly higher levels in rosette leaves, flower, and embryo tissues. Because of its phylogenetic relationship to the CBF genes and the delay in floral bud development, G2513 may be used to delay flowering and increase plant biomass relative to control after the plant flowers.

G2107 (SEQ ID NO: 101) shares 44% sequence identity with CBF1 (SEQ ID NO: 2), 45% sequence identity with CBF2 (SEQ ID NO: 13), and 51%% sequence identity with CBF3 (SEQ ID NO: 15). In the AP2 domain G2107 shares 75%, 74%, and 79% sequence identity with CBF1, CBF2, and CBF3, respectively. G2107 shows sequence similarity, outside of the conserved AP2 domain, with a protein from Nicotiana tabacum (gi12003384 AF211531_{—}1 Avr9/Cf-9 rapidly elicited protein 111B [Nicotiana tabacum]). G2107 corresponds to gene F16M19.17 (AAF18701). No published information is available about the function of G2107.

G2107 expression is detected in floral tissues (including embryo and silique), as well as in rosette leaves, but not in roots or germinating seedlings. Because of its phylogenetic relationship to the CBF genes and the delay in floral bud development, G2107 may be used to delay flowering and increase plant biomass relative to control after the latter flowers or when environmental conditions induce stress in control plants.

G21 (SEQ ID NO: 103) corresponds to gene At2g44940 (AAD32841). G21 corresponds to gene At2g44940 (AAD32841). G2107 shares 57% sequence identity with CBF1 (SEQ ID NO: 2), 58% sequence identity with CBF2 (SEQ ID NO: 13), and 53%% sequence identity with CBF3 (SEQ ID NO: 15). In the AP2 domain G21 shares 76%, 72%, and 74% sequence identity with CBF1, CBF2, and CBF3, respectively. G21 does not show extensive sequence similarity with known genes from other plant species outside of the conserved AP2/EREBP domain.

Overexpression of G21 caused alterations in plant growth and development: 35S::G21 plants were smaller than wild type, often possessed curled, darker green leaves, and showed reduced fertility. No alterations were detected in 35S::G21 plants in the physiological and biochemical analyses that were performed.

G21 is ubiquitously expressed, and appears to be induced by several environmental or physiological conditions, in particular cold and abscisic acid. Because of its phylogenetic relationship to the CBF genes and the delay in floral bud development, G21 may also be used to delay flowering and increase plant biomass relative to control after the latter flowers, or when environmental conditions induce stress in control plants.

Summing up the presently disclosed means by which the levels of a cell protectant in a plant cell or plant may be modified, it has been shown that:

a) CBF1, CBF2, CBF3 are expressed in response to environmental stresses. For example, CBF1 expression increases in response to cold stress.

b) CBF1, CBF2, CBF3 have been shown to modify the levels of cell protectants in plant cells. These cell protectants include proline, sugars and fatty acids.

c) CBF1, CBF2, CBF3 have been shown to confer tolerance to environmental stresses. In Arabidopsis, for example, overexpression of any of these polypeptides results in improved tolerance to cold, high salt and drought.

d) A variety of plant genera and species may be transformed with a recombinant polynucleotide encoding a C-repeat/DRE binding factor (CBF)-related polypeptide; these species include Arabidopsis thaliana, leaf mustard (Brassica juncea), Brassica oleracea (including cabbage, Brussels sprouts, broccoli, kohlrabi, cauliflower, and kale), Brassica rapa (including turnip greens, turnip rape, and field mustard), rapeseed and canola (Brassica rapa, Brassica campestris L., and Brassica napus L), Brassica napus (in addition to rapeseed and canola, also includes rutabaga and Swedish turnip), soybean (Glycine max), radish and clover radish (Raphanus sativus), corn (Zea mays), wheat (Triticum), rice (Oryza sativa), rye (Secale cereale), sorghum (Sorghum bicolor and Sorghum vulgare, and barley (Hordeum vulgare).

e) Different plants may be made more tolerant to environmental stresses. Overexpression of the paralogous genes CBF1, CBF2, and CBF3 in different plants, including Arabidopsis and canola, resulted in increased tolerance to several environmental stresses, including freezing, salt, and drought tolerance.

f) Orthologous sequences to CBF genes have been identified in canola, soybeans, rice, corn and other diverse plant species. This demonstrates that CBF genes are present and likely function in a similar manner in diverse species.

g) Overexpression of paralogs of the CBF genes, including CBF4 (G912), have also been shown to confer improved tolerance to environmental stress, which demonstrates that genes encoding polypeptides with the AP2 domain or a similarly functioning variant are able to confer improved stress resistance.

h) Gao et al. ((2002) Plant Mol. Biol. 49 (5), 459-471) have characterized four CBF transcription factors from Brassica napus that function in a similar manner and show a high degree of sequence similarity to Arabidopsis CBF.

i) Zhou et al. have identified a dehydration responsive element binding protein GenBank Accession No. AAD45623) that shares 88% identity with the conserved domain of CBF1.

j) Durrant et al. (2002; unpublished; GenBank Accession No. AAG43548) have identified Avr9/Cf-9 (rapidly elicited protein 111B) in Nicotiana tabacum, the cDNA expression profiling of which reveals rapid, resistance gene-dependent, active oxygen-independent, gene induction during the plant defense response; this protein shares 64% identity with the CBF1 sequence and 85% (53/62 residues) with the conserved domain of CBF1.

Identifying Polynucleotides or Nucleic Acids by Hybridization

Polynucleotides homologous to the sequences illustrated in the Sequence Listing and tables can be identified, for example, by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in the references cited above.

Encompassed by the invention are polynucleotide sequences that are capable of hybridizing to the claimed transcription factor polynucleotide sequences, and, in particular, to those shown in SEQ ID NO: 1, 12, 14, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 115, 117, 119, 121, 123, 125, and 127, and fragments thereof under various conditions of stringency. (See, for example, Wahl et al. (1987) Methods Enzymol. 152: 399-407; Kimmel (1987) Methods Enzymol. 152: 507-511.) In addition to the nucleotide sequences listed in Tables 4 and 5, full length cDNA, orthologs, and paralogs of the present nucleotide sequences may be identified and isolated using well-known methods. The cDNA libraries orthologs, and paralogs of the present nucleotide sequences may be screened using hybridization methods to determine their utility as hybridization target or amplification probes.

With regard to hybridization, conditions that are highly stringent, and means for achieving them, are well known in the art. See, for example, Sambrook et al. (1989) “Molecular Cloning: A Laboratory Manual” (2nd ed., Cold Spring Harbor Laboratory); Berger and Kimmel, editors, (1987) “Guide to Molecular Cloning Techniques”, In Methods in Enzymology: 152: 467-469; and Anderson and Young (1985) “Quantitative Filter Hybridisation.” In: Hames and Higgins, editors, Nucleic Acid Hybridisation, A Practical Approach. Oxford, IRL Press, pages 73-111.

Stability of DNA duplexes is affected by such factors as base composition, length, and degree of base pair mismatch. Hybridization conditions may be adjusted to allow DNAs of different sequence relatedness to hybridize. The melting temperature (Tₘ) is defined as the temperature when 50% of the duplex molecules have dissociated into their constituent single strands. The melting temperature of a perfectly matched duplex, where the hybridization buffer contains formamide as a denaturing agent, may be estimated by the following equations:

(I) DNA-DNA:

Tₘ(° C.)=81.5+16.6(log[Na+])+0.41(% G+C)−0.62(% formamide−500/L

(II) DNA-RNA:

Tₘ(° C.)=79.8+18.5(log[Na+])+0.58(% G+C)+0.12(% G+C)²−0.5(% formamide)−820/L

(III) RNA-RNA:

Tₘ(° C.)=79.8+18.5(log[Na+])+0.58(% G+C)+0.12(% G+C)²−0.35(% formamide)−820/L

where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson et al. (1985) supra). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency (as described by the formula above). As a general guidelines high stringency is typically performed at Tₘ-5° C. to Tₘ-20° C., moderate stringency at Tₘ-20° C. to Tₘ-35° C. and low stringency at Tₘ-35° C. to Tₘ-50° C. for duplex >150 base pairs. Hybridization may be performed at low to moderate stringency (25-50° C. below Tₘ), followed by post-hybridization washes at increasing stringencies. Maximum rates of hybridization in solution are determined empirically to occur at Tₘ-25° C. for DNA-DNA duplex and Tₘ-15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tₘ) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. More preferably, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, for example, to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, for example, formamide, whereas high stringency hybridization may be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. with formamide present. Varying additional parameters, such as hybridization time, the concentration of detergent, for example, sodium dodecyl sulfate (SDS) and ionic strength, are well known to those skilled in the art. Various levels of stringency are accomplished by combining these various conditions as needed.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps will preferably be less than about 30 mM NaCl and 3 mM trisodium citrate, and most preferably less than about 15 mM NaCl and 1.5 mM trisodium citrate.

Thus, hybridization and wash conditions that may be used to bind and remove polynucleotides with less than the desired homology to the nucleic acid sequences or their complements that encode the present transcription factors include, for example:

6×SSC at 65° C.;

50% formamide, 4×SSC at 42° C.; or

0.5×SSC, 0.1% SDS at 65° C.;

with, for example, two wash steps of 10-30 minutes each.. Useful variations on these conditions will be readily apparent to those skilled in the art.

A person of skill in the art would not expect substantial variation among polynucleotide species encompassed within the scope of the present invention because the highly stringent conditions set forth in the above formulae yield structurally similar polynucleotides.

If desired, one may employ wash steps of even greater stringency, including about 0.2×SSC, 0.1% SDS at 65° C. and washing twice, each wash step being about 30 min, or about 0.1×SSC, 0.1% SDS at 65° C. and washing twice for 30 min. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homolog, wash steps may be performed at a lower temperature, for example, 50° C.

An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over 30 min. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art (see, for example, U.S. patent application No. 20010010913).

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid encoding a transcription factor known as of the filing date of the application. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, for example, a fluorescent label, a colorimetric label, a radioactive label, or the like. Labeled hybridization or PCR probes for detecting related polynucleotide sequences may be produced by oligolabeling, nick translation, end-labeling, or PCR amplification using a labeled nucleotide.

Identifying Polynucleotides or Nucleic Acids with Expression Libraries

In addition to hybridization methods, transcription factor homolog polypeptides can be obtained by screening an expression library using antibodies specific for one or more transcription factors. With the provision herein of the disclosed transcription factor, and transcription factor homolog nucleic acid sequences, the encoded polypeptide(s) can be expressed and purified in a heterologous expression system (for example, E. coli) and used to raise antibodies (monoclonal or polyclonal) specific for the polypeptide(s) in question. Antibodies can also be raised against synthetic peptides derived from transcription factor, or transcription factor homolog, amino acid sequences. Methods of raising antibodies are well known in the art and are described in Harlow and Lane (1988), Antibodies: A Laboratory Manual, Cold Spring Harbor Laboratory, New York. Such antibodies can then be used to screen an expression library produced from the plant from which it is desired to clone additional transcription factor homologs, using the methods described above. The selected cDNAs can be confirmed by sequencing and enzymatic activity.

Sequence Variations

Due to the degeneracy of the genetic code, many different polynucleotides can encode identical and/or substantially similar polypeptides in addition to those sequences illustrated in the Sequence Listing. A “variant” of a transcription factor may have an amino acid sequence that is different by one or more deletions, insertions, or substitutions of amino acid residues which produce a silent change and result in a functionally equivalent transcription factor. Thus, it will be readily appreciated by those of skill in the art, that any of a variety of polynucleotide sequences is capable of encoding the transcription factors and transcription factor homolog polypeptides of the invention. Variant nucleic acids having a sequence that differs from the sequences shown in the Sequence Listing, or complementary sequences, that encode functionally equivalent peptides (i.e., peptides having some degree of equivalent or similar biological activity) but differ in sequence from the sequence shown in the sequence listing due to degeneracy in the genetic code, are also within the scope of the invention.

The polypeptide variant may have “conservative” changes, wherein a substituted amino acid has similar structural or chemical properties. Deliberate amino acid substitutions may thus be made on the basis of similarity in polarity, charge, solubility, hydrophobicity, hydrophilicity, and/or the amphipathic nature of the residues, as long as the functional or biological activity of the transcription factor is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine (for more detail on conservative substitutions, see Table 2). More rarely, a variant may have “non-conservative” changes, for example, replacement of a glycine with a tryptophan. Similar minor variations may also include amino acid deletions or insertions, or both. Guidance in determining which and how many amino acid residues may be substituted, inserted or deleted without abolishing functional or biological activity may be found using computer programs well known in the art, for example, DNASTAR software (see U.S. Pat. No. 5,840,544).

Altered or variant polynucleotide sequences encoding polypeptides include those sequences with deletions, insertions, or substitutions of different nucleotides, resulting in a polynucleotide encoding a polypeptide with at least one functional characteristic of the instant polypeptides. Included within this definition are polymorphisms which may or may not be readily detectable using a particular oligonucleotide probe of the polynucleotide encoding the instant polypeptides, and improper or unexpected hybridization to allelic variants, with a locus other than the normal chromosomal locus for the polynucleotide sequence encoding the instant polypeptides.

Allelic variant refers to any of two or more alternative forms of a gene occupying the same chromosomal locus. Allelic variation arises naturally through mutation, and may result in phenotypic polymorphism within populations. Gene mutations can be silent (i.e., no change in the encoded polypeptide) or may encode polypeptides having altered amino acid sequence. The term allelic variant is also used herein to denote a protein encoded by an allelic variant of a gene. Splice variant refers to alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Those skilled in the art would recognize that SEQ ID NO: 2, for example, represents a single transcription factor; allelic variation and alternative splicing may be expected to occur. Allelic variants of SEQ ID NO: 1 can be cloned by probing cDNA or genomic libraries from different individual organisms according to standard procedures. Allelic variants of the DNA sequence shown in SEQ ID NO: 1, including those containing silent mutations and those in which mutations result in amino acid sequence changes, are within the scope of the present invention, as are proteins which are allelic variants of SEQ ID NO: 2. cDNAs generated from alternatively spliced mRNAs, which retain the properties of the transcription factor are included within the scope of the present invention, as are polypeptides encoded by such cDNAs and mRNAs. Allelic variants and splice variants of these sequences can be cloned by probing cDNA or genomic libraries from different individual organisms or tissues according to standard procedures known in the art (see U.S. Pat. No. 6,388,064).

For example, Table 1 illustrates, for example, that the codons AGC, AGT, TCA, TCC, TCG, and TCT all encode the same amino acid: serine. Accordingly, at each position in the sequence where there is a codon encoding serine, any of the above trinucleotide sequences can be used without altering the encoded polypeptide.

TABLE 1

| Amino acid | | | Possible Codons |
|---|---|---|---|
| Alanine | Ala | A | GCA GCC GCG GCU |
| Cysteine | Cys | C | TGC TGT |
| Aspartic acid | Asp | D | GAC GAT |
| Glutamic acid | Glu | E | GAA GAG |
| Phenylalanine | Phe | F | TTC TTT |
| Glycine | Gly | G | GGA GGC GGG GGT |
| Histidine | His | H | CAC CAT |
| Isoleucine Ile | I | ATA ATC ATT | |
| Lysine | Lys | K | AAA AAG |
| Leucine | Leu | L | TTA TTG CTA CTC CTG CTT |
| Methionine Met | M | ATG | |
| Asparagine Asn | N | AAC AAT | |
| Proline | Pro | P | CCA CCC CCG CCT |
| Glutamine | Gin | Q | CAA CAG |
| Arginine | Arg | R | AGA AGG CGA CGC CGG CGT |
| Serine | Ser | S | AGC AGT TCA TCC TCG TCT |
| Threonine | Thr | T | ACA ACC ACG ACT |
| Valine | Val | V | GTA GTC GTG GTT |
| Tryptophan Trp | W | TGG | |
| Tyrosine | Tyr | Y | TAC TAT |

Sequence alterations that do not change the amino acid sequence encoded by the polynucleotide are termed “silent” variations. With the exception of the codons ATG and TGG, encoding methionine and tryptophan, respectively, any of the possible codons for the same amino acid can be substituted by a variety of techniques, for example, site-directed mutagenesis, available in the art. Accordingly, any and all such variations of a sequence selected from the above table are a feature of the invention.

In addition to silent variations, other conservative variations that alter one, or a few amino acids in the encoded polypeptide, can be made without altering the function of the polypeptide, these conservative variants are, likewise, a feature of the invention.

For example, substitutions, deletions and insertions introduced into the sequences provided in the Sequence Listing are also envisioned by the invention. Such sequence modifications can be engineered into a sequence by site-directed mutagenesis (Wu (editor) Meth. Enzymol. (1993) vol. 217, Academic Press) or the other methods noted below. Amino acid substitutions are typically of single residues; insertions usually will be on the order of about from 1 to 10 amino acid residues; and deletions will range about from 1 to 30 residues. In preferred embodiments, deletions or insertions are made in adjacent pairs, for example, a deletion of two residues or insertion of two residues. Substitutions, deletions, insertions or any combination thereof can be combined to arrive at a sequence. The mutations that are made in the polynucleotide encoding the transcription factor should not place the sequence out of reading frame and should not create complementary regions that could produce secondary mRNA structure. Preferably, the polypeptide encoded by the DNA performs the desired function.

Conservative substitutions are those in which at least one residue in the amino acid sequence has been removed and a different residue inserted in its place. Such substitutions generally are made in accordance with the Table 2 when it is desired to maintain the activity of the protein. Table 2 shows amino acids which can be substituted for an amino acid in a protein and which are typically regarded as conservative substitutions.

TABLE 2

| Residue | Conservative Substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Similar substitutions are those in which at least one residue in the amino acid sequence has been removed and a different residue inserted in its place. Such substitutions generally are made in accordance with the Table 3 when it is desired to maintain the activity of the protein. Table 3 shows amino acids which can be substituted for an amino acid in a protein and which are typically regarded as structural and functional substitutions. For example, a residue in column 1 of Table 3 may be substituted with residue in column 2; in addition, a residue in column 2 of Table 3 may be substituted with the residue of column 1.

TABLE 3

| Residue | Similar Substitutions |
|---|---|
| Ala | Ser; Thr; Gly; Val; Leu; Ile |
| Arg | Lys; His; Gly |
| Asn | Gln; His; Gly; Ser; Thr |
| Asp | Glu, Ser; Thr |
| Gln | Asn; Ala |
| Cys | Ser; Gly |
| Glu | Asp |
| Gly | Pro; Arg |
| His | Asn; Gln; Tyr; Phe; Lys; Arg |
| Ile | Ala; Leu; Val; Gly; Met |
| Leu | Ala; Ile; Val; Gly; Met |
| Lys | Arg; His; Gln; Gly; Pro |
| Met | Leu; Ile; Phe |
| Phe | Met; Leu; Tyr; Trp; His; Val; Ala |
| Ser | Thr; Giy; Asp; Ala; Val; Ile; His |
| Thr | Ser; Val; Ala; Gly |
| Trp | Tyr; Phe; His |
| Tyr | Trp; Phe; His |
| Val | Ala; Ile; Leu; Gly; Thr; Ser; Glu |

Substitutions that are less conservative than those in Table 2 can be selected by picking residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histidyl, is substituted for (or by) an electronegative residue, for example, glutamyl or aspartyl; or (d) a residue having a bulky side chain, for example, phenylalanine, is substituted for (or by) one not having a side chain, for example, glycine.

Further Modifying Sequences of the Invention—Mutation/Forced Evolution

In addition to generating silent or conservative substitutions as noted, above, the present invention optionally includes methods of modifying the sequences of the Sequence Listing. In the methods, nucleic acid or protein modification methods are used to alter the given sequences to produce new sequences and/or to chemically or enzymatically modify given sequences to change the properties of the nucleic acids or proteins.

Thus, in one embodiment, given nucleic acid sequences are modified, for example, according to standard mutagenesis or artificial evolution methods to produce modified sequences. The modified sequences may be created using purified natural polynucleotides isolated from any organism or may be synthesized from purified compositions and chemicals using chemical means well know to those of skill in the art. For example, Ausubel, supra, provides additional details on mutagenesis methods. Artificial forced evolution methods are described, for example, by Stemmer (1994) Nature 370: 389-391, Stemmer (1994) Proc. Natl. Acad. Sci. 91: 10747-10751, and U.S. Pat. Nos. 5,811,238, 5,837,500, and 6,242,568. Methods for engineering synthetic transcription factors and other polypeptides are described, for example, by Zhang et al. (2000) J. Biol. Chem. 275: 33850-33860, Liu et al. (2001) J. Biol. Chem. 276: 11323-11334, and Isalan et al. (2001) Nature Biotechnol. 19: 656-660. Many other mutation and evolution methods are also available and expected to be within the skill of the practitioner.

Similarly, chemical or enzymatic alteration of expressed nucleic acids and polypeptides can be performed by standard methods. For example, sequence can be modified by addition of lipids, sugars, peptides, organic or inorganic compounds, by the inclusion of modified nucleotides or amino acids, or the like. For example, protein modification techniques are illustrated in Ausubel, supra. Further details on chemical and enzymatic modifications can be found herein. These modification methods can be used to modify any given sequence, or to modify any sequence produced by the various mutation and artificial evolution modification methods noted herein.

Accordingly, the invention provides for modification of any given nucleic acid by mutation, evolution, chemical or enzymatic modification, or other available methods, as well as for the products produced by practicing such methods, for example, using the sequences herein as a starting substrate for the various modification approaches.

For example, optimized coding sequence containing codons preferred by a particular prokaryotic or eukaryotic host can be used for example, to increase the rate of translation or to produce recombinant RNA transcripts having desirable properties, such as a longer half-life, as compared with transcripts produced using a non-optimized sequence. Translation stop codons can also be modified to reflect host preference. For example, preferred stop codons for Saccharomyces cerevisiae and mammals are TAA and TGA, respectively. The preferred stop codon for monocotyledonous plants is TGA, whereas insects and E. coli prefer to use TAA as the stop codon.

The polynucleotide sequences of the present invention can also be engineered in order to alter a coding sequence for a variety of reasons, including but not limited to, alterations which modify the sequence to facilitate cloning, processing and/or expression of the gene product. For example, alterations are optionally introduced using techniques which are well known in the art, for example, site-directed mutagenesis, to insert new restriction sites, to alter glycosylation patterns, to change codon preference, to introduce splice sites, etc.

Furthermore, a fragment or domain derived from any of the polypeptides of the invention can be combined with domains derived from other transcription factors or synthetic domains to modify the biological activity of a transcription factor. For instance, a DNA-binding domain derived from a transcription factor of the invention can be combined with the activation domain of another transcription factor or with a synthetic activation domain. A transcription activation domain assists in initiating transcription from a DNA-binding site. Examples include the transcription activation region of VP16 or GAL4 (Moore et al. (1998) Proc. Natl. Acad. Sci. 95: 376-381; and Aoyama et al. (1995) Plant Cell 7: 1773-1785), peptides derived from bacterial sequences (Ma and Ptashne (1987) Cell 51: 113-119) and synthetic peptides (Giniger and Ptashne (1987) Nature 330: 670-672).

Expression and Modification of Polypeptides

Typically, polynucleotide sequences of the invention are incorporated into recombinant DNA (or RNA) molecules that direct expression of polypeptides of the invention in appropriate host cells, transgenic plants, in vitro translation systems, or the like. Due to the inherent degeneracy of the genetic code, nucleic acid sequences which encode substantially the same or a functionally equivalent amino acid sequence can be substituted for any listed sequence to provide for cloning and expressing the relevant homolog.

Vectors, Promoters, and Expression Systems

The present invention includes recombinant constructs comprising one or more of the nucleic acid sequences herein. The constructs typically comprise a vector, such as a plasmid, a cosmid, a phage, a virus (for example, a plant virus), a bacterial artificial chromosome (BAC), a yeast artificial chromosome (YAC), or the like, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available.

General texts that describe molecular biological techniques useful herein, including the use and production of vectors, promoters and many other relevant topics, include Berger, Sambrook and Ausubel, supra. Any of the identified sequences can be incorporated into a cassette or vector, for example, for expression in plants. A number of expression vectors suitable for stable transformation of plant cells or for the establishment of transgenic plants have been described including those described in Weissbach and Weissbach (1989) Methods for Plant Molecular Biology, Academic Press, and Gelvin et al. (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303: 209, Bevan (1984) Nucl Acid Res. 12: 8711-8721, Klee (1985) Bio/Technology 3: 637-642, for dicotyledonous plants.

Alternatively, non-Ti vectors can be used to transfer the DNA into monocotyledonous plants and cells by using free DNA delivery techniques. Such methods can involve, for example, the use of liposomes, electroporation, microprojectile bombardment, silicon carbide whiskers, and viruses. By using these methods transgenic plants such as wheat, rice (Christou (1991) Bio/Technology 9: 957-962) and corn (Gordon-Kamm (1990) Plant Cell 2: 603-618) can be produced. An immature embryo can also be a good target tissue for monocots for direct DNA delivery techniques by using the particle gun (Weeks et al. (1993) Plant Physiol. 102: 1077-1084; Vasil (1993) Bio/Technology 10: 667-674; Wan and Lemeaux (1994) Plant Physiol. 104: 37-48, and for Agrobacterium-mediated DNA transfer (Ishida et al. (1996) Nature Biotechnol. 14: 745-750).

Typically, plant transformation vectors include one or more cloned plant coding sequence (genomic or cDNA) under the transcriptional control of 5′ and 3′ regulatory sequences and a dominant selectable marker. Such plant transformation vectors typically also contain a promoter (for example, a regulatory region controlling inducible or constitutive, environmentally-or developmentally-regulated, or cell- or tissue-specific expression), a transcription initiation start site, an RNA processing signal (such as intron splice sites), a transcription termination site, and/or a polyadenylation signal.

Examples of constitutive plant promoters which can be useful for expressing the TF sequence include: the cauliflower mosaic virus (CaMV) 35S promoter, which confers constitutive, high-level expression in most plant tissues (see, for example, Odell et al. (1985) Nature 313: 810-812); the nopaline synthase promoter (An et al. (1988) Plant Physiol. 88: 547-552); and the octopine synthase promoter (Fromm et al. (1989) Plant Cell 1: 977-984).

A variety of plant gene promoters that regulate gene expression in response to environmental, hormonal, chemical, developmental signals, and in a tissue-active manner can be used for expression of a TF sequence in plants. Choice of a promoter is based largely on the phenotype of interest and is determined by such factors as tissue (for example, seed, fruit, root, pollen, vascular tissue, flower, carpel, etc.), inducibility (for example, in response to wounding, heat, cold, drought, light, pathogens, etc.), timing, developmental stage, and the like. Numerous known promoters have been characterized and can favorably be employed to promote expression of a polynucleotide of the invention in a transgenic plant or cell of interest. For example, tissue specific promoters include: seed-specific promoters (such as the napin, phaseolin or DC3 promoter described in U.S. Pat. No. 5,773,697), fruit-specific promoters that are active during fruit ripening (such as the dru 1 promoter (U.S. Pat. No. 5,783,393), or the 2A11 promoter (U.S. Pat. No. 4,943,674) and the tomato polygalacturonase promoter (Bird et al. (1988) Plant Mol. Biol. 11: 651-662), root-specific promoters, such as those disclosed in U.S. Pat. Nos. 5,618,988, 5,837,848 and 5,905,186, pollen-active promoters such as PTA29, PTA26 and PTA13 (U.S. Pat. No. 5,792,929), promoters active in vascular tissue (Ringli et al. (1998) Plant Mol. Biol. 37: 977-988), flower-specific (Kaiser et al, (1995) Plant Mol. Biol. 28: 231-243), pollen (Baerson et al. (1994) Plant Mol. Biol. 26: 1947-1959), carpels (Ohl et al. (1990) Plant Cell 2: 837-848), pollen and ovules (Baerson et al. (1993) Plant Mol. Biol. 22: 255-267), auxin-inducible promoters (such as that described in van der Kop et al. (1999) Plant Mol. Biol. 39: 979-990 or Baumann et al. (1999) Plant Cell 11: 323-334), cytokinin-inducible promoter (Guevara-Garcia (1998) Plant Mol. Biol. 38: 743-753), promoters responsive to gibberellin (Shi et al. (1998) Plant Mol. Biol. 38: 1053-1060, Willmott et al. (1998) 38: 817-825) and the like. Additional promoters are those that elicit expression in response to heat (Ainley et al. (1993) Plant Mol. Biol. 22: 13-23), light (for example, the pea rbcS-3A promoter, Kuhlemeier et al. (1989) Plant Cell 1: 471-478, and the maize rbcS promoter, Schaffner and Sheen (1991) Plant Cell 3: 997-1012); wounding (for example, wunI, Siebertz et al. (1989) Plant Cell 1: 961-968); pathogens (such as the PR-1 promoter described in Buchel et al. (1999) Plant Mol. Biol. 40: 387-396, and the PDF1.2 promoter described in Manners et al. (1998) Plant Mol. Biol. 38: 1071-1080), and chemicals such as methyl jasmonate or salicylic acid (Gatz et al. (1997) Ann. Rev. Plant Physiol. Plant Mol. Biol. 48: 89-108). In addition, the timing of the expression can be controlled by using promoters such as those acting at senescence (Gan et al. (1995) Science 270: 1986-1988); or late seed development (Odell et al. (1994) Plant Physiol. 106: 447-458).

Plant expression vectors can also include RNA processing signals that can be positioned within, upstream or downstream of the coding sequence. In addition, the expression vectors can include additional regulatory sequences from the 3′-untranslated region of plant genes, for example, a 3′ terminator region to increase mRNA stability of the mRNA, such as the PI-II terminator region of potato or the octopine or nopaline synthase 3′ terminator regions.

Additional Expression Elements

Specific initiation signals can aid in efficient translation of coding sequences. These signals can include, for example, the ATG initiation codon and adjacent sequences. In cases where a coding sequence, its initiation codon and upstream sequences are inserted into the appropriate expression vector, no additional translational control signals may be needed. However, in cases where only coding sequence (for example, a mature protein coding sequence), or a portion thereof, is inserted, exogenous transcriptional control signals including the ATG initiation codon can be separately provided. The initiation codon is provided in the correct reading frame to facilitate transcription. Exogenous transcriptional elements and initiation codons can be of various origins, both natural and synthetic. The efficiency of expression can be enhanced by the inclusion of enhancers appropriate to the cell system in use.

Expression Hosts

The present invention also relates to host cells which are transduced with vectors of the invention, and the production of polypeptides of the invention (including fragments thereof) by recombinant techniques. Host cells are genetically engineered (i.e., nucleic acids are introduced, for example, transduced, transformed or transfected) with the vectors of this invention, which may be, for example, a cloning vector or an expression vector comprising the relevant nucleic acids herein. The vector is optionally a plasmid, a viral particle, a phage, a naked nucleic acid, etc. The engineered host cells can be cultured in conventional nutrient media modified as appropriate for activating promoters, selecting transformants, or amplifying the relevant gene. The culture conditions, such as temperature, pH and the like, are those previously used with the host cell selected for expression, and will be apparent to those skilled in the art and in the references cited herein, including, Sambrook and Ausubel.

The host cell can be a eukaryotic cell, such as a yeast cell, or a plant cell, or the host cell can be a prokaryotic cell, such as a bacterial cell. Plant protoplasts are also suitable for some applications. For example, the DNA fragments are introduced into plant tissues, cultured plant cells or plant protoplasts by standard methods including electroporation (Fromm et al. (1985) Proc. Natl. Acad. Sci. 82: 5824, infection by viral vectors such as cauliflower mosaic virus (CaMV) (Hohn et al. (1982) Molecular Biology of Plant Tumors, (Academic Press, New York) pp. 549-560; U.S. Pat. No. 4,407,956), high velocity ballistic penetration by small particles with the nucleic acid either within the matrix of small beads or particles, or on the surface (Klein et al. (1987) Nature 327: 70-73), use of pollen as vector (WO 85/01856), or use of Agrobacterium tumefaciens or A. rhizogenes carrying a T-DNA plasmid in which DNA fragments are cloned. The T-DNA plasmid is transmitted to plant cells upon infection by Agrobacterium tumefaciens, and a portion is stably integrated into the plant genome (Horsch et al. (1984) Science 233: 496-498; Fraley et al. (1983) Proc. Natl. Acad. Sci. 80: 4803).

The cell can include a nucleic acid of the invention that encodes a polypeptide, wherein the cell expresses a polypeptide of the invention. The cell can also include vector sequences, or the like. Furthermore, cells and transgenic plants that include any polypeptide or nucleic acid above or throughout this specification, for example, produced by transduction of a vector of the invention, are an additional feature of the invention.

Modified Amino Acid Residues

Polypeptides of the invention may contain one or more modified amino acid residues. The presence of modified amino acids may be advantageous in, for example, increasing polypeptide half-life, reducing polypeptide antigenicity or toxicity, increasing polypeptide storage stability, or the like. Amino acid residue(s) are modified, for example, co-translationally or post-translationally during recombinant production or modified by synthetic or chemical means.

Non-limiting examples of a modified amino acid residue include incorporation or other use of acetylated amino acids, glycosylated amino acids, sulfated amino acids, prenylated (for example, farnesylated, geranylgeranylated) amino acids, PEG modified (for example, “PEGylated”) amino acids, biotinylated amino acids, carboxylated amino acids, phosphorylated amino acids, etc. References adequate to guide one of skill in the modification of amino acid residues are replete throughout the literature.

The modified amino acid residues may prevent or increase affinity of the polypeptide for another molecule, including, but not limited to, polynucleotide, proteins, carbohydrates, lipids and lipid derivatives, and other organic or synthetic compounds.

Identification of Additional Factors

A transcription factor provided by the present invention can also be used to identify additional endogenous or exogenous molecules that can affect a phentoype or trait of interest. On the one hand, such molecules include organic (small or large molecules) and/or inorganic compounds that affect expression of (i.e., regulate) a particular transcription factor. Alternatively, such molecules include endogenous molecules that are acted upon either at a transcriptional level by a transcription factor of the invention to modify a phenotype as desired. For example, the transcription factors can be employed to identify one or more downstream gene with which is subject to a regulatory effect of the transcription factor. In one approach, a transcription factor or transcription factor homolog of the invention is expressed in a host cell, for example, a transgenic plant cell, tissue or explant, and expression products, either RNA or protein, of likely or random targets are monitored, for example, by hybridization to a microarray of nucleic acid probes corresponding to genes expressed in a tissue or cell type of interest, by two-dimensional gel electrophoresis of protein products, or by any other method known in the art for assessing expression of gene products at the level of RNA or protein. Alternatively, a transcription factor of the invention can be used to identify promoter sequences (i.e., binding sites) involved in the regulation of a downstream target. After identifying a promoter sequence, interactions between the transcription factor and the promoter sequence can be modified by changing specific nucleotides in the promoter sequence or specific amino acids in the transcription factor that interact with the promoter sequence to alter a plant trait. Typically, transcription factor DNA-binding sites are identified by gel shift assays. After identifying the promoter regions, the promoter region sequences can be employed in double-stranded DNA arrays to identify molecules that affect the interactions of the transcription factors with their promoters (Bulyk et al. (1999) Nature Biotechnol. 17: 573-577).

The identified transcription factors are also useful to identify proteins that modify the activity of the transcription factor. Such modification can occur by covalent modification, such as by phosphorylation, or by protein-protein (homo- or heteropolymer) interactions. Any method suitable for detecting protein-protein interactions can be employed. Among the methods that can be employed are co-immunoprecipitation, cross-linking and co-purification through gradients or chromatographic columns, and the two-hybrid yeast system.

The two-hybrid system detects protein interactions in vivo and is described in Chien et al. ((1991), Proc. Natl. Acad. Sci. 88: 9578-9582) and is commercially available from Clontech (Palo Alto, Calif.). In such a system, plasmids are constructed that encode two hybrid proteins: one consists of the DNA-binding domain of a transcription activator protein fused to the TF polypeptide and the other consists of the transcription activator protein's activation domain fused to an unknown protein that is encoded by a cDNA that has been recombined into the plasmid as part of a cDNA library. The DNA-binding domain fusion plasmid and the cDNA library are transformed into a strain of the yeast Saccharomyces cerevisiae that contains a reporter gene (for example, lacZ) whose regulatory region contains the transcription activator's binding site. Either hybrid protein alone cannot activate transcription of the reporter gene. Interaction of the two hybrid proteins reconstitutes the functional activator protein and results in expression of the reporter gene, which is detected by an assay for the reporter gene product. Then, the library plasmids responsible for reporter gene expression are isolated and sequenced to identify the proteins encoded by the library plasmids. After identifying proteins that interact with the transcription factors, assays for compounds that interfere with the TF protein-protein interactions can be preformed.

Identification of Modulators

In addition to the intracellular molecules described above, extracellular molecules that alter activity or expression of a transcription factor, either directly or indirectly, can be identified. For example, the methods can entail first placing a candidate molecule in contact with a plant or plant cell. The molecule can be introduced by topical administration, such as spraying or soaking of a plant, and then the molecule's effect on the expression or activity of the TF polypeptide or the expression of the polynucleotide monitored. Changes in the expression of the TF polypeptide can be monitored by use of polyclonal or monoclonal antibodies, gel electrophoresis or the like. Changes in the expression of the corresponding polynucleotide sequence can be detected by use of microarrays, Northerns, quantitative PCR, or any other technique for monitoring changes in mRNA expression. These techniques are exemplified in Ausubel et al., editors, Current Protocols in Molecular Biology, John Wiley & Sons (1998, and supplements through 2001). Such changes in the expression levels can be correlated with modified plant traits and thus identified molecules can be useful for soaking or spraying on fruit, vegetable and grain crops to modify traits in plants.

Essentially any available composition can be tested for modulatory activity of expression or activity of any nucleic acid or polypeptide herein. Thus, available libraries of compounds such as chemicals, polypeptides, nucleic acids and the like can be tested for modulatory activity. Often, potential modulator compounds can be dissolved in aqueous or organic (for example, DMSO-based) solutions for easy delivery to the cell or plant of interest in which the activity of the modulator is to be tested. Optionally, the assays are designed to screen large modulator composition libraries by automating the assay steps and providing compounds from any convenient source to assays, which are typically run in parallel (for example, in microtiter formats on microtiter plates in robotic assays).

In one embodiment, high throughput screening methods involve providing a combinatorial library containing a large number of potential compounds (potential modulator compounds). Such “combinatorial chemical libraries” are then screened in one or more assays, as described herein, to identify those library members (particular chemical species or subclasses) that display a desired characteristic activity. The compounds thus identified can serve as target compounds.

A combinatorial chemical library can be, for example, a collection of diverse chemical compounds generated by chemical synthesis or biological synthesis. For example, a combinatorial chemical library such as a polypeptide library is formed by combining a set of chemical building blocks (for example, in one example, amino acids) in every possible way for a given compound length (i.e., the number of amino acids in a polypeptide compound of a set length). Exemplary libraries include peptide libraries, nucleic acid libraries, antibody libraries (see, for example, Vaughn et al. (1996) Nature Biotechnol. 14: 309-314 and PCT/US96/10287), carbohydrate libraries (see, for example, Liang et al. Science (1996) 274: 1520-1522 and U.S. Pat. No. 5,593,853), peptide nucleic acid libraries (see, for example, U.S. Pat. No. 5,539,083), and small organic molecule libraries (see, for example, benzodiazepines, Baum (1993) Chem Eng. News January 18, p. 33; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337) and the like.

Preparation and screening of combinatorial or other libraries is well known to those of skill in the art. Such combinatorial chemical libraries include, but are not limited to, peptide libraries (see, for example, U.S. Pat. No. 5,010,175; Furka, (1991) Int. J. Pept. Prot. Res. 37: 487-493; and Houghton et al. (1991) Nature 354: 84-88). Other chemistries for generating chemical diversity libraries can also be used.

In addition, as noted, compound screening equipment for high-throughput screening is generally available, for example, using any of a number of well known robotic systems that have also been developed for solution phase chemistries useful in assay systems. These systems include automated workstations including an automated synthesis apparatus and robotic systems utilizing robotic arms. Any of the above devices are suitable for use with the present invention, for example, for high-throughput screening of potential modulators. The nature and implementation of modifications to these devices (if any) so that they can operate as discussed herein will be apparent to persons skilled in the relevant art.

Indeed, entire high-throughput screening systems are commercially available. These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. Similarly, microfluidic implementations of screening are also commercially available.

The manufacturers of such systems provide detailed protocols the various high throughput. Thus, for example, Zymark Corp. provides technical bulletins describing screening systems for detecting the modulation of gene transcription, ligand binding, and the like. The integrated systems herein, in addition to providing for sequence alignment and, optionally, synthesis of relevant nucleic acids, can include such screening apparatus to identify modulators that have an effect on one or more polynucleotides or polypeptides according to the present invention.

In some assays it is desirable to have positive controls to ensure that the components of the assays are working properly. At least two types of positive controls are appropriate. That is, known transcriptional activators or inhibitors can be incubated with cells/plants/etc. in one sample of the assay, and the resulting increase/decrease in transcription can be detected by measuring the resulting increase in RNA/protein expression, etc., according to the methods herein. It will be appreciated that modulators can also be combined with transcriptional activators or inhibitors to find modulators that inhibit transcriptional activation or transcriptional repression. Either expression of the nucleic acids and proteins herein or any additional nucleic acids or proteins activated by the nucleic acids or proteins herein, or both, can be monitored.

In an embodiment, the invention provides a method for identifying compositions that modulate the activity or expression of a polynucleotide or polypeptide of the invention. For example, a test compound, whether a small or large molecule, is placed in contact with a cell, plant (or plant tissue or explant), or composition comprising the polynucleotide or polypeptide of interest and a resulting effect on the cell, plant, (or tissue or explant) or composition is evaluated by monitoring, either directly or indirectly, one or more of: expression level of the polynucleotide or polypeptide, activity (or modulation of the activity) of the polynucleotide or polypeptide. In some cases, an alteration in a plant phenotype can be detected following contact of a plant (or plant cell, or tissue or explant) with the putative modulator, for example, by modulation of expression or activity of a polynucleotide or polypeptide of the invention. Modulation of expression or activity of a polynucleotide or polypeptide of the invention may also be caused by molecular elements in a signal transduction second messenger pathway and such modulation can affect similar elements in the same or another signal transduction second messenger pathway.

Subsequences

Also contemplated are uses of polynucleotides, also referred to herein as oligonucleotides, typically having at least 12 bases, preferably at least 15, more preferably at least 20, 30, or 50 bases, which hybridize under at least highly stringent (or ultra-high stringent or ultra-ultra-high stringent conditions) conditions to a polynucleotide sequence described above. The polynucleotides may be used as probes, primers, sense and antisense agents, and the like, according to methods as noted supra.

Subsequences of the polynucleotides of the invention, including polynucleotide fragments and oligonucleotides are useful as nucleic acid probes and primers. An oligonucleotide suitable for use as a probe or primer is at least about 15 nucleotides in length, more often at least about 18 nucleotides, often at least about 21 nucleotides, frequently at least about 30 nucleotides, or about 40 nucleotides, or more in length. A nucleic acid probe is useful in hybridization protocols, for example, to identify additional polypeptide homologs of the invention, including protocols for microarray experiments. Primers can be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, for example, by the polymerase chain reaction (PCR) or other nucleic-acid amplification methods. See Sambrook and Ausubel, supra.

In addition, the invention includes an isolated or recombinant polypeptide including a subsequence of at least about 15 contiguous amino acids encoded by the recombinant or isolated polynucleotides of the invention. For example, such polypeptides, or domains or fragments thereof, can be used as immunogens, for example, to produce antibodies specific for the polypeptide sequence, or as probes for detecting a sequence of interest. A subsequence can range in size from about 15 amino acids in length up to and including the full length of the polypeptide.

To be encompassed by the present invention, an expressed polypeptide which comprises such a polypeptide subsequence performs at least one biological function of the intact polypeptide in substantially the same manner, or to a similar extent, as does the intact polypeptide. For example, a polypeptide fragment can comprise a recognizable structural motif or functional domain such as a DNA binding domain that binds to a specific DNA promoter region, an activation domain or a domain for protein-protein interactions.

2. Description of the Invention

The present invention relates to a method for modifying the biomass of a plant. By modifying a plant's biomass, any of a number of desirable plant traits can be modified, including plant yield, larger root mass to increase the tolerance to certain environmental stresses, modified leaf area in response to different light intensities, increased leaf mass for human or animal consumption, or the like.

The present invention also relates to a method for increasing the levels of a cell protectant in a cell. By increasing the levels of the cell protectant, a cell's or plant's response to a variety of environmental stresses can be improved. The type of environmental stress that can be modified includes the cold or freezing tolerance of the cell or the drought or salinity tolerance of the cell or plant. The cell protectant may be a cryoprotectant, an osmoprotectant or the like. Exemplary cell protectants include proline, sugars, lipids or the like. The method can also be used to increase levels of a number of cell protectants simultaneously.

The method entails altering the levels of a polynucleotide encoding a CBF-related polypeptide in a transformed plant. The transformed plant may be generated by transforming a plant with an expression vector comprising a polynucleotide sequence encoding a CBF-related polypeptide or by breeding after the initial transformation of a parental plant comprising the expression vector. Transformed plants are then selected that express the polynucleotide. The resulting plants are plants with increased biomass, including, for example, larger leaves and/or larger root systems, or plants that produce higher levels of cell protectants.

The method also entails generating a transformed cell or plant that overexpresses a recombinant polynucleotide encoding a CBF-related polypeptide. The transformed cell or plant may be generated by transforming a cell or plant with an expression vector comprising a polynucleotide sequence encoding a cold-regulatable polypeptide or by breeding after the initial transformation of a parental cell comprising the expression vector. Transformed cells or plants are then selected for expression of the polynucleotide and grown. The resulting cells or plants produce higher levels of cell protectants. Higher levels of cell protectants can be detected either in the absence of or after exposure to cold temperatures (cold acclimation). However, higher levels of cell protectants are typically observed in cells or plants that have been cold acclimated compared with levels observed in cells or plants that have not been cold-acclimated.

By increasing the levels of a single cell protectant or a combination of cell protectants simultaneously in the cell, the cell's tolerance to environmental stresses can be substantially improved, as measured for example by a plant's survival rate after exposure to freezing temperatures or the growth of a plant's roots after exposure to drought conditions or high salinity.

The present invention also relates to a method for producing a cell protectant from a cell or plant. The method entails generating a transformed cell that overexpresses a recombinant polynucleotide encoding a CBF-related polypeptide. Expression of the recombinant polynucleotide in the cell or plant derived from the transformed cell causes metabolic pathways that produce or accumulate certain cell protectants to be turned on so that higher levels of the cell protectants are produced. For example, we have observed that the key proline biosynthetic pathway enzyme, P5CS, is expressed at higher levels when the CBF-related polynucleotide is overexpressed. Then the cell protectant, such as proline, sugars or lipids, can be isolated from the plant using well known isolation and purification methods

The present invention can be applied to modify the biomass or increase cell protectant levels in or improve the environmental stress tolerance of a variety of plant cells or plants in particular, cells or plants monocots, dicots and gymnosperms. In particular the invention may be used for modifying the biomass or environmental stress response of agriculturally important plant species, including but not limited to, crops such as soybean, wheat, corn, potato, cotton, rice, oilseed rape (including canola), sunflower, alfalfa, sugarcane and turf; or fruits and vegetables, such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, watermelon, rosaceous fruits (such as apple, peach, pear, cherry, and plum) and vegetable brassicas (such as broccoli, cabbage, cauliflower, Brussels sprouts, and kohlrabi). Other crops, fruits and vegetables whose phenotype may be changed include barley, currant, avocado, citrus fruits such as oranges, lemons, grapefruit and tangerines, artichoke, cherries, nuts such as the walnut and peanut, endive, leek, roots, such as arrowroot, beet, cassava, turnip, radish, yam, sweet potato and beans. The present invention may also be employed to modify plant biomass and increase cell protectant levels in woody plants, such pine, poplar and eucalyptus.

A. CBF-Related Polypeptide

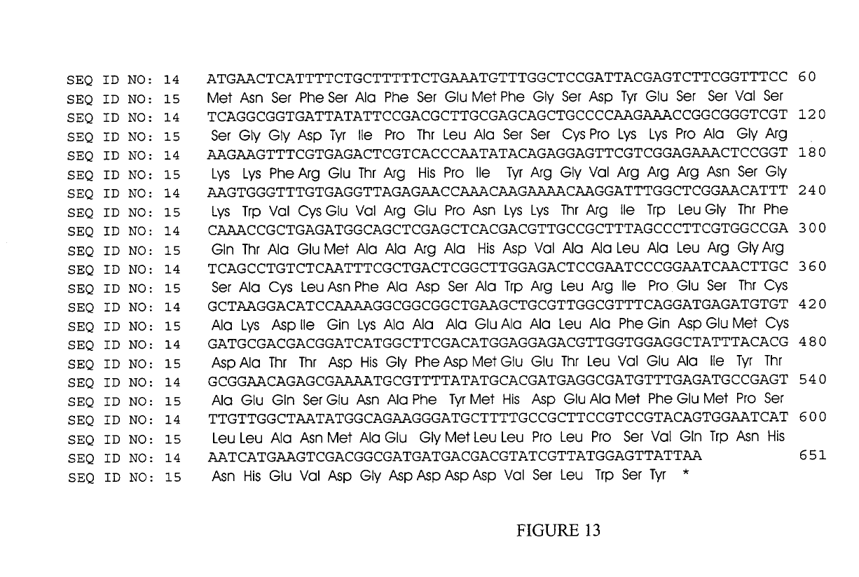

The CBF-related protein may comprise a whole gene coding sequence or a fragment or domain of a coding sequence. A “fragment or domain”, as referred to polypeptides, may be a portion of a polypeptide which performs at least one biological function of the intact polypeptide in substantially the same manner or to a similar extent as does the intact polypeptide. A fragment may comprise, for example, a DNA binding domain that binds to a specific DNA promoter region (such as the AP2 domain), an activation domain or a domain for protein-protein interactions. Fragments may vary in size from as few as 6 amino acids to the length of the intact polypeptide, but are preferably at least 30 amino acids in length and more preferably at least 60 amino acids in length. For example, one can identify any number of 60 amino acid long fragments (1-60, 5-65, 10-70, 15-75, etc.) along the length of the CBF3 polypeptide shown in FIG. 13. In reference to a nucleotide sequence “a fragment” refers to any sequence of at least 18 consecutive nucleotides, preferably at least 30 nucleotides, more preferably at least 50, of any of the sequences provided herein.

The CBF-related polypeptides encompass naturally occurring sequences. Numerous CBF-related proteins have been previously identified and include the genes, CBF1, CBF2, and CBF3 (also known as DREB1b, DREB1c, and DREB1a, respectively), which are located in tandem on chromosome 4 in Arabidopsis (Gilmour et al. (1998) supra; Shinwari et al. (1998) Biochem. Biophys. Res. Commun. 250: 161-170). Additional examples of CBF-related polypeptides include those described in Stockinger et al. PCT publication WO99/38977, and U.S. patent application Ser. No. 09/198,119, entitled “Plant Having Altered Environmental Stress Tolerance”, filed Nov. 23, 1998 and U.S. Provisional Patent Application No. 60/165,860 entitled “Method for Modifying the Cold Resistance of Plants”, filed Nov. 16, 1999.

The CBF-related polypeptides may also encompass non-naturally occurring sequences that are derivatives of the naturally-occurring CBFs described above. For example, a non-naturally occurring sequence using domains of other transcription factors described above fused in frame, but not necessarily adjacent, with functional domains derived from other sequences or sources. Additionally, the invention includes polypeptides derived from shuffling regions of transcription factors described above by methods described in Minshull and Stemmer, U.S. Pat. No. 5,837,458, entitled “Methods and Compositions for Cellular and Metabolic Engineering” and Stemmer and Crameri, U.S. Pat. No. 5,811,238, entitled “Methods for Generating Polynucleotides having Desired Characteristics by Iterative Selection and Recombination”.

Substitutions, deletions and insertions introduced into CBF-related polypeptides are also envisioned by the invention. Such sequence modifications can be engineered into a sequence by site-directed mutagenesis (Wu (editor) Meth. Enzymol. (1993) vol. 217, Academic Press). Amino acid substitutions are typically of single residues and may be conservative (such as serine to threonine) or non-conservative (such as lysine to glutamic acid); insertions usually will be on the order of about from 1 to 10 amino acid residues; and deletions will range about from 1 to 30 residues. In preferred embodiments, deletions or insertions are made in adjacent pairs, for example, a deletion of two residues or insertion of two residues. Substitutions, deletions, insertions or any combination thereof may be combined to arrive at a sequence.

Additionally, the CBF-related polypeptide may encompass a polypeptide sequence that is modified by chemical or enzymatic means. The homologous sequence may be a sequence modified by lipids, sugars, peptides, organic or inorganic compounds, by the use of modified amino acids or the like. Protein modification techniques are illustrated in Ausubel et al., editors, (1998) Current Protocols in Molecular Biology, John Wiley & Sons.

B. Altered Expression of CBF-Related Polypeptide

Any of the identified sequences may be incorporated into a cassette or vector for expression in cells, in particular plant cells. A number of expression vectors suitable for stable transformation of plant cells or for the establishment of transgenic plants have been described including those described in Weissbach and Weissbach, (1989) Methods for Plant Molecular Biology, Academic Press, and Gelvin et al. (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303: 209, Bevan (1984) Nucl. Acids Res. 12: 8711-8721, Klee (1985) Bio/Technology 3: 637-642. Ti-derived plasmids can be transferred into both monocot and dicot species using Agrobacterium-mediated transformation (Ishida et al (1996) Nat. Biotechnol. 14: 745-750; Barton et al. (1983) Cell 32: 1033-1043).

Alternatively, non-Ti vectors can be used to transfer the DNA into plant cells by using free DNA delivery techniques. Such methods may involve, for example, the use of liposomes, electroporation, microprojectile bombardment, silicon carbide whiskers, and viruses. By using these methods transgenic plants such as wheat, rice (Christou (1991) Bio/Technology 9: 957-962) and corn (Gordon-Kamm (1990) Plant Cell 2: 603-618) can be produced. An immature embryo can also be a good target tissue for plants for direct DNA delivery techniques by using the particle gun (Weeks et al. (1993) Plant Physiol. 102: 1077-1084; Vasil (1993) Bio/Technology 10: 667-674; Wan et al. (1994) Plant Physiol. 104: 37-48, and for Agrobacterium-mediated DNA transfer (Ishida et al. (1996) Nature Biotech. 14: 745-750).

Typically, plant transformation vectors include one or more cloned plant coding sequences (genomic or cDNA) under the transcriptional control of 5′ and 3′ regulatory sequences and a dominant selectable marker. Such plant transformation vectors typically also contain a promoter (for example, a regulatory region controlling inducible or constitutive, environmentally-or developmentally-regulated, or cell- or tissue-specific expression), a transcription initiation start site, an RNA processing signal (such as intron splice sites), a transcription termination site, and/or a polyadenylation signal.

Examples of constitutive plant promoters which may be useful for expressing the CBF sequence include: the cauliflower mosaic virus (CaMV) 35S promoter, which confers constitutive, high-level expression in most plant tissues (see, for example, Odell et al. (1985) Nature 313: 810-812); the nopaline synthase promoter (An et al. (1988) Plant Physiol. 88: 547-552); and the octopine synthase promoter (Fromm et al. (1989) Plant Cell 1: 977-984).

A variety of plant gene promoters that regulate gene expression in response to environmental, hormonal, chemical, developmental signals, and in a tissue-active manner can be used for expression of the CBFs in plants, as illustrated by seed-specific promoters (such as the napin, phaseolin or DC3 promoter described in U.S. Pat. No. 5,773,697), root-specific promoters, such as those disclosed in U.S. Pat. Nos. 5,618,988, 5,837,848 and 5,905,186; fruit-specific promoters that are active during fruit ripening (such as the dru 1 promoter (U.S. Pat. No. 5,783,393), or the 2A11 promoter (U.S. Pat. No. 4,943,674) and the tomato polygalacturonase promoter (Bird et al. (1988) supra), root-specific promoters, such as those disclosed in U.S. Pat. Nos. 5,618,988, 5,837,848 and 5,905,186, pollen-active promoters such as PTA29, PTA26 and PTA13 (U.S. Pat. No. 5,792,929), promoters active in vascular tissue (Ringli et al. (1998) Plant Mol. Biol. 37: 977-988), flower-specific (Kaiser et al. (1995) Plant Mol. Biol. 28: 231-243), auxin-inducible promoters (such as that described in van der Kop et al. (1999) Plant Mol. Biol. 39: 979-990 or Baumann et al. (1999) Plant Cell 11: 323-334), cytokinin-inducible promoter (Guevara-Garcia (1998) Plant Mol. Biol. 38: 743-753), promoters responsive to gibberellin (Shi et al. (1998) Plant Mol. Biol. 38: 1053-1060, Willmott et al. (1998) 38: 817-825) and the like. Additional promoters are those that elicit expression in response to light (for example, the pea rbcS-3A promoter, Kuhlemeier et al. (1989) Plant Cell 1: 471-478, and the maize rbcS promoter, Schaffner et al. (1991) Plant Cell 3: 997-1012); wounding (for example, wunI, Siebertz et al. (1989) Plant Cell 1: 961-968); pathogen resistance chemicals such as methyl jasmonate or salicylic acid (Gatz et al. (1997) supra). In addition, the timing of the expression can be controlled by using promoters such as those acting at late seed development (Odell et al. (1994) Plant Physiol. 106: 447-458).

Plant expression vectors may also include RNA processing signals that may be positioned within, upstream or downstream of the coding sequence. In addition, the expression vectors may include additional regulatory sequences from the 3′-untranslated region of plant genes, for example, a 3′ terminator region to increase mRNA stability of the mRNA, such as the PI-II terminator region of potato or the octopine or nopaline synthase 3′ terminator regions.

Finally, as noted above, plant expression vectors may also include dominant selectable marker genes to allow for the ready selection of transformants. Such genes include those encoding antibiotic resistance genes (for example, resistance to hygromycin, kanamycin, bleomycin, G418, streptomycin or spectinomycin) and herbicide resistance genes (for example, phosphinothricin acetyltransferase).

The polynucleotides and polypeptides of this invention may also be expressed in a plant in the absence of an expression cassette by manipulating the activity or expression level of the endogenous gene by other means. For example, by ectopically expressing a gene by T-DNA activation tagging (Ichikawa et al. (1997) Nature 390: 698-701, Kakimoto et al. (1996) Science 274: 982-985). This method entails transforming a plant with a gene tag containing multiple transcriptional enhancers and once the tag has inserted into the genome, expression of a flanking gene coding sequence becomes deregulated. In another example, the transcriptional machinery in a plant may be modified so as to increase transcription levels of a polynucleotide of the invention (See PCT Publications WO9606166 and WO 9853057 which describe the modification of the DNA binding specificity of zinc finger proteins by changing particular amino acids in the DNA binding motif).

The transgenic plant may also comprise the machinery necessary for expressing or altering the activity of a polypeptide encoded by an endogenous gene, for example by altering the phosphorylation state of the polypeptide to maintain it in an activated state.

In some cases, a reduction in plant biomass or the level of cryoprotectants may be desired. In such a case, a reduction of biomass or the cryoprotectant levels may be achieved by decreasing the levels of CBF expression. For example, a reduction of CBF expression in a transgenic plant to modify a plant trait may be obtained by introducing into plants antisense constructs based on the CBF cDNA. For antisense suppression, the CBF cDNA is arranged in reverse orientation relative to the promoter sequence in the expression vector. The introduced sequence need not be the full length CBF cDNA or gene, and need not be identical to the CBF cDNA or a gene found in the plant type to be transformed.

Vectors in which RNA encoded by the CBF cDNA (or variants thereof) is over-expressed may also be used to obtain co-suppression of the endogenous CBF gene in the manner described in U.S. Pat. No. 5,231,020 to Jorgensen. Such co-suppression (also termed sense suppression) does not require that the entire CBF cDNA be introduced into the plant cells, nor does it require that the introduced sequence be exactly identical to the endogenous CBF gene. However, as with antisense suppression, the suppressive efficiency will be enhanced as (1) the introduced sequence is lengthened and (2) the sequence similarity between the introduced sequence and the endogenous CBF gene is increased.

Vectors expressing an untranslatable form of the CBF mRNA may also be used to suppress the expression of endogenous CBF activity to modify a trait. Methods for producing such constructs are described in U.S. Pat. No. 5,583,021 to Dougherty et al. Preferably, such constructs are made by introducing a premature stop codon into the CBF gene. Alternatively, a plant trait may be modified by gene silencing using double-strand RNA (Sharp (1999) Genes Development 13: 139-141) or by simultaneous expression of both sense and antisense RNAs (Waterhouse et al. (1998) Proc. Natl. Acad. Sci. 95: 13959-13964).

Another method for abolishing the expression of a gene is by insertion mutagenesis using the T-DNA of Agrobacterium tumefaciens. After generating the insertion mutants, the mutants can be screened to identify those containing the insertion in a CBF gene. Mutants containing a single mutation event at the desired gene may be crossed to generate homozygous plants for the mutation (Koncz et al. (1992) Methods in Arabidopsis Research. World Scientific).

A plant trait may also be modified by using the cre-lox system (for example, as described in U.S. Pat. No. 5,658,772). A plant genome may be modified to include first and second lox sites that are then contacted with a Cre recombinase. If the lox sites are in the same orientation, the intervening DNA sequence between the two sites is excised. If the lox sites are in the opposite orientation, the intervening sequence is inverted.

C. Transgenic Plants with Modified CBF Expression

Once an expression cassette comprising a polynucleotide encoding a CBF gene of this invention has been constructed, standard techniques may be used to introduce the polynucleotide into a plant in order to modify a trait of the plant. The plant may be any higher plant, including gymnosperms, monocotyledonous and dicotyledenous plants. Suitable protocols are available for Leguminosae (alfalfa (Medicago sativa), soybean (Glycine max), clover (Trifolium), etc.), Umbelliferae (carrot, celery, parsnip), Cruciferae (cabbage, radish, rapeseed (Brassica rapa L and Brassica napus L), broccoli, leaf mustard (Brassica juncea), etc.), Curcurbitaceae (melons and cucumber), Gramineac (wheat (Triticum), corn (Zea mays), rice (Oryza sativa), barley (Hordeum vulgare), rye (Secale cereale), sorghum (Sorghum bicolor and Sorghum vulgare), millet (Panicum miliaceum, Setaria italica, and Eleusine coracana), etc.), Solanaceae (potato, tomato, tobacco, peppers, etc.), and various other crops. See protocols described in Ammirato et al., editors, (1984) Handbook of Plant Cell Culture—Crop Species Macmillan Publ. Co. New York, N.Y.; Shimamoto et al. (1989) Nature 338: 274-276; Fromm et al. (1990) Bio/Technology 8: 833-839; and Vasil et al. (1990) Bio/Technology 8: 429-434.

Transformation and regeneration of both monocotyledonous and dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods may include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumefaciens mediated transformation. Transformation means introducing a nucleotide sequence in a plant in a manner to cause stable or transient expression of the sequence.

Successful examples of the modification of plant characteristics or traits by transformation with cloned sequences which serve to illustrate the current knowledge in this field of technology, and which are herein incorporated by reference, include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

Following transformation, plants are preferably selected using a dominant selectable marker incorporated into the transformation vector. Typically, such a marker will confer antibiotic or herbicide resistance on the transformed plants, and selection of transformants can be accomplished by exposing the plants to appropriate concentrations of the antibiotic or herbicide.

After transformed plants are selected and grown to maturity, those plants with modified plant biomass or high levels of cell protectants are identified and used for any of the purposes described above. Additionally, to confirm that the modified trait is due to changes in expression levels or activity of the polypeptide or polynucleotide of the invention may be determined by analyzing mRNA expression using Northern blots, RT-PCR or microarrays, or protein expression using immunoblots or Western blots or gel shift assays.

The following examples are intended to illustrate but not limit the present invention.

### EXAMPLES

### Example 1

Arabidopsis thaliana Plant Handling Arabidopsis thaliana (L.) Heynh. ecotype Ws-2 and transgenic plants in the Ws-2 background were grown in controlled environment chambers at 20° C. under constant illumination from cool-white fluorescent lights (100-150 μmol m⁻² s⁻¹)) essentially as described (Gilmour et al. (1988) Plant Physiol. 87:745-750) in Baccto planting mix (Michigan Peat, Houston, Tex.). Pots were sub-irrigated with deionized water as necessary. All seeds were cold-treated (5° C.) for 4 days immediately after planting to ensure uniform germination.

### Example 2

RNA Hybridization and cDNA Probes

In the following examples, unless otherwise specified, total RNA was extracted from Arabidopsis plants as described previously (Gilmour et al. (1988) Plant Physiol. 87: 745-750). Northern transfers were prepared and hybridized as described (Hajela et al. (1990) Plant Physiol. 93: 1246-1252) using high stringency wash conditions (Stockinger et al. (1997) supra). ³²P-labeled probes were prepared by random priming (Feinberg and Vogelstein (1983) Anal. Biochem. 132: 6-13). A gene-specific probe to CBF3 was made to the 3′ end of the cDNA clone by PCR as described previously (Gilmour et al. (1998) supra). Arabidopsis cDNA clones encoding Arabidopsis sucrose synthase (182C20T7), corresponding to the SUS1 gene (Martin et al. (1993) Plant J. 4: 367-377) and Δ′-pyrroline-5-carboxylate synthase (125M17T7), corresponding to the P5CS2 gene (Strizhov, et al. (1997) Plant J. 12: 557-569), were obtained from the Arabidopsis Biological Resource Center at Ohio State University. Probes for P5CS2 transcripts should cross-hybridize with the highly similar P5CS1 transcripts and thus is a measure of total P5CS transcripts.

### Example 3

Activation of Transcription in Yeast Containing C-Repeat/DRE Using CBF1, CBF2 and CBF3

This example shows that CBF1, CBF2, and CBF3 activate transcription in yeast containing CRT/DREs upstream of a reporter gene. The CBFs were expressed in yeast under control of the ADC1 promoter on a 2μ plasmid (pDB20.1; Berger et al. (1992) Cell 70: 251-265). Constructs expressing the different CBFs were transformed into yeast reporter strains that had the indicated CRT/DRE upstream of the lacZ reporter gene. Copy number of the CRT/DREs and its orientation relative to the direction of transcription from each promoter is indicated by the direction of the arrow.

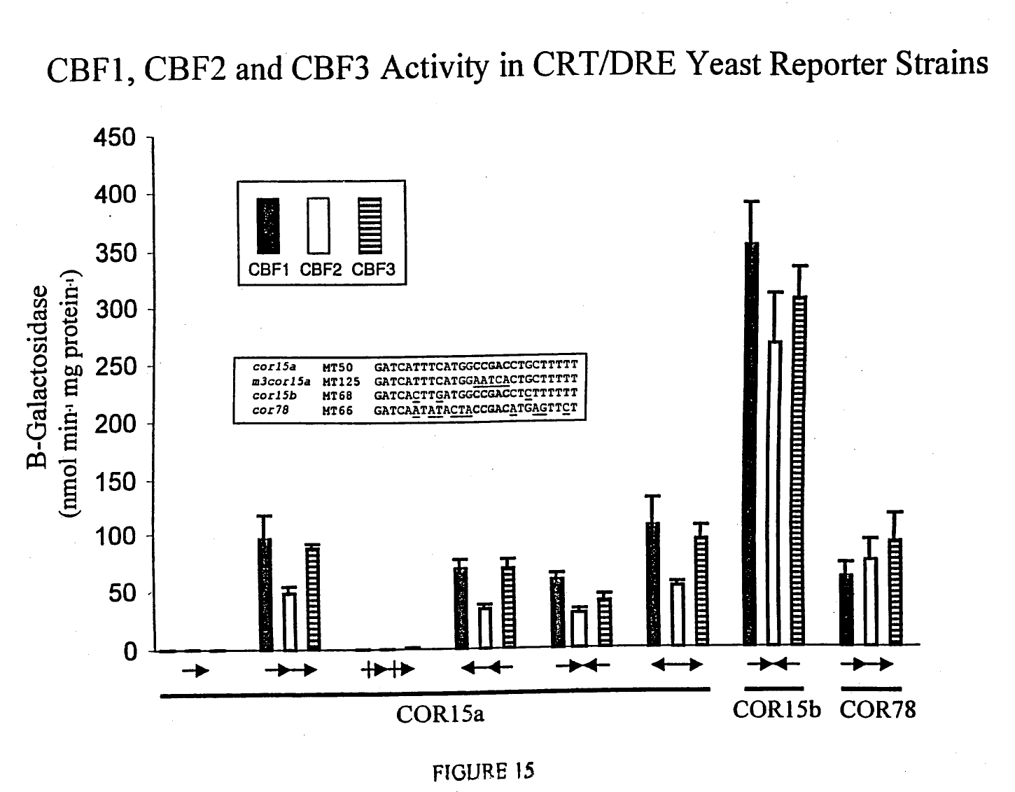

FIG. 15 is a graph showing transcription regulation of CRT/DRE containing reporter genes by CBF1, CBF2, and CBF3 genes in yeast. In FIG. 15, the vertical lines across the arrows of the COR15a construct represent the m3cor15a mutant CRT/DRE construct. Each CRT/DRE-lacZ construct was integrated into the URA3 locus of yeast. Error bars represent the standard deviation derived from three replicate transformation events with the same CBF activator construct into the respective reporter strain. Quantitative beta-gal assays were performed as described by Rose and Botstein (Rose et al. (1983) Methods Enzymol. 101: 167-180).

### Example 4

Isolation and Analysis of Arabidopsis thaliana cDNA Clone (CBF1) Encoding C-Repeat/DRE Binding Factor

The following example describes the isolation of an Arabidopsis thaliana cDNA clone that encodes a C-repeat/DRE binding factor, CBF1 (C-repeat/DRE Binding Factor 1). Expression of CBF1 in yeast was found to activate transcription of reporter genes containing the C-repeat/DRE (CCGAC) as an upstream activator sequence. Meanwhile, CBF1 did not activate transcription of mutant versions of the CCGAC binding element, indicating that CBF1 is a transcription factor that binds to the C-repeat/DRE. Binding of CBF1 to the C-repeat/DRE was also demonstrated in gel shift assays using recombinant CBF1 protein expressed in Escherichia coli. Analysis of the deduced CBF1 amino acid sequence indicated that the protein has a potential nuclear localization sequence, a possible acidic activation domain and an AP2 domain, a DNA-binding motif of about 60 amino acids that is similar to those present in Arabidopsis proteins APETALA2, AINTEGUMENTA and TINY, the tobacco ethylene response element binding proteins, and numerous other plant proteins of unknown function.

Cold Treatment.

Plants were treated by placing pots in a cold room at 2.5° C. under cool constant illumination with white florescent lamps (25 μmol m⁻²s⁻¹) for the indicated times.

Arabidopsis cDNA Expression Library.

The Arabidopsis pACT cDNA expression library was constructed by John Walker and colleagues (NSF/DOE/USDA Collaborative Research in Plant Biology Program grant USDA 92-37105-7675) and deposited in the Arabidopsis Biological Resource Center (stock #CD4-10).

Yeast Reporter Strains.

Oligonucleotides (Table 4) (synthesized at the MSU Macromolecular Structure Facility) encoding either wild-type or mutant versions of the C-repeat/DRE were ligated into the BglII site of the lacZ reporter vector pBgl-lacZ (Li et al. (1993) Science 262: 1870-1874); kindly provided by Joachim Li). The resulting reported constructs were integrated into the ura3 locus of Saccharomyces cerevisiae strain GGY1 (MAT gal4 gal80 ura3 leu2 his3 ade2 tyr) (Li et al. (1993) supra; provided by Joachim Li) by transformation and selection for uracil prototrophy.

E. coli Strains

Escherichia coli strain GM2163 containing plasmid pEJS251 was deposited under the Budapest Treaty on May 17, 1996 with the American Type Culture Collection, Rockville, Md. as ATCC 98063. It is available by name and number pursuant to the provisions of the Budapest Treaty.

TABLE 4
Oligonucleotides encoding wild type and mutant versions of the
C-repeat/DRE

| | C-repeat/ | | |
|---|---|---|---|
| Oligonucleotide | DRE* | Sequence | SEQ ID NO: |
| MT50 | COR15a | GatcATTTCATGGCCGACCTGCTTTTT | 3 |
| MT52 | M1COR15a | CACAATTTCAaGaattcaCTGCTTTTTT | 4 |
| MT80 | M2COR15a | GatcATTTCATGGtatgtCTGCTTTTT | 5 |
| MT125 | M3COR15a | GatcATTTCATGGaatcaCTGCTTTTT | 6 |
| MT68 | COR15b | GatcACTTGATGGCCGACCTCTTTTTT | 7 |
| MT66 | C0R78-1 | GatcAATATACTACCGACATGAGTTCT | 8 |
| MT86 | C0R78-2 | ACTACCGACATGAGTTCCAAAAAGC | 9 |

* The C-repeat/DRE sequences tested are either wild-type found in the promoters of COR15a (Baker et al. (1994) Plant Mol. Biol. 24: 701-713), COR15b or COR78/RD29a (Horvath et al. (1993) Plant Physiol. 103: 1047-1053; Yamaguchi-Shinozaki et al. (1994) Plant Cell 6: 251-264) or are mutant versions of the COR15a C-repeat/DRE (M1COR15a, M2COR15a and M3COR15a).

# Uppercase letters designate bases in wild type C-repeat/DRE sequences. The core CCGAC sequence common to the above sequences is indicated in bold type. Lowercase letters at the beginning of a sequence indicate bases added to facilitate cloning. The lowercase letters that are underlined indicate the mutations in the C-repeat/DRE sequence of COR15a.

Screen of Arabidopsis cDNA Library.

The Arabidopsis pACT cDNA expression library was screened for clones encoding C-repeat/DRE biomass and environmental stress response regulatory elements by the following method. The cDNA library, harbored in Escherichia coli BNN132, was amplified by inoculating 0.5 ml of the provided glycerol stock into 1 L of M9 minimal glucose medium (Sambrook et al. (1989) supra) and shaking the bacteria for 20 h at 37° C. Plasmid DNA was isolated and purified by cesium chloride density gradient centrifugation (Sambrook et al. (1989) supra) and transformed into the yeast GGY1 reporter strains selecting for leucine prototrophy. Yeast transformants that had been grown for 2 or 3 days at 30° C. were overlaid with either a nitrocellulose membrane filter (Schleicher and Schuell, Keene, N.H.) or Whatman #50 filter paper (Hillsboro, Oreg.) and incubated overnight at 30° C. The yeast impregnated filters were then lifted from the plate and treated with X-gal (5-bromo-4-chloro-3-indolyl-D-galactosidase) to assay colonies for beta-galactosidase activity (Li et al. (1993) supra). Plasmid DNA from “positive” transformants (those forming blue colonies on the X-gal-treated filters) was recovered (Strathern et al. (1991) Methods Enzymol. 194: 319-329), propagated in E. coli DH5α and transformed back into the yeast reporter strains to confirm activity.

Yeast Transformation and Quantitative Beta-galactosidase Assays.

Yeast were transformed by either electroporation (Becker et al. (1991) Methods Enzymol. 194: 182-187) or the lithium acetate/carrier DNA method (Schiestl et al. (1989) Current Genetics 16: 339-346). Quantitative in vitro beta-galactosidase assays were done as described (Rose et al. (1983) Methods Enzymol. 101: 167-180).

Expression of CBF1 Protein in E. coli and Yeast.

CBF1 was expressed in E. coli using the pET-28a(+) vector (Novagen, Madison, Wis.). The BglII-BclI restriction fragment of pACT-11 encoding CBF1 was ligated into the BamHI site of the vector bringing CBF1 under control of the T7 phage promoter. The construct resulted in a “histidine tag,” a thrombin recognition sequence and a “T7 epitope tag” being fused to the amino terminus of CBF1. The construct was transformed into E. coli BL21 (DE3) and the recombinant CBF1 protein was expressed as recommended by the supplier (Novagen). Expression of CBF1 in yeast was accomplished by ligating restriction fragments encoding CBF1 (the BclI-BglII and BglII-BglII fragments from pACT-11) into the BglII site of pDB20.1 (Berger et al. (1992) Cell 70: 251-265) bringing CBF1 under control of the constitutive ADC1 (alcohol dehydrogenase constitutive 1) promoter.

Gel Shift Assays.

The presence of expressed protein that binds to a C-repeat/DRE binding domain was evaluated using the following gel shift assay. Total soluble E. coli protein (40 ng) was incubated at room temperature in 10 μl of 1× binding buffer [15 mM HEPES (pH 7.9), 1 mM EDTA, 30 mM KCl, 5% glycerol, 5% BSA, 1 mM DTT) plus 50 ng poly(dI-dC):poly(dI-dC) (Pharmacia, Piscataway, N.J.) with or without 100 ng competitor DNA. After 10 min, probe DNA (1 ng) that was ³²P-labeled by end-filling (Sambrook et al. supra) was added and the mixture incubated for an additional 10 min. Samples were loaded onto polyacrylamide gels (4% w/v) and fractionated by electrophoresis at 150V for 2 h (Sambrook et al. supra). Probes and competitor DNAs were prepared from oligonucleotide inserts ligated into the BamHI site of pUC118 (Vicira et al. (1987) Methods Enzymol. 153: 3-11). The orientation and concatenation number of the inserts were determined by dideoxy DNA sequence analysis (Sambrook et al. (1989), supra). Inserts were recovered after restriction digestion with EcoRI and HindIII and fractionation on polyacrylamide gels (12% w/v) (Sambrook et al. (1989) supra).

Northern and Southern Analysis.

Northern and Southern analysis was performed as follows. Total RNA was isolated from Arabidopsis (Gilmour et al. (1988) Plant Physiol. 87: 745-750) and the poly(A)⁺ fraction purified using oligo dT cellulose (Sambrook et al. (1989), supra). Northern transfers were prepared and hybridized as described (Hajela et al. (1990) Plant Physiol. 93: 1246-1252) except that high stringency wash conditions were at 50 C in 0.1×SSPE [1.0×SSPE is 3.6 M NaCl, 20 mM EDTA, 0.2 M Na₂—HPO₄ (pH7.7)], 0.5% SDS. Membranes were stripped in 0.1×SSPE, 0.5% SDS at 95° C. for 15 min prior to re-probing. Total Arabidopsis genomic DNA was isolated (Stockinger et al. (1996) J. Heredity 87: 214-218) and Southern transfers prepared (Sambrook et al. supra) using nylon membranes (MSI, Westborough, Mass.). High stringency hybridization and wash conditions were as described by Walling (Walling et al. (1988) Nucleic Acids Res. 16: 10477-10492). Low stringency hybridization was in 6×SSPE, 0.5% SDS, 0.25% low fat dried milk at 60° C. Low stringency washes were in 1×SSPE, 0.5% SDS at 50° C. Probes used for the entire CBF1 coding sequence and 3′ end of CBF1 were the BclI/BglII and EcoRV/BglII restriction fragments from pACT-11, respectively, that had been gel purified (Sambrook et al. (1989), supra). DNA probes were radiolabeled with ³²P-nucleotides by random priming (Sambrook et al. (1989), supra). Autoradiography was performed using hyperfilm-MP (Amersham, Arlington Heights Ill.). Radioactivity was quantified using a Betascope 603 blot analyzer (Betagen Corp., Waltham Mass.).

Screen of Arabidopsis cDNA Library for Sequence Encoding a C-Repeat/DRE Binding Domain.

The “one-hybrid” strategy (Li et al. (1993) supra) was used to screen for Arabidopsis cDNA clones encoding a C-repeat/DRE binding domain. In brief, yeast strains were constructed that contained a lacZ reporter gene with either wild-type or mutant C-repeat/DRE sequences in place of the normal UAS (upstream activator sequence) of the GAL1 promoter.

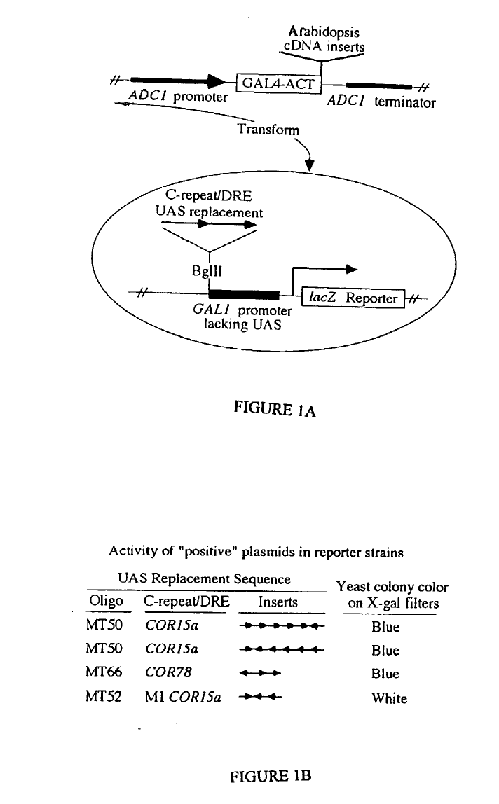

FIGS. 1A and 1B show how the yeast reporter strains were constructed. FIG. 1A is a schematic diagram showing the screening strategy. Yeast reporter strains were constructed that carried C-repeat/DRE sequences as UAS elements fused upstream of a lacZ reporter gene with a minimal GAL1 promoter. The strains were transformed with an Arabidopsis expression library that contained random cDNA inserts fused to the GAL4 activation domain (GAL4-ACT) and screened for blue colony formation on X-gal-treated filters. FIG. 1B is a chart showing activity of the “positive” cDNA clones in yeast reporter strains. The oligonucleotides (oligos) used to make the UAS elements, and their number and direction of insertion, are indicated by the arrows.

Yeast strains carrying these reporter constructs produced low levels of beta-galactosidase and formed white colonies on filters containing X-gal. The reporter strains carrying the wild-type C-repeat/DRE sequences were transformed with a DNA expression library that contained random Arabidopsis cDNA inserts fused to the acidic activator domain of the yeast GAL4 transcription factor, “GAL4-ACT” (FIG. 1A). The notion was that some of the clones might contain a cDNA insert encoding a C-repeat/DRE binding domain fused to GAL4-ACT and that such a hybrid protein could potentially bind upstream of the lacZ reporter genes carrying the wild type C-repeat/DRE sequence, activate transcription of the lacZ gene and result in yeast forming blue colonies on X-gal-treated filters.

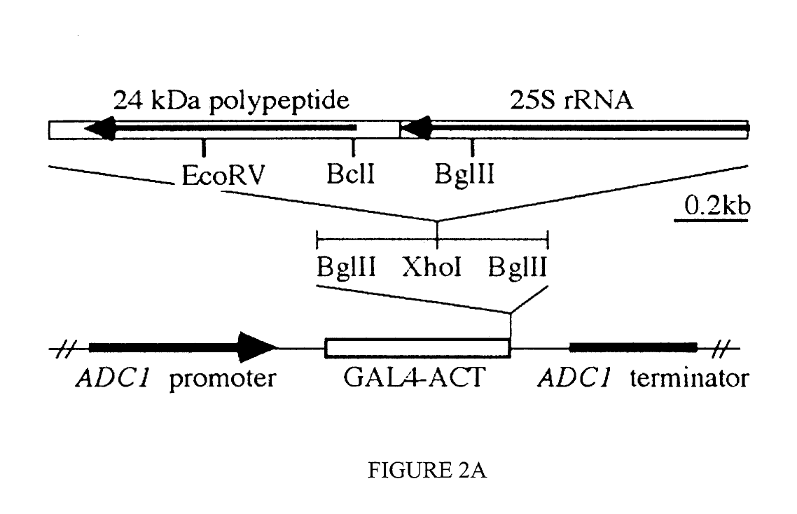

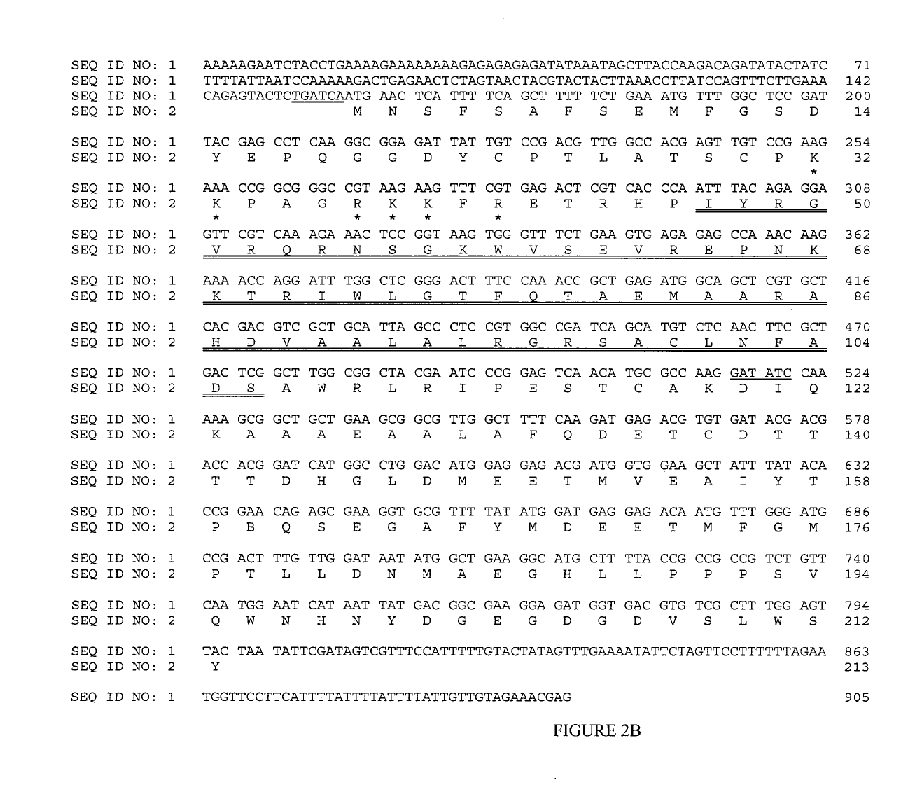

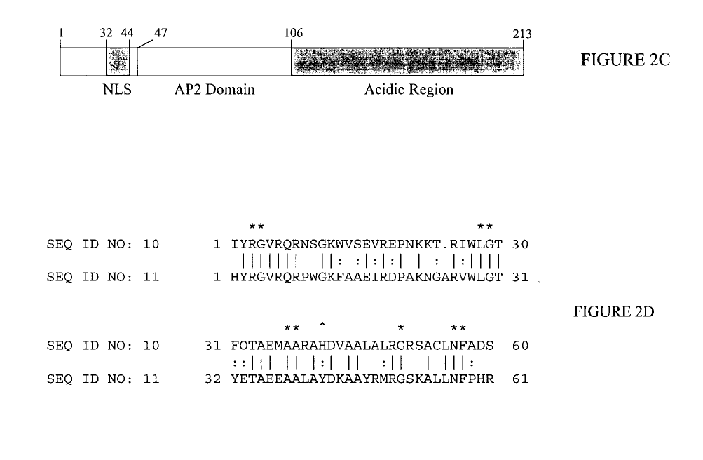

Upon screening about 2×10⁶ yeast transformants, three “positive” cDNA clones were isolated; i.e., clones that caused yeast strains carrying lacZ reporters fused to wild-type C-repeat/DRE inserts to form blue colonies on X-gal-treated filters (FIG. 1B). The three cDNA clones did not cause a yeast strain carrying a mutant C-repeat/DRE fused to LacZ to turn blue (FIG. 1B). Thus, activation of the reporter genes by the cDNA clones appeared to be dependent on the C-repeat/DRE sequence. Restriction enzyme analysis and DNA sequencing indicated that the three cDNA clones had an identical 1.8 kb insert (FIG. 2A). One of the clones, designated pACT-11, was chosen for further study.

Identification of 24 kDa Polypeptide with an AP2 Domain Encoded by pACT-11.

FIGS. 2A, 2B, 2C and 2D provide an analysis of the pACT-11 cDNA clone. FIG. 2A is a schematic drawing of the pACT-11 cDNA insert indicating the location and 5′ to 3′ orientation of the 24 kDa polypeptide and 25s rRNA sequences. The cDNA insert was cloned into the XhoI site of the pACT vector. FIG. 2B is a DNA and amino acid sequence of the 24 kDa polypeptide (SEQ ID NO: 1 and SEQ ID NO: 2). The AP2 domain is indicated by a double underline. The basic amino acids that potentially act as a nuclear localization signal are indicated with asterisks. The BclI site immediately upstream of the 24 kDa polypeptide used in subcloning the 24 kDa polypeptide and the EcoRV site used in subcloning the 3′ end of CBF1 are indicated by single underlines. FIG. 2C is a schematic drawing indicating the relative positions of the potential nuclear localization signal (NLS), the AP2 domain and the acidic region of the 24 kDa polypeptide. Numbers indicate amino acid residues. FIG. 2D is a chart showing comparison of the AP2 domain of the 24 kDa polypeptide (SEQ ID NO: 10) with that of the tobacco DNA binding protein EREBP2 (Okme-Takagi et al. (1995) Plant Cell 7: 173-182; SEQ ID NO: 11). Identical amino acids are indicated with single lines; similar amino acids are indicated by double dots; amino acids that are invariant in AP2 domains are indicated with asterisks (Klucher et al. (1996) Plant Cell 8: 137-153); and the histidine residues present in CBF1 and TINY (Wilson et al. (1996) Plant Cell 8: 659-671) that are tyrosine residues in all other described AP2 domains are indicated with a caret. A single amino acid gap in the CBF1 sequence is indicated by a single dot.

Our expectation was that the cDNA insert in pACT-11 would have a C-repeat/DRE binding domain fused to the yeast GAL4-ACT sequence. However, DNA sequence analysis indicated that an open reading frame of only nine amino acids had been added to the C-terminus of GAL4-ACT. It seemed highly unlikely that such a short amino acid sequence could comprise a DNA binding domain. Also surprising was the fact that about half of the cDNA insert in pACT-11 corresponded to 25s rRNA sequences (FIG. 2A). Further analysis, however, indicated that the insert had an open reading frame, in opposite orientation to the GAL4-ACT sequence, deduced to encode a 24 kDa polypeptide (FIGS. 2A-2C). The polypeptide has a basic region that could potentially serve as a nuclear localization signal (Raikhel (1992) Plant Physiol. 100: 1627-1632) and an acidic C-terminal half (pI of 3.6) that could potentially act as an acidic transcription activator domain (Hahn (1993) Cell 72: 481-483). A search of the nucleic acid and protein sequence databases indicated that there was no previously described homology of the 24 kDa polypeptide. However, the polypeptide did have an AP2 domain (Jofuku et al. (1994) Plant Cell 6: 1211-1225 (FIGS. 2B, 2D), a DNA binding motif of about 60 amino acids (Ohme-Takagi et al. (1994) Plant Cell 7: 173-182) that is present in numerous plant proteins including the APETALA2 (Jofuku et al. (1994) Plant Cell 6: 1211-1225), AINTEGUMENTA (Klucher et al. (1996) Plant Cell 8: 137-153; Elliot et al. (1996) Plant Cell 8: 155-168) and TINY (Wilson et al. (1996) Plant Cell 8: 659-671) proteins of Arabidopsis and the EREBPs (ethylene response element binding proteins) of tobacco (Ohme-Takagi et al. (1995) Plant Cell 7: 173-182).

24 kDa Polypeptide Binds to the C-Repeat/DRE and Activates Transcription in Yeast.

We hypothesized that the 24 kDa polypeptide was responsible for activating the lacZ reporter genes in yeast. To test this, the BclI-BglII fragment of pACT-11 containing the 24 kDa polypeptide, and the BglII-BglII fragment containing the 24 kDa polypeptide plus a small portion of the 25s rRNA sequence, was inserted into the yeast expression vector pDB20.1

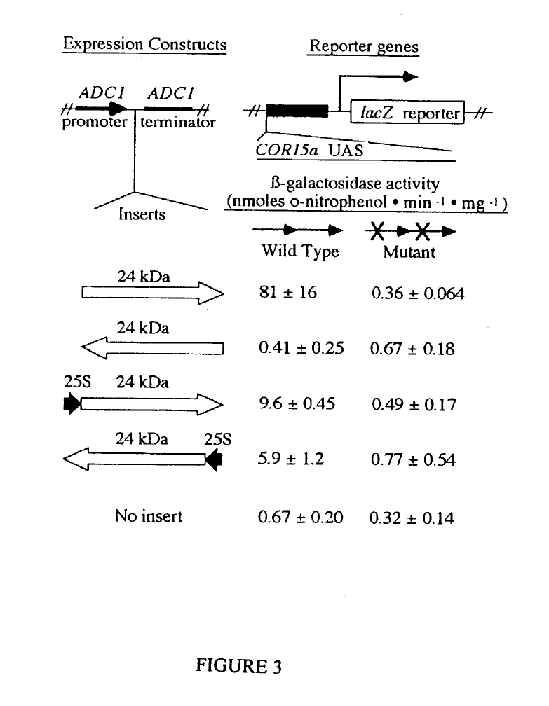

FIG. 3 is a chart showing activation of reporter genes by the 24 kDa polypeptide. Restriction fragments of pACT-11 carrying the 24 kDa polypeptide (BclI-BglII) or the 24 kDa polypeptide plus a small amount of 25s RNA sequence (BglII-BglII) were inserted in both orientations into the yeast expression vector pDB20.1 (see FIGS. 2A and 2B for location of BclI and BglII restriction sites). These “expression constructs” were transformed into yeast strains carrying the lacZ reporter gene fused to direct repeat dimers of either the wild-type COR15a C-repeat/DRE (oligonucleotide MT50) or the mutant M2COR15a C-repeat/DRE (oligonucleotide MT80). The specific activity of beta-galactosidase (nmoles o-nitrophenol produced/min⁻¹×mg protein⁻¹) was determined from cultures grown in triplicate. Standard deviations are indicated. Abbreviations: pADC1, ADC1 promoter; tADC 1, ADC1 terminator.

Plasmids containing either insert in the same orientation as the ADC1 promoter stimulated synthesis of beta-galactosidase when transformed into yeast strains carrying the lacZ reporter gene fused to a wild-type COR15a C-repeat/DRE (FIG. 3). The plasmids did not, however, stimulate synthesis of beta-galactosidase when transformed into yeast strains carrying lacZ fused to a mutant version of the COR15a C-repeat/DRE (FIG. 3). These data indicated that the 24 kDa polypeptide could bind to the wild-type C-repeat/DRE and activate expression for the lacZ reporter gene in yeast. Additional experiments indicated that the 24 kDa polypeptide could activate expression of the lacZ reporter gene fused to either a wild-type COR78 C-repeat/DRE (dimer of MT66) or a wild-type COR15b C-repeat/DRE (dimer of MT 68) (not shown). A plasmid containing the BclI-BglII fragment (which encodes only the 24 kDa polypeptide) cloned in opposite orientation to the ADC1 promoter did not stimulate synthesis of beta-galactosidase in reporter strains carrying the wild-type COR15a C-repeat/DRE fused to lacZ (FIG. 3). In contrast, a plasmid carrying the BglII-BglII fragment (containing the 24 kDa polypeptide plus some 25s rRNA sequences) cloned in opposite orientation to the ADC1 promoter produced significant levels of beta-galactosidase in reporter strains carrying the wild-type COR15a C-repeat/DRE (FIG. 3). Thus, a sequence located closely upstream of the 24 kDa polypeptide was able to serve as a cryptic promoter in yeast, a result that offered an explanation for how the 24 kDa polypeptide was expressed in the original pACT-11 clone.

Gel Shift Analysis Indicates that the 24 kDa Polypeptide Binds to the C-Repeat/DRE.

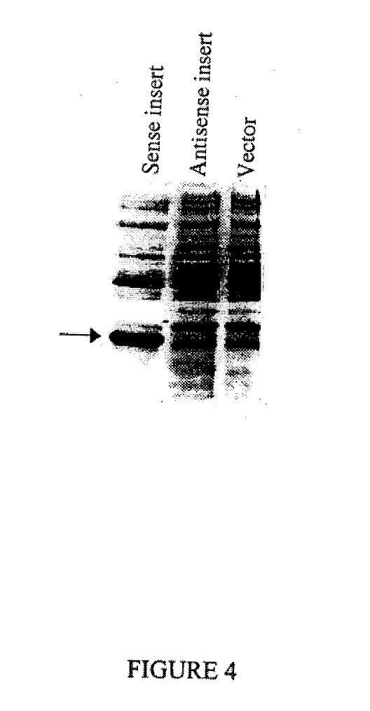

Gel shift experiments were conducted to demonstrate further that the 24 kDa polypeptide bound to the C-repeat/DRE. Specifically, the open reading frame for the 24 kDa polypeptide was inserted into the pET-28a(+) bacterial expression vector and the resulting 28 kDa fusion protein was expressed at high levels in E. coli. (FIG. 4).

FIG. 4 is a photograph of an electrophoresis gel showing expression of the recombinant 24 kDa polypeptide in E. coli. Shown are the results of SDS-PAGE analysis of protein extracts prepared from E. coli harboring either the expression vector alone (vector) or the vector plus an insert encoding the 24 kDa polypeptide in sense (sense insert) or antisense (antisense insert) orientation. The 28 kDa fusion protein (see Materials and Methods) is indicated by an arrow.

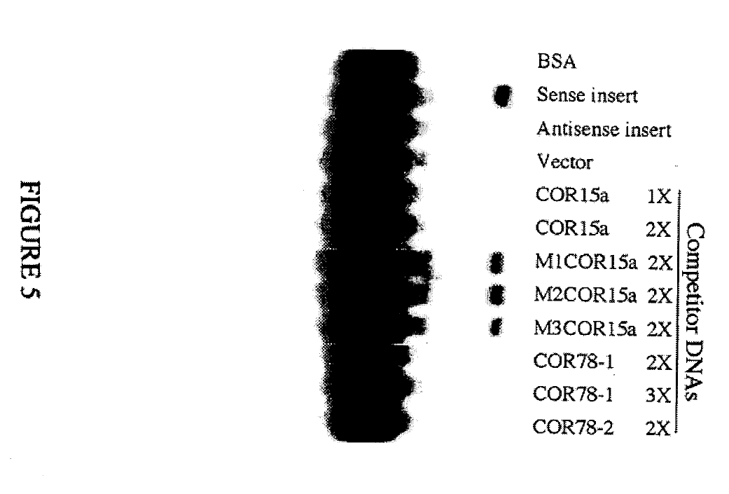

FIG. 5 is a photograph of a gel for shift assays indicating that CBF1 binds to the C-repeat/DRE. The C-repeat/DRE probe (1 ng) used in all reactions was a ³²P-labeled dimer of the oligonucleotide MT50 (wild type C-repeat/DRE from COR15a). The protein extracts used in the first four lanes were either bovine serum albumin (BSA) or the indicated CBF1 sense, antisense and vector extracts described in FIG. 4. The eight lanes on the right side of the figure used the CBF1 sense protein extract plus the indicated competitor C-repeat/DRE sequences (100 ng). The numbers 1×, 2× and 3× indicate whether the oligonucleotides were monomers, dimers or trimers, respectively, of the indicated C-repeat/DRE sequences.

Protein extracts prepared from E. coli expressing the recombinant protein produced a gel shift when a wild-type COR15a C-repeat/DRE was used as probe (FIG. 5). No shift was detected with BSA or E. coli extracts prepared from strains harboring the vector alone, or the vector with an antisense insert for the 24 kDa polypeptide. Oligonucleotides encoding wild-type C-repeat/DRE sequences from COR15a or COR78 competed effectively for binding to the COR15a C-repeat/DRE probe, but mutant version of the COR15a C-repeat/DRE did not (FIG. 5). These in vitro results corroborated the in vivo yeast expression studies indicating that the 24 kDa polypeptide binds to the C-repeat/DRE sequence. The 24 kDa polypeptide was thus designated CBF1 (C-repeat/DRE binding factor 1) and the gene encoding it named CBF1.

CBF1 is a Unique or Low Copy Number Gene.

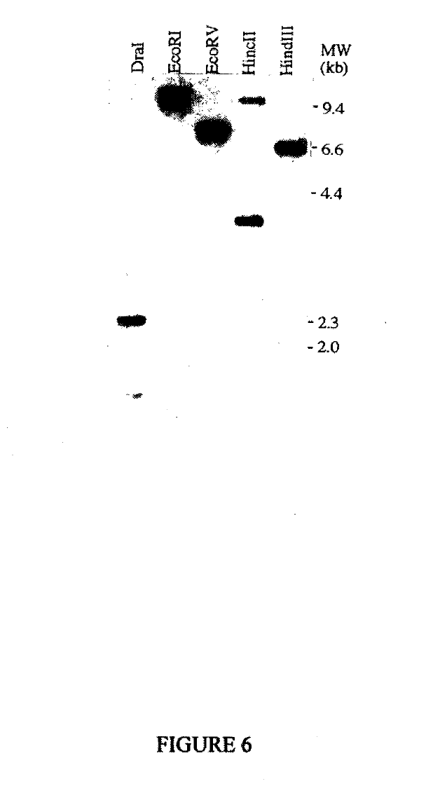

FIG. 6 is a photograph of a southern blot analysis indicating CBF1 is a unique or low copy number gene. Arabidopsis DNA (1 μg) was digested with the indicated restriction endonucleases and southern transfers were prepared and hybridized with a ³²P-labeled probe encoding the entire CBF1 polypeptide.

The hybridization patterns observed in southern analysis of Arabidopsis DNA using the entire CBF1 gene as probe were relatively simple indicating that CBF1 is either a unique or low copy number gene (FIG. 6). The hybridization patterns obtained were not altered if only the 3′ end of the gene was used as the probe (the EcoRV/BglII restriction fragment from pACT-11 encoding the acidic region of CBF1, but not the AP2 domain) or if hybridization was carried out at low stringency (not shown).

CBF1 Transcript Level Response to Low Temperature.

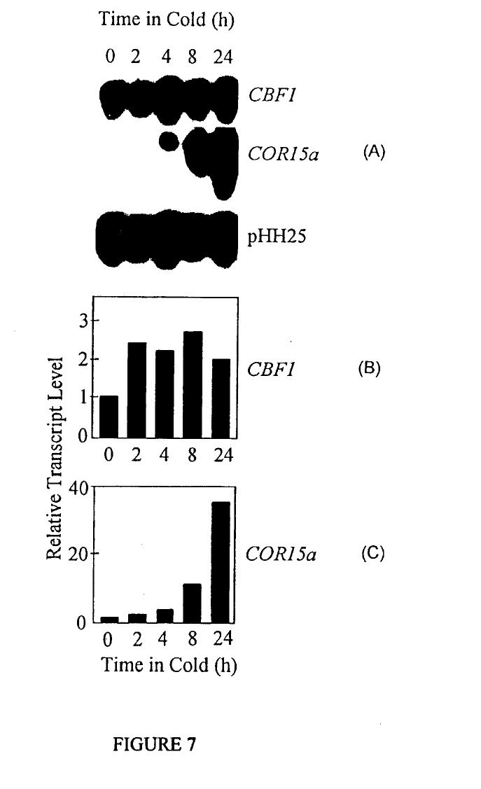

FIGS. 7A, 7B and 7C relate to CBF1 transcripts in control and cold-treated Arabidopsis. FIG. 7A is a photograph of a membrane RNA isolated from Arabidopsis plants that were grown at 22° C. or grown at 22° C. and transferred to 2.5° C. for the indicated times. FIGS. 7B and 7C are graphs showing relative transcript levels of CBF1 and COR15a in control and cold-treated plants. The radioactivity present in the samples described in FIG. 7A were quantified using a Betascope 603 blot analyzer and plotted as relative transcript levels (the values for the 22° C. grown plants being arbitrarily set as 1) after adjusting for differences in loading using the values obtained with the pHH25 probe.

Based on the original data used to prepare FIGS. 7A-7C, northern analysis indicated that the level of CBF1 transcripts increased about 2 to 3 fold in response to low temperature (FIG. 7B). Following this analysis, it was determined that CBF genes are induced rapidly (within 15 min of treatment) by mechanical agitation as well as cold temperatures (Gilmour et al. (1998) Plant J. 16: 433-443), and that CBF genes are not expressed at significant levels in non-stressed plants. The detectable level of CBF1 transcripts at the “0” time point in FIGS. 7A and 7B was subsequently shown to be due to agitation, and that CBF1 transcript levels actually increased significantly more in response to low temperature than 2 to 3 fold over control plants, when agitation was eliminated as a inducer of CBF genes in all of the plants.

The transcript levels for COR15a increased approximately 35 fold in cold-treated plants (FIG. 7C). Only a singly hybridizing band was observed for CBF1 at either high or low stringency with probes for either the entire CBF1 coding sequence or the 3′ end of the gene (the EcoRV/BglII fragment of pACT-11) (not shown). The size of the CBF1 transcripts was about 1.0 kb.

The above example regarding CBF1 represents the first identification of a gene sequence that encodes a protein capable of binding to the C-repeat/DRE sequence CCGAC. The experimental results presented evidence that CBF1 binds to the C-repeat/DRE both in vitro via gel shift assays and in vivo via yeast expression assays. Further, the results demonstrate that CBF1 can activate transcription of reporter genes in yeast that contain the C-repeat/DRE.

The results of the southern analysis indicate that CBF1 is a unique or low copy number gene in Arabidopsis. However, the CBF1 protein contains a 60 amino acid motif, the AP2 domain that is evolutionary conserved in plants (Weigel (1995) Plant Cell 7: 388-389). It is present in the APETALA2 (Jofuku et al. (1994) Plant Cell 6: 1211-1225), AINTEGUMENTA (Klucher et al. (1996) Plant Cell 8: 137-153; and Elliot et al. (1996) Plant Cell 8: 155-168), TINY (Wilson et al. Plant Cell 8: 659-671 (1996)) and cadmium-induced (Choi et al. (1995) Plant Physiol. 108: 849) proteins of Arabidopsis and the EREBPs of tobacco (Ohme-Takagi et al. (1995) Plant Cell 7: 173-182). In addition, a search of the GenBank expressed sequence tagged cDNA database indicates that there is one cDNA from B. napus, two from Ricinus communis, and more than 25 from Arabidopsis and 15 from rice, that are deduced to encode proteins with AP2 domains. The results of Ohme-Takagi and Shinshi (Ohme-Takagi et al. supra) indicate that the function of the AP2 domain is DNA-binding; this region of the putative tobacco transcription factor EREBP2 is responsible for its binding to the cis-acting ethylene response element referred to as the GCC-repeat. As discussed by Ohme-Takagi and Shinshi (Ohme-Takagi et al. supra), the DNA-binding domain of EREBP2 (the AP2 domain) contains no significant amino acid sequence similarities or obvious structural similarities with other known transcription factors or DNA binding motifs. Thus, the domain appears to be a novel DNA-binding motif that to date, has only been found in plant proteins.

It is generally believed that that the CCGAC core sequence is a member of family of core sequences having the common subsequence CCG, and that the binding of CBF1 to the C-repeat/DRE involves the AP2 domain. In this regard, it is germane to note that the tobacco ethylene response element, AGCCGCC, closely resembles the C-repeat/DRE sequences present in the promoters of the Arabidopsis genes COR15a, GGCCGAC, and COR78/RD29A, TACCGAC. Applicants believe that CBF1, the EREBPs and other AP2 domain proteins are members of a superfamily of DNA binding proteins that recognize a family of cis-acting regulatory elements having CCG as a common core sequence. Differences in the sequence surrounding the CCG core element could result in recruitment of different AP2 domain proteins which, in turn, could be integrated into signal transduction pathways activated by different environmental, hormonal and developmental cues. Such a scenario is akin to the situation that exists for the ACGT-family of cis-acting elements (Foster et al. (1994) FASEB J. 8: 192-200). In this case, differences in the sequence surrounding the ACGT core element result in the recruitment of different bZIP transcription factors involved in activating transcription in response to a variety of environmental and developmental signals. Applicants believe that other C-repeat/DRE regulatory sequences exist which belong to a broader CCG family of regulatory sequences. By screening plant genomes according to the methodology taught herein using other members of the CCG family, additional regulatory sequences as well as the binding proteins which bind to these regulatory sequences can be identified. For example, plants which are known to have modified biomass or exhibit a form of environmental stress tolerance can be screened according to the blue colony assay and other screening methodologies used in the present invention with other members of the CCG family in order to identify other binding proteins and their gene sequences. Examples of other members of the CCG family include, but are not limited to, biomass or environmental stress response regulatory elements which include one of the following sequences: CCGAA, CCGAT, CCGAC, CCGAG, CCGTA, CCGTT, CCGTC, CCGTG, CCGCA, CCGCT, CCGCG, CCGCC, CCGGA, CCGGT, CCGGC, CCGGG, AACCG, ATCCG, ACCCG, AGCCG, TACCG, TTCCG, TCCCG, TGCCG, CACCG, CTCCG, CGCCG, CCCCG, GACCG, GTCCG, GCCCG, GGCCG, ACCGA, ACCGT, ACCGC, ACCGG, TCCGA, TCCGT, TCCGC, TCCGG, CCCGA, CCCGT, CCCGC, CCCGG, GCCGA, GCCGT, GCCGC, and GCCGG (see U.S. Pat. No. 6,417,428).

The results of the yeast transformation experiments indicate that CBF1 has a domain that can serve as a transcriptional activator. The most likely candidate for this domain is the acidic C-terminal half of the polypeptide. Indeed, random acidic amino acid peptides from E. coli have been shown to substitute for the GAL4 acidic activator domain of GAL4 in yeast (Ma et al. (1987) Cell 51: 113-199). Moreover, acidic activator domains have been found to function across kingdoms (Hahn (1993) Cell 72: 481-483); the yeast GAL4 acidic activator, for instance, can activate transcription in tobacco (Ma et al. (1988) Nature 334: 631-633). It has also been shown that certain plant transcription factors, such as Vp1 (McCarty et al. (1991) Cell 66: 895-905), have acidic domains that function as transcriptional activators in plants. Significantly, the acidic activation domains of the yeast transcription factors VP16 and GCN4 require the “adaptor” proteins ADA2, ADA3, and GCN5 for full activity (see Guarente (1995) Trends Biochem. Sci. 20: 517-521). These proteins form a heteromeric complex (Horiuchi et al. (1995) Mol. Cell Biol. 15: 1203-1209) that bind to the relevant activation domains. The precise mechanism of transcriptional activation is not known, but appears to involve histone acetylation: there is a wealth of evidence showing a positive correlation between histone acetylation and the transcriptional activity of chromatin (Wolffe (1994) Trends Biochem. Sci. 19: 240-244) and recently, the GCN5 protein has been shown to have histone acetyltransferase activity (Brownell et al. (1996) Cell 84: 843-851). Genetic studies indicate that CBF1, like VP16 and GCN4, requires ADA2, ADA3 and GCN5 to function optimally in yeast. The fundamental question thus raised is whether plants have homologs of ADA2, ADA3 and GCN5 and whether these adaptors are required for CBF1 function (and function of other transcription factors with acidic activator regions) in Arabidopsis.

### Example 5

Identification of Modified Phenotypes in Overexpression or Gene Knockout Plants

Experiments were performed to identify those transformants or knockouts that exhibited modified cell protectant levels. Among the biochemicals that were assayed were sugars, proline and fatty acids.

Proline levels in leaves were measured by preparing lyophilized leaf material; 30 mg samples of the lyophilized material were then extracted with 3 ml deionized water at 80° C. for 15 min. The samples were shaken for approximately 1 hour at room temperature and then allowed to stand overnight at 4° C. The extracts were filtered through glass wool and analyzed for proline content using the acid ninhydrin reaction (Troll and Lindsley (1955) J. Biol. Chem. 215:655-660). Proline levels in certain samples were confirmed by amino acid analysis using an amino acid analyzer at the Macromolecular Structure Facility in the Biochemistry Department at Michigan State University.

Total soluble sugars (for example, sucrose, glucose, and fructose among others) were extracted from lyophilized leaf material (20 mg) in 80% ethanol (2 ml) at 80° C. for 15 min. The samples were shaken for approximately 1 hr at room temperature and allowed to stand overnight at 4° C. Extracts were filtered through glass wool and chlorophyll removed by shaking samples (0.4 ml) with water (0.4 ml) and chloroform (0.4 ml). The aqueous extract was tested for sugar content using the phenol-sulfuric acid assay (Dubois et al. (1956) Anal. Chem. 28:350-356). Certain samples were dried down, suspended in water and the sugars analyzed by HPLC using a sugar column (Shodex, Shoko Co. Ltd., Japan) with a refractive index detector as previously described (Gao et al. (1999) Physiol. Plant 106:1-8). Retention times were compared to those of standard glucose, fructose and sucrose, and the peaks integrated using Millennium-32 software (Waters Corp.).

The fatty acid composition of plant cells and tissues may be altered by transcriptional control of fatty acid biosynthesis. The presently disclosed transcription factors and variants thereof may be able to modify the expression of fatty acid biosynthetic pathways, which may, in turn, alter cell protectant levels within a plant. A number of individual fatty acids in the leaves of transgenic plants are presently of interest as cell protectants. These fatty acids of interest represent either end products or intermediates within one or more biosynthetic pathways. Modifications of the levels of intermediates generally affect the throughput and yield of an entire pathway, and thus measurements of individual fatty acid metabolites are often representative of changes to an entire biosynthetic pathway. For example, malonyl-CoA is a common intermediate in fatty acid biosynthesis of anthroquinones, cuticular waxes, flavonoids, and fatty acids. The formation of malonyl-CoA may be regulated by the action of acetyl-CoA carboxylase, which catalyzes ATP-dependent carboxylation of acetyl-CoA to malonyl-CoA. Control of malonyl-CoA synthesis at the enzymatic or transcriptional level has a profound impact on various fatty acid levels via a number of pathways (Higuchi, T. (1997) Biochemistry and Molecular Biology of Wood, Springer-Verlag, Berlin, pages 238-242, and Buchanan et al (2000) Biochemistry and Molecular Biology of Plants, American Society of Plant Physiology, Rockville, Md., pages 465-471) for example, saturated fatty acids such as 18:1, 18:2 and/or 18:3 fatty acids that may protect plants freezing stress). Thus, analysis of both total lipids and of individual fatty acids that affect entire pathways is central to understanding how fatty acid biosynthesis and composition can act to modulate a plant's cell protectant levels.

Total lipids from Arabidopsis leaves were measured by extraction, hydrolysis, and methylation essentially as described by Benning and Somerville ((1992) J. Bacteriol. 174: 2352-2360). Triplicate samples of leaf material (approximately 20 mg fresh weight) were placed in TEFLON-lined glass screw cap tubes with 1 ml 1N HCl in methanol and heated at 80° C. for 40 min. Myristic acid (14:0) (5 μg) was added as an internal standard to each sample. The resulting fatty acid methyl esters were partitioned into 0.9% NaCl in hexane (1 ml), the hexane phase concentrated to a small volume and the entire sample separated by gas chromatography as detailed (Rossak et al. 1997 Arch. Biochem. Biophys. 340:219-230). The individual fatty acids were quantified using AGP_TOP software (Hewlett Packard). Experiments were performed to identify those transformants or knockouts that exhibited an improved environmental stress tolerance, including cold or freezing stress, drought stress or salt stress, as described in the following examples. For such studies, the transformants were exposed to a variety of environmental stresses. Plants were exposed to chilling stress (6 hour exposure to 4-8° C.), heat stress (6 hour exposure to 32-37° C.), high salt stress (6 hour exposure to 200 mM NaCl), drought stress (168 hours after removing water from trays), or osmotic stress (6 hour exposure to 3 M mannitol).

### Example 6

Use of CBF1 to Induce Cold Regulated Gene Expression in Non-Cold Acclimated Arabidopsis Plants.

The following example demonstrates that increased expression of CBF1 induces COR gene expression in non-cold acclimated Arabidopsis plants. Transgenic Arabidopsis plants that overexpress CBF1 were created by placing a cDNA encoding CBF1 under the control of the strong cauliflower mosaic virus (CaMV) 35S promoter and transforming the chimeric gene into Arabidopsis ecotype RLD plants (Standard procedures were used for plasmid manipulations (Sambrook et al. (1989), supra). The CBF1-containing AseI-BglII fragment from pACT-Bgl+ (Stockinger et al. (1997) Proc. Natl. Acad. Sci. 94: 1035) was gel-purified, BamHI linkers were ligated to both ends and the fragment was inserted into the BamHI site in pCIB710 (Rothstein et al. (1987) Gene 53: 153-161) which contains the CaMV 35S promoter and terminator. The chimeric plasmid was linearized at the KpnI site and inserted into the KpnI site of the binary vector pCIB10g (Ciba-Geigy, Research Triangle Park, N.C.). The plasmid was transformed into Agrobacterium tumefaciens strain C58C1 (pMP90) by electroporation. Arabidopsis plants were transformed by the vacuum infiltration procedure (Bechtold et al. (1993) Acad. Sci. Paris, Life Sci. 316: 1194-1199) as modified (van Hoof et al. (1996) Plant Journal 10: 415-424). Initial screening gave rise to two transgenic lines, A6 and B16, that accumulated CBF1 transcripts at elevated levels.

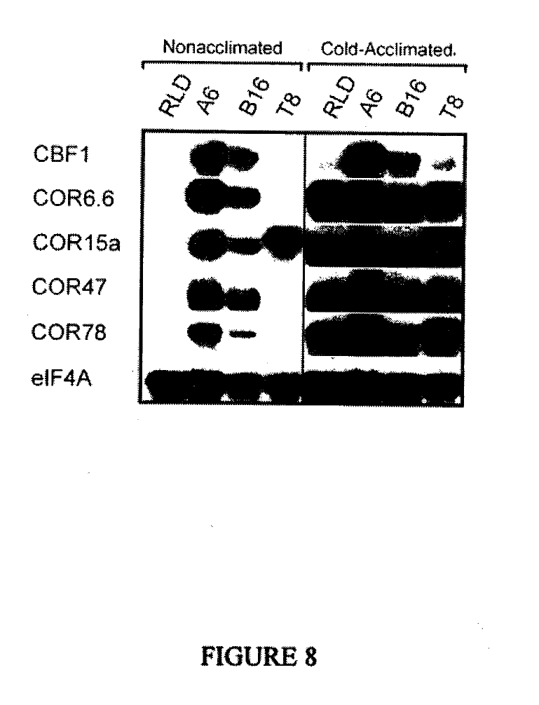

FIG. 8 is a Northern blot showing CBF1 and COR transcript levels in RLD and transgenic Arabidopsis plants. Leaves from non-cold acclimated and three-day cold-acclimated plants (Arabidopsis thaliana ecotype RLD plants were grown in pots under continuous light (100 μE/m²/sec) at 22 C for 18-25 days as described (Gilmour et al. Plant Physiol. 87: 735 (1988)). In some cases, plants were then cold-acclimated by placing them at 2.5° C. under continuous light (50 μE/m²/sec) for varying amounts of time. Leaves were harvested and total RNA prepared and analyzed for CBF1 and COR transcripts by RNA blot analysis using ³²P-radiolabeled probes (Total RNA was isolated from plant leaves and subjected to RNA blot analysis using high stringency hybridization and wash conditions as described (Stockinger et al. (1997) Proc. Natl. Acad. Sci. 94: 1035-1040; and Gilmour et al. (1988) Plant Physiol. 87: 745-750).

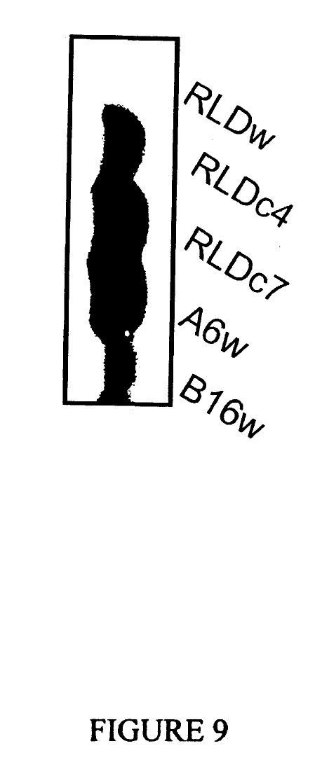

FIG. 9 is an immunoblot showing COR15am protein levels in RLD and transgenic Arabidopsis plants. Total soluble protein (100 μg) was prepared from leaves of the non-cold acclimated RLD (RLDw), 4-day cold-acclimated RLD (RLDc4), 7-day cold-acclimated RLD (RLDc7) and non-cold acclimated A6 and B16 plants and the levels of COR15am determined by immunoblot analysis using antiserum raised against the COR15am polypeptide (Total soluble protein was isolated from plant leaves, fractionated by tricine SDS-PAGE and transferred to 0.2 micron nitrocellulose as previously described (Artus et al. (1996) Proc. Natl. Acad. Sci. 93: 13404). COR15am protein was detected using antiserum raised to purified COR15am and protein A conjugated alkaline phosphatase (Sigma-Aldrich, St. Louis, Mo.) (Artus et al. (1996) supra). No reacting bands were observed with pre-immune serum (not shown).

Southern analysis indicated that the A6 line had a single DNA insert while the B16 line had multiple inserts (not shown). Examination of fourth generation homozygous A6 and B16 plants indicated that CBF1 transcript levels were higher in non-cold acclimated A6 and B16 plants than they were in non-cold acclimated RLD plants, the levels in A6 being about three fold higher than in B16 (FIG. 8).

CBF1 overexpression resulted in strong induction of COR gene expression (FIG. 8). Specifically, the transcript levels of COR6.6, COR15a, COR47 and COR78 were dramatically elevated in non-cold acclimated A6 and B16 plants as compared to non-cold acclimated RLD plants. The effect was greater in the A6 line, where COR transcript levels in non-cold acclimated plants approximated those found in cold-acclimated RLD plants. The finding that COR gene expression was greater in A6 plants than in B16 plants was consistent with CBF1 transcript levels being higher in the A6 plants (FIG. 7A). Immunoblot analysis indicated that the levels of the COR15am (FIG. 9) and COR6.6 (not shown) polypeptides were also elevated in the A6 and B16 lines, the level of expression again being higher in the A6 line. Attempts to identify the CBF1 protein in either RLD or transgenic plants were unsuccessful. Overexpression of CBF1 had no effect on the transcript levels for eIF4A (eukaryotic initiation factor 4A) (Metz et al. (1992) Gene 120: 313-314), a constitutively expressed gene that is not responsive to low temperature (FIG. 8) and had no obvious effects on plant growth and development.

The results from this example demonstrate that overexpression of the Arabidopsis transcriptional activator CBF1 induces expression of an Arabidopsis COR “regulon” composed of genes carrying the CRT/DRE DNA regulatory element. It appears that CBF1 binds to the CRT/DRE DNA regulatory elements present in the promoters of these genes and activates transcription that is consistent with the notion of CBF1 having a role in COR gene regulation. Significantly, there was a strong correlation between CBF1 transcript levels and the magnitude of COR gene induction in non-cold acclimated A6, B16, and RLD plants (FIG. 8). However, upon low temperature treatment the level of CBF1 transcripts remained relatively low in RLD plants, while COR gene expression was induced to about the same level as that in non-cold acclimated A6 plants (FIG. 8). Thus, it appears that CBF1 or an associated protein becomes “activated” in response to low temperature.

### Example 7

CBF Overexpression Resulted in a Marked Increase in Plant Freezing Tolerance

A. Experiments with Aribidopsis thaliana

The following example describes a comparison of the freezing tolerance of non-cold acclimated Arabidopsis plants that overexpress CBF1 to that of cold-acclimated wild-type plants. As described below, the freezing tolerance of non-cold acclimated Arabidopsis plants overexpressing CBF1 significantly exceeded that of non-cold acclimated wild-type Arabidopsis plants and approached that of cold-acclimated wild-type plants.

Freezing tolerance was determined using the electrolyte leakage test (Sukumaran (1972) et al. Hort. Science 7: 467). Detached leaves were frozen to various subzero temperatures and, after thawing, cellular damage (due to freeze-induced membrane lesions) was estimated by measuring ion leakage from the tissues.

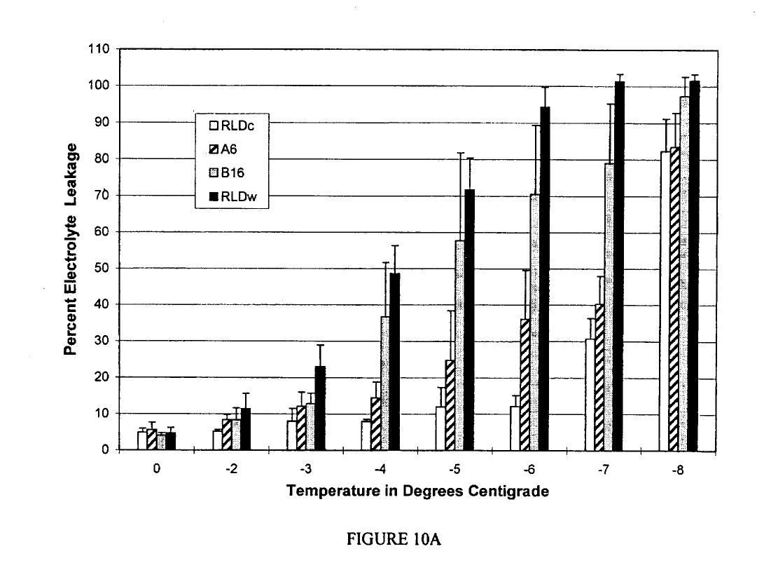

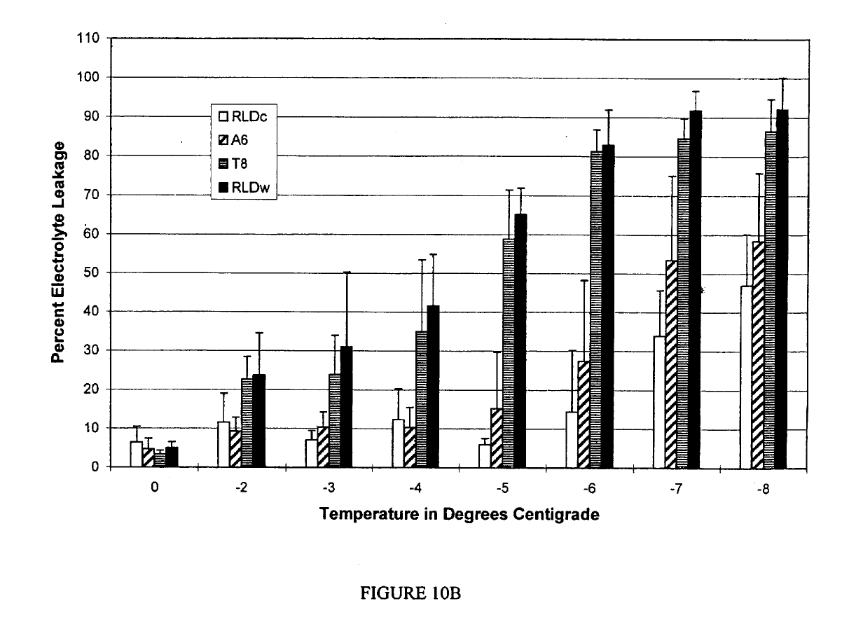

FIGS. 10A and 10B are graphs showing freezing tolerance of leaves from RLD and transgenic Arabidopsis plants. Leaves from non-cold acclimated RLD (RLDw) plants, cold-acclimated RLD (RLDc) plants and non-cold acclimated A6, B16 and T8 plants were frozen at the indicated temperatures and the extent of cellular damage was estimated by measuring electrolyte leakage (Electrolyte leakage tests were conducted as described (Sukumaran et al. (1972) supra; and Gilmour et al. (1988) Plant Physiol. 87: 735) with the following modifications. Detached leaves (2-4) from non-cold acclimated or cold-acclimated plants were placed in a test tube and submerged for 1 hour in a −2° C. water-ethylene glycol bath in a completely randomized design, after which ice crystals were added to nucleate freezing. After an additional hour of incubation at −2° C., the samples were cooled in decrements of 1° C. each hour until −8° C. was reached. Samples (five replicates for each data point) were thawed overnight on ice and incubated in 3 ml distilled water with shaking at room temperature for 3 hours. Electrolyte leakage from leaves was measured with a conductivity meter. The solution was then removed, the leaves frozen at −80° C. (for at least one hour), and the solution returned to each tube and incubated for 3 hours to obtain a value for 100% electrolyte leakage. In FIG. 10A and 10B, the RLDc plants were cold-acclimated for 10 and 11 days, respectively. Error bars indicate standard deviations.

As can be seen from FIG. 10A and 10B, CBF1 overexpression resulted in a marked increase in plant freezing tolerance. The experiment presented in FIG. 10A indicates that the leaves from both non-cold acclimated A6 and B16 plants were more freezing tolerant than those from non-cold acclimated RLD plants. Indeed, the freezing tolerance of leaves from non-cold acclimated A6 plants approached that of leaves from cold-acclimated RLD plants. The results also indicate that the leaves from non-cold acclimated A6 plants were more freezing tolerant than those from non-cold acclimated B16 plants, a result that is consistent with the greater level of CBF1 and COR gene expression in the A6 line.

The results presented in FIG. 10B further demonstrate that the freezing tolerance of leaves from non-cold acclimated A6 plants was greater than that of leaves from non-cold acclimated RLD plants and that it approached the freezing tolerance of leaves from cold-acclimated RLD plants. In addition, the results indicate that overexpression of CBF1 increases freezing tolerance to a much greater extent than overexpressing COR15a alone. This conclusion comes from comparing the freezing tolerance of leaves from non-cold acclimated A6 and T8 plants (FIG. 10B). T8 plants (Artus (1996) supra) are from a transgenic line that constitutively expresses COR15a (under control of the CaMV 35S promoter) at about the same level as in A6 plants (FIG. 1). However, unlike in A6 plants, other CRT/DRE-regulated COR genes are not constitutively expressed in T8 plants (FIG. 8).

A comparison of EL₅₀ values (the freezing temperature that results in release of 50% of tissue electrolytes) of leaves from RLD, A6, B16 and T8 plants is presented in Table 5.

EL₅₀ values were calculated and compared by analysis of variance curves fitting up to third order linear polynomial trends were determined for each electrolyte leakage experiment. To insure unbiased predictions of electrolyte leakage, trends significantly improving the model fit at the 0.2 probability level were retained. EL₅₀ values were calculated from the fitted models. In Table 2, an unbalanced one-way analysis of variance, adjusted for the different numbers of EL₅₀ values for each plant type, was determined using SAS PROC GLM [SAS Institute, Inc. (1989), SAS/STAT User's Guide, Version 6, Cory, N.C.)]. EL₅₀ values±SE (n) are presented on the diagonal line for leaves from non-cold acclimated RLD (RLDw), cold-acclimated (7 to 10 days) RLD (RLDc) and non-cold acclimated A6, B16 and T8 plants. P values for comparisons of EL₅₀ values are indicated in the intersecting cells.

TABLE 5
EL₅₀ values

| | RLDw | RLDc | A6 | B16 | T8 |
|---|---|---|---|---|---|
| RLDw | −3.9 ± 0.21 (8) | P < 0.0001 | P < 0.0001 | P = 0.0014 | P = 0.7406 |
| RLDc | | −7.6 ± 0.30 (4) | P = 0.3261 | P < 0.0001 | P < 0.0001 |
| A6 | | | −7.2 ± 0.25 (6) | P < 0.0001 | P < 0.0001 |
| B16 | | | | −5.2 ± 0.27 (5) | P = 0.0044 |
| T8 | | | | | −3.8 ± 0.35 (3) |

The data confirm that: 1) the freezing tolerance of leaves from both non-cold acclimated A6 and B16 plants is greater than that of leaves from both non-cold acclimated RLD and T8 plants; and 2) that leaves from non-cold acclimated A6 plants are more freezing tolerant than leaves from non-cold acclimated B16 plants. No significant difference was detected in EL₅₀ values for leaves from non-cold acclimated A6 and cold-acclimated RLD plants or from non-cold acclimated RLD and T8 plants.

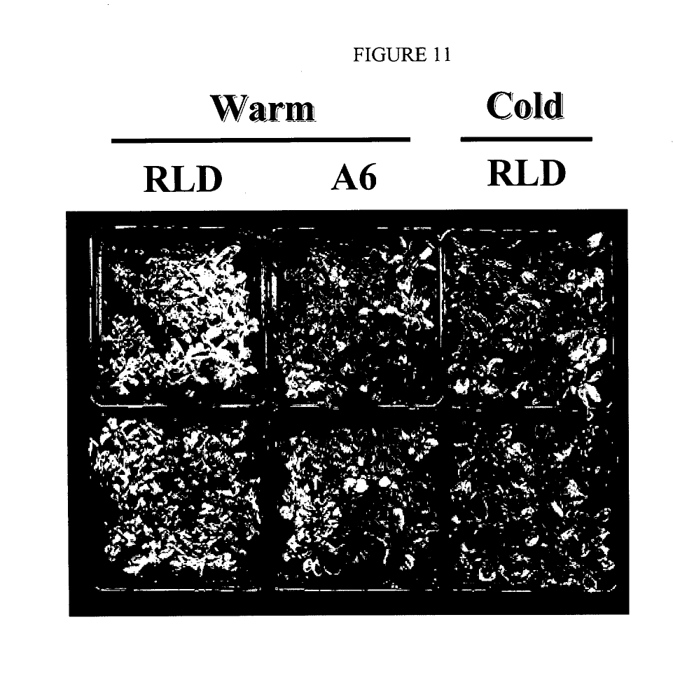

The enhancement of freezing tolerance in the A6 line was also apparent at the whole plant level. FIG. 11 is a photograph showing freezing survival of RLD and A6 Arabidopsis plants. Non-cold acclimated (WARM) RLD and A6 plants and 5-day cold-acclimated (COLD) RLD plants were frozen at −5° C. for 2 days and then returned to a growth chamber at 22° C. (Pots (3.5 inch) containing about 40 non-cold acclimated Arabidopsis plants (20 day old) and 4 day cold-acclimated plants (25 days old) (Arabidopsis thaliana ecotype RLD plants were grown in pots under continuous light (100 μE/m²/sec) at 22° C. for 18-25 days as described (Gilmour et al. (1988) Plant Physiol. 87: 735). In some cases, plants were then cold-acclimated by placing them at 2.5° C. under continuous light (50 μE/m²/sec) for varying amounts of time) were placed in a completely randomized design in a −5° C. cold chamber in the dark. After 1 hour, ice chips were added to each pot to nucleate freezing. Plants were removed after 2 days and returned to a growth chamber at 22° C.). A photograph of the plants after 7 days of regrowth is shown.

Although the magnitude of the difference varied from experiment to experiment, non-cold acclimated A6 plants consistently displayed greater freezing tolerance in whole plant freeze tests than did non-cold acclimated RLD plants (FIG. 11). No difference in whole plant freeze survival was detected between non-cold acclimated B16 and RLD plants or non-cold acclimated T8 and RLD plants (not shown).

The results of this experiment show that CBF1-induced expression of CRT/DRE-regulated COR genes result in a dramatic increase in freezing tolerance and confirms the belief that COR genes play a major role in plant cold acclimation. The increase in freezing tolerance brought about by expressing the battery of CRT/DRE-regulated COR genes was much greater than that brought about by overexpressing COR15a alone indicating that COR genes in addition to COR15a have roles in freezing tolerance.

Traditional plant breeding approaches have met with limited success in improving the freezing tolerance of agronomic plants (Thomashow (1990) Adv. Genet 28: 99-131). For instance, the freezing tolerance of the best wheat varieties today is essentially the same as the most freezing-tolerance varieties developed in the early part of the 20^{th} Century. Thus, in recent years there has been considerable interest that biotechnology might offer new strategies to improve the freezing tolerance of agronomic plants. By the results of the present invention, Applicants demonstrate the ability to enhance the freezing tolerance of non-cold acclimated Arabidopsis plants by increasing the expressing of the Arabidopsis regulatory gene CBF1. As described throughout this application, the ability of the present invention to modify the expression of environmental stress tolerance genes such as COR genes has wide ranging implications since the CRT/DRE DNA regulatory element is not limited to Arabidopsis (Jiang et al. (1996) Plant Mol. Biol. 30: 679-684). Rather, CBF1 and homologous genes can be used to manipulate expression of CRT/DRE-regulated COR genes in important crop species and thereby improve their freezing tolerance. By transforming modified versions of CBF1 (or homologs) into such plants, it will extend their safe growing season, increase yield and expand areas of production.

B. Experiments with Lycopersicon esculentum

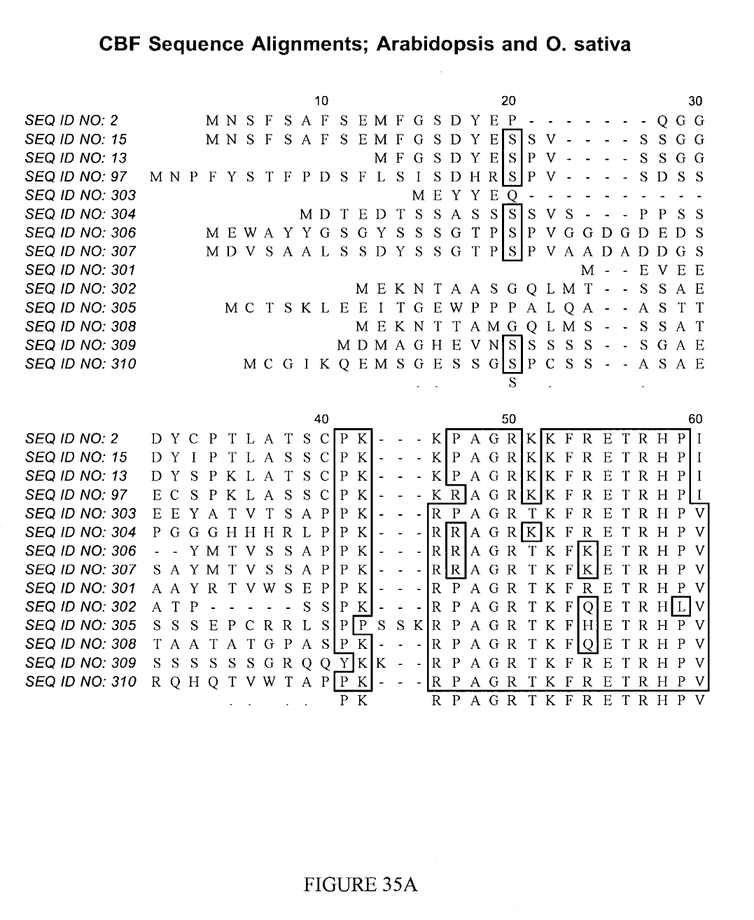

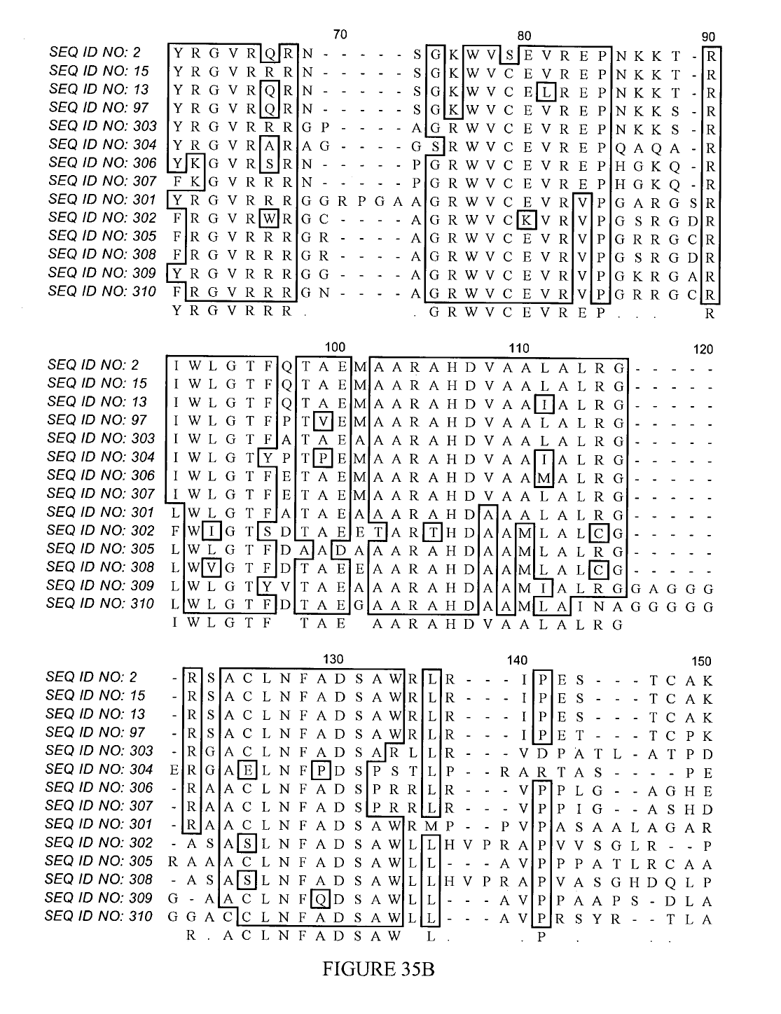

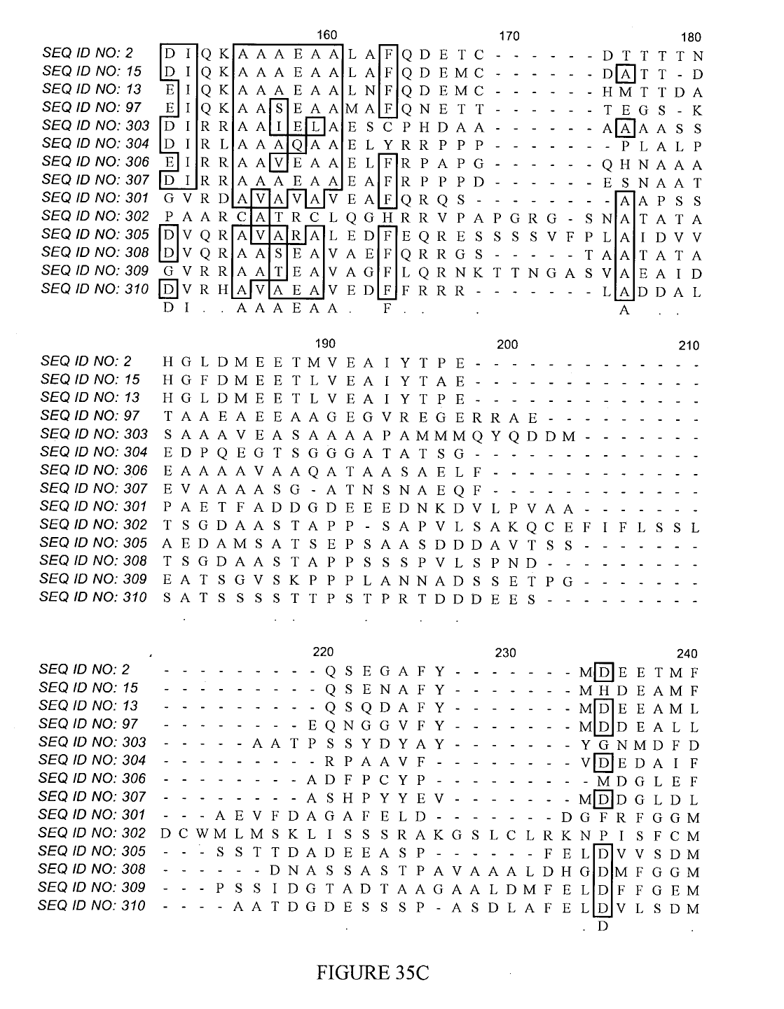

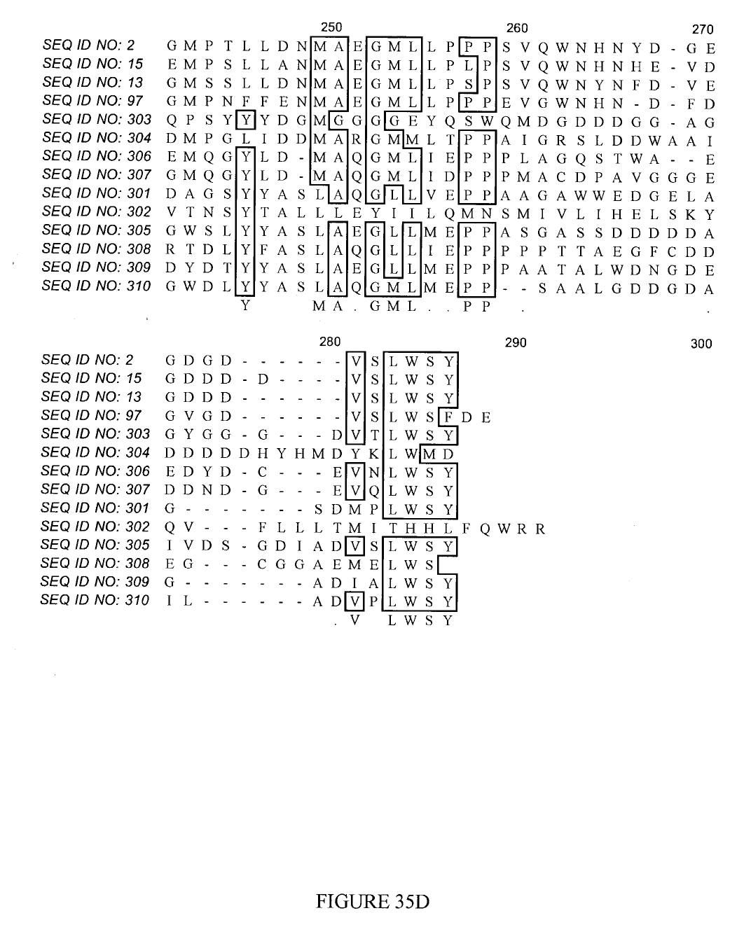

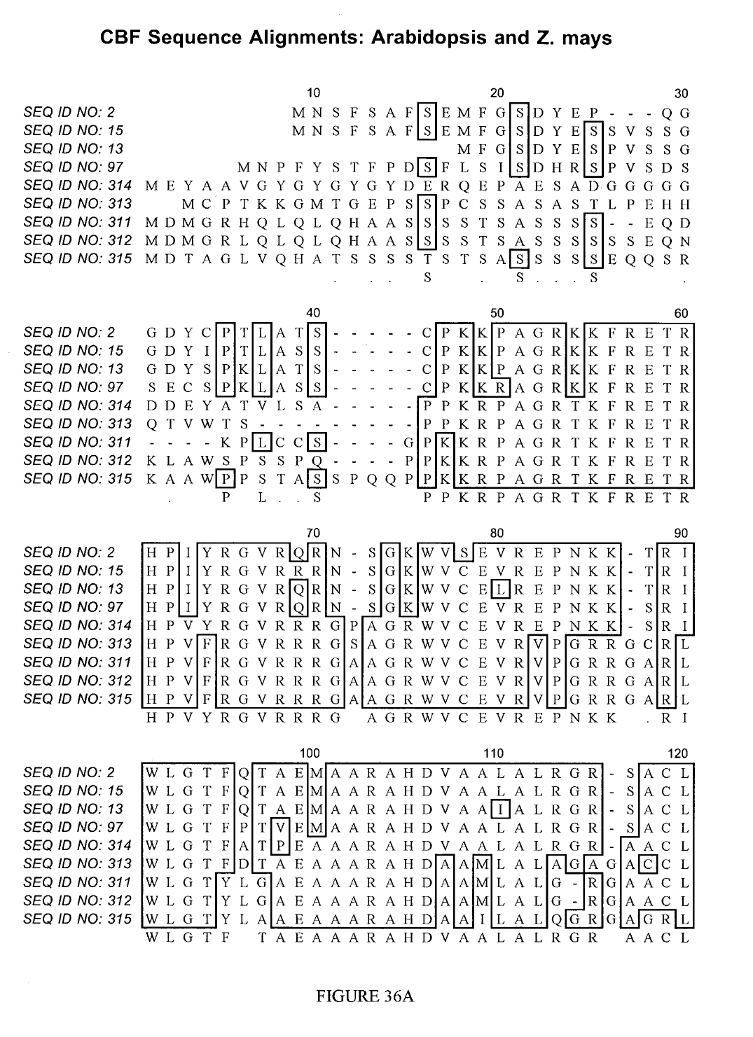

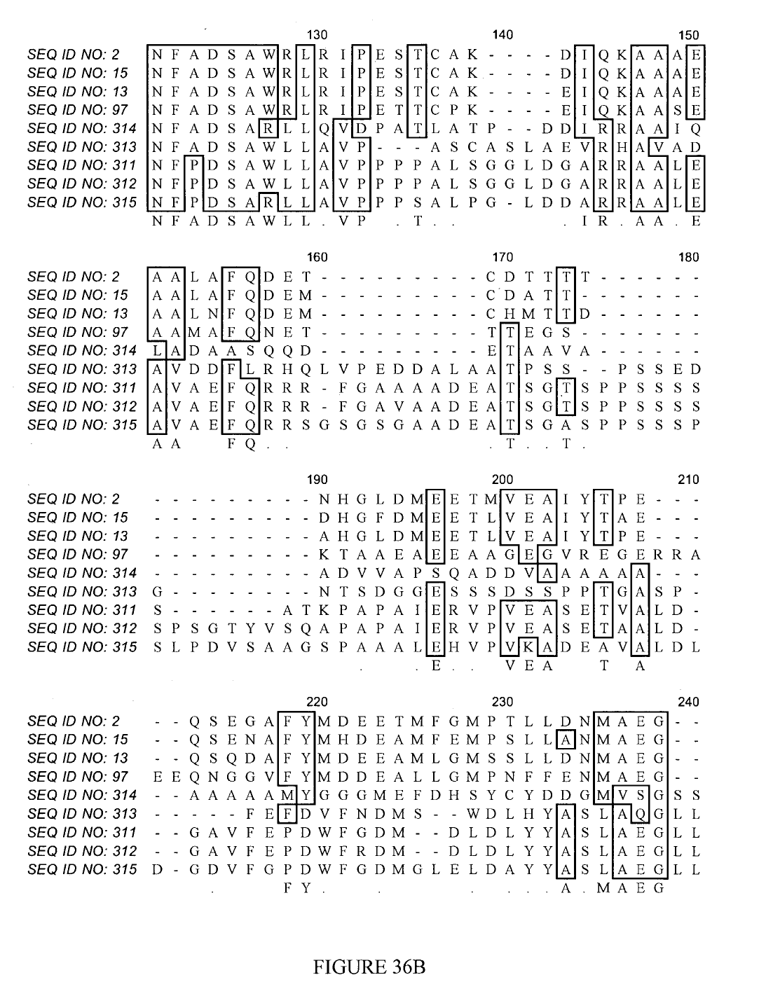

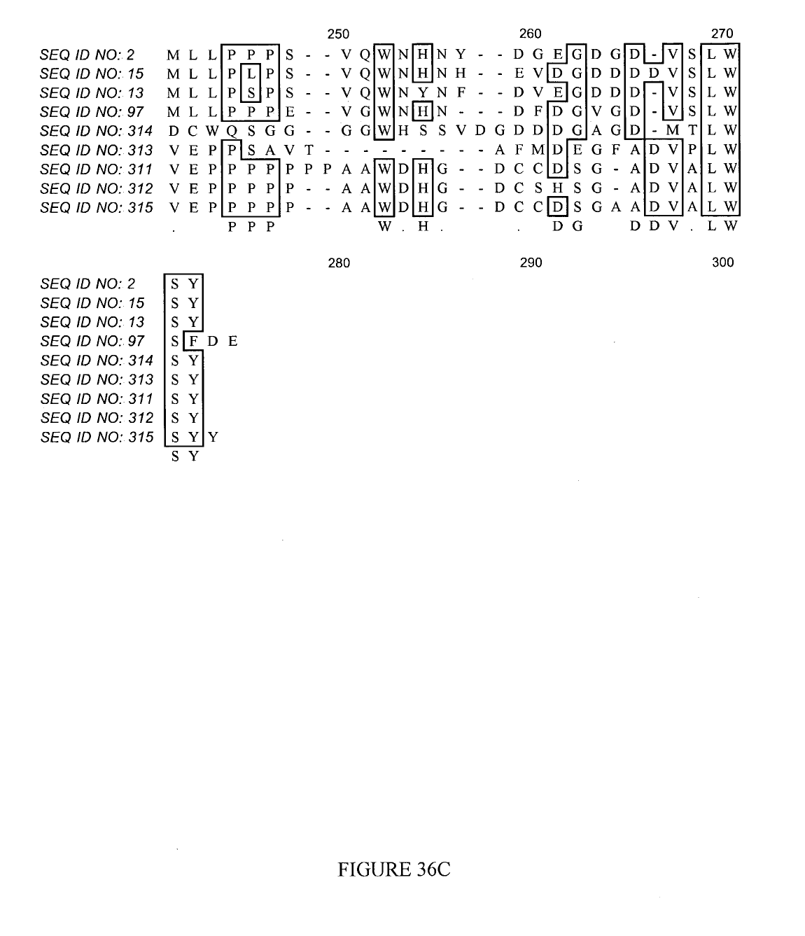

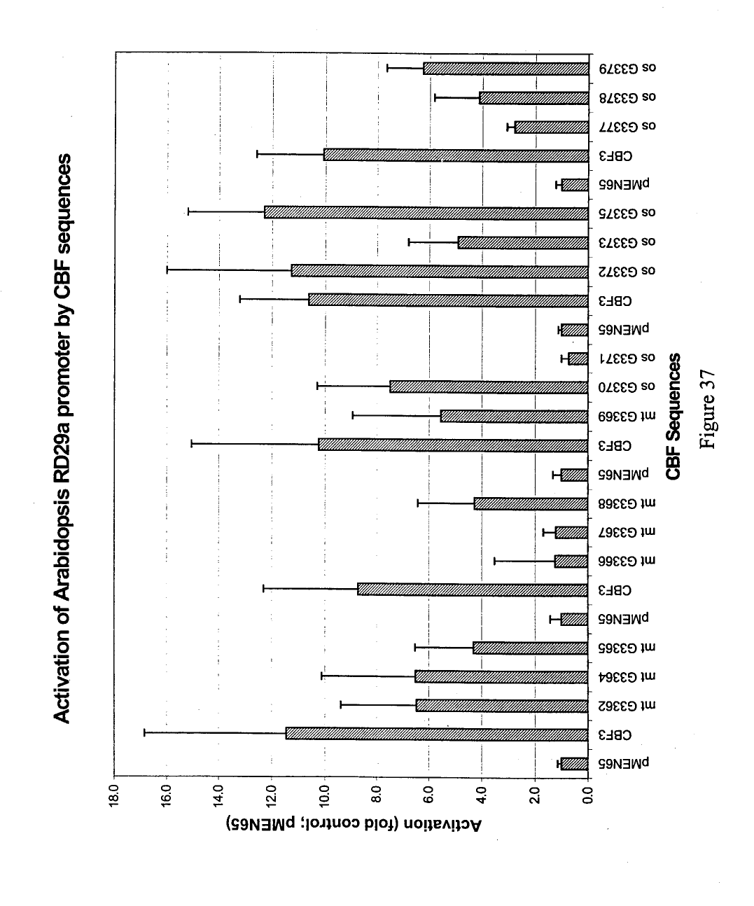

Two CBF sequences were isolated from tomato and were transformed into Arabidopsis plants using the methods described herein (Examples 4 and 6). These tomato CBF-related polypeptides, LeCBF1 (SEQ ID NO: 331) and LeCBF2 (SEQ ID NO: 332), share amino acid sequences with SEQ ID NO:6 (SEQ ID NO: 2; see FIG. 35). Similar freezing tolerance leakage experiments to those conducted with Arabidopsis plants, as described above in Example 7a, demonstrated that CBF-induced expression of CRT/DRE-regulated COR genes from tomato are endogenously induced when wild-type tomato plants are exposed to cold-stress (FIG. 36). When tomato CBF1 was expressed as a transgene in Arabidopsis plants, expression of COR genes, including both mRNA expression and protein expression, was activated (FIG. 37). This overexpression resulted in a dramatic increase in freezing tolerance, as measured by percentage electrolyte leakage (FIG. 38).

The results of this experiment show that CBF1-induced expression of CRT/DRE-regulated COR genes result in a dramatic increase in freezing tolerance and confirms the belief that COR genes play a major role in plant cold acclimation. The increase in freezing tolerance brought about by expressing the battery of CRT/DRE-regulated COR genes was much greater than that brought about by overexpressing COR15a alone indicating that COR genes in addition to COR15a have roles in freezing tolerance.

Traditional plant breeding approaches have met with limited success in improving the freezing tolerance of agronomic plants (Thomashow (1990) Adv. Genet 28: 99-131). For instance, the freezing tolerance of the best wheat varieties today is essentially the same as the most freezing-tolerance varieties developed in the early part of the 20^{th} Century. Thus, in recent years there has been considerable interest that biotechnology might offer new strategies to improve the freezing tolerance of agronomic plants. By the results of the present invention, Applicants demonstrate the ability to enhance the freezing tolerance of non-cold acclimated Arabidopsis plants by increasing the expressing of the Arabidopsis regulatory gene CBF1. As described throughout this application, the ability of the present invention to modify the expression of environmental stress tolerance genes such as COR genes has wide ranging implications since the CRT/DRE DNA regulatory element is not limited to Arabidopsis (Jiang et al. (1996) Plant Mol. Biol. 30: 679-684). Rather, CBF1 and homologous genes can be used to manipulate expression of CRT/DRE-regulated COR genes in important crop species and thereby improve their freezing tolerance. By transforming modified versions of CBF1 (or homologs) into such plants, it will extend their safe growing season, increase yield and expand areas of production.

B. Experiments with Lycopersicon esculentum

CBF sequences were isolated from tomato and were transformed into Arabidopsis plants using the methods described herein (Examples 4 and 6). One tomato CBF (CBF1; SEQ ID NO: 331) shares 56 % identity (121/215 aminom acid residues) with SEQ ID NO:2 (G40). Similar freezing tolerance leakage experiments to those conducted with Arabidopsis plants, as described above in Example 7a, demonstrate that CBF-induced expression of CRT/DRE-regulated COR genes from tomato are endogenously induced when wild-type tomato plants are exposed to cold-stress. When tomato CBF1 is expressed as a transgene in Arabidopsis plants, expression of COR genes, including both mRNA expression and protein expression, is activated. This overexpression results in a dramatic increase in freezing tolerance, as measured by percentage electrolyte leakage.

These experiments demonstrate that CBF genes in diverse species play a major role in plant cold acclimation.

### Example 8

Selection of Promoters to Control Expression of CBF1 in Plants.

The following examples describe the isolation of different promoters from plant genomic DNA, construction of the plasmid vectors carrying the CBF1 gene and the inducible promoters, transformation of Arabidopsis cells/plants with these constructs, and regeneration of transgenic plants that have altered biomass or increased tolerance to environmental stresses compared to non-transformed plants

Isolation of inducible promoters from plant genomic DNAs. Inducible promoters from different plant genomic DNAs were identified and isolated by PCR amplification using primers designed to flank the promoter region and contain suitable restriction sites for cloning into the expression vector. The following genes were used to BLAST search GenBank to find the inducible promoters: Dreb2a; P5CS; Rd22; Rd29a; Rd29b; Rab18; Cor47. Table 6 lists the accession numbers and positions of these promoters. Table 7 lists the forward and reverse primers that were used to isolate the promoters.

TABLE 6

| Gene Name | Accession No. | Position | Length (bps) |
|---|---|---|---|
| Dreb2a | AB010692 | 51901-53955 | 2054 |
| P5CS | AC003000 | 45472-47460 | 1988 |
| Rd22 | D10703 | 17-1046 | 1029 |
| Rd29a | D13044 | 3870-5511 | 1641 |
| Rd29b | D13044 | 90-1785 | 1695 |
| Rab18 | AB013389 | 8070-9757 | 1687 |
| Cor47 | AB004872 | 1-1370 | 1370 |

TABLE 7

| | | | SEQ |
|---|---|---|---|
| Promoter name | Primer name | Cloning sites | ID NO: |
| Dreb2a | Dreb2a-reverse | HindIII (AAGCTT) | 19 |
| | Dreb2a-forward | BglII (AGATCT) | 20 |
| P5CS | P5CS-reverse | HindIII (AAGCTT) | 21 |
| | P5CS-forward | BglII (AGATCT) | 22 |
| Rd22 | Rd22-reverse | HindIII (AAGCTT) | 23 |
| | Rd22-forward | KpnI (GGTACC) | 24 |
| Rd29a | Rd29a-reverse | HindIII (AAGCTT) | 25 |
| | Rd29a-forward | KpnI (GGTACC) | 26 |
| Rd29b | Rd29b-reverse | HindIII (AAGCTT) | 27 |
| | Rd29b-forward | KpnI (GGTACC) | 28 |
| Rab 18 | Rab18-reverse | HindIII (AAGCTT) | 29 |
| | Rab18-forward | BglII (AGATCT) | 30 |
| Cor47 | Cor47-reverse | HindIII (AAGCTT) | 31 |
| | Cor47-forward | BglII (AGATCT) | 32 |

(1) Dreb2a Promoter

A cDNA encoding DRE (C-repeat) binding protein (DREB2A) has been recently identified (Liu et al. (1998) Plant Cell 10: 1391-1406). The transcription of the DREB2A gene is activated by dehydration and high-salt stress, but not by cold stress. The upstream untranslated region (166 bps) of dreb2a was used to BLAST-search the public database. A region containing the DREB2A promoter was identified in chromosome 5 of Arabidopsis (Accession No. AB010692) between nucleotide positions 51901-53955 (Table 6).

Two PCR primers designed to amplify the promoter region from Arabidopsis thaliana genomic DNA are as follows:

dreb2a-reverse: 5′-GCCCAAGCTTCAAGTTTAGTGAGCACTATGTGCTCG-3′ [SEQ ID NO: 19];

and dreb2a-forward: 5′-GGAAGATCTCCTTCCCAGAAACAACACAATCTAC-3′ [SEQ ID NO: 20].

The dre2ba-reverse primer includes a Hind III (AAGCTT) restriction site near the 5′-end of the primer and dreb2a-forward primer has a Bgl II (AGATCT) restriction site at near 5′-end of the primer. These restriction sites may be used to facilitate cloning of the fragment into an expression vector.

Total genomic DNA may be isolated from Arabidopsis thaliana (ecotype colombia) by using the CTAB method (Ausubel et al. (1992) Current Protocols in Molecular Biology (Greene & Wiley, New York)). Ten nanograms of the genomic DNA can be used as a template in a PCR reaction under conditions suggested by the manufacturer (Boehringer Mannheim). The reaction conditions that may be used in this PCR experiment are as follows: Segment 1: 94° C., 2 minutes; Segment 2: 94° C., 30 seconds; 60° C., 1 minute; 72° C., 3 minutes, for a total of 35 cycles; Segment 3: 72° C. for 10 minutes. A PCR product of 2054 bp is expected.

The PCR products can be subject to electrophoresis in a 0.8% agarose gel and visualized by ethidium bromide staining. The DNA fragments containing the inducible promoter will be excised and purified using a QIAQUICK gel extraction kit (Qiagen, Valencia, Calif.).

(2) P5CS Promoter

A cDNA for delta 1-pyrroline-5-carboxylate synthetase (P5CS) has been isolated and characterized (Yoshiba et al. (1995) Plant J. 7: 751-760). The cDNA encodes an enzyme involved in the biosynthesis of proline under osmotic stress (drought/high salinity). The transcription of the P5CS gene was found to be induced by dehydration, high salt, and treatment with plant hormone ABA, while it did not respond to heat or cold treatment.

A genomic DNA containing a promoter region of P5CS was identified by a BLAST search of GenBank using the upstream untranslated region (106 bps) of the P5CS sequence (Accession No. D32138). The sequence for the P5CS promoter is located in the region between from nucleotide positions 45472 to 47460 (Accession No. AC003000; Table 6).

Reverse and forward PCR primers designed to amplify this promoter region from Arabidopsis thaliana genomic DNA are

P5CS-reverse primer 5′-GCCCAAGCTTGTTTCATTTTCTCCATGAAGGAGAT-3′ [SEQ ID NO: 21]; and

P5CS-forward primer 5′-GGAAGATCTTATCGTCGTCGTCGTCTACCAAAACCACAC-3′ [SEQ ID NO: 22].

Total genomic DNA may be isolated from Arabidopsis thaliana (ecotype colombia) by using the CTAB method (Ausubel et al. (1992) Current Protocols in Molecular Biology (Greene & Wiley, New York)). Ten nanograms of the genomic DNA can be used as a template in a PCR reaction under conditions suggested by the manufacturer (Boehringer Mannheim). The PCR product is expected to be 1988 bps and may be PCR amplified and gel purified following the same protocol described for the dreb2a promoter.

(3) rd22 Promoter

A cDNA clone of rd22 was isolated from Arabidopsis under dehydration conditions (Yamaguchi-Shinozaki et al. (1993) Mol. Gen. Genet. 238: 17-25). Transcripts of rd22 were found to be induced by salt stress, water deficit and endogenous abscisic acid (ABA) but not by cold or heat stress. A promoter region was identified from GenBank by using Nucleotide Search WWW Entrez at the NCBI with the rd22 as a search word. The sequence for the rd22 promoter is located in the region between nucleotide positions 17 to 1046 (Accession No. D10703; Table 6).

Reverse and forward PCR primers designed to amplify this promoter region from Arabidopsis thaliana genomic DNA are

rd22-reverse primer 5′-GCTCTAAGCTTCACAAGGGGTTCGTTTGGTGC-3′ [SEQ ID NO: 23]; and

rd22-forward primer 5′-GGGGTACCTTTTGGGAGTTGGAATAGAAATGGGTTTGATG-3′ [SEQ ID NO: 24].

The rd22-reverse primer includes a Hind III (AAGCTT) restriction site near the 5′-end of primer and rd22-forward primer has a KpnI (GGTACC) restriction site at near 5′-end of primer.

Total genomic DNA may be isolated from Arabidopsis thaliana (ecotype colombia) by using the CTAB method (Ausubel et al. (1992) Current Protocols in Molecular Biology (Greene & Wiley, New York)). Ten nanograms of the genomic DNA can be used as a template in a PCR reaction under conditions suggested by the manufacturer (Boehringer Mannheim). The PCR product is expected to be 1029 bps and may be PCR amplified and gel purified following the same protocol described for the dreb2a promoter.

(4) rd29a Promoter

The rd29a and rb29b genes were isolated and characterized by Shinozaki's group in Japan (Yamaguchi-Shinizaki et al. (1993) Plant Physiol. 101: 1119-1120). Both rd29a and rb29b gene expressions were found to be induced by desiccation, salt stress and exogenous ABA treatment (Yamaguchi-Shinizaki et al. (1993) supra); Ishitani et al. (1998) Plant Cell 10: 1151-1161). The rd29a gene expression was induced within 20 min after desiccation, but rd29b mRNA did not accumulate to a detectable level until 3 hours after desiccation. Expression of rd29a could also be induced by cold stress, whereas expression of rd29b could not be induced by low temperature.

A genomic clone carrying the rd29a promoter was identified by using Nucleotide Search WWW Entrez at the NCBI with the rd29a as a search word. The sequence for the rd29a promoter is located in the region between nucleotide positions 3870 to 5511 (Accession No. D13044, Table 6).

Reverse and forward primers designed to amplify this promoter region from Arabidopsis genomic DNA are:

rd29a-reverse primer 5′-GCCCAAGCTTAATTTTACTCAAAATGTTTTGGTTGC-3′ [SEQ ID NO: 25]; and

rd29a-forward primer 5′-CCGGTACCTTTCCAAAGATTTTTTTCTTTCCAATAGAAGTAATC-3′ [SEQ ID NO: 26].

The rd29a-reverse primer includes a Hind III (AAGCTT) restriction site near the 5′-end of primer and rd29a-forward primer has a KpnI (GGTACC) restriction site near 5′-end of primer.

Total genomic DNA may be isolated from Arabidopsis thaliana (ecotype colombia) by using the CTAB method (Ausubel et al. (1992) Current Protocols in Molecular Biology (Greene & Wiley, New York)). Ten nanograms of the genomic DNA can be used as a template in a PCR reaction under conditions suggested by the manufacturer (Boehringer Mannheim). The PCR product is expected to be 1641 bps and may be PCR amplified and gel purified following the same protocol described for the dreb2a promoter.

(5) rd29b Promoter

A genomic clone carrying the rd29b promoter was identified by using Nucleotide Search WWW Entrez at the NCBI with the rd29b as a search word. The sequence for the rd29a promoter was located in the region between nucleotide positions 90 to 1785 for rd29b (Accession No. D13044; Table 6).

Reverse and forward PCR primers designed to amplify this promoter region from Arabidopsis thaliana genomic DNA are:

rd29b-reverse primer 5′-GCGGAAGCTTCATTTTCTGCTACAGAAGTG-3′ [SEQ ID NO: 27]; and

rd29b-forward primer 5′-CCGGTACCTTTCCAAAGCTGTGTTTTCTCTTTTTCAAGTG-3′ [SEQ ID NO: 28].

Total genomic DNA may be isolated from Arabidopsis thaliana (ecotype colombia) by using the CTAB method (Ausubel et al. (1992) Current Protocols in Molecular Biology (Greene & Wiley, New York)). Ten nanograms of the genomic DNA can be used as a template in a PCR reaction under conditions suggested by the manufacturer (Boehringer Mannheim). The PCR product is expected to be 1695 bps and may be PCR amplified and gel purified following the same protocol described for the dreb2a promoter.

(6) rab18 Promoter

A rab-related (responsive to ABA) gene, rab18 from Arabidopsis has been isolated. The gene encodes a hydrophilic, glycine-rich protein with the conserved serine- and lysine-rich domains. The rab18 transcripts accumulate in plants exposed to water deficit or exogenous abscisic acid (ABA) treatment. A weak induction of rab18 mRNA by low temperature was also observed (Ishitani et al. (1998) Plant Cell 10: 1151-1161).

A genomic DNA containing a promoter region of rab18 was identified by a BLAST search of GenBank using the upstream untranslated region (757 bps) of the rab18 sequence (Accession No. L04173). The sequence of the rab18 promoter is located in the region between nucleotide positions 8070 to 9757 (Accession No. AB013389).

Reverse and forward PCR primers designed and used to amplify this promoter region from Arabidopsis thaliana genomic DNA are:

rab18-reverse primer 5′-GCCCAAGCTTCAAATTCTGAATATTCACATATCAAAAAAGTG-3′ [SEQ ID NO: 29]; and

rab18-forward primer 5′-GGAAGATCTGTTCTTCTTGTCTTAAGCAAACACTTTGAGC-3′ [SEQ ID NO: 30].

The rab18-reverse primer includes a Hind III (AAGCTT) restriction site near the 5′-end of the primer and rab18-forward primer has a Bgl II (AGATCT) restriction site near the 5′-end of the primer.

Total genomic DNA may be isolated from Arabidopsis thaliana (ecotype colombia) by using the CTAB method (Ausubel et al. (1992) Current Protocols in Molecular Biology (Greene & Wiley, New York)). Ten nanograms of the genomic DNA can be used as a template in a PCR reaction under conditions suggested by the manufacturer (Boehringer Mannheim). The PCR product is expected to be 1687 bps and may be PCR amplified and gel purified following the same protocol described for the dreb2a promoter.

(7) Cor47 Promoter

The DNA sequence of cDNA for cold-regulated (cor47) gene of Arabidopsis thaliana was determined. Gilmour et al. (1992) Plant Molec. Biol. 18: 13-21). Expression of cor47 gene was induced by cold stress, dehydration and high NaCl treatment (Ishitani et al. (1998) Plant Cell. 10: 1151-1161). The promoter region of cor47 gene was identified in GenBank by using Nucleotide Search WWW Entrez at the NCBI with the cor47 as a search word. The sequence of the cor47 promoter is located in the region between nucleotide positions 1-1370 (Accession No. AB004872; Table 6).

Reverse and forward PCR primers designed to amplify this promoter region from Arabidopsis thaliana genomic DNA are:

cor47-reverse primer 5′-GCCCAAGCTTTCGTCTGTTATCATACAAGGCACAAAACGAC-3′ [SEQ ID NO: 31]; and

cor47-forward primer 5′-GGAAGATCTAGTTAATCTTGATTTGATTAAAAGTTTATATAG-3′ [SEQ ID NO: 32].

The cor47-reverse primer includes a Hind III (AAGCTT) restriction site near the 5′-end of the primer and cor47-forward primer has a Bgl II (AGATCT) restriction site near the 5′-end of the primer.

Total genomic DNA may be isolated from Arabidopsis thaliana (ecotype colombia) by using the CTAB method (Ausubel et al. (1992) Current Protocols in Molecular Biology (Greene & Wiley, New York)). Ten nanograms of the genomic DNA can be used as a template in a PCR reaction under conditions suggested by the manufacturer (Boehringer Mannheim). The PCR product is expected to be 1370 bps and may be PCR amplified and gel purified following the same protocol described for the dreb2a promoter.

Construction of the plasmids containing CBF1 and inducible promoter. The expression binary vector pMEN020 contains a kanamycin resistance gene (neomycin phosphotransferase) for antibiotic selection of the transgenic plants and a Spc/Str gene used for bacterial or agrobacterial selections. The pMEN020 plasmid is digested with restriction enzymes such as HindIII and BglII to remove the 35S promoter. The 35S promoter is then replaced with an inducible promoter.

(1) Cloning of the Inducible Promoter into pMEN020

The sequences of the inducible promoters that are PCR amplified and gel purified, as well as the plasmid pMEN020, are subject to restriction digestion with their respective restriction enzymes as listed in Table 7. Both DNA samples are purified by using the Qiaquick purification kit (Qiagen) and ligated at a ratio of 3:1 (vector to insert). Ligation reactions using T4 DNA ligase (New England Biolabs, MA) are carried out at 16° C. for 16 hours. The ligated DNAs are transformed into competent cells of the E. coli strain DH5α by using the heat shock method. The transformed cells are plated on LB plates containing 100 μg/ml spectinomycin (Sigma-Aldrich, St. Louis, Mo.). Individual colonies are grown overnight in five milliliters of LB broth containing 100 μg/ml spectinomycin at 37° C.

Plasmid DNAs from transformants are purified by using QIAQUICK Mini Prep kits (Qiagen) according to the manufacturer's instruction. The presence of the promoter insert is verified by restriction mapping with the respective restriction enzymes as listed in Table 7 to cut out the cloned insert. The plasmid DNA is also subject to double-strand DNA sequencing analysis using a vector primer

E9.1 primer 5′-CAAACTCAGTAGGATTCTGGTGTGT-3′ [SEQ ID NO: 33].

(2) Cloning of the cbf1 Gene into the Plasmids Containing the Inducible Promoters

To clone the CBF1 gene into the plasmids, different PCR primers with suitable restriction sites for each plasmid are used to isolate CBF1 gene from Arabidopsis thaliana genomic DNA. The primers that may be used are listed in Table 8.

TABLE 8

| Promoter name | Primer name | Cloning sites | |
|---|---|---|---|
| Dreb2a | Cbf1-reverse1 | BglII | (AGATCT) |
| | Cbf1-forward1 | BamHI | (GGATCC) |
| P5CS | Cbf1-reverse1 | BglII | (AGATCT) |
| | Cbf1-forward1 | BamHI | (GGATCC) |
| Rd22 | Cbf1-reverse2 | KpnI | (GGTACC |
| | Cbf1-forward1 | BamHI | (GGATCC) |
| Rd29a | Cbf1-reverse2 | KpnI | (GGTACC |
| | Cbf1-forward1 | BamHI | (GGATCC) |
| Rd29b | Cbf1-reverse2 | KpnI | (GGTACC |
| | Cbf1-forward1 | BamHI | (GGATCC) |
| Rab18 | Cbf1-reverse1 | BglII | (AGATCT) |
| | Cbf1-forward2 | XbaI | (TCTAGA |
| Cor47 | Cbf1-reverse1 | BglII | (AGATCT) |
| | Cbf1-forward1 | BamHI | (GGATCC) |

Two of the four available PCR primers (Table 8) are used for cloning the at-CBF1 gene into the expression vectors containing each inducible promoter described above. The four primers have these sequences:

cbf1-reverse 1 5′-GGAAGATCTTGAAACAGAGTACTCTGATCAATGAACTC-3′ [SEQ ID NO: 34],

cbf1-forward 1 5′-CGCGGATCCCTCGTTTCTACAACAATAAAATAAAATAAAATG-3′ [SEQ ID NO: 35]

cbf1-reverse 2 5′-GGGGTACCTGAAACAGAGTACTCTGATCAATGAACTC-3′ [SEQ ID NO: 36], and

cbf1-forward 2 5′-GCTCTAGACTCGTTTCTACAACAATAAAATAAAATAAAATG-3′ [SEQ ID NO: 37].

For example, for the Dreb2a, P5CS, and COR47 promoters that are ligated to a BamHI and BglII flanked insert, the cbf1-reverse 1 and cbf1-forward 1 primers [SEQ ID NO: 34 and 35, respectively] are used to isolate CBF1 gene from Arabidopsis thaliana genomic DNA. The cbf1-reverse primer includes a BglII (AGATCT) restriction site near the 5′-end of the primer and cbf1-forward primer has a BamHI (GGATCC) restriction site near the 5′-end of the primer. A PCR product of 764 bp is expected. The genomic DNA (10 ng) is used as a template in a PCR reaction under conditions suggested by the manufacturer (Boehringer Mannheim). The reaction conditions to be used in this PCR experiment are as follows: Segment 1: 94° C., 2 minutes; Segment 2: 94° C., 30 seconds; 55° C., 1 minute; 72° C., 1 minute, for a total of 35 cycles; Segment 3: 72° C. for 10 minutes.

The PCR products are subject to electrophoresis in a 0.8% agarose gel and visualized by ethidium bromide staining. The DNA fragment containing cbf1 is excised and purified by using a Qiaquick gel extraction kit (Qiagen). The purified fragment and the vector pMBI2001 containing the inducible promoter (Table 8) are each digested with BglII and BamHI restriction enzymes at 37° C. for 2 hours. Both DNA samples are purified by using the QIAQUICK purification kit (Qiagen) and ligated at a ratio of 3:1 (vector to insert ratio). Ligation reactions using T4 DNA ligase (New England Biolabs, MA) are carried out at 16° C. for 16 hours. The ligated DNAs are transformed into competent cells of the E. coli strain DH5α by using the heat shock method. The transformation are plated on LB plates containing 100 (g/ml spectinomycin (Sigma-Aldrich).

Individual colonies are grown overnight in five milliliters of LB broth containing 100 g/ml spectinomycin at 37° C. Plasmid DNA are purified by using QIAQUICK Mini Prep kits (Qiagen). The presence of the cbf1 insert is verified by restriction mapping with BglII and BamHI. The plasmid DNA is also subject to double-strand DNA sequencing analysis by using vector primer E9.1

5′-CAAACTCAGTAGGATTCTGGTGTGT-3′ [SEQ ID NO: 33].

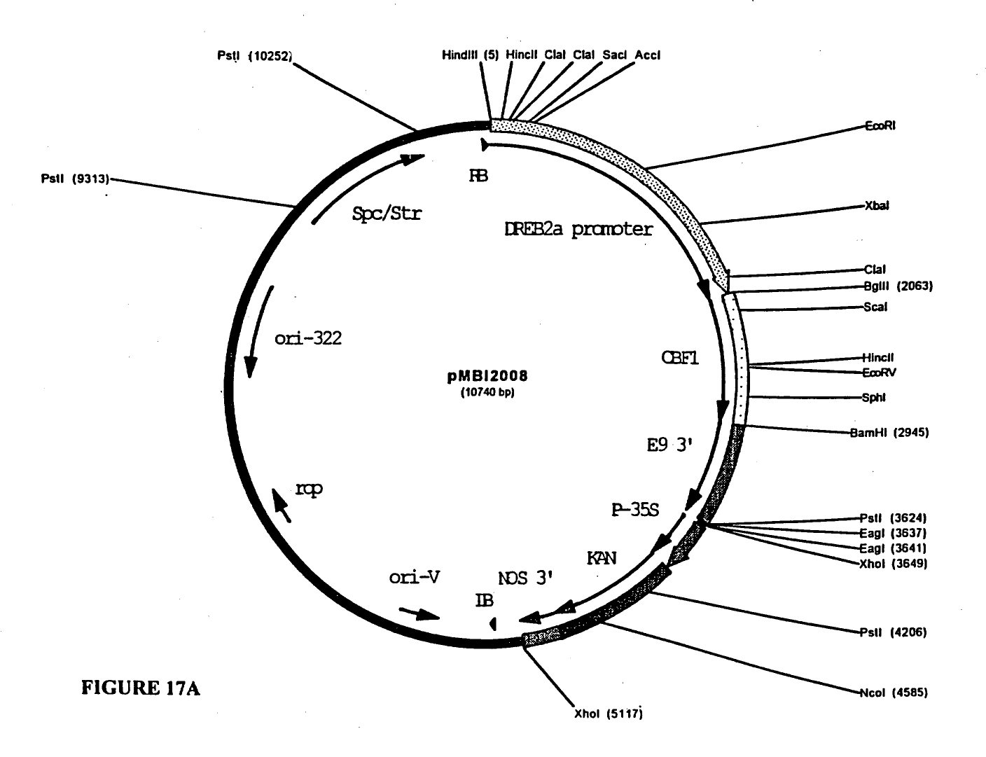

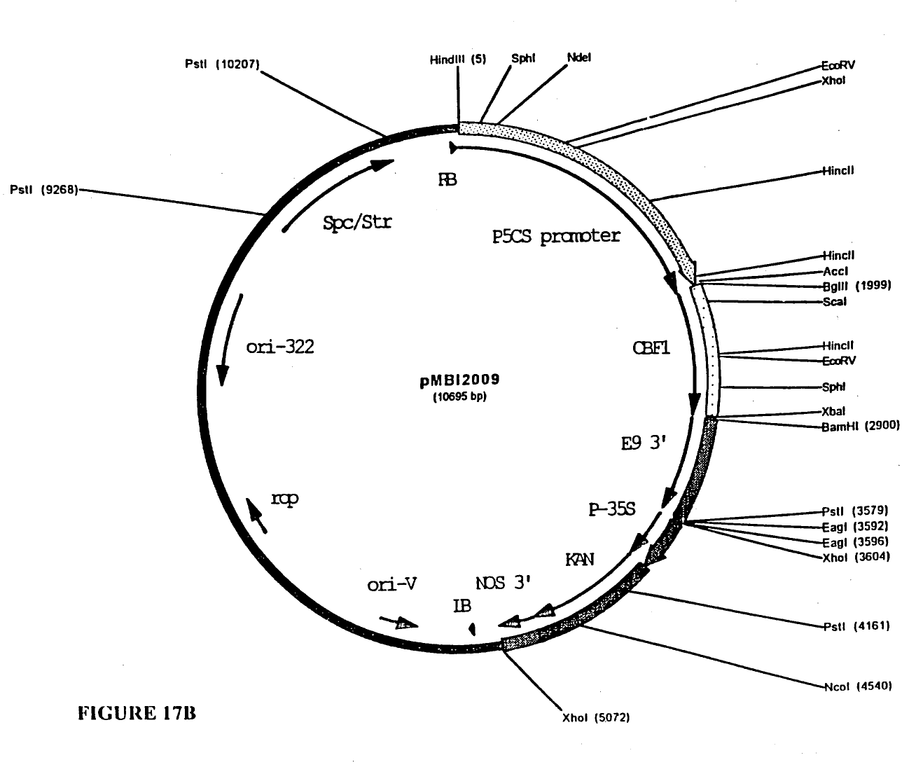

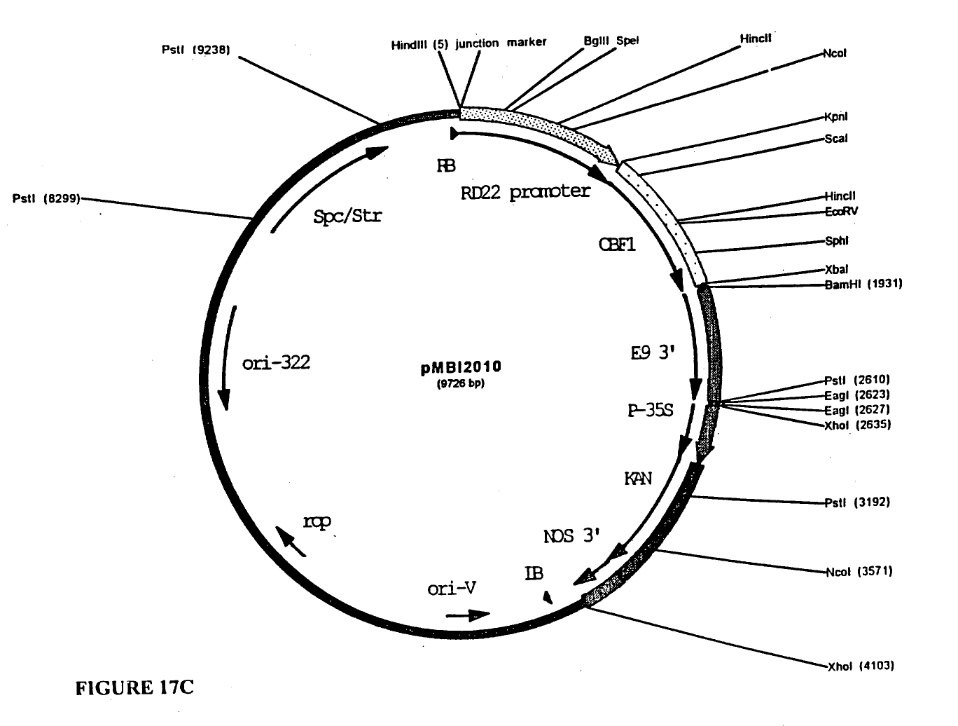

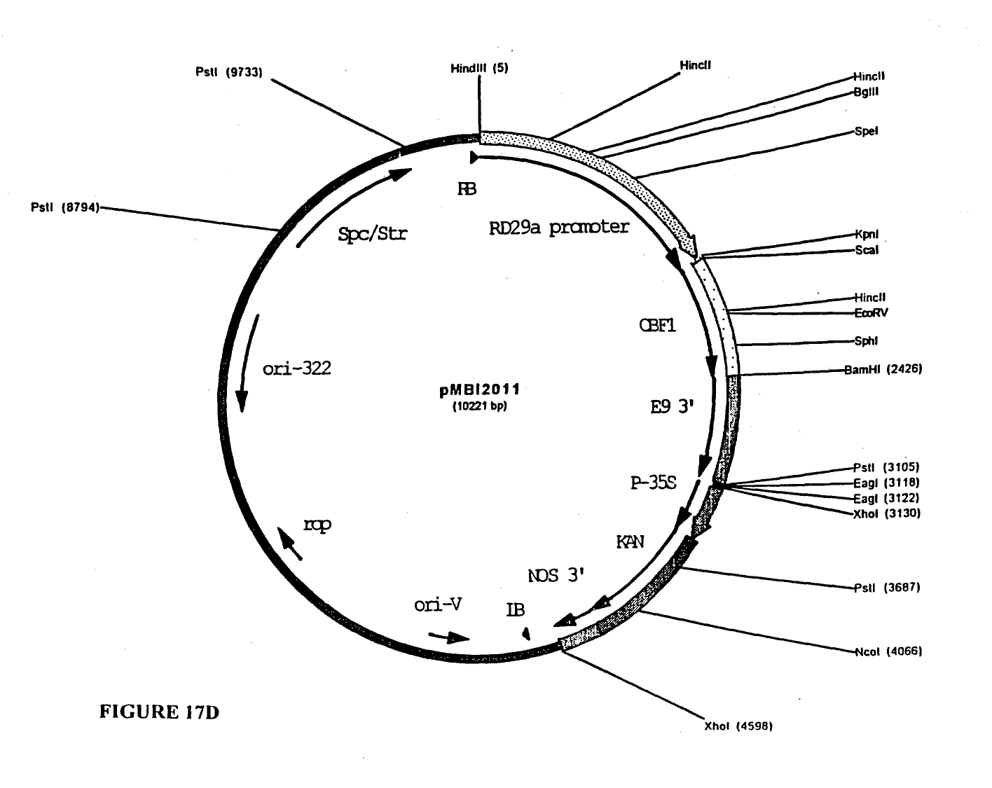

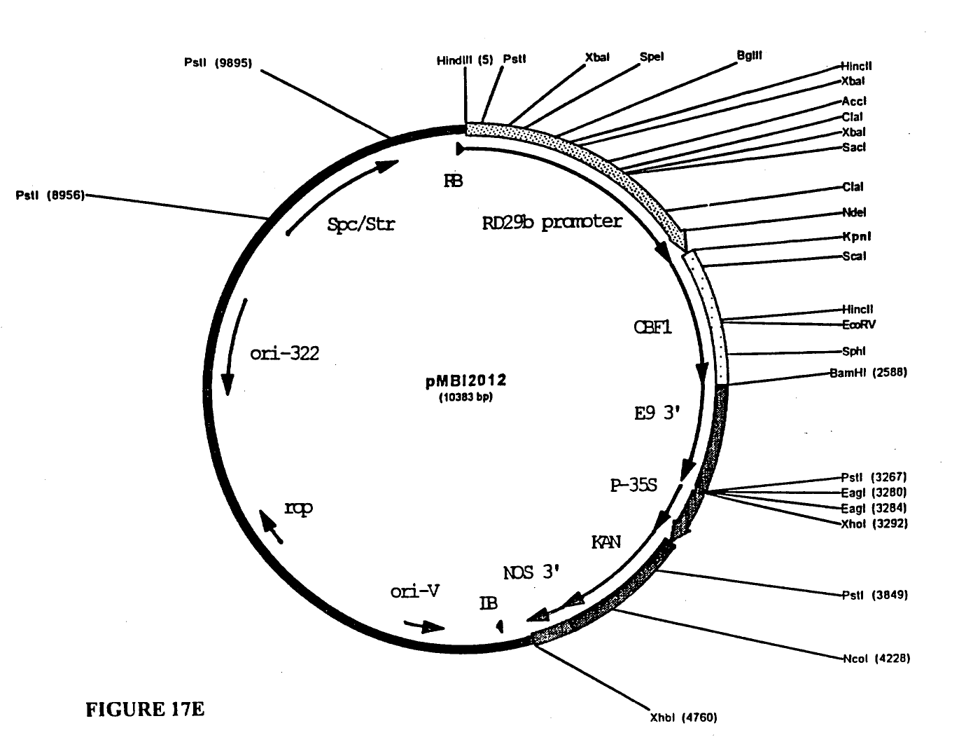

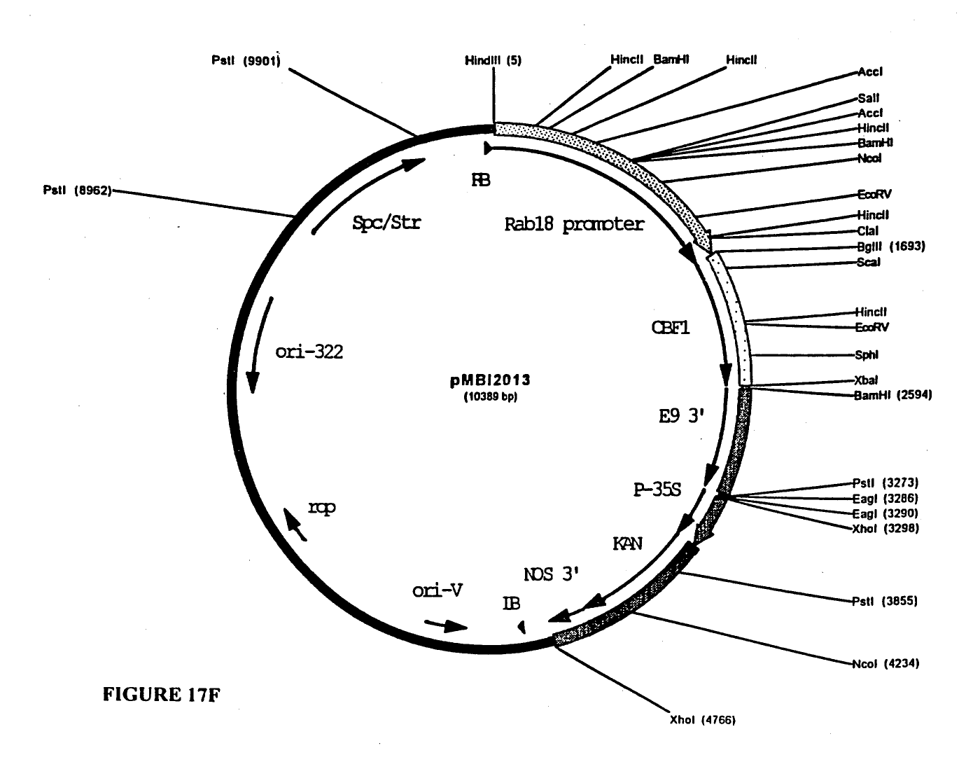

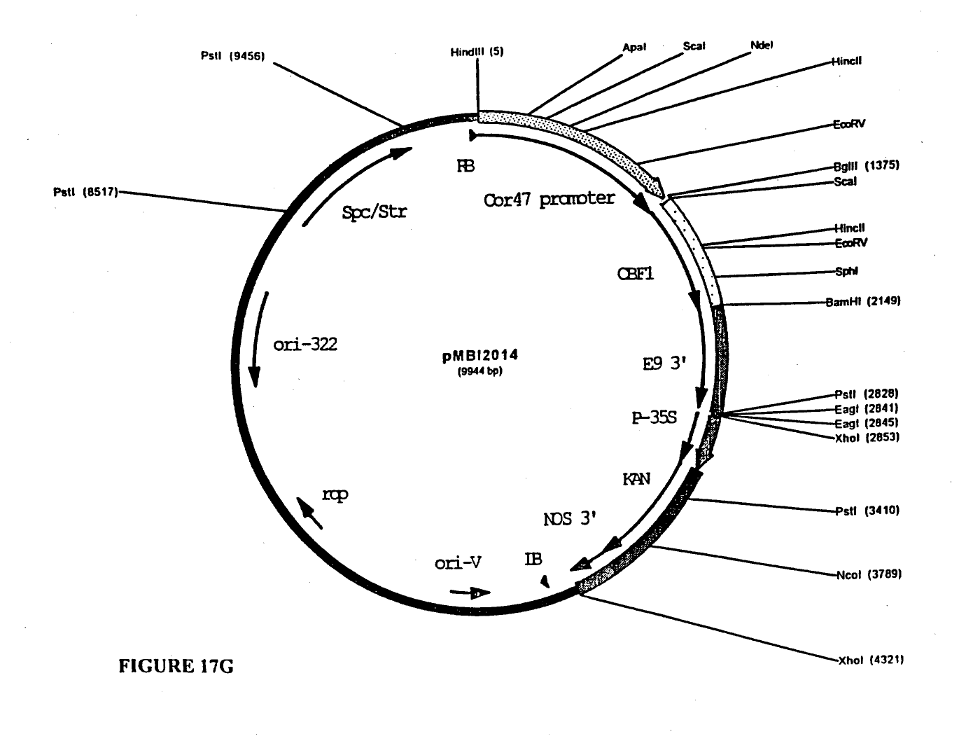

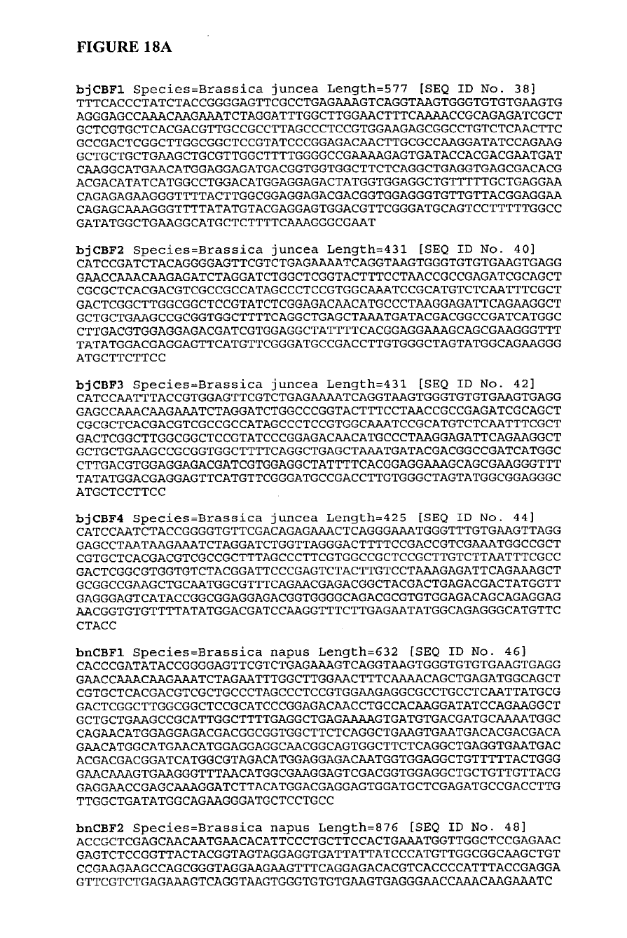

The other primers shown in Table 8 and appropriate restriction enzymes are used in a similar way to clone the CBF1 gene into plasmids containing the other inducible promoters. The resulting plasmids are listed in Table 9 and shown in FIGS. 17A-17G.

A similar cloning strategy may be used to clone other genes, such as cbf2, cbf3, and the other full length CBF genes listed in Table 9 and shown in FIG. 18 (new CBF gene table) into plasmids containing inducible promoters.

TABLE 9

| Construct name | Promoter name | Figure name |
|---|---|---|
| PMBI2008 | Dreb2a | |
| PMBI2009 | P5CS | |
| PMBI2010 | Rd22 | |
| PMBI2011 | Rd29a | |
| PMBI2012 | Rd29b | |
| PMBI2013 | Rab18 | |
| PMBI2014 | Cor47 | FIG. 17G |

### Example 9

Transformation of Agrobacterium with Plasmids Containing CBF1 Gene and Inducible Promoters

After the plasmid vectors containing CBF1 gene and inducible promoters are constructed, these vectors may be used to transform Agrobacterium tumefaciens cells expressing the gene products. The stock of Agrobacterium tumefaciens cells for transformation may be made as described by Nagel et al. (1990) FEMS Microbiol Letts 67: 325-328. Agrobacterium strain ABI may be grown in 250 ml LB medium (Sigma-Aldrich) overnight at 28° C. with shaking until an absorbance (A₆₀₀) of 0.5-1.0 is reached. Cells are harvested by centrifugation at 4,000×g for 15 min at 4 C. Cells are then resuspended in 250 μl chilled buffer (1 mM HEPES, pH adjusted to 7.0 with KOH). Cells are centrifuged again as described above and resuspended in 125 μl chilled buffer. Cells are then centrifuged and resuspended two more times in the same HEPES buffer as described above at a volume of 100 μl and 750 μl, respectively. Resuspended cells are then distributed into 40 μl aliquots, quickly frozen in liquid nitrogen, and stored at −80° C.

Agrobacterium cells are transformed with plasmids formed as described above in Section 4B(2) following the protocol described by Nagel et al. (1990) supra. For each DNA construct to be transformed, 50-100 ng DNA (generally resuspended in 10 mM Tris-HCl, 1 mM EDTA, pH 8.0) is mixed with 40 μl of Agrobacterium cells. The DNA/cell mixture is then transferred to a chilled cuvette with a 2 mm electrode gap and subject to a 2.5 kV charge dissipated at 25 μF and 200 μF using a Gene Pulser II apparatus (Bio-Rad, Hercules Calif.). After electroporation, cells are immediately resuspended in 1.0 ml LB and allowed to recover without antibiotic selection for 2-4 hours at 28° C. in a shaking incubator. After recovery, cells are plated onto selective medium of LB broth containing 100 μg/ml spectinomycin (Sigma-Aldrich) and incubated for 24-48 h at 28° C. Single colonies are then picked and inoculated in fresh medium. The presence of the plasmid construct are verified by PCR amplification and sequence analysis.

### Example 10

Transformation of Arabidopsis Plants with Agrobacterium tumefaciens Carrying Expression Vector for CBF1 Protein

After transformation of Agrobacterium tumefaciens with plasmid vectors containing CBF1 gene and inducible promoters, single Agrobacterium colonies containing each of pMBI2008-pMBI2014 are identified, propagated, and used to transform Arabidopsis plants. Briefly, 500 ml cultures of LB medium containing 100 ug/ml spectinomycin are inoculated with the colonies and grown at 28 C with shaking for 2 days until an absorbance (A₆₀₀) of >2.0 is reached. Cells are then harvested by centrifugation at 4,000×g for 10 min, and resuspended in infiltration medium (½× Murashige and Skoog salts (Sigma-Aldrich), 1× Gamborg's B-5 vitamins (Sigma-Aldrich), 5.0% (w/v) sucrose (Sigma-Aldrich), 0.044 μM benzylamino purine (Sigma-Aldrich), 200 μl/L Silwet L-77 (Lehle Seeds) until an absorbance (A₆₀₀) of 0.8 is reached.

Prior to transformation, Arabidopsis thaliana seeds (ecotype Columbia) are sown at a density of ˜10 plants per 4″ pot onto Pro-Mix BX potting medium (Hummert International) covered with fiberglass mesh (18 mm×16 mm). Plants are grown under continuous illumination (50-75 μE/m²/sec) at 22-23 C with 65-70% relative humidity. After about 4 weeks, primary inflorescence stems (bolts) are cut off to encourage growth of multiple secondary bolts. After flowering of the mature secondary bolts, plants are prepared for transformation by removal of all siliques and opened flowers.

The pots are then immersed upside down in the mixture of Agrobacterium/infiltration medium as described above for 30 sec, and placed on their sides to allow draining into a 1′×2′ flat surface covered with plastic wrap. After 24 h, the plastic wrap is removed and pots are turned upright. The immersion procedure is repeated one week later, for a total of two immersions per pot. Seeds are then collected from each transformation pot and analyzed following the protocol described below.

### Example 11

Identification of Arabidopsis Primary Transformants

Seeds collected from the transformation pots are sterilized essentially as follows. Seeds are dispersed into in a solution containing 0.1% (v/v) Triton X-100 (Sigma-Aldrich) and sterile H₂O and washed by shaking the suspension for 20 min. The wash solution is then drained and replaced with fresh wash solution to wash the seeds for 20 min with shaking. After removal of the second wash solution, a solution containing 0.1% (v/v) Triton X-100 and 70% EtOH (Equistar) is added to the seeds and the suspension is shaken for 5 min. After removal of the ethanol/detergent solution, a solution containing 0.1% (v/v) Triton X-100 and 30% (v/v) bleach (Chlorox) is added to the seeds, and the suspension is shaken for 10 min. After removal of the bleach/detergent solution, seeds are then washed five times in sterile distilled H₂O. The seeds are stored in the last wash water at 4° C. for 2 days in the dark before being plated onto antibiotic selection medium (1× Murashige and Skoog salts (pH adjusted to 5.7 with 1M KOH), 1× Gamborg's B-5 vitamins, 0.9% phytagar (Life Technologies, Rockville, Md.), and 50 μg/L kanamycin). Seeds are germinated under continuous illumination (50-75 μE/m²/sec) at 22-23° C. After 7-10 days of growth under these conditions, kanamycin resistant primary transformants (T₁ generation) are visible and are obtained for each of constructs pMBI2008-pMBI2014. These seedlings are transferred first to fresh selection plates where the seedlings continued to grow for 3-5 more days, and then to soil (Pro-Mix BX potting medium). Progeny seeds (T₂) are collected; kanamycin resistant seedlings selected and analyzed as described above.

### Example 12

Transformation of Cereal Plants with Plasmid Vectors Containing CBF1 Gene and Inducible Promoters.

Cereal plants, such as corn, wheat, rice, sorghum and barley, can also be transformed with the plasmid vectors containing the CBF genes and inducible promoters to modify their biomass or increase their tolerance to environmental stresses. In these cases, the cloning vector, pMEN020, is modified to replace the NptII coding region with the BAR gene of Streptomyces hygroscopicus that confers resistance to phosphinothricin. The KpnI and BglII sites of the Bar gene are removed by site-directed mutagenesis with silent codon changes. After cloning of the inducible promoters into the modified plasmid by the same procedures described above, the at-cbf coding region of cbf1 gene is inserted into the plasmid following the same procedures as described above. The resulting plasmids are listed in Table 10.

TABLE 10

| Promoter name | Construct name |
|---|---|
| Dreb2a | PMBI2015 |
| P5CS | PMBI2016 |
| Rd22 | PMBI2017 |
| Rd29a | PMBI2018 |
| Rd29b | PMBI2019 |
| Rab18 | PMBI2020 |
| Cor47 | PMBI2021 |

It is now routine to produce transgenic plants of most cereal crops (Vasil (1994) Plant Molec. Biol. 25: 925-937) such as corn, wheat, rice, sorghum (Cassas et al. (1993) Proc. Natl. Acad Sci 90: 11212-11216), and barley (Wan et al. (1994). Plant Physiol. 104: 37-48. Other direct DNA transfer methods such as the microprojectile gun or Agrobacterium tumefaciens-mediated transformation can be used for corn (Fromm et al. (1990) Bio/Technology 8: 833-839; Gordon-Kamm et al. (1990) Plant Cell 2: 603-618; Ishida (1990) Nature Biotechnology 14: 745-750), wheat (Vasil et al. (1992) Bio/Technology 10: 667-674; Vasil et al. (1993) Bio/Technology 11: 1553-1558; Weeks et al. (1993) Plant Physiol. 102: 1077-1084), rice (Christou (1991)Bio/Technology 9: 957-962; Hiei et al. (1994) Plant J. 6: 271-282; Aldemita et al. (1996) Planta 199: 612-617; and Hiei et al. (1997) Plant Mol. Biol. 35: 205-218). For most cereal plants, embryogenic cells derived from immature scutellum tissues are the preferred cellular targets for transformation (Hiei et al. (1997) supra; Vasil (1994) Plant Molec. Biol. 25: 925-937).

Plasmids according to the present invention may be transformed into corn embryogenic cells derived from immature scutellar tissue by using microprojectile bombardment, with the A188XB73 genotype as the preferred genotype (Fromm et al. (1990) Bio/Technology 8: 833-839; Gordon-Kamm et al. (1990) Plant Cell 2: 603-618). After microprojectile bombardment the tissues are selected on phosphinothricin to identify the transgenic embryogenic cells (Gordon-Kamm et al. (1990) supra). Transgenic plants are regenerated by standard corn regeneration techniques (Fromm et al. (1990) supra; Gordon-Kamm et al. (1990) supra).

The plasmids prepared as described above can also be used to produce transgenic wheat and rice plants (Christou (1991) supra; Hiei et al. (1994) supra; Aldemita (1996) supra; Hiei et al. (1997) supra) by following standard transformation protocols known to those skilled in the art for rice and wheat Vasil, et al. (1992) supra; Vasil et al. (1993) supra; Weeks et al. (1993) supra), where the BAR gene is used as the selectable marker.

### Example 13

Transformation of Plants with Plasmid Vectors Containing CBF1 Gene and Seed Specific Promoters.

The napin promoter from Brassica campestris (GenBank accession no. M64632) is a seed specific promoter. A fragment of the napin promoter (between nucleotides 1146 to 2148) is identified and isolated by PCR amplification using a 5′ PCR primer containing a HindIII site upstream of the promoter and a 3′ PCR primer containing a BamHI site downstream of the promoter. Deletion of the napin promoter to −211 and −152 have been shown to have reduced levels of expression (Ellerstrom et al. (1996) Plant Mol. Biol. 32: 1019-1027; Stålberg et al. (1996) Planta 199: 515-519; Stålberg et al. (1993) Plant Mol. Biol. 23: 671-683). These 5′ deleted promoters are useful to have reduced levels of CBF1 expression for applications where the larger napin promoter fragment is too large.

Other seed-active promoters or deletions of these promoters can also be isolated from genomic DNA by using the same method described above for the napin promoter. Examples of these promoters include but are not limited to the soybean 7S seed storage protein (Chen et al. (1989) Devel. Gen. 10: 112-122, the bean phaseolin promoter (cited in U.S. Pat. No. 5,003,045), the Arabidopsis 12S globulin (cruciferin) promoter (Pang et al. (1988) Plant Mol. Biol. 11: 805-820, the maize globulin1 promoter (Kriz et al. (1989) Plant Physiol. 91: 636; U.S. Pat. No. 5,773,691). These promoters maybe used for altering COR gene expression in cereals such as corn, barley, wheat, rice and rye seeds.

The binary constructs containing seed-specific napin promoters (pMEN1001.1-4; pMEN1002.1-4; and pMEN1003.1-4) are used to transform canola and rapeseed plants as described (Moloney et al. U.S. Pat. No. 5,750,871), except that the Bar gene selectable marker is used.

These constructs are also used to transform regenerable barley cells by microprojectile bombardment (Wan et al. (1994) Plant Physiol. 104: 37-48). After bombardment the tissues are selected on phosphinothricin by standard barley regeneration techniques (Wan and Lemaux, supra).

### Example 14

Identification of Homologous Sequence to CBF1 in Canola

This example describes the identification of homologous sequences to CBF1 in canola using PCR. Degenerate primers were designed for regions of AP2 binding domain and outside of the AP2 (carboxyl terminal domain). More specifically, the following degenerate PCR primers were used:

Mol 368 (reverse) 5′-CAY CCN ATH TAY MGN GGN GT-3′

Mol 378 (forward) 5′-GGN ARN ARC ATN CCY TCN GCC-3′

Y: C/T, N: A/C/G/T, H: A/C/T, M: A/C, R: A/G)

Primer Mol 368 is in the AP2 binding domain of CBF1 (amino acid sequence: H P I Y R G V) while primer Mol 378 is outside the AP2 domain (carboxyl terminal domain)(amino acid sequence: M A E G M L L P).

The genomic DNA isolated from Brassica Napus was PCR amplified by using these primers following these conditions: an initial denaturation step of 2 min at 93° C.; 35 cycles of 93° C. for 1 min, 55° C. for 1 min, and 72° C. for 1 min; and a final incubation of 7 min at 72° C. at the end of cycling.

The PCR products were separated by electrophoresis on a 1.2% agarose gel and, transferred to nylon membrane and hybridized with the AT CBF1 probe prepared from Arabidopsis genomic DNA by PCR amplification. The hybridized products were visualized by colorimetric detection system (Boehringer Mannheim) and the corresponding bands from a similar agarose gel were isolated (By Qiagen Extraction Kit). The DNA fragments were ligated into the TA clone vector from TOPO TA Cloning Kit (Invitrogen) and transformed into E. coli strain TOP10 (Invitrogen).

Seven colonies were picked and the inserts were sequenced on an ABI 377 machine from both strands of sense and antisense after plasmid DNA isolation. The DNA sequence was edited by sequencer and aligned with the AtCBF1 by GCG software and NCBI blast searching.

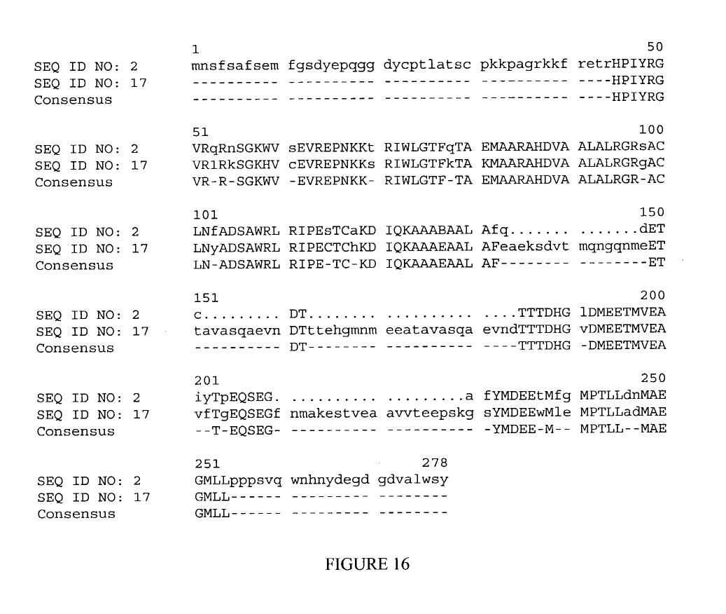

FIG. 16 shows an amino acid sequence of a homolog (CAN1; SEQ ID NO: 17) identified by this process and its alignment to the amino acid sequence of CBF1. The nucleic acid sequence for CAN1 is listed herein as SEQ ID NO: 18.

As illustrated in FIG. 16, the amino acid sequence alignment in four regions of BN-CBF1 shows 82% identity in the AP2 binding domain region and range from 75% to 83% with some alignment gaps due to regions of lesser homology or introns in the genomic sequence. The aligned amino acid sequences show that the polypeptide encoded by the BNCBF1 gene has 88% identity in the AP2 domain region and 85% identity outside the AP2 domain when aligned for two insertion sequences that are outside the AP2 domain. The extra amino acids in the two insertion regions are either due to the presence of introns in this region of the BNCBF1 gene, as it was derived from genomic DNA, or could be due to extra amino acids in these regions of the BNCBF1 gene. Isolation and sequencing of a cDNA of the BNCBF1 gene using the genomic DNA as a probe will resolve this.

### Example 15

Identification of Homologous Sequence to CBF1 in Canola and Other Species

A PCR strategy similar to that described in Example 14 was used to isolate additional CBF homologs from Brassica juncea, Brassica napus, Brassica oleracea, Brassica rapa, Glycine max, Raphanus sativus and Zea mays. The nucleotide (for example, bjCBF1) and peptide sequences (for example, BJCBF1-PEP) of these isolated CBF homologs are shown in FIGS. 18A and 18B, respectively. Table 11 lists the sequence names and SEQ ID NOs of these isolated CBF homologs. The PCR primers are internal to the gene so partial gene sequences are initially obtained. The full length sequences of some of these genes were further isolated by inverse PCR or ligated linker PCR. One skilled in the art can use the conserved regions in these genes to design PCR primers to isolate additional CBF genes.

TABLE 11

| | SEQ | | | % |
|---|---|---|---|---|
| DNA SEQ Name | ID NO: | Peptide SEQ Name | SEQ ID NO: | ID* |
| bjCBF1 | 38 | BJCBF1-PEP | 39 | 87 |
| bjCBF2 | 40 | BJCBF2-PEP | 41 | 85 |
| bjCBF3 | 42 | BJCBF3-PEP | 43 | 85 |
| bjCBF4 | 44 | BJCBF4-PEP | 45 | 93 |
| bnCBF1 | 46 | BNCBF1-PEP | 47 | 88 |
| bnCBF2 | 48 | BNCBF2-PEP | 49 | 87 |
| bnCBF3 | 50 | BNCBF3-PEP | 51 | 87 |
| bnCBF4 | 52 | BNCBF4-PEP | 53 | 88 |
| bnCBF5 | 54 | BNCBF5-PEP | 55 | 88 |
| bnCBF6 | 56 | BNCBF6-PEP | 57 | 88 |
| bnCBF7 | 58 | BNCBF7-PEP | 59 | 87 |
| bnCBF8 | 60 | BNCBF8-PEP | 61 | 88 |
| bnCBF9 | 62 | BNCBF9-PEP | 63 | 85 |
| boCBF1 | 64 | BOCBF1-PEP | 65 | 88 |
| boCBF2 | 66 | BOCBF2-PEP | 67 | 87 |
| boCBF3 | 68 | BOCBF3-PEP | 69 | 88 |
| boCBF4 | 70 | BOCBF4-PEP | 71 | 88 |
| boCBF5 | 72 | BOCBF5-PEP | 73 | 87 |
| brCBF1 | 74 | BRCBF1-PEP | 75 | 88 |
| brCBF2 | 76 | BRCBF2-PEP | 77 | 88 |
| brCBF3 | 78 | BRCBF3-PEP | 79 | 88 |
| brCBF4 | 80 BRCBF4-PEP | 81 | 88 | |
| brCBF5 | 82 | BRCBF5-PEP | 83 | 88 |
| brCBF6 | 84 | BRCBF6-PEP | 85 | 88 |
| brCBF7 | 86 | BRCBF7-PEP | 87 | 88 |
| gmCBF1 | 88 | GMCBF1-PEP | 89 | 87 |
| rsCBF1 | 90 | RSCBF1-PEP | 91 | 88 |
| rsCBF2 | 92 | RSCBF2-PEP | 93 | 88 |
| zmCBF1 | 94 | ZMCBF1-PEP | 95 | 80 |

*Percentage identity to the CBF1 (SEQ ID NO:2) AP2 region of derived polypeptide, wherein the AP2 region is defined by the AP2 consensus sequence and bounded by: HPXYXGVR---ADS

FIG. 19A shows an amino acid alignment of the AP2 domains of the CBF proteins listed in Table 11 with their consensus sequences highlighted. FIG. 19A also provides a comparison of the consensus sequence with that of the tobacco DNA binding protein EREBP2 (SEQ ID NO: 168; Okme-Takagi et al. (1995) supra). The AP2 domain sequences of these CBF proteins are: atcbf2 (SEQ ID NO:137); atcbf3 (SEQ ID NO: 138); atcbf1 (SEQ ID NO: 139); bjcbf4 (SEQ ID NO: 140); bocbf2 (SEQ ID NO: 141); rscbf2 (SEQ ID NO: 142); bjcbf2 (SEQ ID NO: 143); bncbf7 (SEQ ID NO: 144); bjcbf3 (SEQ ID NO: 145); bncbf2 (SEQ ID NO: 146); bncbf3 (SEQ ID NO: 147); bncbf1 (SEQ ID NO: 148); bncbf6 (SEQ ID NO: 149); bncbf8 (SEQ ID NO: 150); bocbf3 (SEQ ID NO: 151); brcbf2 (SEQ ID NO: 152); bncbf4 (SEQ ID NO: 153); bncbf5 (SEQ ID NO: 154); bocbf4 (SEQ ID NO: 155); brcbf1 (SEQ ID NO: 156); brcbf3 (SEQ ID NO: 157); brcbf4 (SEQ ID NO: 158); brcbf5 (SEQ ID NO: 159); brcbf6 (SEQ ID NO: 160); brcbf7 (SEQ ID NO: 161); rscbf1 (SEQ ID NO: 162); bocbf1 (SEQ ID NO: 163); bocbf5 (SEQ ID NO: 164); bncbf9 (SEQ ID NO: 165); zmcbf1 (SEQ ID NO: 166); and gmcbfl (SEQ ID NO: 167).

As can be seen from the consensus sequence shown in FIG. 19A, a significant portion of the AP2 domain is conserved among the different CBF proteins. In view of this data, Applicants use the conserved sequence in the AP2 domain to define a class of AP2 domain proteins comprising this conserved sequence.

FIG. 19B shows an amino acid alignment of the AP2 domains shown in FIG. 19A and the AP2 doamins of dreb2a (SEQ ID NO:169) and dreb2b (SEQ ID NO: 170) and a consensus sequence between the proteins highlighted. As can be seen, a very high degree of homology exists between AP2 domains shown in FIG. 19A and dreb2a and dreb2b. Applicants employ the conserved sequence in the AP2 domain shown in FIG. 19B to define a broader class of AP2 domain proteins that are capable of binding to CCG regulatory region.

FIG. 19C shows an amino acid alignment of the AP2 domains shown in FIG. 19B and the AP2 domain of tiny (SEQ ID NO: 171) and a consensus sequence between the proteins highlighted. As can be seen, a very high degree of homology exists between AP2 domains shown in FIG. 19A, dreb2a (SEQ ID NO: 169), dreb2b (SEQ ID NO:170), and tiny (SEQ ID NO:171). Applicants employ the conserved sequence in the AP2 domain shown in FIG. 19C to define a yet broader class of AP2 domain proteins that are capable of binding to CCG regulatory region.

FIG. 19D shows a consensus sequence corresponding to the difference between the consensus sequence shown in FIG. 19A and tiny. Applicants employ the highlighted portion of the conserved sequence shown in FIG. 19D to define a group of amino acid residues that may be critical to binding to a CCG regulatory region.

FIG. 19E shows a consensus sequence corresponding to the difference between the consensus sequence shown in FIG. 19B and tiny. Applicants employ the highlighted portion of the conserved sequence shown in FIG. 19E to define another group of amino acid residues that may be critical to binding to a CCG regulatory region.

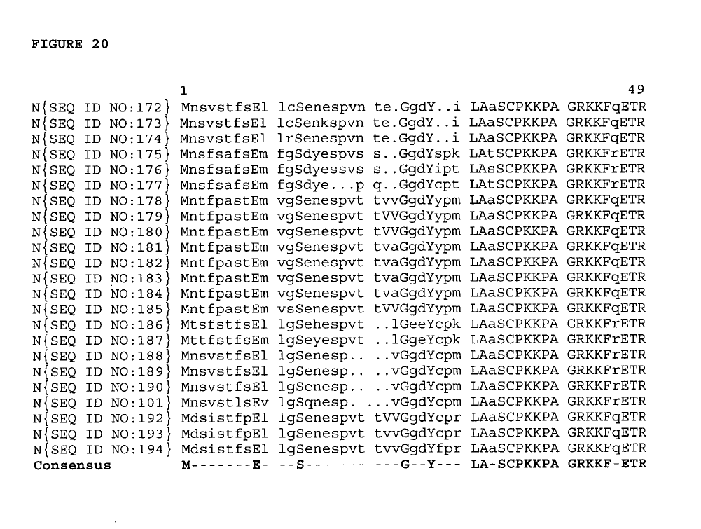

FIG. 20 shows the amino acid alignment of the amino terminus of the CBF proteins with their consensus sequence highlighted. The sequences of these CBF proteins are: brcbf3 (SEQ ID NO: 172); brcbf6 (SEQ ID NO: 173); bncbf5 (SEQ ID NO: 174); atcbf2 (SEQ ID NO: 175); atcbf3 (SEQ ID NO: 176); atcbf1 (SEQ ID NO: 177); bncbf2 (SEQ ID NO: 178); bncbf6 (SEQ ID NO: 179); bocbf3 (SEQ ID NO: 180); bncbf3 (SEQ ID NO: 181); bncbf8 (SEQ ID NO: 182); bncbf9 (SEQ ID NO: 183); brcbf2 (SEQ ID NO: 184); bocbf5 (SEQ ID NO: 185); bocbf2 (SEQ ID NO: 186); rscbf2 (SEQ ID NO: 187); bncbf4 (SEQ ID NO: 188); bncbf7 (SEQ ID NO: 189); bocbf4 (SEQ ID NO: 190); brcbf7 (SEQ ID NO: 191); brcbf4 (SEQ ID NO: 192); brcbf5 (SEQ ID NO: 193); and rscbf1 (SEQ ID NO: 194).

As can be seen from the consensus sequence shown in FIG. 20, a significant portion of the amino terminus of CBF proteins is conserved among the different CBF proteins. In view of this data, Applicants employ the conserved sequence in the amino terminus domain to define a class of proteins comprising this conserved sequence. Of note, the conserved sequence corresponding to amino acids 31-37 of SEQ ID NO: 2, PKKPAGR; SEQ ID NO: 326), amino acids 35-40 of SEQ ID NO: 2 (AGRKKF; SEQ ID NO: 325), or amino acids 42-46 of SEQ ID NO: 2 (ETRHP; SEQ ID NO: 321), can define the class of CBF proteins. In addition, the consensus sequence corresponding to amino acids 31-37 of SEQ ID NO: 2 PKXXAGR; SEQ ID NO: 319), amino acids 35-40 of SEQ ID NO: 2 (AGRXKF; SEQ ID NO: 320), or amino acids 42-46 of SEQ ID NO: 2 (ETRHP; SEQ ID NO: 321), wherein X is any amino acid residue, also can define the class of CBF proteins.

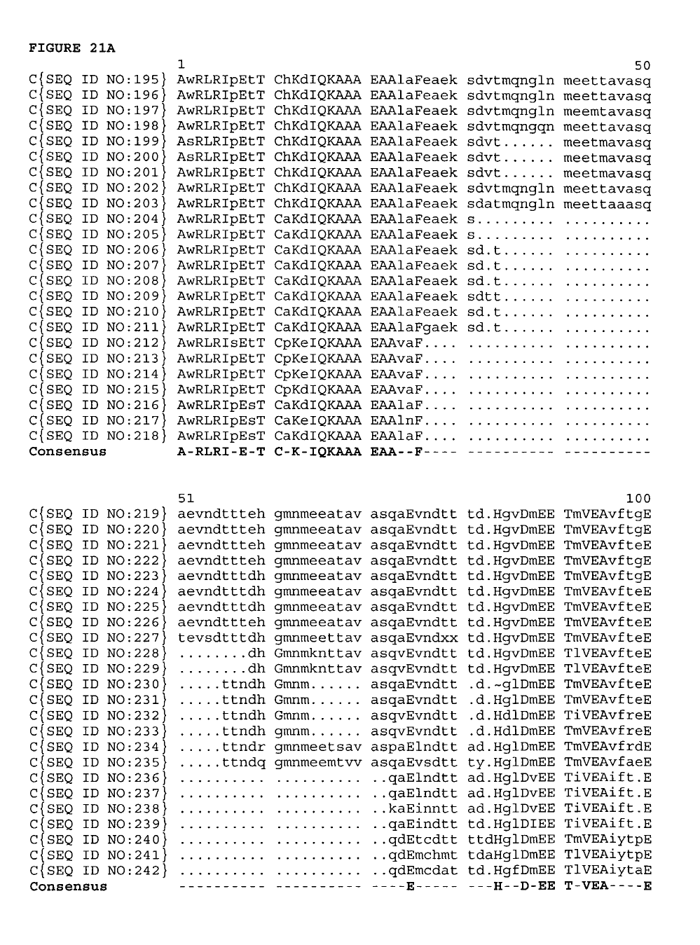

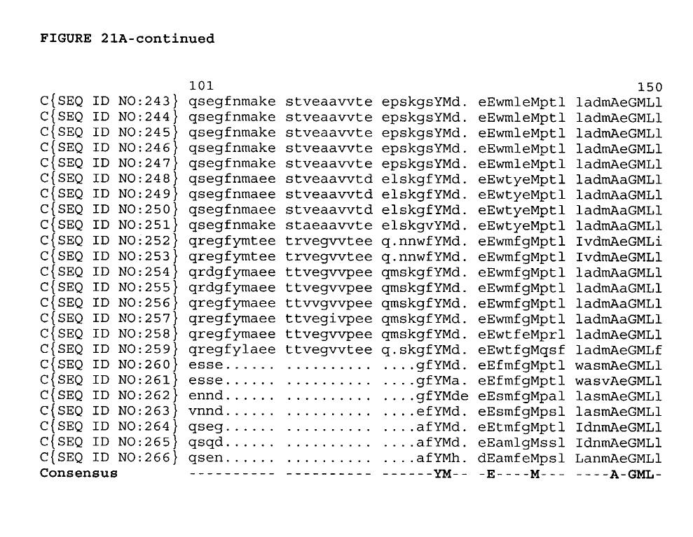

FIG. 21A shows the amino acid alignment of the carboxy terminus of 24 CBF proteins with their consensus sequences highlighted. The sequences of these CBF proteins are: bncbf3 (SEQ ID NO: 195; SEQ ID NO: 219; SEQ ID NO: 243); bncbf9 (SEQ ID NO: 196; SEQ ID NO: 220; SEQ ID NO: 244); brcbf2 (SEQ ID NO: 197; SEQ ID NO: 221; SEQ ID NO: 245); bncbf1 (SEQ ID NO: 198; SEQ ID NO: 222; SEQ ID NO: 246); bncbf8 (SEQ ID NO: 199; SEQ ID NO: 223; SEQ ID NO: 247); bncbf6 (SEQ ID NO: 200; SEQ ID NO: 224; SEQ ID NO: 248); bocbf3 (SEQ ID NO: 201; SEQ ID NO: 225; SEQ ID NO: 249); bncbf2 (SEQ ID NO: 202; SEQ ID NO: 226; SEQ ID NO: 250); bocbf5 (SEQ ID NO: 203; SEQ ID NO: 227; SEQ ID NO: 251); brcbf5 (SEQ ID NO: 204; SEQ ID NO: 228; SEQ ID NO: 252); rscbf1 (SEQ ID NO: 205; SEQ ID NO: 229; SEQ ID NO: 253); bncbf4 (SEQ ID NO: 206; SEQ ID NO: 230; SEQ ID NO: 254); bocbf4 (SEQ ID NO: 207; SEQ ID NO: 231; SEQ ID NO: 255); bncbf5 (SEQ ID NO: 208; SEQ ID NO: 232; SEQ ID NO: 256); brcbf7 (SEQ ID NO: 209; SEQ ID NO: 233; SEQ ID NO: 257); brcbf6 (SEQ ID NO: 210; SEQ ID NO: 234; SEQ ID NO: 258); bocbf1 (SEQ ID NO: 211; SEQ ID NO: 235; SEQ ID NO: 259); bjcbf2 (SEQ ID NO: 212; SEQ ID NO: 236; SEQ ID NO: 260); bjcbf3 (SEQ ID NO: 213; SEQ ID NO: 237; SEQ ID NO: 261); bncbf7 (SEQ ID NO: 214; SEQ ID NO: 238; SEQ ID NO: 262); rscbf2 (SEQ ID NO: 215; SEQ ID NO: 239; SEQ ID NO: 263); atcbf1 (SEQ ID NO: 216; SEQ ID NO: 240; SEQ ID NO: 264); atcbf2 (SEQ ID NO:217; SEQ ID NO: 241; SEQ ID NO: 265); and atcbf3 (SEQ ID NO: 218; SEQ ID NO: 242; SEQ ID NO: 266).

As can be seen from the consensus sequence shown in FIG. 21A, a significant portion of the carboxy terminus of CBF proteins is conserved among the different CBF proteins. In view of this data, Applicants employ the conserved sequence in the carboxy terminus domain to define a class of proteins comprising this conserved sequence.

FIG. 21B shows the amino acid alignment of the carboxy terminus of 9 CBF proteins with their consensus sequences highlighted. The sequences of these CBF proteins are: bncbf3 (SEQ ID NO: 267; SEQ ID NO: 276; SEQ ID NO: 285); bncbf9 (SEQ ID NO: 268; SEQ ID NO: 277; SEQ ID NO: 286); brcbf2 (SEQ ID NO: 269; SEQ ID NO: 278; SEQ ID NO: 287); bncbfl (SEQ ID NO: 270; SEQ ID NO: 279; SEQ ID NO: 288); bncbf8 (SEQ ID NO: 272; SEQ ID NO: 280; SEQ ID NO: 289); bncbf6 (SEQ ID NO: 273; SEQ ID NO: 281; SEQ ID NO: 290); bocbf3 (SEQ ID NO: 274; SEQ ID NO: 282; SEQ ID NO: 291); bncbf2 (SEQ ID NO: 274; SEQ ID NO: 283; SEQ ID NO: 292); bocbf5 (SEQ ID NO: 275; SEQ ID NO: 284; SEQ ID NO: 293).

As can be seen from the consensus sequence shown in FIG. 21B, a greater portion of the carboxy terminus is conserved when these nine CBF proteins are used. In view of this data, Applicants employ the conserved sequence in the carboxy terminus domain to define another class of proteins comprising this conserved sequence.

### Example 16

Homologous CBF Encoding Genes in other Plants.

This example shows that homologous sequences to CBF1 are present in other plants. The presence of these homologous sequences suggest that the same or similar cold regulated environmental stress response regulatory elements such as the C-repeat/DRE of Arabidopsis (CCGAC) exist in other plants. This example serves to indicate that genes with significant homology to CBF1, CBF2 and CBF3 exist in a wide range of plant species.

Total plant DNAs from Arabidopsis thaliana, Nicotiana tabacum, Lycopersicon pimpinellifolium, Prunus avium, Prunus cerasus, Cucumis sativus, and Oryza sativa were isolated according to Stockinger al (Stockinger et al. (1996) J. Heredity 87: 214-218). Approximately 2 to 10 μg of each DNA sample was restriction digested, transferred to nylon membrane (Micron Separations, Westboro Mass.) and hybridized according to Walling et al. (Walling et al. (1988) Nucleic Acids Res. 16: 10477-10492). Hybridization conditions were: 42° C. in 50% formamide, 5×SSC, 20 mM phosphate buffer 1× Denhardt's, 10% dextran sulfate, and 100 μg/ml herring sperm DNA. Four low stringency washes at RT in 2×SSC, 0.05% Na sarcosyl and 0.02% Na₄ pyrophosphate were performed prior to high stringency washes at 55° C. in 0.2×SSC, 0.05% Na sarcosyl and 0.01% Na₄ pyrophosphate. High stringency washes were performed until no counts were detected in the washout. The BclI-BglII fragment of CBF1 (Stockinger et al. (1997) Proc Natl Acad Sci 94: 1035-1040) was gel isolated (Sambrook et al. (1989), supra) and direct prime labeled (Feinberg et al. (1982) Anal. Biochem. 132: 6-13) using the primer MT117

(TTGGCGGCTACGAATCCC; SEQ ID NO: 16).

Specific activity of the radiolabelled fragment was approximately 4×10⁸ cpm/μg. Autoradiography was performed using HYPERFILM-MP (Amersham) at −80° C. with one intensifying screen for 15 hours.

Autoradiography of the gel showed that DNA sequences from Arabidopsis thaliana, Nicotiana tabacum, Lycopersicon pimpinellifolium, Prunus avium, Prunus cerasus, Cucumis sativus, and Oryza sativa hybridized to the labeled BclI, BglII fragment of CBF1. These results suggest that homologous CBF encoding genes are present in a variety of other plants.

### Example 17

Identification of CBF1 Homologs CBF2 and CBF3 Using CBF1

This example describes two homologs of CBF1 from Arabidopsis thaliana and named them CBF2 and CBF3.

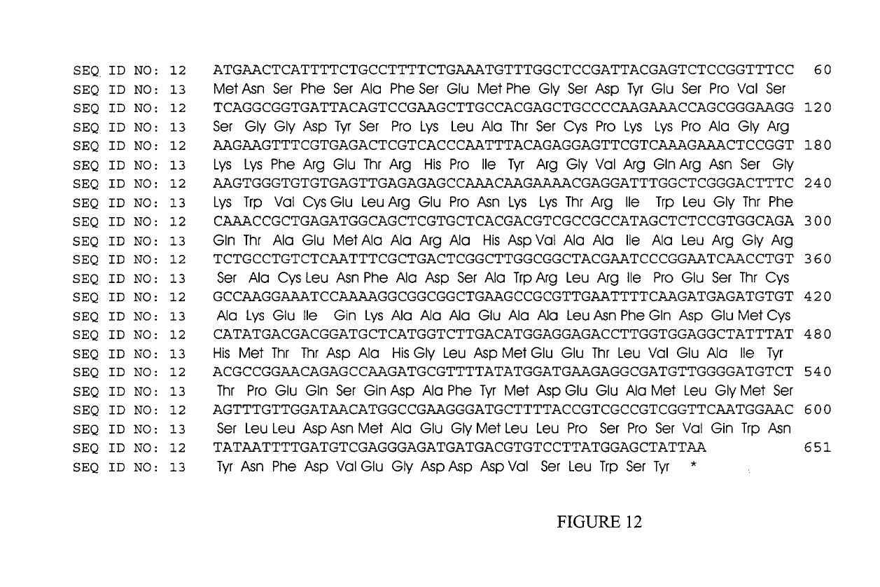

CBF2 and CBF3 have been cloned and sequenced as described below. The sequences of the DNA and encoded proteins are set forth in SEQ ID NOs: 12 and 13, 14 and 15, and FIGS. 12 and 13.

A lambda cDNA library prepared from RNA isolated from Arabidopsis thaliana ecotype Columbia (Lin et al. (1992) Plant Physiol. 99: 519-525) was screened for recombinant clones that carried inserts related to the CBF1 gene (Stockingeret al. (1997) Proc Natl Acad Sci 94: 1035-1040). CBF1 was ³²P-radiolabeled by random priming (Sambrook et al. (1989) supra) and used to screen the library by the plaque-lift technique using standard stringent hybridization and wash conditions (Hajela et al. (1990) Plant Physiol.93: 1246-1252; Sambrook et al. (1989), supra; 6×SSPE buffer, 60° C. for hybridization and 0.1×SSPE buffer and 60° C. for washes). Twelve positively hybridizing clones were obtained and the DNA sequences of the cDNA inserts were determined at the MSU-DOE Plant Research Laboratory sequencing facility. The results indicated that the clones fell into three classes. One class carried inserts corresponding to CBF1. The two other classes carried sequences corresponding to two different homologs of CBF1, designated CBF2 and CBF3. The nucleic acid sequences and predicted protein coding sequences for CBF1 (SEQ ID NO:1 and SEQ ID NO:2, respectively), CBF2 (SEQ ID NO:12 and SEQ ID NO:13, respectively), and CBF3 (SEQ ID NO:14 and SEQ ID NO:15, respectively) appear in the Sequence Listing.

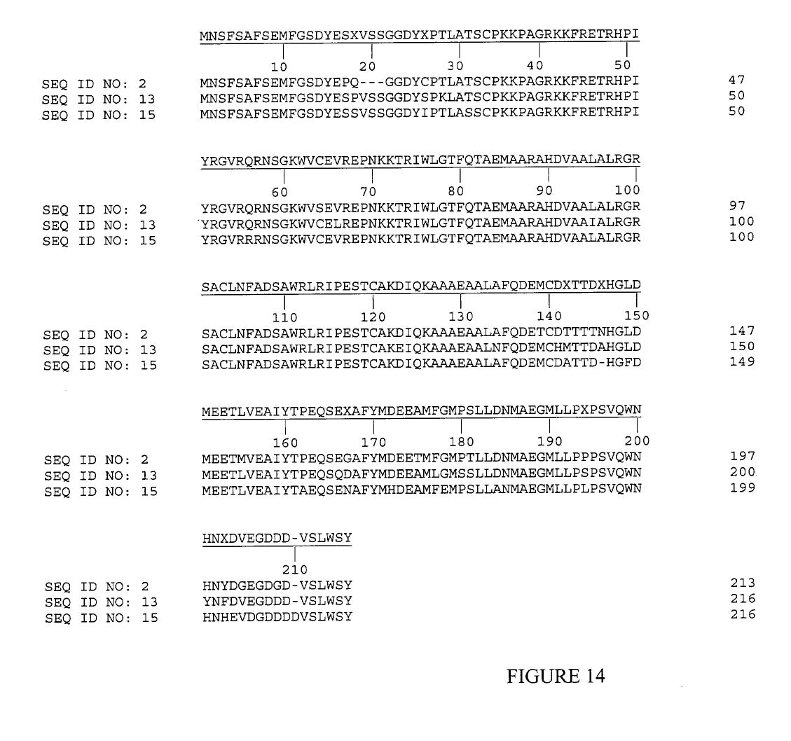

A comparison of the nucleic acid sequences of CBF1, CBF2 and CBF3 indicate that they are 83 to 85% identical as shown in Table 12. FIG. 14 shows the amino acid alignment of proteins CBF1, (SEQ ID NO:2), CBF2 (SEQ ID NO:13), and CBF3 (SEQ ID NO:15).

TABLE 12

Percent identity^{a}

| | DNA^{b} | Polypeptide |
|---|---|---|
| cbf1/cbf2 | 85 | 86 |
| cbf1/cbf3 | 83 | 84 |
| cbf2/cbf3 | 84 | 85 |

^{a}Percent identity was determined using the Clustal algorithm from the Megalign program (DNASTAR, Inc.).
^{b}Comparisons of the nucleic acid sequences of the open reading frames are shown.

Similarly, the amino acid sequences of the three CBF polypeptides range from 84 to 86% identity. An alignment of the three amino acidic sequences reveals that most of the differences in amino acid sequence occur in the acidic C-terminal half of the polypeptide. This region of CBF1 serves as an activation domain in both yeast and Arabidopsis (not shown).

Residues 47 to 106 of CBF1 correspond to the AP2 domain of the protein, a DNA binding motif that, to date, has only been found in plant proteins. A comparison of the AP2 domains of CBF1, CBF2, and CBF3 indicates that there are a few differences in amino acid sequence. These differences in amino acid sequence might have an effect on DNA binding specificity.

### Example 18

Activation of Transcription in Yeast Containing C-Repeat/DRE Using CBF1, CBF2, and CBF3

This example shows that CBF1, CBF2, and CBF3 activate transcription in yeast containing CRT/DREs upstream of a reporter gene. The CBFs were expressed in yeast under control of the ADC1 promoter on a 2μ plasmid (pDB20.1; Berger et al. (1992) Cell 70: 251-265). Constructs expressing the different CBFs were transformed into yeast reporter strains that had the indicated CRT/DRE upstream of the lacZ reporter gene. Copy number of the CRT/DREs and its orientation relative to the direction of transcription from each promoter is indicated by the direction of the arrow.

FIG. 15 is a graph showing transcription regulation of CRT/DRE containing reporter genes by CBF1, CBF2, and CBF3 genes in yeast. In FIG. 15, the vertical lines across the arrows of the COR15a construct represent the m3cor15a mutant CRT/DRE construct. Each CRT/DRE-lacZ construct was integrated into the URA3 locus of yeast. Error bars represent the standard deviation derived from three replicate transformation events with the same CBF activator construct into the respective reporter strain. Quantitative B-gal assays were performed as described by Rose and Botstein (Rose et al. (1983) Methods Enzymol. 101: 167-180).

### Example 19

Identification and Isolation of Novel CBF-Related Polypeptides

Additionally, we identified novel CBF-related polypeptides from soybean, wheat, rice, and rye plants.

Soybean seeds were bought from a local supermarket (packaged by JAMECO Co, San Francisco, Calif.). DNA and mRNA were isolated using standard procedures (Ausubel et al. (1998) Current Protocols in Molecular Biology (Greene & Wiley, New York)). A soybean seedling cDNA library was also constructed using standard procedures. Based on the sequence of the Arabidopsis CBF1 gene (SEQ ID NO: 1), degenerate primers

O368 (CAYCCNATHTAYMGNGGNGT (SEQ ID NO: 104));

O376 (GCNGCYTCNGCNGCNGCYTTYTGDAT (SEQ ID NO: 105)); and

O2953 (AARAARTTYMGNGARACNMGNCAY (SEQ ID NO: 106))

were designed. O376 and O2953 were first used in a PCR experiment using soybean genomic DNA as template. The product from this reaction was excised from the gel, purified (Ausubel et al. (1998) supra), and used as a template in a second round of PCR using primers O368 and O376. Then, the PCR product was cloned into pGEM-T and sequenced using T7 and sp6 primers (Promega Corp).

Based on the sequence of the cloned soybean clone, 3′ rapid amplification of cDNA ends (RACE) was performed using the MARATHON cDNA amplification kit (Clontech, Palo Alto, Calif.). Generally, the method entailed first isolating poly(A) mRNA, synthesize first and second strand cDNA to generate double stranded cDNA, blunting cDNA ends, followed by ligation of the Marathon™ Adaptor to the cDNA to form a library of adaptor-ligated double-stranded cDNA. Gene-specific primers were designed to be used along with adaptor specific primers for 3′ RACE reactions. Often, nested primers were used to increase PCR specificity. In this case, the 3′ nested primers were

O5436 (GGAGGAACACGGATAAGTGGGTAAG (SEQ ID NO: 107)) and

O5437 (AGGATTTGGCTGGGGACTTTTCC (SEQ ID NO: 108)).

The resulting RACE fragment was cloned into the pGEM-T vector (Promega Corp) and sequenced using T7 and sp6 primers. The cloned insert was then labeled using the DIG DNA Labeling and Detection Kit following the manufacture's instructions (Boehringer Mannheim), and the labeled probe was used to screen the soybean cDNA library using standard procedures and hybridization conditions (Ausubel et al. (1998) supra). SEQ ID NO: 127 was isolated in this manner.

Rice seeds were obtained from the laboratory of Dr. Pam Ronald at UC Davis. Corn, wheat, and rye seeds were obtained from the USDA, ARS National Small Grains Research Facility, Aberdeen, Id. DNA and mRNA were isolated using standard procedures. Seedling cDNA libraries were also constructed using standard procedures.

In order to isolate CBF1 homologs from monocotyledon species, CBF1 polypeptide sequence was used to identify related sequences from public plant sequence databases. The tblastn sequence analysis program was employed. A rice homolog (Ace. No. AB023482) was identified as having a P value of 6.3e-17. Based on its sequence, primers

O18016 (ACGCGTCGACCCATCATCACCGAGATCGACTCGAC (SEQ ID NO: 109)) and

O18017 (ATAAGAATGCGGCCGCTCATTGTTCGCTCACTGGGAG (SEQ ID NO: 110)

were synthesized, and the rice gene was isolated from rice genomic DNA by a standard PCR procedure using those primers. The amplified fragment was cloned into the pGEM-T vector following the manufacture's protocol (Promega Corp). The clone was sequenced using O18016, O18017,

O18035 (GCTGACAGAACGGGTGCCGA (SEQ ID NO: 111)) and

O18036 (TGACCGTTTCTGGATAGGCA (SEQ ID NO: 112)).

Based on the rice sequence, primers

O18065 (GGCCGGCGGGGCGAACCAAGTTCC (SEQ ID NO: 113)) and

O18066 (AGGCAGAGTCGGCGAAGTTGAGGC (SEQ ID NO: 114))

were synthesized. These primers were used to isolate rye and wheat CBF gene fragments by PCR from their respective cDNA libraries. For some of the PCR reactions outlined above, a PCR optimization kit (Boehringer Mannheim) was used. The PCR product was cloned into the pGEM-T vector (Promega Corp). To isolate full-length rye cDNAs, the rye fragment was then labeled with ³²P dCTP using the High Prime DNA Labeling Kit (Boehringer Mannheim). Purified radiolabeled probes were used to probe a rye cDNA library using standard conditions (see Ausubel et al. Current Protocols in Molecular Biology, supra section 6.3). SEQ ID NO: 115, 117, 119, 121 and 123, which are rye CBF-related sequences, were isolated in this manner. SEQ ID NO: 125 is a CBF-related peptide sequence identified from wheat.

The percent sequence identity of the AP2 regions of polypeptide SEQ ID NO: 116, 118, 120, 122, 124 and 126, with CBF1 (SEQ ID NO: 2), are shown in Table 13. The percent sequence identity between the different polypeptides provided in the Sequence Listing varied from 53% to 96% over most of the length of the sequences. Generally, these sequences comprise an AP2 domain comprising amino acids 45, 46, 48, 50-52, 54, 59, 60, 62, 64, 65, 67, 68, 71-73, 75-77, 79, 81, 83-91, 93-96, 99, 101, 102 and 104-106 of SEQ ID NO: 2 or comprise one or more of the following peptides: PKXXAGR (SEQ ID NO:319; amino acids 31-37 of SEQ ID NO: 2), or AGRXKF (SEQ ID NO: 320; amino acids 35-40 of SEQ ID NO: 2) or ETRHP (SEQ ID NO: 321; amino acids 42-46 of SEQ ID NO: 2).

TABLE 13

| | | | Peptide | | | % |
|---|---|---|---|---|---|---|
| DNA SEQ Name | | SEQ ID NO: | SEQ Name | | SEQ ID NO: | ID* |
| Rye | CBF20 | 115 | Rye | CBF20 | 116 | 69 |
| | | | | PEP | | |
| Rye | CBF28 | 117 | Rye | CBF28- | 118 | 70 |
| | | | | PEP | | |
| Rye | CBF46 | 119 | Rye | CBF46- | 120 | 71 |
| | | | | PEP | | |
| Rye | CBF7 | 121 | Rye | CBF7- | 122 | 67 |
| | | | | PEP | | |
| Rye | CBF71 | 123 | Rye | CBF71- | 124 | 71 |
| | | | | PEP | | |
| Wheat | CBF | 125 | Wheat | CBF- | 126 | 71 |
| | | | | PEP | | |

*Percentage identity to the CBF1 (SEQ ID NO: 2) AP2 region of derived polypeptide, wherein the AP2 region is defined by the AP2 consensus sequence and bounded by HPXYXGVR-ADS.

### Example 20

Identification and Isolation of Additional CBF Orthologs from other Plants

Medicago truncatula

Orthologous CBF sequences from Medicago truncatula were identified by BLAST analysis (TBLASTN 2.1.3 [Apr. 1, 2001]). Medicago truncatula ESTs in the Genbank public database were queried using the conserved domain from Arabidopsis CBF1, CBF2, and CBF3. The Arabidopsis sequences included the entire AP2/EREBP conserved domain flanked by the CBF signature sequences PKK/RPAGRxKFxETRHP and DSAWR of Arabidopsis CBF1, CBF2, CBF3, and CBF4 (SEQ ID NOs: 327, 328, 329, and 330, respectively). EST sequences were considered positive hits if they had an HSP BLAST score of at least 100 bits over at least 95% of the query sequence.

All positive EST sequences were organized into contigs using the SEQUENCHER assembly program (SEQUENCHER version 3.1, Gene Codes Corporation, Ann Arbor Mich.). At least seven separate sequences were identified (SEQ ID NOs: 294, 295, 296, 297, 298, 299, and 300). Those that had complete predicted coding regions with a start and a stop codon were cloned directly from cDNA or genomic DNA by PCR using primers in the 5′ and 3′ flanking regions. RACE (SMART RACE, BD Clontech) was used to identify the start and stop codons of genes that did not have full-length coding sequences available in the public database. These sequences were then cloned as above. All sequences (SEQ ID NOs: 294, 295, 296, 297, 298, 299, and 300) were cloned into the transformation vector pMEN65, or a modified version with GATEWAY compatible sites (GATEWAY Technology, Invitrogen Life Technologies, Carlsbad Calif.).

Oryza sativa

Rice orthologs were identified in the GenBank public database as described above for Medicago truncatula. Positive hits were organized into contigs using Sequencher. Ten full length sequences (SEQ ID NOs: 301, 302, 303, 304, 305, 306, 307, 308, 309, and 310) were identified and cloned from genomic DNA by PCR using primers in the 5′ and 3′ untranslated regions. All genes were cloned into the transformation vector pMEN65, or a modified version with GATEWAY compatible sites.

Medicago saliva

No putative alfalfa orthologs were identified in GenBank public database using BLAST analysis as described above for M. truncatula. However, because M. truncatula and M. sativa species are so closely related, three alfalfa CBF orthologs (SEQ ID NOs: 316, 317, and 318) were cloned directly from alfalfa genomic DNA using the M. truncatula cloning primers. All genes were cloned into the transformation vector pMEN65.

Zea mays

Maize orthologs were identified using BLAST analysis as described for M. truncatula in a proprietary cDNA database. Five orthologs (SEQ ID NOs: 311, 312, 313, 314, and 315) were cloned with PCR primers in the 5′ and 3′ untranslated regions and inserted into pMEN65.

Alignment with Arabidopsis CBFs

Polynucleotides isolated from Medicago truncatula (SEQ ID NOs: 294-300) which encoded CBF-related peptide sequences are shown compared with Arabidopsis CBF1, CBF2, and CBF3 in FIG. 39 (SEQ ID NOs: 2, 15, 13, and 97, respectively). Polynucleotides isolated from Oryza sativa (SEQ ID NOs: 301-310) which encoded CBF-related peptide sequences are shown compared with Arabidopsis CBF1, CBF2, and CBF3 in FIG. 40 (SEQ ID NOs: 2, 15, 13, and 97, respectively). Polynucleotides isolated from Zea mays (SEQ ID NOs: 311-315) which encoded CBF-related peptide sequences are shown compared with Arabidopsis CBF1, CBF2, CBF3, and CBF4 in FIG. 41 (SEQ ID NOs: 2, 15, 13, and 97, respectively).

### Example 21

Testing of CBF Orthologs in a Transient Assay

Preparation of Transformant Vectors

Seven clones from M. truncatula, ten clones from rice, and three clones from alfalfa (M. sativa) were tested for CBF activity in a transient assay. CBF activity was determined by activation of the RD29a promoter, quantitated by GUS activity. Results are shown on FIG. 42. In addition, rice CBF G3376 (SEQ ID NO:307) activated transcription from the RD29a promoter by greater than four-old when compared with the plasmid pMEN 65 vector control.

CBF clones were introduced into the pMEN 65 vector under the transcriptional control of the 35S promoter to create a 35S::CBF clone.

35S::CBF clones transformed into Agrobacterium cells were grown up overnight in LB medium (supplemented with kanamycin 75 mg/l; spectinomycin 100 mg/l; chloramphenicol 25 mg/l) at 28° C. with shaking. The resulting culture (between 1% and 10% original volume) was then inoculated once more into LB (supplemented with kanamycin 75 mg/l; spectinomycin 100 mg/l; chloramphenicol 25 mg/l; 20 μM acetosyringone; and 10 mM MES) and grown overnight at 28° C. with shaking.

Plasmid P511, which contains the GUS reporter gene under the control of the RD29a promoter, was transformed and amplified as described for the 35S::CBF clone in this example. Plasmid vector pMEN 65 was similarly innoculated and amplified to provide the vector control.

The overnight cultures were then pelleted by low-speed centrifugation, 1500×g (3000 rpm in Sorvall RT7) for 25 minutes. Cell pellets were resuspended in ⅕ the original culture volume of freshly made Infiltration Media (IM: 10 mM MES; 10 mM MgCl₂; 150 μM acetosyringone) and resuspended cell pellets were kept at ambient temperature, with occasional mixing, for at least two hours before use. The resuspensed cell pellet was diluted with IM to attain an final absorption value at 600 nm of 1.0 in a 1 cm lightpath (A₆₀₀, O.D.=1.00).

Equal amounts of the P511 culture and the 35S::CBF culture were mixed by inversion to a final volume of 0.8 ml per tube.

Transient Transformation of Nicotiniana benthamiana Leaf Tissue

The plant was watered from the top two hours prior to the infiltration procedure. Healthy symetrical leaves that had good turgor were chosen. A total of ten ˜0.5 cm-diameter circles were marked on the axial side of each leaf. A positive control was included on at least one leaf of each plant. Negative controls were included in the assay on every leaf. Control suspensions were made up for half the reporter construct mix and for the other half of the mix a binary vector without insert was used.

The culture mix was taken up into a 1 ml disposable syringe and approximately 0.1 ml was expeled onto each circle on the leaf. The plants were then cultivated for five days until sample collection.

Extraction and GUS Assay

Each leaf disk was excised from the plant using a cork borer and placed in 300 μl of Buffer 1 (50 mM Na+ Phosphate, pH 7.9; 16.7 mM EDTA) in a tube in a cluster tube rack (Catalogue No. 4413, Corning Inc.) which also contained a single stainless steel bead (Type 440C stainless stell balls 3/16″, Catalogue No. 9529K13, McMaster-Carr Supply Co.).

One hundred microlitres of Buffer 2 (50 mM Na+ Phosphate, pH 7.9; 50 mM β-mercaptoethanol) was then added to each tube, the tube was capped, and the rack shaken in a model MM300 shaker-mixer (F. Kurt Retch Gmbh & Co. K G, Haan, Germany) for 1.5 minutes at 28 beats/minute. The rack was rotated half a turn and shaken again for 1.5 minutes. The rack was briefly centrifuged.

One hundred microlitres of Buffer 3 (50 mM Na+ Phosphate, pH 7.9; 0.5% lauroylsarcosine, sodium salt; 0.5% TRITON X-100) was then added to each tube and mixed by inversion. The tube was allowed to stand for two minutes at ambient temperature. The rack was centreifuged at 3000 rpm in a table top centrifuge for 15 minutes at 4° C.

Forty microliters of Extraction Buffer (3 parts Buffer 1 mixed with 1 part Buffer 2 with 1 part Buffer 3) were added to each well in a 96-well reaction plate (Microseal 96 Skirted V-bottom Polypropylene Microplate; Catalogue No. MSP-9601, Natural; MJ Research, Inc.) Ten microlitres of the supernatant from each tube was added to each well.

Ten miligrams of 4-methylumbelliferyl β-D-glucuronide (MUG; Catalogue No. M-5664, Sigma-Aldrich Chemical Co.) were disolved in 14.5 ml of Extraction Buffer to approximately 2 mM. 50 μl of this MUG solution was added to each sample in the reaction plate and the plate was incubated at 37° C. for one hour.

Two hundred microlitres of Stop Buffer (0.2 M Na₂CO₃) were added to a black 96-well plate (Polyfiltronics 96-well, 300 μl, black, flat-bottom microplates; Catalogue No. PF030-PBX8, Phenix Research Products, Hayward Calif.), then 8 μl of each reaction transferred to each Stop Buffer plate well and mixed. The samples were then read using a Synergy HT plate reader (BIOTECK Instruments, Inc., Winoosk, Vt.).

### Example 22

Overexpression of CBF1 or CBF2 Increases Arabidopsis Drought or High Salt Tolerance

Soil studies were done by growing seedlings for 10 days with water, and then letting the soil dry out (no further watering) until the plants were severely dehydrated. The soil was then watered and then recovery of the plants was measured. The transgenic lines were alternated with the control wild type lines, with a one-inch spacing between plants in two inches of soil. No detrimental effects were observed but the beneficial effects seen need further testing with drought inducible promoter lines.

Two separate root elongation assays were performed to evaluate the drought resistance phenotypes of the transgenic plants. First plants were grown on MS agar plates for two weeks, and then transferred to MS agar plates containing either 300 mM mannitol or 150 mM NaCl. Those concentrations were chosen because preliminary testing showed that wild type plants showed the most dramatic reduction in root growth in those conditions. The root lengths were then measured after seven days, and the data summarized in Table 14. The growth on sucrose (0.3% w/v), the non-inhibition control and the growth on either salt or mannitol is shown. The control line lacking a CBF gene is the 643-3 line. When the ratio of the CBF line to the 643-3 line (CBF/wild type) is significantly above 1.0, this is an indication of drought or salt tolerance.

From these, we concluded that the overexpression of the CBF genes did provide growth benefit under high osmotic pressure and high salt, particularly for the CBF2 lines tested.

TABLE 14
Mannitol (M) and Salt (S) Root Elongation Assay (mm)

| | Ave | Std | CBF/wt |
|---|---|---|---|
| CBF1#1 | 38.5 | 12.9 | 0.80 |
| 643-3 | 48.2 | 10.4 | |
| CBF1#1 (S) | 20.0 | 7.1 | 1.30 |
| 643-3 (S) | 15.4 | 3.3 | |
| CBF1#1 (M) | 21.7 | 6.1 | 1.63 |
| 643-3 (M) | 13.3 | 3.6 | |
| CBF1#6 | 43.7 | 16.8 | 0.94 |
| 643-3 | 46.5 | 14.7 | |
| CBF1#6 (S) | 16.5 | 4.0 | 1.03 |
| 643-3 (S) | 16.0 | 3.8 | |
| CBF1#6 (M) | 23.2 | 7.7 | 1.25 |
| 643-3 (M) | 18.5 | 3.6 | |
| CBF2#10 | 48.3 | 7.2 | 0.89 |
| 643-3 | 54.2 | 5.3 | |
| CBF2#10 (S) | 15.8 | 3.7 | 1.08 |
| 643-3 (S) | 14.7 | 2.6 | |
| CBF2#10 (M) | 21.0 | 5.4 | 1.31 |
| 643-3 (M) | 16.1 | 3.4 | |
| CBF2#14 | 40.5 | 11.3 | 0.89 |
| 643-3 | 45.3 | 13.0 | |
| CBF2#14 (S) | 21.5 | 3.9 | 1.50 |
| 643-3 (S) | 14.4 | 3.0 | |
| CBF2#14 (M) | 24.0 | 5.3 | 1.67 |
| 643-3, (M) | 14.4 | 2.6 | |

### Example 23

Identification of Transgenic Arabidopsis Plants that Express CBF3

Total soluble protein was obtained essentially as described (Gilmour et al. (1996) Plant Physiol. 111: 293-299) by grinding leaf material (about 100 mg) in 0.4 ml extraction buffer (50 mM PIPES pH 7.0, 25 mM EDTA) containing 2.5% (w/v) polyvinyl-polypyrrolidone and removing insoluble material by centrifugation (16000 g×20 min). Protein concentration in the supernatant was measured using the dye-binding method of Bradford (1976) with BSA as the standard. Protein samples (50 μg total protein) were fractionated by tricine SDS/PAGE (Schägger et al. (1987) Anal. Biochem. 166: 368-379) and transferred to 0.2 micron nitrocellulose membranes by electroblotting. COR15am and COR6.6 were detected using the ECL kit (Amersham) with antiserum raised to recombinant COR15am and COR6.6 (Gilmour et al. (1996) Plant Physiol. 111: 293-299).

Transgenic Arabidopsis plants that overexpress CBF3 at normal growth temperature were created by placing the CBF3 coding sequence under control of the cauliflower mosaic virus (CaMV) 35S promoter and transforming the construct into Arabidopsis plants using the floral dip transformation procedure. Transgenic Arabidopsis plants that overexpress CBF3 were generated by transforming the chimeric genes into Arabidopsis ecotype Ws-2 plants.

A 910 bp BamHI/HindIII fragment from a cDNA clone containing the whole coding region of CBF3 (Gilmour et al. (1998) Plant J. 16: 433-442) was inserted into the BglII and HindIII sites of the binary transformation vector pGA643. PGA643 has a CaMV 35S promoter and the terminator from gene 7 of pTiA6 (An (1988) “Binary Vectors”, in Gynheung et al., editors, (1995) Plant Molecular Biology Manual, Kluwer Acad. Publishers). The resulting plasmid, pMPS13, which contains the CBF3 coding sequence under control of the CaMV 35S promoter, was transformed into Agrobacterium tumefaciens strain GV3101 by electroporation (Koncz et al. (1986) Mol. Gen. Gen. 204: 383). Arabidopsis plants were transformed with plasmid pMPS13 or the transformation vector pGA643 using the floral dip method (Clough et al. (1998) Plant J. 16: 735-743). Transformed plants were selected on the basis of kanamycin resistance. Homozygous T3 or T4 plants were used in all experiments.

Standard procedures were used for plasmid manipulations (Sambrook et al. (1989), supra). Prior to transformation, Arabidopsis thaliana seeds were sown at a density of ˜10 plants per 4″ pot onto Bacto planting mix (Michigan Peat Co., Houston, Tex.) covered with fiberglass mesh (18 mm×18 mm). Plants were grown under continuous illumination (100-150 μE/m²/sec) at 20-22° C. with 65-70% relative humidity. Plants were used when the primary inflorescences were approximately 10-12 cm high. The pots were then immersed upside down in the mixture of Agrobacterium infiltration medium as described above (Clough et al. (1998) supra) for 2-3 seconds, and placed on their sides to allow draining into a 1′×2′ flat surface covered with plastic wrap. After 24 hours, the plastic wrap is removed and the pots were turned upright. Seeds were then collected from each transformation pot and analyzed following the protocol described below

A construct comprising a cold-regulatable polypeptide gene coding sequence (CBF3) operably linked to a constitutive promoter was generated.

Twenty-two independent lines were identified in which the T2 plants segregated 3:1 for kanamycin resistance (the selectable marker carried on the transformation vector). These lines presumably carried a single active T-DNA locus. The kanamycin resistant T2 plants were then screened by western analysis for constitutive expression of COR15a, a target gene of the CBF transcriptional activators.

Three independent transgenic lines—A28, A30 and A40—were identified that produced the COR15am polypeptide at high levels uniformly among plants grown at normal temperatures. Northern blot analysis indicated that the transcript levels for CBF3 were about equal in non-cold acclimated and cold-treated A28, A30 and A40 plants and were much greater than those observed in either non-cold acclimated or cold-treated control plants (i.e. non-transformed plants or transgenic plants carrying the transformation vector without an insert). The transcript levels for two target COR genes, COR15a and COR6.6, were also nearly equal in non-cold acclimated and cold-treated A28, A30 and A40 plants and approximated the levels observed in cold-acclimated control plants. Western blot analysis indicated that the proteins encoded by COR15a and COR6.6 were present in both non-cold acclimated and cold-acclimated A28, A30 and A40 plants at 3 to 5 fold-higher levels than those found in cold-acclimated control plants.

### Example 24

Overexpression of the CBF-Related Polypeptide in Arabidopsis Affects Physiological and Morphological Characteristics of the Plant

After the same number of days of vegetative growth at normal temperature, physiological and morphological characteristics of the CBF3-expressing plants were compared with those of wild type plants. The size was less than that of the control plants. Additional differences in growth characteristics were also evident. One was that the CBF3-expressing plants had a pronounced prostrate growth habit; whereas the leaves of the control plants generally had an upright stature, those of the transgenic plants laid flat to the soil. The CBF3-expressing plants also had much shorter petioles when compared to those of the control plants and the leaves had a slight bluish-green tint. Also, there was a substantial difference in time to flowering between the control and CBF3-expressing plants; i.e., control plants bolted and formed flowers well before the CBF3-expressing plants did. In one experiment, for instance, the control plants began to bolt at 17 days while A40, A30 and A28 plants took 21, 26 and 28 days, respectively, to initiate bolting (Table 15).

TABLE 15
Effects of CBF3 Expression on Time to Flowering and
Rosette Leaf Number.

| Plants | Time to Flowering (d) | Rosette Leaves Per Plant |
|---|---|---|
| Ws-2 | 17 | 4.5 |
| B6 | 17 | 4.6 |
| A40 | 21 | 6.0 |
| A30 | 26 | 9.7 |
| A28 | 28 | 12.5 |

The CBF3-expressing plants went on to form flowers and set seed, though the final plant mass and seed yield was considerably less than that obtained with control plants. The lower yield of seed was due at least in part to the CBF3-expressing plants producing fewer axillary shoots. Significantly, the delay in flowering observed in the CBF3-expressing plants did not “simply” involve a slower overall growth rate, but appeared to involve a developmental delay in flowering. In one experiment, for instance, the control plants produced an average of 4.5 and 4.6 leaves per rosette while the A40, A30, and A28 transgenic plants produced 6.0, 9.7, and 12.5 leaves per rosette, respectively (Table 15).

In some plants, including Arabidopsis, the transition to flowering is responsive to vernalization, a long period (weeks) of low temperature treatment. In “facultative” plants such as Arabidopsis, the effect of vernalization is to shorten the time to flowering (the magnitude of the effect varies greatly among Arabidopsis ecotypes). If CBF3 expression at warm temperature was fully mimicking exposure to low temperature, then one might think that if it had any effect on flowering, that it would decrease the transition time and cause a corresponding decrease in the number of rosette leaves per plant. Our results, however, indicate that CBF3 expression had the opposite effect; it increased the time to flowering and increased the number of rosette leaves per plant.

When plants expressed a CBF-related polypeptide behind an inducible promoter, the plants were late flowering and their leaf number when measured at the time of bolting (i.e. at a comparable developmental stage) was increased compared to the control as was the above ground biomass at the developmental stage as shown in FIGS. 27A through 27C. The Dreb2a::CBF1 plants grew more slowly, i.e. when plants of same age were compared the biomass of the transgenics is slightly smaller (˜30%) than that of the control plants. But as the plants flowered later they grow for a longer period of time prior to bolting and at this developmental stage they had more leaves and higher biomass than the control plant at the same developmental stage (which the control plant reached a few days earlier).

### Example 25

Vegetative Growth of Canola after Transformation with Plasmids Containing CBF1, CBF2, or CBF3

Canola was transformed with a plasmid containing the Arabidopsis CBF1, CBF2, or CBF3 genes cloned into the vector pGA643 (An (1987) Methods Enzymol. 253: 292). In these constructs the CBF genes were expressed constitutively under the CaMV 35S promoter. In addition, the CBF1 gene was cloned under the control of the Arabidopsis COR15 promoter in the same vector pGA643. Each construct was transformed into Agrobacterium strain GV3101. Transformed agrobacteria was grown for 2 days in Minimal AB medium containing the appropriate antibiotics.

Spring canola (B. napus cv. Westar) was transformed using the protocol of Moloney (Moloney et. al. (1989) Plant Cell Reports 8: 238) with some modifications as described. Briefly, seeds were sterilized and plated on half strength MS medium, containing 1% sucrose. Plates were incubated at 24° C. under 60-80 uE/m²s light using a16 hour light/8 hour dark photoperiod. Cotyledons from 4-5 day old seedlings were collected, the petioles cut and dipped into the Agrobacterium solution. The dipped cotyledons were placed on co-cultivation medium at a density of 20 cotyledons/plate and incubated as described above for 3 days. Explants were transferred to the same media, but containing 300 mg/l timentin (GlaxoSmithKline, PA) and thinned to 10 cotyledons/plate. After 7 days explants were transferred to Selection/Regeneration medium. Transfers were continued every 2-3 weeks (2 or 3 times) until shoots had developed. Shoots were transferred to Shoot-Elongation medium every 2-3 weeks. Healthy looking shoots were transferred to rooting medium. Once good roots had developed, the plants were placed into moist potting soil.

The transformed plants were then analyzed for the presence of the NPTII gene/kanamycin resistance by Elisa, using the Elisa NPTII kit from 5Prime-3Prime Inc. (Boulder, Colo.). Approximately 70% of the screened plants were NPTII positive. Only those plants were further analyzed.

After the same number of days of vegetative growth at normal temperature, physiological and morphological characteristics of the CBF-expressing plants were compared with those of wild type plants. Our results indicated that CBF3 increased the time to flowering and increased the number of rosette leaves per plant. This phenotype was most obvious in transgenic canola plants overexpressing CBF1, CBF2, or CBF3 under control of the 35S promoter.

### Example 26

Overexpression of CBF1, CBF2, or CBF3 Affects Stress Tolerance in Plants

In addition to modification to the biomass of plants that occurs due to overexpression of CBF1, CBF2, or CBF3 genes shown in the above examples, plants that have been transformed and overexpress these genes may also be made more resistant to environmental stresses. Thus, when grown under adverse conditions, the biomass of plant transformed with CBF1, CBF2, or CBF3 genes may be significantly greater than that of a plant that is more susceptible to the stress(es) and fails to thrive.

Overexpression of CBF1, CBF2, or CBF3 Increases Freezing Tolerance in Arabidopsis.

Ws-2 and A30 seedlings were grown (13 days and 20 days, respectively) on Gamborg's B-5 medium containing 0.2% sucrose under sterile conditions in Petri dishes. The plants were tested for freezing tolerance by first placing the plates at −2° C. in the dark for 24 hours followed by ice nucleation with sterile ice chips for 3 hours. The temperature of the freezer was then turned down to −6° C. and the plates were left at this temperature for an additional 24 hours. The plates were taken from the freezer and placed at 4° C. in the dark for 18 hours, followed by 2 days at 22° C. under cool white fluorescent lights (40-50 μmol m⁻² s⁻¹) with an 18 hour photoperiod. The plates were scored 2 days later for freezing damage.

Electrolyte leakage freeze tests were performed essentially as described (Uemura et al. (1995) Plant Physiol. 109: 15-30) with minor modifications. Tubes (16×125 mm) containing 3-4 leaves were placed in a low temperature bath set at −2° C. in a randomized design. The randomization was performed with the aid of the SAS system (SAS Institute Inc, Cary N.C.). Ice chips were added to each tube after 1 hour incubation. Each tube was capped with foam plugs and incubated a further 1 hour at −2° C. The bath temperature was then lowered one degree C every 20 minutes. Tubes were removed at each temperature and incubated an additional hour at that same temperature in a separate bath. Tubes were placed on ice after removal from the bath until all tubes had been removed. The samples were then thawed overnight at 2.5° C. and electrolyte leakage was measured as described (Gilmour et al. (1988) Plant Physiol. 87: 745-750).

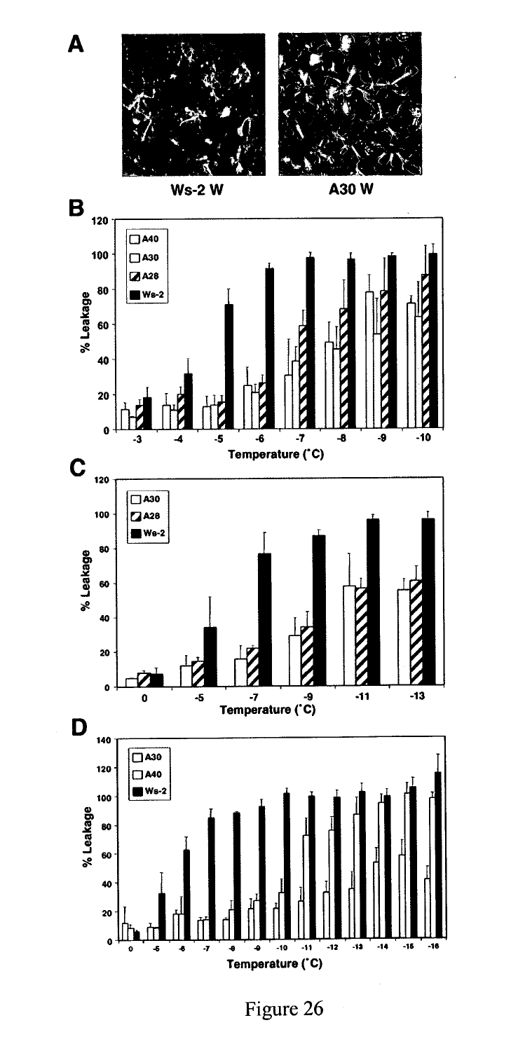

Non-cold acclimated control plants were killed by freezing at −6° C. for 24 hours while non-cold acclimated CBF3-expressing plants were not. Results for Ws-2 and A30 plants are shown in FIG. 26A. Electrolyte leakage tests indicated that the freezing tolerance of non-cold acclimated CBF3-expressing plants was about 3 to 4° C. greater than that of the non-cold acclimated control plants. Specifically, non-cold acclimated control plants had an EL₅₀ (temperature that caused a 50% leakage of electrolytes) of about −4.5° C. while the three CBF3 expressing lines had EL₅₀ values of about −8° C. (FIG. 26B). Significantly, the freezing tolerance of cold-acclimated CBF3-expressing plants was considerably greater than that of cold-acclimated control plants. Control plants that had been cold-acclimated for 7 days had an EL₅₀ value of about −6° C. while 7 days cold-acclimated CBF3-expressing plants had EL₅₀ values of −11° C. or lower (FIGS. 26C and 26D).

Overexpression of CBF1, CBF2, or CBF3 Increases Salt Tolerance in Canola.

Canola was transformed with a plasmid containing the Arabidopsis CBF1, CBF2, or CBF3 genes cloned into the vector pGA643 (An (1987) Methods Enzymol. 253: 292). In these constructs the CBF genes were expressed constitutively under the CaMV 35S promoter. In addition, the CBF1 gene was cloned under the control of the Arabidopsis COR15 promoter in the same vector pGA643. Each construct was transformed into Agrobacterium strain GV3101. Transformed agrobacteria was grown for 2 days in Minimal AB medium containing the appropriate antibiotics.

Spring canola (B. napus cv. Westar) was transformed using the protocol of Moloney (Moloney et al. (1989) Plant Cell Reports 8: 238) with some modifications as described. Briefly, seeds were sterilized and plated on half strength MS medium, containing 1% sucrose. Plates were incubated at 24° C. under 60-80 uE/m²s light using a16 hour light/8 hour dark photoperiod. Cotyledons from 4-5 day old seedlings were collected, the petioles cut and dipped into the Agrobacterium solution. The dipped cotyledons were placed on co-cultivation medium at a density of 20 cotyledons/plate and incubated as described above for 3 days. Explants were transferred to the same media, but containing 300 mg/L timentin (GlaxoSmithKline, PA) and thinned to 10 cotyledons/plate. After 7 days explants were transferred to Selection/Regeneration medium. Transfers were continued every 2-3 weeks (2 or 3 times) until shoots had developed. Shoots were transferred to Shoot-Elongation medium every 2-3 weeks. Healthy looking shoots were transferred to rooting medium. Once good roots had developed, the plants were placed into moist potting soil.

The transformed plants were then analyzed for the presence of the NPTII gene/kanamycin resistance by Elisa, using the Elisa NPTII kit from 5Prime-3Prime Inc. (Boulder, Colo.). Approximately 70% of the screened plants were NPTII positive. Only those plants were further analyzed.

From Northern blot analysis of the plants that were transformed with the constitutively expressing constructs, showed expression of the CBF genes and all CBF genes were capable of inducing the Brassica napus cold-regulated gene BN115 (homolog of the Arabidopsis COR15 gene). Most of the transgenic plants appear to exhibit a normal growth phenotype. As expected, the transgenic plants are more freezing tolerant than the wild-type plants. Using the electrolyte leakage of leaves test, the control showed a 50% leakage at −2 to −3° C. Spring canola transformed with either CBF1 or CBF2 showed a 50% leakage at −6 to −7° C. Spring canola transformed with CBF3 shows a 50% leakage at about −10 to −15° C. Winter canola transformed with CBF3 may show a 50% leakage at about −16 to −20° C. Furthermore, if the spring or winter canola are cold acclimated the transformed plants may exhibit a further increase in freezing tolerance of at least −2° C.

To test salinity tolerance of the transformed plants, plants were watered with 150 mM NaCl. Plants overexpressing CBF1, CBF2, or CBF3 grew better compared with plants that had not been transformed with CBF1, CBF2, or CBF3.

### Example 27

Overexpression of CBF3 Affects Proline Metabolism in Arabidopsis

Proline levels in leaf samples were analyzed by methods described in Example 5.

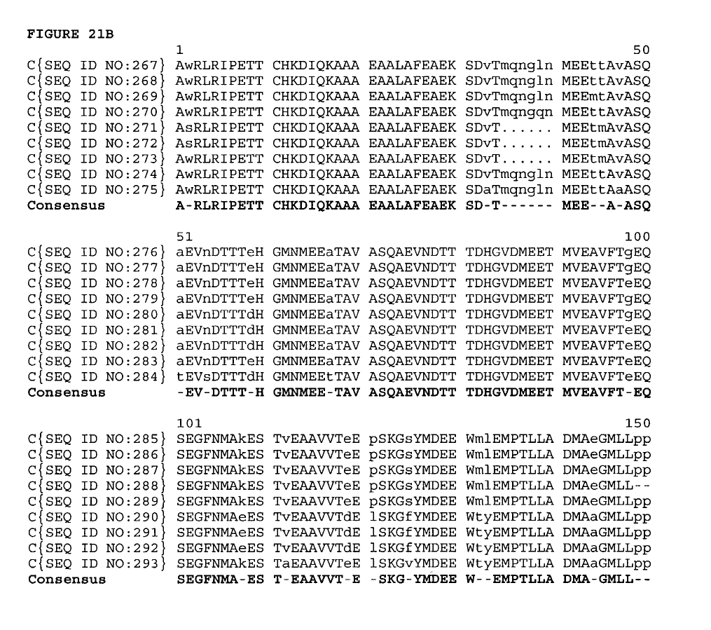

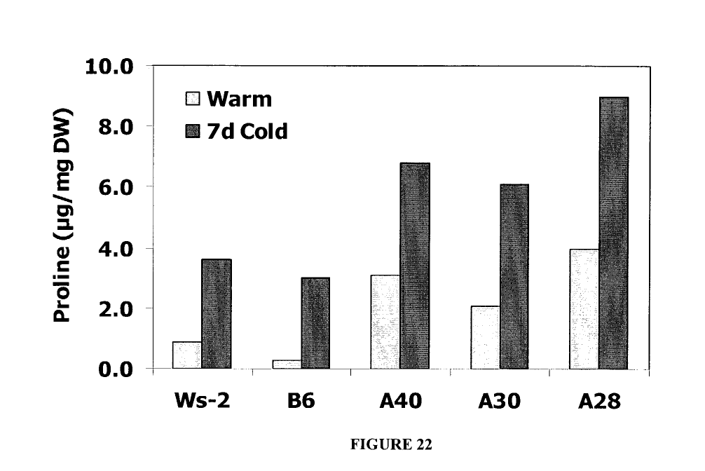

Under non-cold acclimating growth conditions, the free proline levels in the CBF3-expressing plants were about 5-fold higher than they were in the control plants, levels which were about the same as those found in cold-acclimated control plants (FIG. 22). The proline levels in the CBF3-expressing plants increased further (about 2-fold) upon cold acclimation and were 2-3 fold higher than those found in the cold-acclimated control plants (FIG. 22).

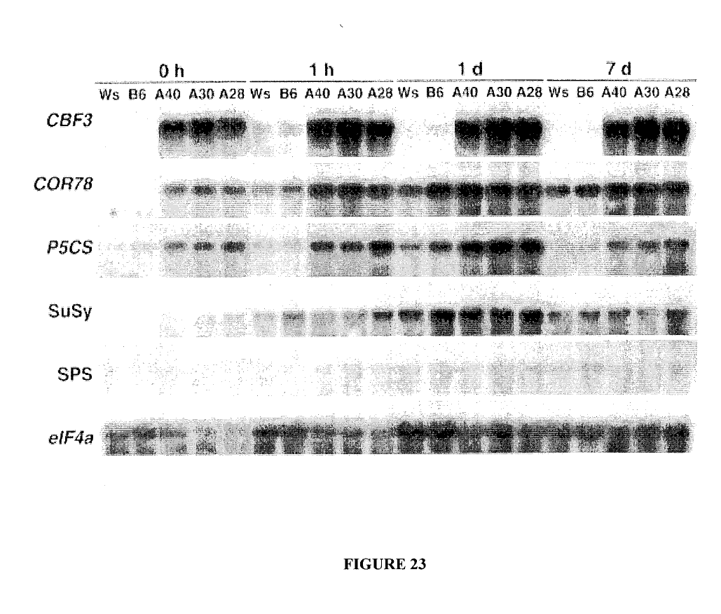

The proline biosynthetic enzyme Δ′-pyrroline-5-carboxylate synthase has a key role in determining proline levels in plants (Yoshiba et al. (1997) Plant Cell Physiol. 38: 1095-1102). Because of this, and that P5CS transcript levels have been shown to increase in Arabidopsis in response to low temperature (Xin et al. (1998) Proc. Natl. Acad. Sci. 95: 7799-7804), it was of interest to determine whether P5CS transcript levels were elevated in the CBF3-expressing plants. Northern analysis indicated that they were; P5CS transcript levels were about 4 fold higher in non-cold acclimated CBF3-expressing plants than they were in non-cold acclimated control plants and were about equal to those found in the control plants that had been cold-treated for 1 day (FIG. 23). The P5CS transcript levels in 7-day cold-acclimated CBF3-expressing plants were 2 to 3 fold higher than in cold-acclimated control plants (FIG. 23), a finding that was consistent with the 2 to 3 fold higher levels of proline found in the cold-acclimated CBF3-expressing plants (FIG. 22).

### Example 28

Overexpression of CBF3 Affects Sugar Metabolism in Arabidopsis

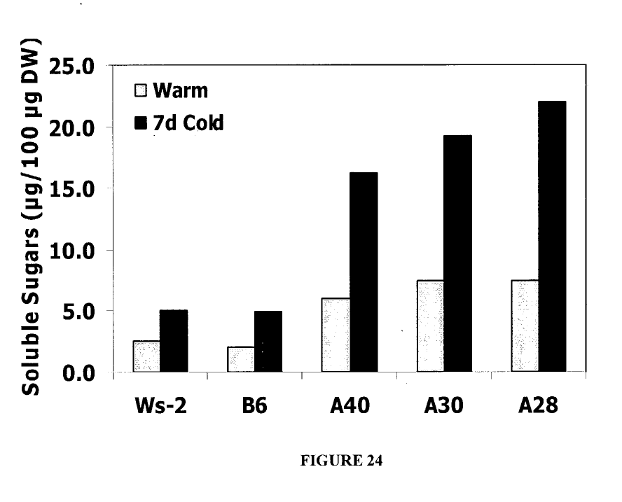

Total soluble sugars (for example, sucrose, glucose, and fructose among others) were extracted from lyophilized leaf material as described in Example 5. The results showed that CBF3 expression affected the sugar levels in plants. Total soluble sugars in control and CBF3-expressing plants at both non-cold acclimating and cold acclimating temperatures were measured. The results show (FIG. 24) that the levels of total sugars in non-cold acclimated CBF3-expressing plants were about 3-fold greater than those in non-cold acclimated control plants. Upon cold acclimation, sugar levels went up in both the control and CBF3-expressing plants about 2-fold, and remained about 3-fold higher in the CBF3-expressing plants. Analysis of the sugars by HPLC indicated that CBF3 expression affected the levels of sucrose; in non-cold acclimated control plants, sucrose levels were about 0.3 μg/100 μg dry weight (DW), while in non-cold acclimated CBF3-expressing plants they were about 1.5 μg/100 μg DW.

### Example 29

Overexpression of CBF3 Affects Lipid Composition

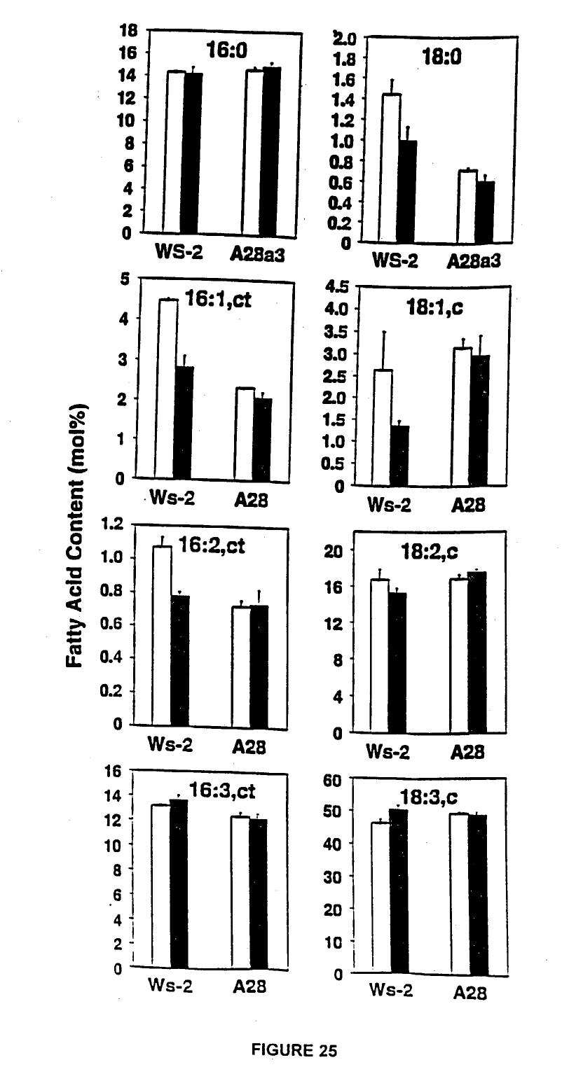

Total lipids were extracted from Arabidopsis leaves and measured as described in Example 5. The results indicated that CBF3 expression affected lipid composition. The representative results of Ws-2 and A28 are presented in FIG. 25. They indicate that expression of CBF3 had little or no effect on the relative amounts (mol %) of 16:0, 16:3, 18:2 or 18:3 fatty acids in non-cold acclimated plants. Significantly, no appreciable change in the relative amounts of these fatty acids occurred during cold acclimation either. Cold acclimation did result in sizable decreases (30 to 50%) in the relative amounts of 16:1, 16:2, 18:0 and 18:1 fatty acids and in three of these cases, specifically 16:1, 16:2, and 18:0 fatty acids, CBF3 expression caused similar decreases to occur in non-cold acclimated plants. In the case of 18:1 fatty acids, CBF3 expression had an opposite effect from cold acclimation; it resulted in a slight increase in the relative levels of this fatty acid in non-cold acclimated transgenic plants and about a 50% increase in cold-acclimated transgenic plants. Taken together, these results indicate that overexpression of CBF3 has an effect on fatty acid composition and that certain of the changes mimic those that occur with cold acclimation.

### Example 30

Overexpression of CBF3 Increases Arabidopsis Freezing Tolerance

Ws-2 and A30 seedlings were grown (13 days and 20 days, respectively) on Gamborg's B-5 medium containing 0.2% sucrose under sterile conditions in Petri dishes. The plants were tested for freezing tolerance by first placing the plates at −2° C. in the dark for 24 hours followed by ice nucleation with sterile ice chips for 3 hours. The temperature of the freezer was then turned down to −6° C. and the plates were left at this temperature for an additional 24 hours. The plates were taken from the freezer and placed at 4° C. in the dark for 18 hours, followed by 2 days at 22° C. under cool white fluorescent lights (40-50 μmol m⁻² s⁻¹) with an 18 hour photoperiod. The plates were scored 2 days later for freezing damage.

Electrolyte leakage freeze tests were performed essentially as described (Uemura et al. (1995) Plant Physiol. 109: 15-30) with minor modifications. Tubes (16×125 mm) containing 3-4 leaves were placed in a low temperature bath set at −2° C. in a randomized design. The randomization was performed with the aid of the SAS system (SAS Institute Inc, Cary N.C.). Ice chips were added to each tube after 1 hour incubation. Each tube was capped with foam plugs and incubated a further 1 hour at −2° C. The bath temperature was then lowered one degree C. every 20 minutes. Tubes were removed at each temperature and incubated an additional hour at that same temperature in a separate bath. Tubes were placed on ice after removal from the bath until all tubes had been removed. The samples were then thawed overnight at 2.5° C. and electrolyte leakage was measured as described (Gilmour et al. (1988) Plant Physiol. 87: 745-750).

Non-cold acclimated control plants were killed by freezing at −6° C. for 24 hours while non-cold acclimated CBF3-expressing plants were not. Results for Ws-2 and A30 plants are shown in FIG. 26A. Electrolyte leakage tests indicated that the freezing tolerance of non-cold acclimated CBF3-expressing plants was about 3 to 4° C. greater than that of the non-cold acclimated control plants. Specifically, non-cold acclimated control plants had an EL₅₀ (temperature that caused a 50% leakage of electrolytes) of about −4.5° C. while the three CBF3 expressing lines had EL₅₀ values of about −8° C. (FIG. 26B). Significantly, the freezing tolerance of cold-acclimated CBF3-expressing plants was considerably greater than that of cold-acclimated control plants. Control plants that had been cold-acclimated for 7 days had an EL₅₀ value of about −6° C. while 7 days cold-acclimated CBF3-expressing plants had EL₅₀ values of −11° C. or lower (FIGS. 26C and 26D).

### Example 31

Transformation and Expression in Arabidopsis Plants with CBF-Related Polynucleotides from other Plants

SEQ ID NOs: 294 through 318 were transformed into Arabidopsis plants as described in Examples 7 through 11. Transformed plants are screened for traits as described in Examples 7 through 11.

### Example 32

Transformation and Expression in Alfalfa Plants with CBF Polynucleotides

SEQ ID NOs: 294 through 318 were transformed into Medicago sativa plants as described. For alfalfa transformation a well established protocol from Deborah Samac's laboratory was used which is based on a system described by Austin et al., (Austin et al. (1995) Euphytica 85: 381-393). This system gives a regeneration rate of callus pieces producing embryos and later on transgenic plantlets of more than 75%. Transformation of the callus to the generation of transgenic plantlets took from between 9-12 weeks, and the transgenic plantlets were then propagated by cuttings. Leaf explants from the alfalfa cultivar Regen-SY are best suited for rapid production of embryos (Bingham (1991) Crop Sci. 31: 1098).

For transformation, young leaves (top second to fifth node) from soil grown plants were surface sterilized by a brief rinse in 70% ethanol followed by gentle agitation in a 20% bleach solution for 90 seconds. After three rinses in sterile water, leaflet margins were trimmed away and the remaining piece was cut in half. 50 leaf pieces were generated and placed in 12 ml of liquid SH medium without hormones (Schenk and Hildebrandt (1972) Can. J. Bot. 50: 199-204). The day before transformation a 4 ml overnight culture (YEP liquid media containing 25 mg/l rifampicin) of A. tumefaciens strain LBA4404 containing the binary vector of interest was inoculated. For the transformation, 3 ml of this overnight culture were added to the leaf pieces and incubated at room temperature for 10-15 minutes. After that, the leaf pieces were removed, blotted briefly on sterile filter paper, and placed on plates containing solid callus-inducing B5 media with vitamins (Brown and Atanassov (1985) Plant Cell Tissue Organ Cult. 4:111-122): B5 salts and vitamins (Gamborg et al. (1968) Exp. Cell Res. 50: 151-158), 30 g/l sucrose, 0.5 g/l KNO₃, 0.25 g/l MgSO₄-7H₂O, 0.5 g/l proline, 798 mg/l L-glutamine, 99.6 mg/l serine, 0.48 mg/l adenine, 9.6 mg/l glutathione, 1 mg/l 2,4-D, 0.1 mg/l kinetin and 8 g/l Phytagar (Life Technologies, Inc.), pH 5.7). The plates were sealed with gas-permeable tape (#394 3M Venting Tape) and incubated for 7 days at 24° C. with a light intensity of approximately 100 μmol m⁻² sect⁻¹.

After 7 days co-cultivation the leaf pieces were rinsed 3-4 times in sterile water, and transferred to fresh selection plates (B5 media with vitamins containing 25 mg/l kanamycin and 100 mg/l ticarcillin or timentin). Calli and a few embryos formed after about two weeks, and were then transferred to MMS medium (MMS: MS salts, 30 g/l sucrose, vitamins (Nitsch and Nitsch (1969) Science 163: 85-87), 100 mg/l myo-inositol, 7 g/l phytagar plus 25 mg/l kanamycin and 100 mg/l ticarcillin or timentin) and cultured for an additional 2-3 weeks. As cotyledonary stage embryos grew out from the calli, they were cut off and transferred to fresh MMS medium with antibiotics for conversion to plantlets. Plantlets that formed a primary leaf were transferred to Magenta boxes containing fresh MMS media without kanamycin. This allowed for further root and shoot development. After 2-5 weeks plants that formed roots were removed from the boxes and placed in a test tube containing water. They were kept on the lab bench for 12-48 hours to condition the plants and which reduced leaf loss due to desiccation after transplanting. Individual plants were transplanted to a soil mix and maintained in a growth chamber under conditions to maximize leaf production: 24° C./19° C. day/night temperature, 16/8 hours of light/dark cycle with a light intensity of approximately 300 μmol m⁻² sec⁻¹). Plants were watered daily and were fertilized weekly with a complete fertilizer.

Plants from individual lines were propagated by cuttings and transferred to soil. Original transformants were maintained in Magenta boxes on selection, while the working plant material was generated by cuttings from the soil explants.

Up to 100 independent plants were tested by PCR for stable integration of the transgene to ensure at least 20 independent transgenic plants were generated per construct. As control for successful setup of the transformation capabilities a 35s::intGUS construct was used. An empty vector control was included with the batch of 4 CBF constructs (total of 6 constructs). The initial transformants (at least 20 per construct) were propagated by cuttings after they developed 5-8 internodes (roughly 7 weeks after transfer to soil). Each cut internode was placed in vermiculite for 1-2 weeks for root formation. 4-7 plants per transgenic line were transplanted in SC-10 Super Cell Cone-tainers (Hummert; cones: 3.8 cm diameter, 21 cm depth; 98 cones per 61×30.5 cm² tray) and grown under a 16/8 hours light/dark cycle either in the growth room at 24° C. or in a growth chamber at 24° C./19° C. day/night temperature (or constant at 4° C. for cold acclimation experiments) for further analysis.

Transformed plants are screened for traits as described in Examples 7 through 11.

While the present invention is disclosed by reference to the preferred embodiments and examples detailed above, it is to be understood that these examples are intended in an illustrative rather than limiting sense, as it is contemplated that modifications will readily occur to those skilled in the art, which modifications will be within the spirit of the invention and the scope of the appended claims.

What is claimed is:

1. A transgenic plant having an altered trait as compared to a wild-type or untransformed plant, wherein said altered trait is selected from the group consisting of:
(a) altered levels of at least one cell protectant in cells of said transgenic plant; and
(b) altered biomass;
wherein said transgenic plant is transformed with a recombinant polynucleotide comprising a nucleotide sequence that encodes a CBF, and said CBF comprises an AP2 domain;
wherein the altered levels of at least one cell protectant is selected from the group consisting of
(i) increasing; and (ii) decreasing levels; and
wherein the altered biomass is selected from the group consisting of:
(i) increasing; and (ii) decreasing biomass.

2. The transgenic plant of claim 1, wherein said nucleotide sequence is selected from the group consisting of SEQ ID NO: 1, 12, 18, 38, 40, 42, 44, 46, 48, 50, 52, 54, 56, 58, 60, 62, 64, 66, 68, 70, 70, 74, 76, 78, 80, 82, 84, 86, 88, 90, 92, 94, 96, 115, 117, 119, 121, 123, 125, 127, orthologs, paralogs, and variants thereof.

3. The transgenic plant of claim 1, wherein said CBF is selected from the group consisting of SEQ ID NO: 2, 10, 13, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 116, 118, 120, 122, 124, 126, 128, orthologs, paralogs, variants, and fragments thereof.

4. The transgenic plant of claim 1, wherein said nucleotide sequence specifically hybridizes under highly stringent conditions to SEQ ID NO: 1 or its complement under the conditions of 6×SSC and 65° C.

5. The transgenic plant of claim 1, wherein expression of said CBF modifies the level of said at least one cell protectant in said cells of said transgenic plant.

6. The transgenic plant of claim 1, wherein said transgenic plant is a crop plant selected from the group consisting of Brassica juncea, Brassica napus, Brassica oleracea, Brassica rapa, Brassica rapa L, Brassica napus L, Glycine max, Raphanus sativus, Zea mays, Triticum, Oryza sativa, Secale cereale, Sorghum bicolor, Sorghum vulgare, and Hordeum vulgare.

7. The transgenic plant of claim 1, wherein said transgenic plant has increased tolerance to an environmental stress.

8. The transgenic plant of claim 7, wherein said environmental stress is selected from the group consisting of drought, cold, freezing, and high salt.

9. A transgenic plant having an altered trait as compared to a wild-type or untransformed plant, wherein said altered trait is selected from the group consisting of:
(a) altered levels of at least one cell protectant in cells of said transgenic plant; and (b) altered biomass;
wherein said transgenic plant is transformed with a recombinant polynucleotide comprising a nucleotide sequence that encodes a CBF, and said CBF comprises a sequence selected from the group consisting of:
(a) an AP2 domain comprising consecutive amino acid residues 45-106 of SEQ ID NO: 2;
(b) SEQ ID NO: 326;
(c) SEQ ID NO: 325; or
(d) SEQ ID NO: 321;
wherein amino acids 45, 46, 48, 50-52, 54, 59, 60, 62, 64, 65, 67, 68, 71-73, 75-77, 79, 81, 83-91, 93-96, 99, 101, 102, and 104-106 of SEQ ID NO:2 are conserved in the AP2 domain of the CBF; and
wherein the alteration in levels of the at least one cell protectant is selected from the group consisting of (i) increasing; and (ii) decreasing levels; and
wherein the altered biomass is selected from the group consisting of (i) increasing; and (ii) decreasing biomass.

10. The transgenic plant of claim 9, wherein said nucleotide sequence is selected from the group consisting of SEQ ID NO: 1, 12, 18, 38, 40, 42, 44, 46, 48, 50, 52, 54, 56, 58, 60, 62, 64, 66, 68, 70, 70, 74, 76, 78, 80, 82, 84, 86, 88, 90, 92, 94, 96, 115, 117, 119, 121, 123, 125, 127, orthologs, paralogs, and variants thereof.

11. The transgenic plant of claim 9, wherein said CBF is selected from the group consisting of SEQ ID NO: 2, 10, 13, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 116, 118, 120, 122, 124, 126, 128, orthologs, paralogs, variants, and fragments thereof

12. A transgenic rapeseed plant having altered biomass as compared to a wild-type or untransformed plant, wherein said transgenic rapeseed plant is transformed with a recombinant polynucleotide comprising a nucleotide sequence that encodes a CBF comprising an AP2 domain; and
wherein the altered biomass is selected from the group consisting of (i) increasing; and (ii) decreasing biomass.

13. The transgenic rapeseed plant of claim 12, wherein said nucleotide sequence is selected from the group consisting of SEQ ID NO: 1, 12, 18, 38, 40, 42, 44, 46, 48, 50, 52, 54, 56, 58, 60, 62, 64, 66, 68, 70, 70, 74, 76, 78, 80, 82, 84, 86, 88, 90, 92, 94, 96, 115, 117, 119, 121, 123, 125, 127, orthologs, paralogs, and variants thereof.

14. The transgenic rapeseed plant of claim 12, wherein said CBF is selected from the group consisting of SEQ ID NO: 2, 10, 13, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 116, 118, 120, 122, 124, 126, 128, orthologs, paralogs, variants, and fragments thereof

15. The transgenic rapeseed plant of claim 12, wherein said nucleotide sequence specifically hybridizes under highly stringent conditions to SEQ ID NO: 1 or its complement under the conditions of 6×SSC and 65° C.

16. The transgenic rapeseed plant of claim 12, wherein said nucleotide sequence further comprises a regulatory region operably linked to the sequence encoding the CBF.

17. The transgenic rapeseed plant of claim 16, wherein said regulatory region is a constitutive promoter, an inducible promoter, a tissue specific promoter, or a developmental stage specific promoter.

18. A transgenic rapeseed plant having altered biomass, said transgenic rapeseed plant comprising a recombinant polynucleotide comprising a nucleotide sequence encoding a CBF comprises a sequence selected from the group consisting of:
(a) an AP2 domain comprising consecutive amino acid residues 45-106 of SEQ ID NO: 2;
(b) SEQ ID NO: 326;
(c) SEQ ID NO: 325; or
(d) SEQ ID NO: 321;
wherein amino acid residues 45, 46, 48, 50-52, 54, 59, 60, 62, 64, 65, 67, 68, 71-73, 75-77, 79, 81, 83-91, 93-96, 99, 101, 102, and 104-106 of SEQ ID NO:2 are conserved in the AP2 domain of the CBF; and
wherein said biomass is altered compared to that of a plant that has not been transformed with said recombinant polynucleotide; and
wherein the altered biomass is selected from the group consisting of (i) increasing; and (ii) decreasing biomass.

19. A transgenic rapeseed plant having increased environmental stress tolerance, said transgenic rapeseed plant comprising a recombinant polynucleotide comprising a nucleotide sequence encoding a CBF comprising an AP2 domain.

20. The transgenic rapeseed plant of claim 19, wherein said nucleotide sequence is selected from the group consisting of SEQ ID NO 1, 12, 18, 38, 40, 42, 44, 46, 48, 50, 52, 54, 56, 58, 60, 62, 64, 66, 68, 70, 70, 74, 76, 78, 80, 82, 84, 86, 88, 90, 92, 94, 96, 115, 117, 119, 121, 123, 125, 127, orthologs, paralogs, and variants thereof.

21. The transgenic rapeseed plant of claim 19, wherein said CBF is selected from the group consisting of SEQ ID NO: 2, 10, 13, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 116, 118, 120, 122, 124, 126, 128, orthologs, paralogs, variants, and fragments thereof.

22. The transgenic rapeseed plant of claim 19, wherein said nucleotide sequence specifically hybridizes under highly stringent conditions to SEQ ID NO: 1 or its complement under the conditions of 6×SSC and 65° C.

23. The transgenic rapeseed plant of claim 19, wherein said CBF comprises a sequence selected from the group consisting of:
(a) an AP2 domain comprising consecutive amino acid residues 45-106 of SEQ ID NO: 2;
(b) SEQ ID NO: 326;
(c) SEQ ID NO: 325; or
(d) SEQ ID NO: 321;
wherein amino acid residues 45, 46, 48, 50-52, 54, 59, 60, 62, 64, 65, 67, 68, 71-73, 75-77, 79, 81, 83-91, 93-96, 99, 101, 102, and 104-106 of SEQ ID NO:2 are conserved in the AP2 domain of the CBF.

24. The transgenic rapeseed plant of claim 19, wherein said recombinant polynucleotide further comprises a regulatory region operably linked to the sequence encoding the CBF.

25. The transgenic rapeseed plant of claim 24, wherein said regulatory region is a constitutive promoter, an inducible promoter, a tissue specific promoter or a developmental stage specific promoter.

26. The transgenic rapeseed plant of claim 19, wherein said environmental stress is selected from the group consisting of dehydration stress, cold stress, and freezing stress.

27. A cold-inducible promoter comprising a nucleotide sequence selected from the group consisting of nucleotides 1-1695 of SEQ ID NO: 129 and nucleotides 1-1952 of SEQ ID NO: 130.

28. The cold-inducible promoter of claim 27, wherein said nucleotide sequence is selected from the group consisting of nucleotides 1470-1520 of SEQ ID NO: 129 and nucleotides 460-520 of SEQ ID NO: 130.

29. A method for modifying the levels of a cell protectant in a cell, said method comprising:
(a) transforming the cell with a recombinant polynucleotide comprising a nucleotide sequence encoding a CBF; and
(b) expressing said CBF in the transformed cell;
wherein said transformed cell has altered cell protectant levels compared with a cell that has not been transformed with said recombinant polynucleotide.

30. The method of claim 29, wherein said nucleotide sequence is selected from the group consisting of SEQ ID NO: 1, 12, 18, 38, 40, 42, 44, 46, 48, 50, 52, 54, 56, 58, 60, 62, 64, 66, 68, 70, 70, 74, 76, 78, 80, 82, 84, 86, 88, 90, 92, 94, 96, 115, 117, 119, 121, 123, 125, 127, orthologs, paralogs, and variants thereof.

31. The method of claim 29, wherein said CBF is selected from the group consisting of SEQ ID NO: 2, 10, 13, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 116, 118, 120, 122, 124, 126, 128, orthologs, paralogs, variants, and fragments thereof

32. The method of claim 29, wherein said nucleotide sequence specifically hybridizes under highly stringent conditions to SEQ ID NO: 1 or its complement under the conditions of 6×SSC and 65° C.

33. The method of claim 29, wherein said cell protectant is selected from the group consisting of proline, sucrose, and a fatty acid.

34. The method of claim 29, wherein said cell protectant is a fatty acid selected from the group consisting of 16:1, 16:2, or 18:0, and 18:1 fatty acids.

35. The method of claim 29, wherein said CBF binds to a member of a class of DNA regulatory sequences that includes a subsequence selected from the group consisting of CCGAA, CCGAT, CCGAC, CCGAG, CCGTA, CCGTT, CCGTC, CCGTG, CCGCA, CCGCT, CCGCG, CCGCC, CCGGA, CCGGT, CCGGC, CCGGG, AACCG, ATCCG, ACCCG, AGCCG, TACCG, TTCCG, TCCCG, TGCCG, CACCG, CTCCG, CGCCG, CCCCG, GACCG, GTCCG, GCCCG, GGCCG, ACCGA, ACCGT, ACCGC, ACCGG, TCCGA, TCCGT, TCCGC, TCCGG, CCCGA, CCCGT, CCCGC, CCCGG, GCCGA, GCCGT, GCCGC, and GCCGG.

36. The method of claim 29, wherein said CBF binds to a cold or dehydration transcription-regulating region comprising the sequence CCG.

37. The method of claim 29, wherein said CBF comprises a sequence selected from the group consisting of:
(a) an AP2 domain comprising consecutive amino acid residues 45-106 of SEQ ID NO: 2;
(b) SEQ ID NO: 326;
(c) SEQ ID NO: 325; or
(d) SEQ ID NO: 321;
wherein amino acid residues 45, 46, 48, 50-52, 54, 59, 60, 62, 64, 65, 67, 68, 71-73, 75-77, 79, 81, 83-91, 93-96, 99, 101, 102, and 104-106 of SEQ ID NO:2 are conserved in the AP2 domain of the CBF.

38. The method of claim 29, wherein said CBF is CBF3.

39. The method of claim 29, further comprising cold-acclimating said transformed cell.

40. The method of claim 29, wherein said recombinant polynucleotide further comprises a regulatory region operably linked to the sequence encoding the CBF.

41. The method of claim 40, wherein said regulatory region is a constitutive promoter, an inducible promoter, a tissue specific promoter, or a developmental stage specific promoter.

42. The method of claim 29, wherein said CBF comprises an amino acid sequence homologous to a sequence selected from an amino acid sequence depicted in FIG. 19A, 19B, 19C, 19D, or 19E that binds to a DNA regulatory sequence that induces expression of an environmental stress tolerance gene and modifies cell protectant levels.

43. The method of claim 29, wherein the amino acid sequence comprises consecutive amino acid residues of Thr-Xaa₍₁₃₎-Ala-Xaa₍₁₂₎-Ser, wherein Xaa represents any amino acid residue.

44. The method of claim 29, wherein the amino acid sequence comprises consecutive amino acid residues of Asn-Xaa₍₁₂₎-Thr-Xaa₍₁₃₎-Ala-Leu-Arg-Xaa₍₈₎-Ala-Xaa-Ser, wherein Xaa represents any amino acid residue.

45. The method of claim 29, wherein the amino acid sequence comprises consecutive amino acid residues of Gly-Val-Arg-Xaa-Arg-Tyr-Xaa₍₄₋₅₎-Trp-Val-Xaa-Glu-Xaa-Arg-Glu-Xaa₍₆₎-Arg-Glu-Xaa-Asn-Lys-Xaa₍₂₎-Arg-Ile-Trp-Xaa-Gly-Thr-Phe-Xaa₍₅₎-Ala-Ala-Xaa-Ala-Xaa-Asp-Xaa-Ala-Ala-Xaa₍₄₎-Gly-Xaa₍₂₎-Ala-Xaa-Leu-Asn, wherein Xaa represents any amino acid residue.

46. The method of claim 29, wherein the amino acid sequence comprises consecutive amino acid residues of Gly-Val-Arg-Xaa-Arg-Tyr-Xaa₍₄₋₅₎-Trp-Val-Xaa-Glu-Xaa-Arg-Glu-Xaa₍₆₎-Arg-Glu-Xaa-Asn-Lys-Xaa₍₂₎-Arg-Ile-Trp-Xaa-Gly-Thr-Phe-Xaa-Thr-Xaa₍₃₎-Ala-Ala-Xaa-Ala-Xaa-Asp-Xaa-Ala-Ala-Xaa-Ala-Xaa₍₂₎-Gly-Xaa₍₂₎-Ala-Xaa-Leu-Asn-Xaa₍₃₎-Ser, wherein Xaa represents any amino acid residue.

47. The method of claim 29, wherein the amino acid sequence comprises consecutive amino acid residues of His-Pro-Xaa-Tyr-Gly-Val-Arg-Xaa-Arg-Tyr-Xaa₍₄₋₅₎-Trp-Val-Xaa-Glu-Xaa-Arg-Glu-Xaa-Asn-Lys-Xaa₍₂₎-Arg-Glu-Xaa-Asn-Lys-Xaa₍₂₎-Arg-Ile-Trp-Xaa-Gly-Thr-Phe-Xaa-Thr-Xaa-Glu-Xaa-Ala-Ala-Arg-Ala-Asp-His-Asp-Val-Ala-Ala-Xaa-Ala-Leu-Arg-Gly-Xaa₍₂₎-Ala-Xaa-Leu-Asn-Xaa-Ala-Asp-Ser, wherein Xaa represents any amino acid residue.

48. The method of claim 29, wherein said recombinant polynucleotide encodes a polypeptide that elevates cold-regulated gene levels in the absence of cold acclimation compared with cold-regulated gene levels in a plant lacking said recombinant polynucleotide.

49. A method for modifying the levels of a cell protectant in a plant cell, said method comprising:
(a) transforming the plant cell with a recombinant polynucleotide comprising a nucleotide sequence encoding a CBF;
wherein said CBF is sufficiently homologous to a consensus sequence shown in depicted in FIG. 19A, 19B, 19C, 19D, or 19E that the CBF is capable of binding to a CCG regulatory sequence and that the nucleotide sequence that encodes the CBF can hybridize to SEQ ID NO: 1 under the conditions of 6×SSC and 65° C.; and
(b) expressing said CBF in the transformed plant cell;
wherein said transformed plant cell has altered cell protectant levels compared with a plant cell that has not been transformed with said recombinant polynucleotide.

50. A method for modifying proline, sucrose or fatty acid levels in a plant cell, said method comprising:
a) transforming the plant cell with a recombinant polynucleotide comprising a nucleotide sequence encoding a CBF; and
(b) expressing said CBF in the transformed cell;
whereby expression of said CBF modifies proline levels in the plant cell.

51. The method of claim 50, wherein said nucleotide sequence is selected from the group consisting of SEQ ID NO: 1, 12, 18, 38, 40, 42, 44, 46, 48, 50, 52, 54, 56, 58, 60, 62, 64, 66, 68, 70, 70, 74, 76, 78, 80, 82, 84, 86, 88, 90, 92, 94, 96, 115, 117, 119, 121, 123, 125, 127, orthologs, paralogs, and variants thereof.

52. The method of claim 50, wherein said CBF is selected from the group consisting of SEQ ID NO: 2, 10, 13, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 116, 118, 120, 122, 124, 126, 128, orthologs, paralogs, variants, and fragments thereof.

53. The method of claim 50, wherein said nucleotide sequence specifically hybridizes under highly stringent conditions to SEQ ID NO: 1 or its complement under the conditions of 6×SSC and 65° C.

54. The method of claim 50, wherein said CBF comprises a sequence selected from the group consisting of:
(a) an AP2 domain comprising consecutive amino acid residues 45-106 of SEQ ID NO: 2;
(b) SEQ ID NO: 326;
(c) SEQ ID NO: 325; or
(d) SEQ ID NO: 321;
wherein amino acid residues 45, 46, 48, 50-52, 54, 59, 60, 62, 64, 65, 67, 68, 71-73, 75-77, 79, 81, 83-91, 93-96, 99, 101, 102 and 104-106 of SEQ ID NO:2 are conserved in the AP2 domain of the CBF.

55. The method of claim 50, wherein said CBF is CBF3.

56. The method of claim 50, wherein said recombinant polynucleotide further comprises a regulatory region operably linked to the sequence encoding the CBF.

57. The method of claim 56, wherein said regulatory region is a constitutive promoter, an inducible promoter, a tissue specific promoter, or a developmental stage specific promoter.

58. A method for improving the tolerance of a plant cell to an environmental stress, said method comprising
(a) transforming the plant cell with a recombinant polynucleotide comprising a nucleotide sequence encoding a CBF;
wherein said CBF is sufficiently homologous to a consensus sequence shown in depicted in FIG. 19A, 19B, 19C, 19D, or 19E that the CBF is capable of binding to a CCG regulatory sequence and that the nucleotide sequence that encodes the CBF can hybridize to SEQ ID NO: 1 under highly stringent conditions of 6×SSC and 65° C.; and
(b) expressing said CBF in the transformed plant cell;
whereby expression of said CBF increases cell protectant levels at least 1.5 fold in the transformed plant cell compared with cell protectant levels in an untransformed plant cell and the increased cell protectant levels in the transformed plant cell improve the tolerance of the transformed plant cell to the environmental stress, and
wherein the environmental stress is selected from the group consisting of:
cold, freezing, dehydration, and drought; and
wherein the improved environmental stress tolerance is selected from the group consisting of:
a) a decrease in the extent of a plant's injury;
b) a decrease in the extent of a cell's injury;
c) a decrease in the extent of a plant's growth inhibition;
d) a decrease in the extent of a cell's growth inhibition;
e) an increase in survival rate after exposure to cold temperatures;
f) an increase in survival rate after exposure to freezing temperatures;
g) an increase in the extent of survival of a plant after exposure to drought conditions;
h) an increase in the extent of survival of a cell after exposure to drought conditions;
i) an increase in the extent of survival of a plant after exposure to high salt conditions; and
j) an increase in the extent of survival of a cell after exposure to high salt conditions.

59. The method of claim 58, wherein said nucleotide sequence is selected from the group consisting of SEQ ID NO: 1, 12, 18, 38, 40, 42, 44, 46, 48, 50, 52, 54, 56, 58, 60, 62, 64, 66, 68, 70, 70, 74, 76, 78, 80, 82, 84, 86, 88, 90, 92, 94, 96, 115, 117, 119, 121, 123, 125, 127, orthologs, paralogs, and variants thereof.

60. The method of claim 58, wherein said CBF is selected from the group consisting SEQ ID NO: 2, 10, 13, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 116, 118, 120, 122, 124, 126, 128, orthologs, paralogs, variants, and fragments thereof.

61. The method of claim 58, wherein said nucleotide sequence specifically hybridizes under highly stringent conditions to SEQ ID NO: 1 or its complement under the conditions of 6×SSC and 65° C.

62. The method of claim 58, wherein said cell protectant is selected from the group consisting of proline and sucrose.

63. The method of claim 58, wherein said CBF comprises a sequence selected from the group consisting of:
(a) an AP2 domain comprising consecutive amino acid residues 45-106 of SEQ ID NO: 2;
(b) SEQ ID NO: 326;
(c) SEQ ID NO: 325; or
(d) SEQ ID NO: 321;
wherein amino acid residues 45, 46, 48, 50-52, 54, 59, 60, 62, 64, 65, 67, 68, 71-73, 75-77, 79, 81, 83-91, 93-96, 99, 101, 102, and 104-106 of SEQ ID NO:2 are conserved in the AP2 domain of the CBF.

64. The method of claim 58, wherein said CBF is CBF3.

65. The method of claim 58, wherein said recombinant polynucleotide further comprises a regulatory region operably linked to the sequence encoding the CBF.

66. The method of claim 65, wherein said regulatory region is a constitutive promoter, an inducible promoter, a tissue specific promoter or a developmental stage specific promoter.

67. The method of claim 58, further comprising cold-acclimating said transformed cell.

68. A method of screening for a CBF, said method comprising:
(a) constructing yeast reporter strains by transforming yeast cells with a vector comprising C-repeat/DRE sequences as upstream activator sequence elements fused upstream of a reporter gene;
(b) transforming said yeast reporter strains with an expression library containing random cDNA inserts each of which are fused to an activation domain;
(c) growing said yeast reporter strains on differential media; and
(c) screening for yeast colonies that comprise cDNA inserts encoding C-repeat/DRE binding domains fused to said activation domains.

69. The method of claim 68, wherein said reporter gene is a lacZ reporter gene, said reporter gene is operably linked to a GAL1 promoter, and said activation domain is a GAL4 activation domain;
wherein clones that contain a cDNA insert encoding a C-repeat/DRE binding domain fused to GAL4-activation domains bind upstream of the lacZ reporter gene carrying the wild type C-repeat/DRE sequence, activating transcription of the lacZ reporter gene.
and wherein said differential media comprises X-gal treated filters, and said yeast colonies on said X-gal-treated filters are screened by their blue color.

70. The method of clain 68, wherein said CBF binds to a member of a class of DNA regulatory sequences that includes a subsequence selected from the group consisting of CCGAA, CCGAT, CCGAC, CCGAG, CCGTA, CCGTT, CCGTC, CCGTG, CCGCA, CCGCT, CCGCG, CCGCC, CCGGA, CCGGT, CCGGC, CCGGG, AACCG, ATCCG, ACCCG, AGCCG, TACCG, TTCCG, TCCCG, TGCCG, CACCG, CTCCG, CGCCG, CCCCG, GACCG, GTCCG, GCCCG, GGCCG, ACCGA, ACCGT, ACCGC, ACCGG, TCCGA, TCCGT, TCCGC, TCCGG, CCCGA, CCCGT, CCCGC, CCCGG, GCCGA, GCCGT, GCCGC, and GCCGG.

71. A method for identifying a CBF, said method comprising:
(a) identifying sequences by BLAST analysis in which ESTs in a database are queried using SEQ ID NO: 327, 328, 329, 330, or 331 as the query sequence
(b) selecting positive EST sequences with an HSP BLAST score of at least 100 bits over at least 95% of the query sequence; and
(c) organizing said positive EST sequences into contigs; thereby identifying a CBF sequence.

72. The method of claim 71, further comprising:
identifying complete predicted coding regions with a start and a stop codon; and
cloning said CBF sequence directly from cDNA or genomic DNA by PCR using primers in the 5′ and 3′ flanking regions.

73. The method of claim 71, further comprising cloning said CBF sequence into a transformation vector.

74. The method of claim 73, wherein said transformation vector is pMEN65 or a transformation vector modified from pMEN65.

75. A method for enhancing the cold tolerance of a plant, said method comprising:
(a) transforming a plant with a recombinant polynucleotide comprising a nucleotide sequence encoding a CBF;
(b) expressing the recombinant polynucleotide and
(c) cold acclimating said transformed plant;
wherein said cold acclimated transformed plant has an improved cold tolerance compared with a non-transformed plant that has been cold-acclimated; and
wherein the enhanced cold tolerance is selected from the group consisting of: a) a decrease in the extent of a plant's injury; b) a decrease in the extent of a cell's injury; c) a decrease in the extent of a plant's growth inhibition; d) a decrease in the extent of a cell's growth inhibition; e) an increase in survival rate after exposure to cold temperatures; and f) an increase in survival rate after exposure to freezing temperatures.

76. The method of claim 75, wherein said plant is a rapeseed plant.

77. The method of claim 75, wherein said polynucleotide comprises a nucleotide sequence selected from the group consisting of SEQ ID NO: 1, 12, 18, 38, 40, 42, 44, 46, 48, 50, 52, 54, 56, 58, 60, 62, 64, 66, 68, 70, 70, 74, 76, 78, 80, 82, 84, 86, 88, 90, 92, 94, 96, 115, 117, 119, 121, 123, 125, 127, orthologs, paralogs, and variants thereof.

78. The method of claim 75, wherein said CBF is selected from the group consisting SEQ ID NO: 2, 10, 13, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 116, 118, 120, 122, 124, 126, 128, orthologs, paralogs, variants, and fragments thereof.

79. The method of claim 75, wherein said nucleotide sequence specifically hybridizes under highly stringent conditions to SEQ ID NO: 1 or its complement under the conditions of 6×SSC and 65° C.

80. The method of claim 75, wherein said CBF comprises a sequence selected from the group consisting of:
(a) an AP2 domain comprising consecutive amino acid residues 45-106 of SEQ ID NO: 2;
(b) SEQ ID NO: 326;
(c) SEQ ID NO: 325; or
(d) SEQ ID NO: 321;
wherein amino acid residues 45, 46, 48, 50-52, 54, 59, 60, 62, 64, 65, 67, 68, 71-73, 75-77, 79, 81, 83-91, 93-96, 99, 101, 102, and 104-106 of SEQ ID NO:2 are conserved in the AP2 domain of the CBF.

81. The method of claim 75, wherein the cold tolerance is enhanced by at least 2° C. compared with the cold tolerance of the non-transformed plant that has been cold-acclimated.

82. The method of claim 75, wherein the cold tolerance is enhanced by at least 5° C. compared with the cold tolerance of the non-transformed plant that has been cold-acclimated.

83. The method of claim 75, wherein the cold tolerance is enhanced by at least 8° C. compared with the cold tolerance of the non-transformed plant that has been cold-acclimated.

84. The method of claim 75, wherein said recombinant polynucleotide further comprises a regulatory region operably linked to the sequence encoding the CBF.

85. The method of claim 84, wherein said regulatory region is a constitutive promoter, an inducible promoter, a tissue specific promoter, or a developmental stage specific promoter.

86. The method of claim 85, wherein said inducible promoter is a cold-inducible promoter comprising a sequence selected from the group consisting of a sequence comprising nucleotides 1470-1520 of SEQ ID NO: 129 and a sequence comprising nucleotides 460-520 of SEQ ID NO: 130.

87. A method for altering the biomass of a plant, said method comprising:
(a) transforming said plant with a recombinant polynucleotide comprising a nucleotide sequence encoding a CBF; and
(b) expressing said CBF in said transformed plant;
wherein said transformed plant has a altered biomass compared with a plant that has not been transformed with said recombinant polynucleotide.

88. The method of claim 87, wherein said nucleotide sequence is selected from the group consisting of SEQ ID NO: 1, 12, 18, 38, 40, 42, 44, 46, 48, 50, 52, 54, 56, 58, 60, 62, 64, 66, 68, 70, 70, 74, 76, 78, 80, 82, 84, 86, 88, 90, 92, 94, 96, 115, 117, 119, 121, 123, 125, 127, orthologs, paralogs, and variants thereof.

89. The method of claim 87, wherein said CBF is selected from the group consisting of SEQ ID NO: 2, 10, 13, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 116, 118, 120, 122, 124, 126, 128, orthologs, paralogs, variants, and fragments thereof.

90. The method of claim 87, wherein said nucleotide sequence specifically hybridizes under highly stringent conditions to SEQ ID NO: 1 or its complement under the conditions of 6×SSC and 65° C.

91. The method of claim 87, wherein said altered biomass is an increase in the leaf number, leaf size, or root mass of a plant.

92. The method of claim 87, wherein said CBF comprises a sequence selected from the group consisting of:
(a) an AP2 domain comprising consecutive amino acid residues 45-106 of SEQ ID NO: 2;
(b) SEQ ID NO: 326;
(c) SEQ ID NO: 325; or
(d) SEQ ID NO: 321;
wherein amino acid residues 45, 46, 48, 50-52, 54, 59, 60, 62, 64, 65, 67, 68, 71-73, 75-77, 79, 81, 83-91, 93-96, 99, 101, 102 and 104-106 of SEQ ID NO:2 are conserved in the AP2 domain of the CBF.

93. The method of 87, wherein said CBF binds to a cold or dehydration transcription-regulating region comprising the sequence CCG.

94. The method of claim 87, wherein said CBF binds to a member of a class of DNA regulatory sequences which includes a subsequence selected from the group consisting of CCGAA, CCGAT, CCGAC, CCGAG, CCGTA, CCGTT, CCGTC, CCGTG, CCGCA, CCGCT, CCGCG, CCGCC, CCGGA, CCGGT, CCGGC, CCGGG, AACCG, ATCCG, ACCCG, AGCCG, TACCG, TTCCG, TCCCG, TGCCG, CACCG, CTCCG, CGCCG, CCCCG, GACCG, GTCCG, GCCCG, GGCCG, ACCGA, ACCGT, ACCGC, ACCGG, TCCGA, TCCGT, TCCGC, TCCGG, CCCGA, CCCGT, CCCGC, CCCGG, GCCGA, GCCGT, GCCGC, and GCCGG.

95. The method of claim 87, wherein said recombinant polynucleotide further comprises a regulatory region operably linked to the sequence encoding the CBF.

96. The method of claim 95, wherein said regulatory region is a constitutive promoter, an inducible promoter, a tissue specific promoter, or a developmental stage specific promoter.

97. The method of claim 87, wherein said plant is exposed to adverse environmental conditions, and said biomass is increased relative to an untransformed plant exposed to the same conditions.

98. The method of claim 87, wherein said plant cell is derived from a crop plant selected from the group consisting of Brassica juncea, Brassica napus, Brassica oleracea, Brassica rapa, Brassica rapa L, Brassica napus L, Glycine max, Raphanus sativus, Zea mays, Triticum, Oryza sativa, Secale cereale, Sorghum bicolor, Sorghum vulgare, and Hordeum vulgare.

99. A method for altering the biomass of a plant, said method comprising:
(a) transforming said plant with a recombinant polynucleotide comprising a nucleotide sequence encoding a CBF;
wherein said CBF is sufficiently homologous to a consensus sequence shown in depicted in FIG. 19A, 19B, 19C, 19D, or 19E that the CBF is capable of binding to a CCG regulatory sequence and that the nucleotide sequence that encodes the CBF can hybridize to SEQ ID NO: 1 under the conditions of 6×SSC and 65° C.; and
(b) expressing said CBF in said transformed plant;
wherein said transformed plant has a altered biomass compared with a plant that has not been transformed with said recombinant polynucleotide.

100. A method for altering the biomass of a plant, said method comprising:
(a) altering the levels of a polynucleotide comprising a nucleotide sequence encoding a CBF in a plant; and
(b) identifying a plant so altered;
wherein said altered levels of said polynucleotide modifies the biomass of the plant compared to a plant that does not comprise said altered polynucleotide levels.

101. The method of claim 100, wherein said nucleotide sequence is selected from the group consisting of SEQ ID NO: 1, 12, 18, 38, 40, 42, 44, 46, 48, 50, 52, 54, 56, 58, 60, 62, 64, 66, 68, 70, 70, 74, 76, 78, 80, 82, 84, 86, 88, 90, 92, 94, 96, 115, 117, 119, 121, 123, 125, 127, orthologs, paralogs, and variants thereof.

102. The method of claim 100, wherein said CBF is selected from the group consisting of SEQ ID NO: 2, 10, 13, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 116, 118, 120, 122, 124, 126, 128, orthologs, paralogs, variants, and fragments thereof.

103. The method of claim 100, wherein said nucleotide sequence specifically hybridizes under highly stringent conditions to SEQ ID NO: 1 or its complement under the conditions of 6×SSC and 65° C.

104. The method of claim 100, wherein said altered biomass is an increase in leaf number, leaf size, or root mass.

105. The method of claim 100, wherein said CBF comprises a sequence selected from the group consisting of:
(a) an AP2 domain comprising consecutive amino acid residues 45-106 of SEQ ID NO: 2;
(b) SEQ ID NO: 326;
(c) SEQ ID NO: 325; or
(d) SEQ ID NO: 321;
wherein amino acid residues 45, 46, 48, 50-52, 54, 59, 60, 62, 64, 65, 67, 68, 71-73, 75-77, 79, 81, 83-91, 93-96, 99, 101, 102 and 104-106 of SEQ ID NO:2 are conserved in the AP2 domain of the CBF.

106. The method of claim 100, wherein said CBF binds to a cold or dehydration transcription-regulating region comprising the sequence CCG.

107. The method of claim 100, wherein said CBF binds to a member of a class of DNA regulatory sequences that includes a subsequence selected from the group consisting of CCGAA, CCGAT, CCGAC, CCGAG, CCGTA, CCGTT, CCGTC, CCGTG, CCGCA, CCGCT, CCGCG, CCGCC, CCGGA, CCGGT, CCGGC, CCGGG, AACCG, ATCCG, ACCCG, AGCCG, TACCG, TTCCG, TCCCG, TGCCG, CACCG, CTCCG, CGCCG, CCCCG, GACCG, GTCCG, GCCCG, GGCCG, ACCGA, ACCGT, ACCGC, ACCGG, TCCGA, TCCGT, TCCGC, TCCGG, CCCGA, CCCGT, CCCGC, CCCGG, GCCGA, GCCGT, GCCGC, and GCCGG.

108. The method of claim 100, wherein said recombinant polynucleotide further comprises a regulatory region operably linked to the sequence encoding the CBF.

109. The method of claim 108, wherein said regulatory region is a constitutive promoter, an inducible promoter, a tissue specific promoter, or a developmental stage specific promoter.

110. The method of claim 100, wherein said plant is exposed to adverse environmental conditions, and said biomass is increased relative to an untransformed plant exposed to the same conditions.

111. The method of claim 100, wherein said plant cell is derived from a crop plant selected from the group consisting of Brassica juncea, Brassica napus, Brassica oleracea, Brassica rapa, Brassica rapa L, Brassica napus L, Glycine max, Raphanus sativus, Zea mays, Triticum, Oryza sativa, Secale cereale, Sorghum bicolor, Sorghum vulgare, and Hordeum vulgare.
